SEQUENCE LISTING (6 sequences)

SEQ ID NO: 1 - DNA, 21 bp, Artificial Sequence
  source: /note=“Description of Artificial Sequence Synthetic oligonucleotide”
auaaggcuaugaagagauatt

SEQ ID NO: 2 - RNA, 21 nt, Artificial Sequence
  source: /note=“Description of Artificial Sequence Synthetic oligonucleotide”
uaucucuucauagccuuauuu

SEQ ID NO: 3 - DNA, 21 bp, Artificial Sequence
  source: /note=“Description of Artificial Sequence Synthetic oligonucleotide”
cuuuaacaauuccugaaautt

SEQ ID NO: 4 - RNA, 21 nt, Artificial Sequence
  source: /note=“Description of Artificial Sequence Synthetic oligonucleotide”
auuucaggaauuguuaaaguu

SEQ ID NO: 5 - DNA, 21 bp, Artificial Sequence
  source: /note=“Description of Artificial Sequence Synthetic oligonucleotide”
ggaucaucucaagucuuactt

SEQ ID NO: 6 - DNA, 21 bp, Artificial Sequence
  source: /note=“Description of Artificial Sequence Synthetic oligonucleotide”
guaagacuugagaugaucctt

(19) United States
(12) Patent Application Publication
(10) Pub. No.: US 2014/0308304 A1
(43) Pub. Date: Oct. 16, 2014

(54) LIPIDS FOR THE DELIVERY OF ACTIVE AGENTS

(71) Applicant: ALNYLAM PHARMACEUTICALS, INC., Cambridge, MA (US)

(72) Inventors: Muthiah Manoharan, Cambridge, MA (US); Kallanthottathil G. Rajeev, Cambridge, MA (US); Muthusamy Jayaraman, Cambridge, MA (US); Akin Akinc, Cambridge, MA (US); Shigeo Matsuda, Cambridge, MA (US); Martin Maier, Cambridge, MA (US)

(21) Appl. No.: 14/362,848

(22) PCT Filed: Dec. 7, 2012
(86) PCT No.: PCT/US2012/068516

§ 371 Date: Jun. 4, 2014

Related U.S. Application Data
(60) Provisional application No. 61/568,106, filed on Dec. 7, 2011.
(60) Provisional application No. 61/568,078, filed on Dec. 7, 2011.
(60) Provisional application No. 61/596,093, filed on Feb. 7, 2012.

(51) Int. Cl. A61K 47/18; A61K 9/14; C12N 15/113; C07C 229/26; A61K 47/24; C07C 237/16; C07C 327/30; C07F 9/50; A61K 47/20; C07C 237/10; C07C 229/30

(52) U.S. Cl. CPC A61K 47/18; C07C 237/10; A61K 9/14; C12N 15/113; C07C 229/26; C07C 229/30; C07C 237/16; C07C 327/30; C07F 9/5036; C07F 9/5004; C07F 9/5013; A61K 47/20; A61K 47/24
USPC 424/184.1; 554/106; 514/785; 514/44 A; 514/788; 554/107; 560/169; 558/251; 554/84; 554/37; 435/375

(57) ABSTRACT

The present invention relates to novel cationic lipids that can be used in combination with other lipid components such as cholesterol and PEG-lipids to form lipid nanoparticles with oligonucleotides, to facilitate the cellular uptake and endosomal escape, and to knockdown target mRNA both in vitro and in vivo. The invention also relates to lipid particles comprising a neutral lipid, a lipid capable of reducing aggregation, a cationic lipid of the present invention, and optionally, a sterol. The lipid particle may further include a therapeutic agent such as a nucleic acid.

This application claims the benefit of U.S. Provisional Application Nos. 61/568,078, filed Dec. 7, 2011, 61/568,106, filed Dec. 7, 2011, and 61/596,093, filed Feb. 7, 2012, each of which is incorporated by reference in its entirety.

### TECHNICAL FIELD

The present invention relates to novel cationic lipids that can be used in combination with other lipid components such as cholesterol and PEG-lipids to form lipid nanoparticles with oligonucleotides, to facilitate the cellular uptake and endosomal escape, and to knockdown target mRNA both in vitro and in vivo.

### BACKGROUND

Therapeutic nucleic acids include, e.g., small interfering RNA (siRNA), micro RNA (miRNA), antisense oligonucleotides, ribozymes, plasmids, immune stimulating nucleic acids, antisense, antagomir, antimir, microRNA mimic, supermir, U1 adaptor, and aptamer. In the case of siRNA or miRNA, these nucleic acids can down-regulate intracellular levels of specific proteins through a process termed RNA interference (RNAi). The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models. In addition, siRNA constructs are currently being evaluated in clinical studies.

However, two problems currently faced by siRNA or miRNA constructs are, first, their susceptibility to nuclease digestion in plasma and, second, their limited ability to gain access to the intracellular compartment where they can bind the protein RISC when administered systemically as the free siRNA or miRNA. Lipid nanoparticles formed from cationic lipids with other lipid components, such as cholesterol and PEG lipids, and oligonucleotides (such as siRNA and miRNA) have been used to facilitate the cellular uptake of the oligonucleotides.

There remains a need for improved cationic lipids and lipid nanoparticles for the delivery of oligonucleotides. Preferably, these lipid nanoparticles would provide high drug:lipid ratios, protect the nucleic acid from degradation and clearance in serum, be suitable for systemic delivery, and provide intracellular delivery of the nucleic acid. In addition, these lipid-nucleic acid particles should be well-tolerated and provide an adequate therapeutic index, such that patient treatment at an effective dose of the nucleic acid is not associated with significant toxicity and/or risk to the patient.

### SUMMARY

The present invention relates to a cationic lipid suitable for forming nucleic acid-lipid particles. The cationic lipids may contain one or more biodegradable groups. The biodegradable groups are located in the mid- or distal section of a lipidic moiety (e.g., a hydrophobic chain) of the cationic lipid. These cationic lipids may be incorporated into a lipid particle for delivering an active agent, such as a nucleic acid (e.g., an siRNA). The incorporation of the biodegradable group(s) into the cationic lipid results in faster metabolism and removal of the cationic lipid from the body following delivery of the active agent to a target area. As a result, these cationic lipids have lower toxicity than similar cationic lipids without the biodegradable groups.

1) Cationic Lipids that Include an Amino Acid Group and One or More Biodegradable Groups.

In one embodiment, the cationic lipid is a compound of formula (I):

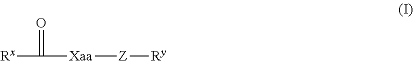

or a salt thereof (e.g., a pharmaceutically acceptable salt thereof), wherein

Xaa is a D- or L-amino acid residue having the formula —NR^{N}—CR¹R²—(C═O)—, or a peptide of amino acid residues having the formula —{NR^{N}—CR¹R²—(C═O)}ₙ—, wherein n is 2 to 20;

R¹ is independently, for each occurrence, a non-hydrogen, substituted or unsubstituted side chain of an amino acid;

R² and R^{N} are independently, for each occurrence, hydrogen, an organic group consisting of carbon, oxygen, nitrogen, sulfur, and hydrogen atoms, or any combination of the foregoing, and having from 1 to 20 carbon atoms, C₍₁₋₅₎alkyl, cycloalkyl, cycloalkylalkyl, C₍₃₋₅₎alkenyl, C₍₃₋₅₎alkynyl, C₍₁₋₅₎alkanoyl, C₍₁₋₅₎alkanoyloxy, C₍₁₋₅₎alkoxy, C₍₁₋₅₎alkoxy-C₍₁₋₅₎alkyl, C₍₁₋₅₎alkoxy-C₍₁₋₅₎alkoxy, C₍₁₋₅₎alkyl-amino-C₍₁₋₅₎alkyl-, C₍₁₋₅₎dialkyl-amino-C₍₁₋₅₎alkyl-, nitro-C₍₁₋₅₎alkyl, cyano-C₍₁₋₅₎alkyl, aryl-C₍₁₋₅₎alkyl, 4-biphenyl-C₍₁₋₅₎alkyl, carboxyl, or hydroxyl;

Z is NH, O, S, —CH₂S—, —CH₂S(O)—, or an organic linker consisting of 1-40 atoms selected from hydrogen, carbon, oxygen, nitrogen, and sulfur atoms (preferably, Z is NH or O);

R^{x} and R^{y} are, independently, (i) a lipophilic tail derived from a lipid (which can be naturally-occurring or synthetic), phospholipid, glycolipid, triacylglycerol, glycerophospholipid, sphingolipid, ceramide, sphingomyelin, cerebroside, or ganglioside, wherein the tail optionally includes a steroid; (ii) an amino acid terminal group selected from hydrogen, hydroxyl, amino, and an organic protecting group; or (iii) a substituted or unsubstituted C₍₃₋₂₂₎alkyl, C₍₆₋₁₂₎cycloalkyl, C₍₆₋₁₂₎cycloalkyl-C₍₃₋₂₂₎alkyl, C₍₃₋₂₂₎alkenyl, C₍₃₋₂₂₎alkynyl, C₍₃₋₂₂₎alkoxy, or C₍₆₋₁₂₎-alkoxy-C₍₃₋₂₂₎alkyl;

one of R^{x} and R^{y} is a lipophilic tail as defined above and the other is an amino acid terminal group, or both R^{x} and R^{y} are lipophilic tails;

at least one of R^{x} and R^{y} is interrupted by one or more biodegradable groups (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)— or

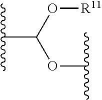

(wherein R¹¹ is a C₂-C₈ alkyl or alkenyl), in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently H or C₁-C₄ alkyl)); and

R^{x} and R^{y} each, independently, optionally have one or more carbon-carbon double bonds.

In another embodiment, the cationic lipid is a compound of formula (IA):

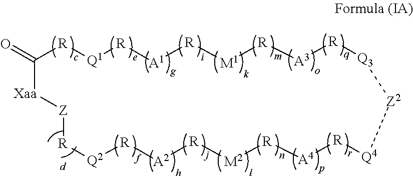

or a salt thereof (e.g., a pharmaceutically acceptable salt thereof), wherein

Z and Xaa are as defined with respect to formula (I) (the variables which are used in the definition of Xaa, namely R^{N}, R¹ and R², are also as defined in formula (I));

each occurrence of R is, independently, —(CR³R⁴)—;

each occurrence of R³ and R⁴ are, independently H, halogen, OH, alkyl, alkoxy, —NH₂, alkylamino, or dialkylamino (in one preferred embodiment, each occurrence of R³ and R⁴ are, independently H or C₁-C₄ alkyl);

or R³ and R⁴, together with the carbon atom to which they are directly attached, form a cycloalkyl group, wherein no more than three R groups in each chain between the —Z-Xaa-C(O)— and Z² moieties are cycloalkyl (e.g., cyclopropyl);

Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;

Q³ and Q⁴ are each, independently, H, —(CR³R⁴)—, cycloalkyl, heterocyclyl, heterocyclylalkyl, aryl, heteroaryl, or a cholesterol moiety;

each occurrence of A¹, A², A³ and A⁴ is, independently, —(CR⁵R⁵—CR⁵═CR⁵)—;

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

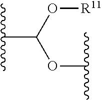

(wherein R¹¹ is a C₂-C₈ alkyl or alkenyl));

each occurrence of R⁵ is, independently, H or alkyl (e.g., C₁-C₄ alkyl);

Z² is absent, alkylene or —O—P(O)(OH)—O—;

each ------ attached to Z² is an optional bond, such that when Z² is absent, Q³ and Q⁴ are not directly covalently bound together;

c, d, e, f, i, j, m, n, q and r are each, independently, 0, 1, 2, 3, 4, 5, 6, 7, 8, 9, or 10;

g and h are each, independently, 0, 1 or 2;

k and l are each, independently, 0 or 1, wherein at least one of k and l is 1;

o and p are each, independently, 0, 1 or 2; and

Q³ and Q⁴ are each, independently, separated from the —Z-Xaa-C(O)— moiety by a chain of 8 or more atoms (e.g., 12 or 14 or more atoms).

Yet another embodiment is a cationic lipid of the formula (IB):

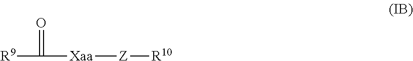

or a salt thereof (e.g., a pharmaceutically acceptable salt thereof), wherein

Z and Xaa are as defined with respect to formula (I) (the variables which are used in the definition of Xaa, namely R^{N}, R¹ and R², are also as defined in formula (I)); and

each of R⁹ and R¹⁰ are, independently, C₁₂-C₂₄ alkyl (e.g., C₁₂-C₂₀ alkyl), C₁₂-C₂₄ alkenyl (e.g., C₁₂-C₂₀ alkenyl), or C₁₂-C₂₄ alkoxy (e.g., C₁₂-C₂₀ alkoxy) having one or more biodegradable groups;

each biodegradable group independently interrupts the C₁₂-C₂₄ alkyl, alkenyl, or alkoxy group or is substituted at the terminus of the C₁₂-C₂₄ alkyl, alkenyl, or alkoxy group; wherein

(i) the terminus of R⁹ is separated from the carbonyl group of the —C(O)-Xaa-Z— moiety by a chain of 8 or more atoms (e.g., 12 or 14 or more atoms); and

(ii) the terminus of R¹⁰ is separated from the Z group of the —C(O)-Xaa-Z— moiety by a chain of 8 or more atoms (e.g., 12 or 14 or more atoms).

Yet another embodiment is a cationic lipid of the formula (IC):

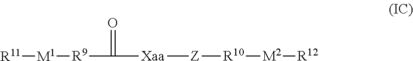

or a salt thereof (e.g., a pharmaceutically acceptable salt thereof), wherein

Z and Xaa are as defined with respect to formula (I) (the variables which are used in the definition of Xaa, namely R^{N}, R¹ and R², are also as defined in formula (I));

each of R⁹ and R¹⁰ are, independently, alkylene or alkenylene;

each of R¹¹ and R¹² are, independently, alkyl or alkenyl, optionally terminated by COOR¹³ wherein each R¹³ is independently unsubstituted alkyl (e.g., C₁-C₄ alkyl such as methyl or ethyl), substituted alkyl (such as benzyl), or cycloalkyl;

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or or

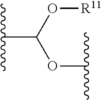

(wherein R¹¹ is a C₂-C₈ alkyl or alkenyl), in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently H or C₁-C₄ alkyl));

R⁹, M¹, and R¹¹ are together at least 8 carbon atoms in length (e.g., 12 or 14 carbon atoms or longer); and

R¹⁰, M², and R¹² are together at least 8 carbon atoms in length (e.g., 12 or 14 carbon atoms or longer).

In a preferred embodiment of the compound of formula (IC), R⁹ and R¹⁰ are each independently C₄-C₁₂ alkylene or C₄-C₁₂ alkenylene, M¹ and M² are —C(O)O— or —O(CO)—, and R¹¹ and R¹² are C₄-C₁₂ alkylene or C₄-C₁₂ alkenylene. In one embodiment, R⁹, M¹, and R¹¹ are together 12 to 24 carbon atoms in length. In another embodiment, R⁹, M¹, and R¹¹ are together 14 to 18 carbon atoms in length. In one embodiment, R¹⁰, M², and R¹² are together 12 to 24 carbon atoms in length. In another embodiment, R¹⁰, M², and R¹² are together 14 to 18 carbon atoms in length.

Yet another embodiment is a cationic lipid of the formula (ID):

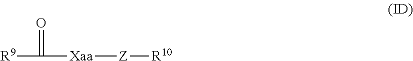

or a salt thereof (e.g., a pharmaceutically acceptable salt thereof), wherein

Z and Xaa are as defined with respect to formula (I) (the variables which are used in the definition of Xaa, namely R^{N}, R¹ and R², are also as defined in formula (I)); and

each of R⁹ and R¹⁰ are independently C₁₂-C₂₄ alkyl or C₁₂-C₂₄ alkenyl substituted at its terminus with a biodegradable group, such as —COOR¹³ where each R¹³ is independently alkyl (preferably C₁-C₄ alkyl such as methyl or ethyl).

In a preferred embodiment of the compound of formula (ID), R⁹ and R¹⁰ are each independently C₁₄-C₁₈ alkyl or C₁₄-C₁₈ alkenyl substituted at its terminus with a biodegradable group.

In another preferred embodiment, the biodegradable group is —COOR¹³ where R¹³ is C₁-C₄ alkyl (such as methyl or ethyl).

In another embodiment, the cationic lipid is a compound of the formula II:

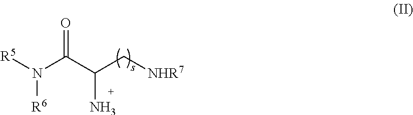

or a salt thereof (e.g., a pharmaceutically acceptable salt thereof), wherein

s is 1, 2, 3 or 4; and

R⁷ is selected from lysyl, ornithyl, 2,3-diaminobutyryl, histidyl and an acyl moiety of the formula:

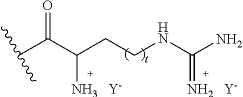

t is 1, 2 or 3;

the NH₃⁺ moiety in the acyl moiety in R⁷ is optionally absent;

each occurrence of Y⁻ is independently a pharmaceutically acceptable anion (e.g., halide, such as chloride);

R⁵ and R⁶ are each, independently a lipophilic tail derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid; or a substituted or unsubstituted C₍₃₋₂₂₎alkyl, C₍₆₋₁₂₎cycloalkyl, C₍₆₋₁₂₎cycloalkyl-C₍₃₋₂₂₎alkyl, C₍₃₋₂₂₎alkenyl, C₍₃₋₂₂₎alkynyl, C₍₃₋₂₂₎alkoxy, or C₍₆₋₁₂₎alkoxy-C₍₃₋₂₂₎alkyl;

at least one of R⁵ and R⁶ is interrupted by one or more biodegradable groups (e.g., —SC(O)—, —C(O)S—, —OC(S)—, —C(S)O—, —S—S—, —C(O)(NR^{a})—, —N(R^{a})C(O)—, —C(S)(NR^{a})—, —N(R^{a})C(O)—, —N(R^{a})C(O)N(R^{a})—, or —OC(O)O—);

each occurrence of R^{a} is, independently, H or alkyl; and

R⁵ and R⁶ each, independently, optionally contain one or more carbon-carbon double bonds.

In another embodiment, the cationic lipid is a compound of the formula (11A):

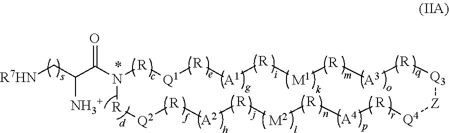

or a salt thereof (e.g., a pharmaceutically acceptable salt thereof), wherein

R⁷ and s are as defined with respect to formula (II);

each occurrence of R is, independently, —(CR³R⁴)—;

each occurrence of R³ and R⁴ are, independently H, halogen, OH, alkyl, alkoxy, —NH₂, alkylamino, or dialkylamino (in one preferred embodiment, each occurrence of R³ and R⁴ are, independently H or C₁-C₄ alkyl);

or R³ and R⁴, together with the carbon atom to which they are directly attached, form a cycloalkyl group, wherein no more than three R groups in each chain attached to the nitrogen N* are cycloalkyl (e.g., cyclopropyl);

Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;

Q³ and Q⁴ are each, independently, H, —(CR³R⁴)—, aryl, cycloalkyl, heterocyclyl, heterocyclylalkyl, heteroaryl, or a cholesterol moiety;

each occurrence of A¹, A², A³ and A⁴ is, independently, —(CR⁵R⁵—CR⁵═CR⁵)—;

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

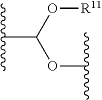

(wherein R¹¹ is a C₂-C₈ alkyl or alkenyl));

each occurrence of R⁵ is, independently, H or alkyl;

Z is absent, alkylene or —O—P(O)(OH)—O—;

each ------ attached to Z is an optional bond, such that when Z is absent, Q³ and Q⁴ are not directly covalently bound together;

c, d, e, f, i, j, m, n, q and r are each, independently, 0, 1, 2, 3, 4, 5, 6, 7, 8, 9, or 10;

g and h are each, independently, 0, 1 or 2;

k and l are each, independently, 0 or 1, where at least one of k and l is 1; and

o and p are each, independently, 0, 1 or 2.

In one embodiment of the compound of formula (IIA), Q³ and Q⁴ are each, independently, separated from the nitrogen atom marked with an asterisk (*) by a chain of 8 or more atoms (e.g., 12 or 14 or more atoms).

Yet another embodiment is a cationic lipid of the formula (IIB):

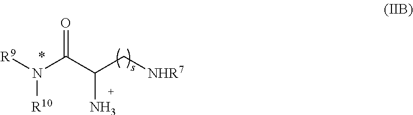

or a salt thereof (e.g., a pharmaceutically acceptable salt thereof), wherein

R⁷ and s are as defined with respect to formula (II); and

each of R⁹ and R¹⁰ are independently C₁₂-C₂₄ alkyl (e.g., C₁₂-C₂₀ alkyl), C₁₂-C₂₄ alkenyl (e.g., C₁₂-C₂₀ alkenyl), or C₁₂-C₂₄ alkoxy (e.g., C₁₂-C₂₀ alkoxy) having one or more biodegradable groups; each biodegradable group independently interrupts the alkyl, alkenyl, or alkoxy group or is substituted at the terminus of the alkyl, alkenyl, or alkoxy group.

In one embodiment of the compound of formula (IIB):
(i) the terminus of R⁹ is separated from the nitrogen atom marked with an asterisk (*) by a chain of 8 or more carbon atoms (e.g., 12 or 14 or more carbon atoms); and
(ii) the terminus of R¹⁰ is separated from the nitrogen atom marked with an asterisk (*) by a chain of 8 or more carbon atoms (e.g., 12 or 14 or more carbon atoms).

Yet another embodiment is a cationic lipid of the formula (IIC):

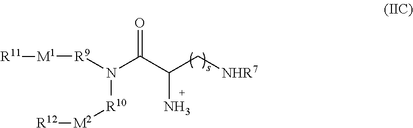

or a salt thereof (e.g., a pharmaceutically acceptable salt thereof), wherein

R⁷ and s are as defined with respect to formula (II);

each of R⁹ and R¹⁰ are independently alkyl (e.g., C₁₂-C₂₄ alkyl) or alkenyl (e.g., C₁₂-C₂₄ alkenyl);

each of R¹¹ and R¹² are independently alkyl or alkenyl, optionally terminated by COOR¹³ where each R¹³ is independently alkyl (e.g., C₁-C₄ alkyl such as methyl or ethyl);

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

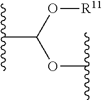

(wherein R¹¹ is a C₂-C₈ alkyl or alkenyl); in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently, H or C₁-C₄ alkyl));

R⁹, M¹, and R¹¹ are together at least 8 carbons atoms in length (e.g., 12 or 14 carbon atoms or longer); and

R¹⁰, M², and R¹² are together at least 8 carbons atoms in length (e.g., 12 or 14 carbon atoms or longer).

In a preferred embodiment of the compound of formula (IIC), R⁹ and R¹⁰ are each independently C₄-C₁₂ alkylene or C₄-C₁₂ alkenylene, M¹ and M² are —C(O)O— or —OC(O)—, and R¹¹ and R¹² are C₄-C₁₂ alkylene or C₄-C₁₂ alkenylene. In one embodiment, R⁹, M¹, and R¹¹ are together 12 to 24 carbons atoms in length. In another embodiment, R⁹, M¹, and R¹¹ are together 14 to 18 carbons atoms in length. In one embodiment, R¹⁰, M², and R¹² are together 12 to 24 carbons atoms in length. In another embodiment, R¹⁰, M² and R¹² are together 14 to 18 carbons atoms in length.

Yet another embodiment is a cationic lipid of the formula (IID):

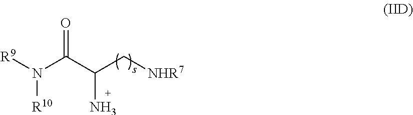

or a salt thereof (e.g., a pharmaceutically acceptable salt thereof), wherein

R⁷ and s are as defined with respect to formula (II); and

each of R⁹ and R¹⁰ are independently C₁₂-C₂₄ alkyl or C₁₂-C₂₄ alkenyl substituted at its terminus with a biodegradable group, such as —COOR¹³ where each R¹³ is independently alkyl (preferably C₁-C₄ alkyl such as methyl or ethyl).

In a preferred embodiment of the compound of formula (IID), R⁹ and R¹⁰ are each independently C₁₄-C₁₈ alkyl or C₁₄-C₁₈ alkenyl. In another preferred embodiment, the biodegradable group is —COOR¹³ where R¹³ is C₁-C₄ alkyl (such as methyl or ethyl).

In another preferred embodiment, a carbon atom alpha or beta to a biodegradable group (e.g., —C(O)O—) in any of the formulas recited herein may be substituted with one or two alkyl groups (e.g., one C₁-C₄ alkyl group, such as a —CH₃ substituent, or two C₁-C₄ alkyl groups, such as two —CH₃ substituents) or have a spirocyclic group (e.g., a C₃-C₅ cycloalkyl such as a C₃ cycloalkyl). For example, a carbon atom alpha or beta to a biodegradable group can be independently selected from

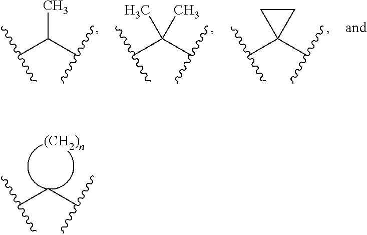

where n is 4-6.

In one embodiment, the biodegradable group (e.g., the M¹ or M² group in Formula (IA) or (IIA)) and neighboring variable(s) form the group:

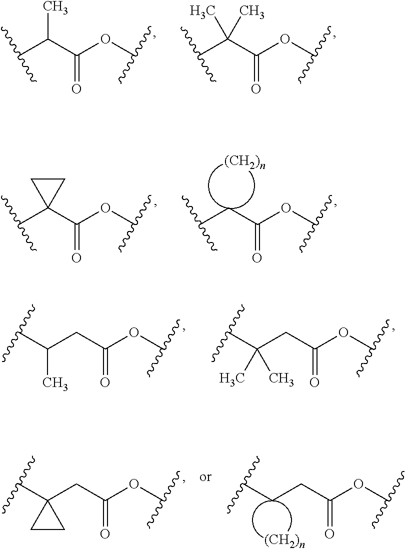

where n is 4-6.

In yet another embodiment, the cationic lipid is a compound selected from compounds of formulas III-XXIV:

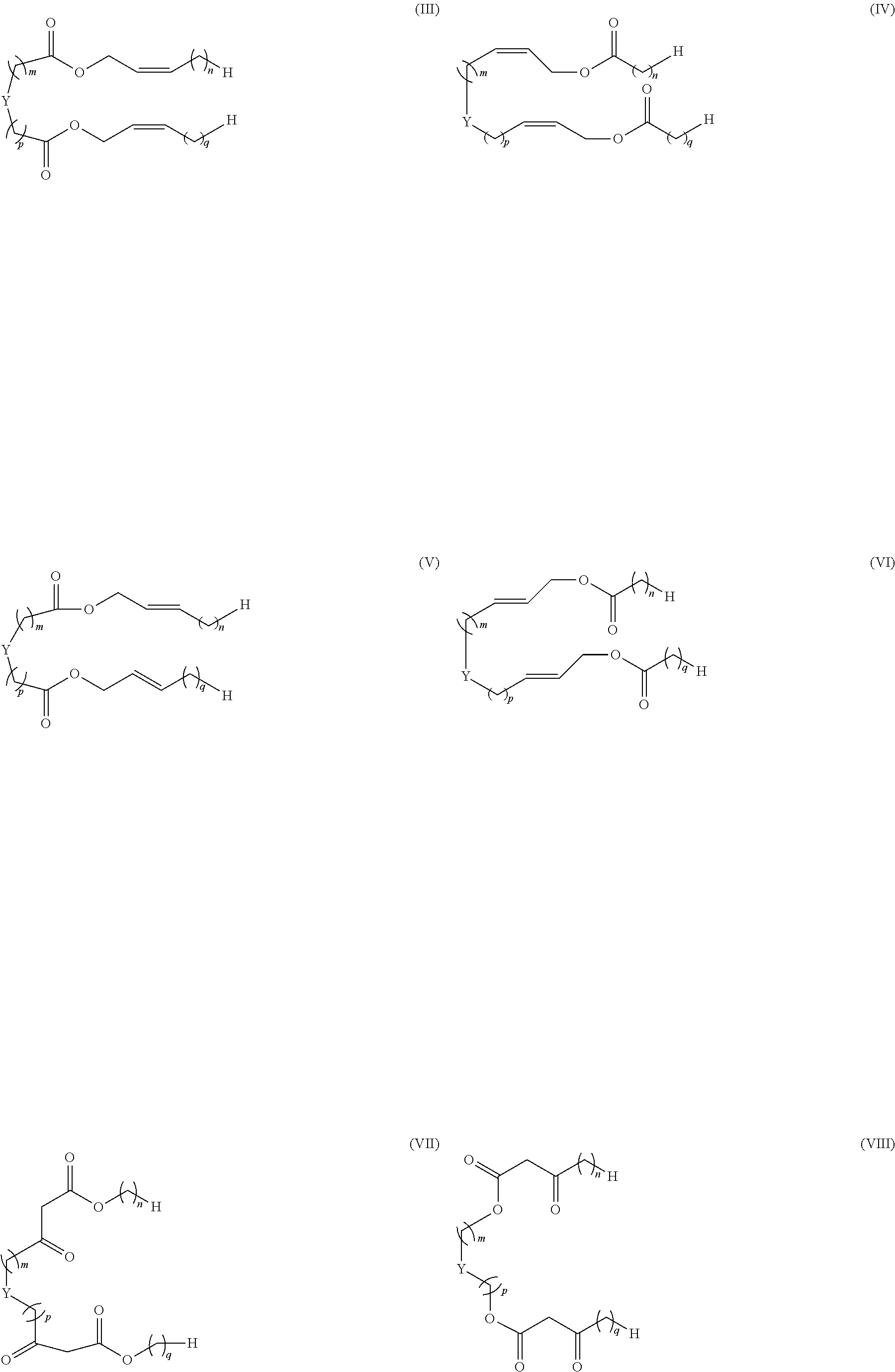

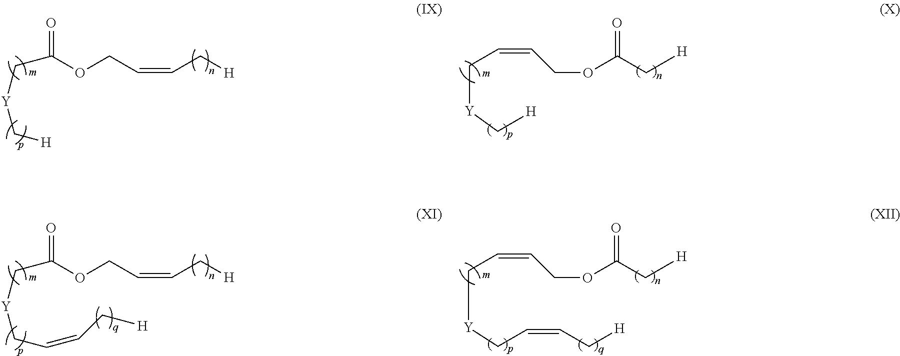

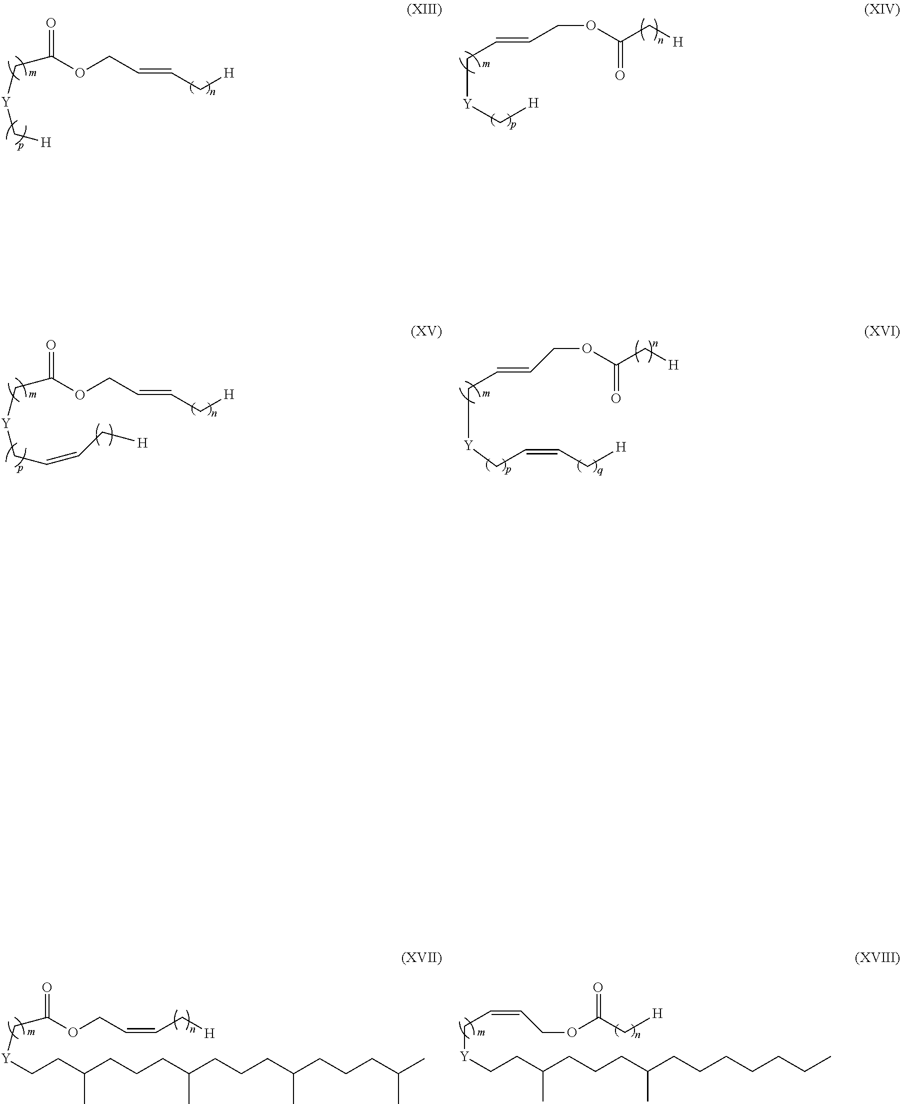

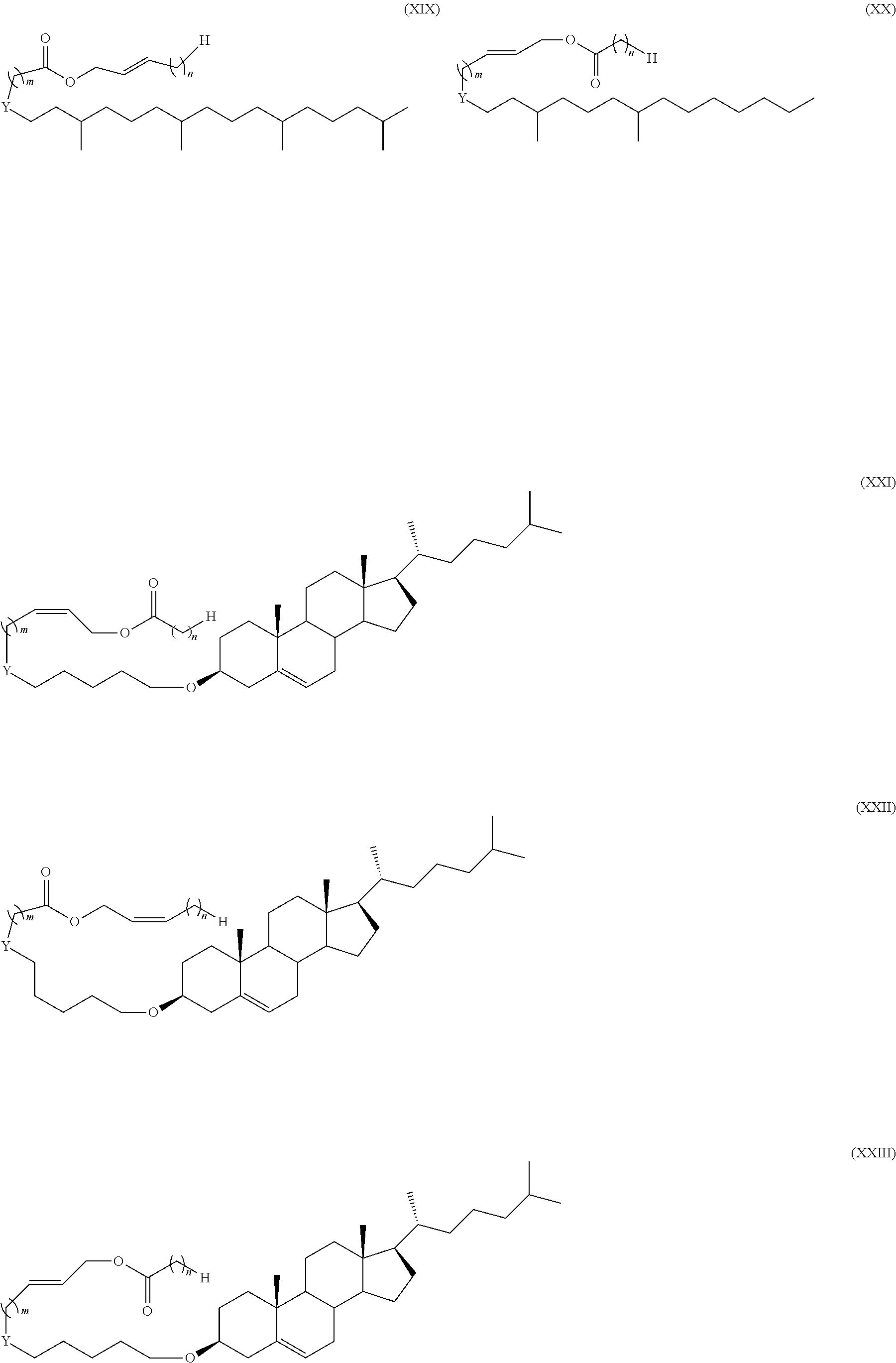

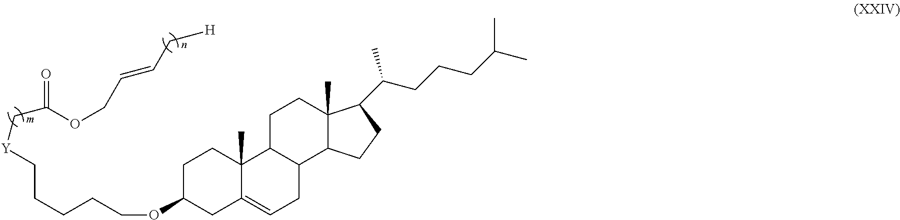

and salts thereof (e.g., pharmaceutically acceptable salts thereof), wherein

Y, in each case, independently is —C(O)-Xaa-Z—, —Z-Xaa-C(O)—, or

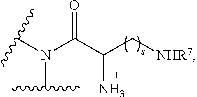

wherein Xaa and Z are defined with respect to formula (I) and R⁷ and s are defined with respect to formula (II);

m, n, p and q are each, individually, 1-25, with the proviso that:
(i) in Formulas (III), (V), (VII) and (VIII), m and p are both 4 or greater;
(ii) in Formulas (IX), (XI), (XIII), (XV), (XVII), (XIX), (XXII) and (XXIV), m is 4 or greater; and
(iii) in Formulas (IX), (X), (XIII) and (XIV), p is 8 or greater (e.g., 12 or 14 or greater).

In another embodiment, the nitrogen atom of the amino acid is within a pyrrolidinyl group. For example, the cationic lipid can be a compound selected from compounds of formulas I-7:

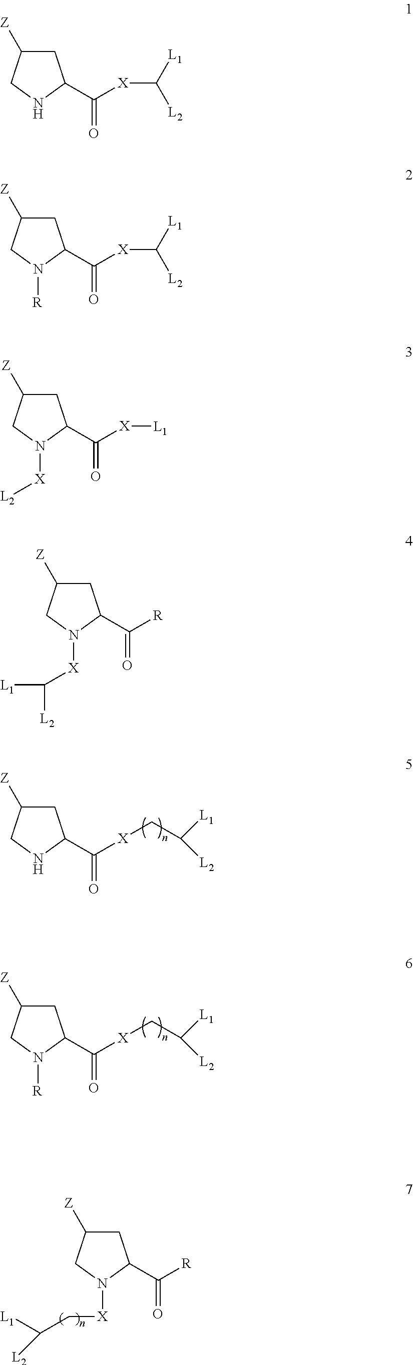

or a salt thereof (e.g., a pharmaceutically acceptable salt thereof), wherein

Z is —NH₂, —N(C₁-C₄ alkyl)₂ (e.g., —NMe₂), —OH, —OC(O)CH₂(CH₂)ₘCH₂N(C₁-C₄ alkyl)₂ (e.g., —OC(O)CH₂(CH₂)ₘCH₂N(Me)₂), —C(O)OCH₂(CH₂),CH₂N(C₁-C₄ alkyl)₂ (e.g., —C(O)OCH₂(CH₂)ₘCH₂N(Me)₂) or —NH—Y—CH₂(CH₂),CH₂N(C₁-C₄ alkyl)₂ (e.g., —NH—Y—CH₂(CH₂)ₘCH₂N(Me)₂);

R is —OH, —OC₁-C₄ alkyl (e.g., —OCH₃), —O(CH₂)ₘCH₂N(C₁-C₄ alkyl)₂ (e.g., —O(CH₂)ₘCH₂N(CH₃)₂), —N(R⁵)(CH₂)ₘCH₂N(C₁-C₄ alkyl)₂ (e.g., —N(R⁵)(CH₂)ₘCH₂N(CH₃)₂), —C(O)C₁-C₄ alkyl (e.g., —C(O)CH₃), C(O)CH₂(CH₂)ₘCH₂N(C₁-C₄ alkyl)₂ (e.g., —C(O)CH₂(CH₂)ₘCH₂N(CH₃)₂) or —C(O)OCH₂(CH₂)ₘCH₂N(C₁-C₄ alkyl)₂ (e.g., —C(O)OCH₂(CH₂)ₘCH₂N(CH₃)₂);

Y is —C(O)—, —OC(O)— or —C(O)O—;

each occurrence of m is, independently, 0, 1, 2, 3, 4, 5 or 6;

n is 1-6;

X is —C(O)—, —OC(O)—, —C(O)O—, —NH— or —N(C₁-C₄ alkyl)-; and

L₁ and L₂ are each, independently, C₁₂-C₂₄ alkyl (e.g., C₁₂-C₂₀ alkyl), C₁₂-C₂₄ alkenyl (e.g., C₁₂-C₂₀ alkenyl), or C₁₂-C₂₄ alkoxy (e.g., C₁₂-C₂₀ alkoxy);

L₁ and L₂ are each, independently, optionally interrupted by —O—, —S—, —NH— or —N(C₁-C₄ alkyl)-;

L₁ and L₂ each, independently, optionally contain one or more carbon-carbon double bonds;

L₁ and L₂ are each, independently, optionally interrupted by one or more biodegradable groups or are substituted at the terminus of the C₁₂-C₂₄ alkyl, alkenyl, or alkoxy group by a biodegradable group (e.g., —OC(O)—, —C(O)O—, —SC(O)—, —C(O)S—, —OC(S)—, —C(S)O, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—);

at least one of L₁ and L₂ includes at least one biodegradable group;

each occurrence of R⁵ is, independently, H or alkyl; and

each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently H or C₁-C₄ alkyl)).

In one embodiment, Z is —OC(O)CH₂(CH₂)ₘCH₂N(C₁-C₄ alkyl)₂ (e.g., —OC(O)CH₂(CH₂)ₘCH₂N(Me)₂), —C(O)OCH₂(CH₂)ₘCH₂N(C₁-C₄ alkyl)₂ (e.g., —C(O)OCH₂(CH₂)ₘCH₂N(Me)₂) or —NH—Y—CH₂(CH₂)ₘCH₂N(C₁-C₄ alkyl)₂ (e.g., —NH—Y—CH₂(CH₂)ₘCH₂N(Me)₂).

In another embodiment, Z is —NH—Y—CH₂(CH₂)ₘCH₂N(C₁-C₄ alkyl)₂ (e.g., —NH—Y—CH₂(CH₂)ₘCH₂N(Me)₂ such as —NH—C(O)—CH₂(CH₂)ₘCH₂N(Me)₂).

In one embodiment, the compounds of formulas I-7 are represented by subformulae 1′-7′, respectively:

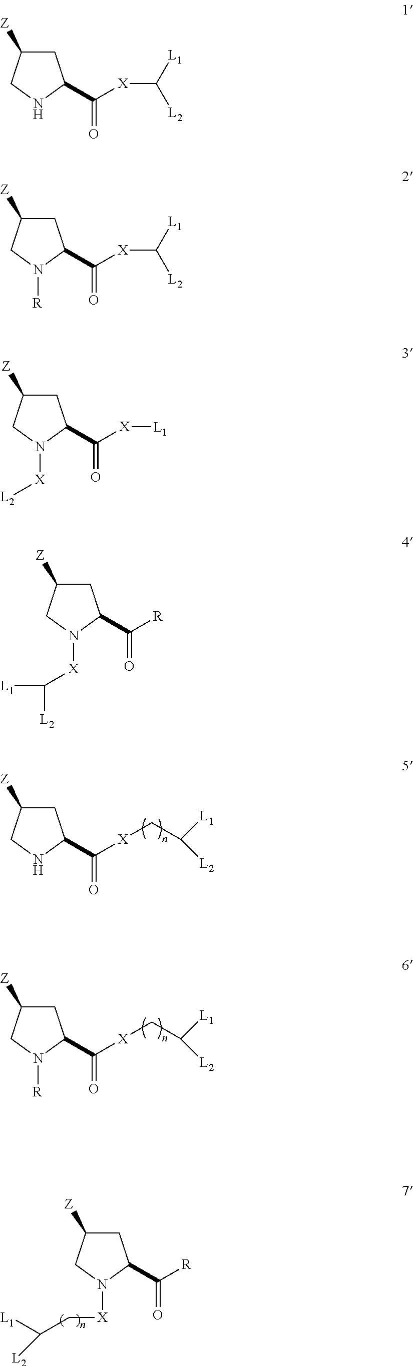

wherein X, Z, L¹, L² and n are as defined for formulas 1-7.

In another embodiment, the cationic lipid is a compound selected from compounds of formulas 8-18:

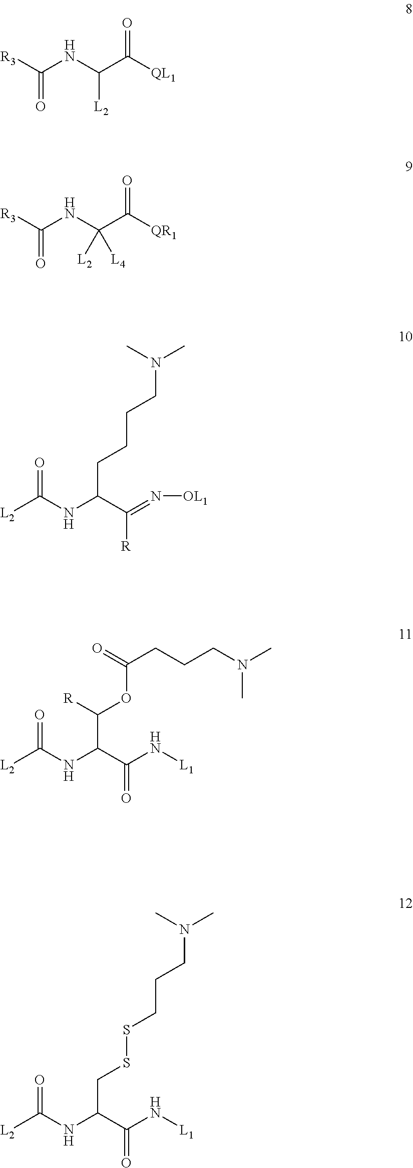

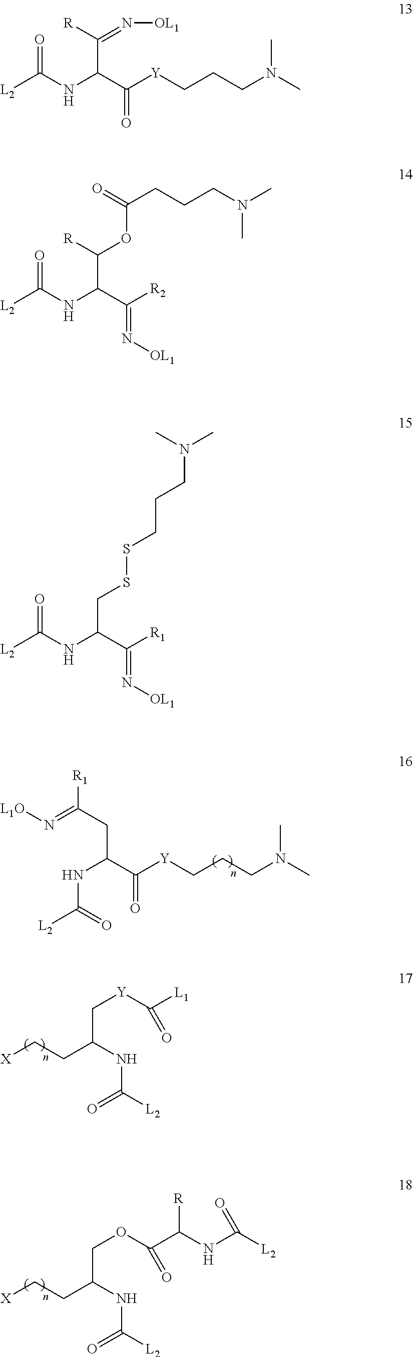

or a salt thereof (e.g., a pharmaceutically acceptable salt thereof), wherein

Q is —O—, —NH— or —N(C₁-C₄ alkyl);

L₁, L₂, and L₄ are each, independently, C₁₂-C₂₄ alkyl (e.g., C₁₂-C₂₀ alkyl), C₁₂-C₂₄ alkenyl (e.g., C₁₂-C₂₀ alkenyl), or C₁₂-C₂₄ alkoxy (e.g., C₁₂-C₂₀ alkoxy);

L₁, L₂, and L₄ are each, independently, optionally interrupted by —O—, —S—, —NH— or —N(C₁-C₄ alkyl)-;

L₁, L₂, and L₄ each, independently, optionally contain one or more carbon-carbon double bonds; and

L₁, L₂, and L₄ are each, independently, optionally interrupted by one or more biodegradable groups or are substituted at the terminus of the C₁₂-C₂₄ alkyl, alkenyl, or alkoxy group by a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—); in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently H or C₁-C₄ alkyl));

at least one L₁, L₂, and L₄ includes at least one biodegradable group;

R₃ is (C₁-C₄ alkyl)₂N(CH₂)ₘ—P— in which m is 0, 1, 2, 3, 4, 5 or 6 and P is absent, —C(O)—, —C(O)O—, —OC(O)—, —NH—C(O)O—, —OC(O)—NH— or —C(CH₃)═N—O— (e.g., R₃ is (CH₃)₂N—(CH₂)₃—C(O)O—, (CH₃)₂N—(CH₂)₂—NH—C(O)O—, (CH₃)₂N—(CH₂)₂—OC(O)—NH—, or (CH₃)₂N—(CH₂)₃—C(CH₃)═N—O—);

R₁ and R₂ is H or C₁-C₄ alkyl;

R is H or a non-hydrogen substituted or unsubstituted side chain of an amino acid;

n is 0, 1, 2, 3, 4, 5 or 6;

Y is —O—, —NH— or —N(C₁-C₄ alkyl); and

X is NR⁶R⁷ in which R⁶ and R⁷ are each, individually hydrogen or C₁-C₄ alkyl, or R⁶ and R⁷, together with the nitrogen atom to which they are attached, form an optionally substituted heterocylic ring (e.g., an optionally substituted 5- or 6-membered heterocyclic ring).

In another embodiment, the cationic lipid is a compound selected from a compound of formulas 19-25:

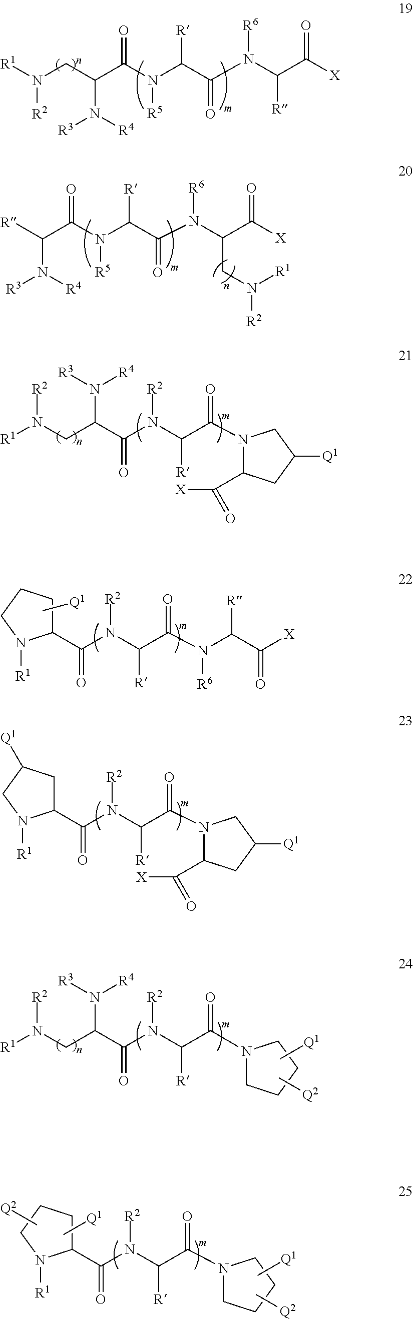

or a salt thereof (e.g., a pharmaceutically acceptable salt thereof), wherein

R′ an R″ are each, independently, a substituted or unsubstituted side chain of an amino acid,

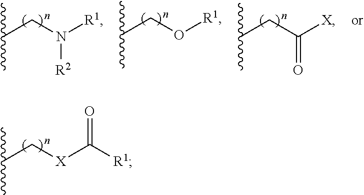

each occurrence of n is, independently, 0, 1, 2, 3, 4, 5, 6, 7, 8, 9, 10, 11 or 12;

each occurrence of m is, independently, 0, 1, 2, 3, 4, 5, 6, 7, 8, 9, 10, 11, 12, 13, 14, 15, 16, 17, 18, 19 or 20;

each occurrence of X is, independently, —OR¹ or —N(R¹)(R²);

each occurrence of R¹, R², R³, R⁴, R⁵ and R⁶ is, independently, H, C₁-C₄ alkyl (e.g., methyl), —OH, —N(C₁-C₄ alkyl)₂ (e.g., —NMe₂), —N(R^{x})—C(═NR^{x})—N(R^{x})(R^{x}), —COOH, —COO(R^{x}), —CON(R^{x})(R^{x}),

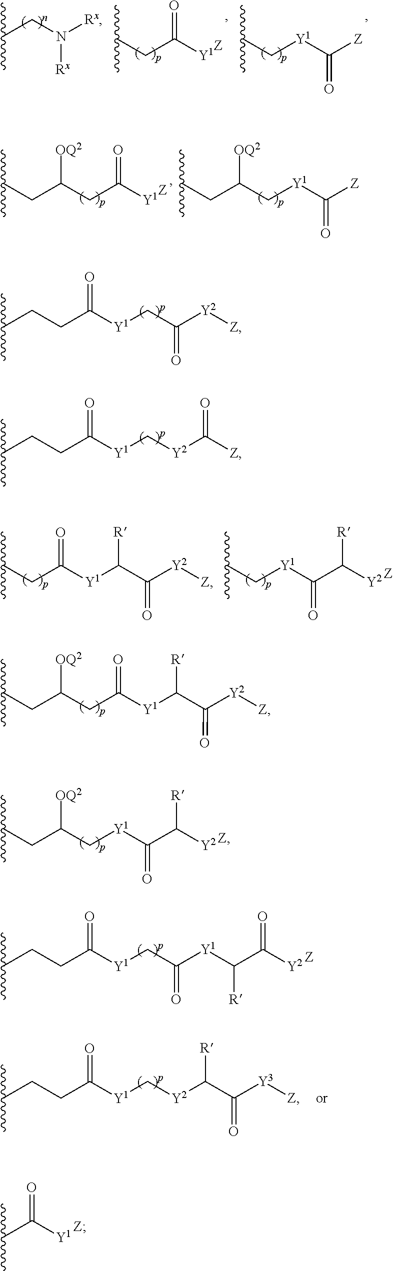

each occurrence of Q¹ and Q² is, independently, R′, R″, X or —C(O)X—;

each occurrence of p is, independently, 0, 1, 2, 3, 4, 5, 6, 7, 8, 9, 10, 11, 12, 13, 14, 15, 16, 17, 18, 19 or 20;

each occurrence of Y¹, Y² and Y³ is, independently, —O— or —NR^{x}—;

each occurrence of R^{x} is, independently, H or C₁-C₄ alkyl;

each occurrence of Z is, independently, —(CH₂)_{q}CH₃, —(CH₂)_{q}C(O)O(R¹), —(CH₂)_{q}C(O)N(R¹)(R²), —[(CH₂)_{q}C(R^{x})═C(R^{x})]ᵣ—CH₃, —[(CH₂)_{q}C(R^{x})]ᵣ—C(O)O(R¹), or —[(CH₂)_{q}C(R^{x})═C(R^{x})]ᵣ—C(O)N(R¹)(R²);

each occurrence of r is, independently, 0, 1, 2, 3, 4, 5, 6, 7, 8, 9, 10, 11, 12, 13, 14, 15, 16, 17, 18, 19 or 20; and

each occurrence of q is, independently, 0, 1, 2, 3, 4, 5, 6, 7, 8, 9, 10, 11, 12, 13, 14, 15, 16, 17, 18, 19 or 20,

wherein the compound contains at least one lipophilic moiety (e.g., a moiety containing at least 12 carbon atoms), and at least one of said lipohilic moieties in the compound contains at least one biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—); in which each occurrence of R⁵ is, independently, H or alkyl; and each occurrence of R³ and R⁴ are, independently H, halogen, OH, alkyl, alkoxy, —NH₂, alkylamino, or dialkylamino; or R³ and R⁴, together with the carbon atom to which they are directly attached, form a cycloalkyl group (in one preferred embodiment, each occurrence of R³ and R⁴ are, independently H or C₁-C₄ alkyl)).

In another embodiment, the present invention relates to a cationic lipid or a salt thereof having:

(i) a central carbon or nitrogen atom,

(ii) an amino acid containing head group directly bound to the central carbon or nitrogen atom, and

(iii) two hydrophobic tails directly bound to the central carbon or nitrogen atom, each hydrophobic tail comprising a C₈ or greater aliphatic group (preferably a C₁₄ or greater aliphatic group) attached to the central carbon or nitrogen atom, where one or both of the aliphatic group(s) (a) is interrupted by a biodegradable group such that there is a chain of at least four carbon atoms between the biodegradable group and the central carbon or nitrogen atom, or (b) includes a biodegradable group at the terminal end of the hydrophobic tail. For instance, the biodegradable group is selected from —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, and —OC(O)O—.

In one embodiment, the amino acid is an L-amino acid. In another embodiment, the amino acid is an D-amino acid. In one preferred embodiment, the amino acid is an α-amino acid, such as an L-amino acid.

2) Cationic lipids that Include One or More Biodegradable Groups.

In one embodiment, the cationic lipid is a compound of the formula:

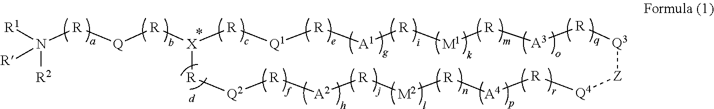

or a salt thereof (e.g., a pharmaceutically acceptable salt thereof),
wherein

X is N or P;

R′ is absent, hydrogen, or alkyl (e.g., C₁-C₄ alkyl);

with respect to R¹ and R²,
(i) R¹ and R² are each, independently, optionally substituted alkyl, alkenyl, alkynyl, cycloalkyl, cycloalkylalkyl, heterocycle, or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 member heterocyclic ring or heteroaryl (e.g., a 6-member ring) with (a) the adjacent nitrogen atom and (b) the (R)ₐ group adjacent to the nitrogen atom;

each occurrence of R is, independently, —(CR³R⁴)—;

each occurrence of R³ and R⁴ are, independently H, halogen, OH, alkyl, alkoxy, —NH₂, alkylamino, or dialkylamino (in one preferred embodiment, each occurrence of R³ and R⁴ are, independently H or C₁-C₄ alkyl);

or R³ and R⁴, together with the carbon atom to which they are directly attached, form a cycloalkyl group, wherein no more than three R groups in each chain attached to the atom X* are cycloalkyl (e.g., cyclopropyl);

each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;

Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;

Q³ and Q⁴ are each, independently, H, —(CR³R⁴)—, aryl, or a cholesterol moiety;

each occurrence of A¹, A², A³ and A⁴ is, independently, —(CR⁵R⁵—CR⁵═CR⁵)—;

each occurrence of R⁵ is, independently, H or alkyl;

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—);

Z is absent, alkylene or —O—P(O)(OH)—O—;

each ------ attached to Z is an optional bond, such that when Z is absent, Q³ and Q⁴ are not directly covalently bound together;

a is 1, 2, 3, 4, 5 or 6;

b is 0, 1, 2, or 3;

c, d, e, f, i, j, m, n, q and r are each, independently, 0, 1, 2, 3, 4, 5, 6, 7, 8, 9, or 10;

g and h are each, independently, 0, 1 or 2;

k and l are each, independently, 0 or 1, where at least one of k and l is 1; and

o and p are each, independently, 0, 1 or 2,

wherein

Q³ and Q⁴ are each, independently, separated from the tertiary atom marked with an asterisk (X*) by a chain of 8 or more atoms (e.g., 12 or 14 or more atoms).

In one embodiment, (i) R¹ and R² are each, independently, optionally substituted alkyl, alkenyl, alkynyl, cycloalkyl, cycloalkylalkyl, or heterocycle; or (ii) R¹ and R², together with the nitrogen atom to which they are attached, form an optionally substituted heterocylic ring.

In a preferred embodiment of the compound of formula (I),
(a) when Q¹ is a biodegradable group (e.g., —C(O)O—), then c is at least 4;
(b) when Q² is a biodegradable group, then d is at least 4; and
(c) Q³ and Q⁴ are each, independently, separated from the tertiary atom marked with an asterisk (X*) by a chain of 10 or more atoms (e.g., 12 or 14 or more atoms).

In another preferred embodiment, a carbon atom alpha or beta to a biodegradable group (e.g., —C(O)O—) in formula (I) may be substituted with one or two alkyl groups (e.g., one C₁-C₄ alkyl group, such as a —CH₃ substituent, or two C₁-C₄ alkyl groups, such as two —CH₃ substituents) or have a spirocyclic group (e.g., a C₃-C₅ cycloalkyl such as a C₃ cycloalkyl). For example, a carbon atom alpha or beta to a biodegradable group can be independently selected from

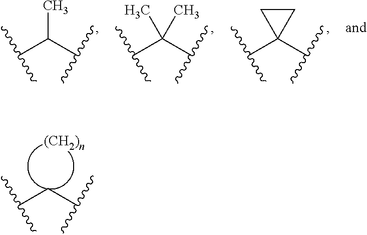

(where n is 4-6).

In one embodiment, the M¹ or M² group and neighboring variable(s) form the group:

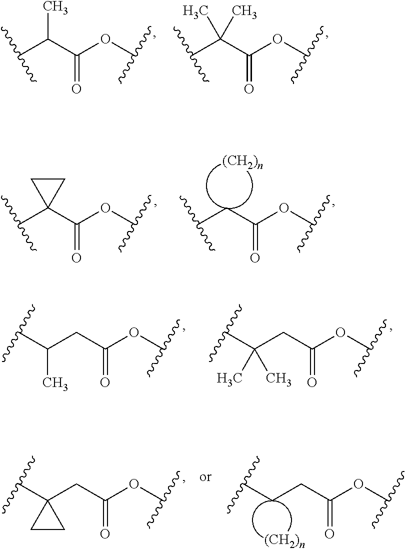

(where n is 4-6).

Yet another embodiment is a cationic lipid of the formula

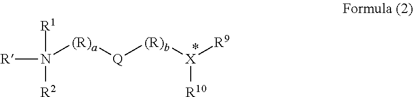

or a salt thereof (e.g., a pharmaceutically acceptable salt thereof), wherein

X is N or P;

R¹, R², R, a, and b are as defined with respect to formula (I);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;

R′ is absent, hydrogen, or alkyl (e.g., C₁-C₄ alkyl); and

each of R⁹ and R¹⁰ are independently C₁₂-C₂₄ alkyl (e.g., C₁₂-C₂₀ alkyl), C₁₂-C₂₄ alkenyl (e.g., C₁₂-C₂₀ alkenyl), or C₁₂-C₂₄ alkoxy (e.g., C₁₂-C₂₀ alkoxy) having one or more biodegradable groups; each biodegradable group independently interrupts the C₁₂-C₂₄ alkyl, alkenyl, or alkoxy group or is substituted at the terminus of the C₁₂-C₂₄ alkyl, alkenyl, or alkoxy group,

wherein

the terminus of R⁹ and R¹⁰ is separated from the tertiary atom marked with an asterisk (X*) by a chain of 8 or more atoms (e.g., 12 or 14 or more atoms).

In another embodiment, the cationic lipid is a compound of the formula:

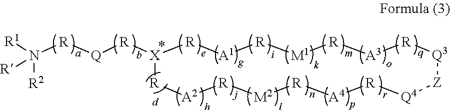

or a salt thereof (e.g., a pharmaceutically acceptable salt thereof), wherein

X is N or P;

R′ is absent, hydrogen, or alkyl (e.g., C₁-C₄ alkyl);

R¹ and R² are each, independently, optionally substituted C₁-C₄ alkyl, C₂-C₄ alkenyl, C₂-C₄ alkynyl, C₃-C₆ cycloalkyl, (C₃-C₆ cycloalkyl)C₁-C₄ alkyl, or a monocyclic heterocycle; or

R¹ and R², together with the nitrogen atom to which they are attached, form an optionally substituted 5- or 6-membered heterocylic ring (e.g., a C₅ or C₆ heterocyclic ring);

each occurrence of R is, independently, —(CR³R⁴)—;

each occurrence of R³ and R⁴ are, independently H, halogen, OH, alkyl, alkoxy, —NH₂, alkylamino, or dialkylamino (in one preferred embodiment, each occurrence of R³ and R⁴ are, independently H or C₁-C₄ alkyl);

or R³ and R⁴, together with the carbon atom to which they are directly attached, form a C₃-C₆ cycloalkyl group, wherein no more than three R groups in each chain attached to the atom X* are cycloalkyl (e.g., cyclopropyl);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;

Q³ and Q⁴ are each, independently, H, —(CR³R⁴)—, aryl, or a cholesterol moiety;

each occurrence of A¹, A², A³ and A⁴ is, independently, —(CR⁵R⁵—CR⁵═CR⁵)—; i

each occurrence of R⁵ is, independently, H or alkyl;

M¹ and M² are each, independently, —C(O)—O—, —OC(O)—, —C(R⁵)═N—, —C(R⁵)═N—O—, —O—C(O)O—, —C(O)N(R⁵)—, —C(O)S—, —C(S)O—, —OSi(R⁵)₂O—, —C(O)(CR³R⁴)C(O)O—, or —OC(O)(CR³R⁴)C(O)—;

Z is absent, alkylene or —O—P(O)(OH)—O—;

each ------ attached to Z is an optional bond, such that when Z is absent, Q³ and Q⁴ are not directly covalently bound together;

a is 1, 2, 3, 4, 5 or 6;

b is 0, 1, 2, or 3;

d, e, i, j, m, n, q and r are each, independently, 0, 1, 2, 3, 4, 5, 6, 7, 8, 9, or 10;

g and h are each, independently, 0, 1 or 2;

the sum of d+3h is at least 4, and the sum of e+3g is at least 4;

k and l are each, independently, 0 or 1, where at least one of k and l is 1; and

o and p are each, independently, 0, 1 or 2,

wherein Q³ and Q⁴ are each, independently, separated from the tertiary atom marked with an asterisk (X*) by a chain of 8 or more atoms (e.g., 12 or 14 or more atoms).

In one embodiment, R′ in formula (3) is absent or hydrogen. In one embodiment, R′ in formula (3) is absent or alkyl (e.g., methyl).

In one embodiment, R¹ and R² in formula (3) are each, independently, C₁-C₄ alkyl (e.g., methyl or ethyl).

In one embodiment, each occurrence of R in formula (3) is, independently, —CH₂— or —CH(CH₃)—.

In one embodiment, Q³ and Q⁴ in formula (3) are each, independently, H, aryl, or a cholesterol moiety.

In one embodiment, each occurrence of A¹, A², A³ and A⁴ in formula (3) is, independently, —(CH₂—CH═CH)—;

In one embodiment, M¹ and M² in formula (3) are each —C(O)—O—.

In one embodiment of the compound of formula (3), Z is absent and each is absent (i.e., Q³ and Q⁴ are not directly covalently bound together).

In one embodiment, the sum of e+3g+i+m+3o+q in formula (3) is from about 8 to about 20. In another embodiment, the sum of e+3g+i+m+3o+q in formula (3) is from about 12 to about 20.

In one embodiment, the sum of d+3h+j+n+3p+r in formula (3) is from about 8 to about 20. In another embodiment, the sum of d+3h+j+n+3p+r in formula (3) is from about 12 to about 20.

In another embodiment, the cationic lipid is a compound of the formula

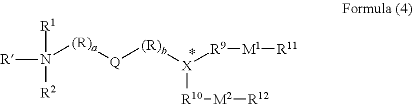

wherein

X is N or P;

R¹, R², R, a, b, M¹, and M² are as defined with respect to formula (I);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;

R′ is absent, hydrogen, or alkyl (e.g., C₁-C₄ alkyl);

each of R⁹ and R¹⁰ are independently alkylene, or alkenylene; and

each of R¹¹ and R¹² are independently alkyl or alkenyl, optionally terminated by COOR¹³ where each R¹³ is independently alkyl (e.g., C₁-C₄ alkyl such as methyl or ethyl);

R⁹, M¹, and R¹¹ are together at least 8 carbons atoms in length (e.g., 12 or 14 carbon atoms or longer); and

R¹⁰, M², and R¹² are together at least 8 carbons atoms in length (e.g., 12 or 14 carbon atoms or longer).

In a preferred embodiment of the compound of formula (4), R⁹ and R¹⁰ are each independently C₄-C₁₂ alkylene or C₄-C₁₂ alkenylene, M¹ and M² are —C(O)O—, and R¹¹ and R¹² are C₄-C₁₂ alkylene or C₄-C₁₂ alkenylene. In one embodiment, R⁹, M¹, and R¹¹ are together at 12 to 24 carbons atoms in length. In another embodiment, R⁹, M¹, and R¹¹ are together at 14 to 18 carbons atoms in length. In one embodiment, R¹⁰, M², and R¹² are together at 12 to 24 carbons atoms in length. In another embodiment, R¹⁰, M², and R¹² are together at 14 to 18 carbons atoms in length.

The R′R¹R²N—(R)ₐ-Q-(R)_{b}— group can be any of the head groups described herein, including those shown in Table 1 below, and salts thereof. In one preferred embodiment, R′R¹R²N—(R)ₐ-Q-(R)_{b}— is (CH₃)₂N—(CH₂)₃—C(O)O—, (CH₃)₂N—(CH₂)₂—NH—C(O)O—, (CH₃)₂N—(CH₂)₂—OC(O)—NH—, or (CH₃)₂N—(CH₂)₃—C(CH₃)═N—O—.

In yet another embodiment, the cationic lipid is a compound of the formula

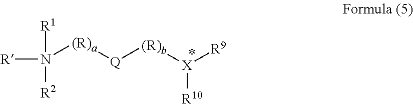

wherein

X is N or P;

R¹, R², R, a, and b are as defined with respect to formula (I);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;R′ is absent, hydrogen, or alkyl (e.g., C₁-C₄ alkyl);

each of R⁹ and R¹⁰ are independently C₁₂-C₂₄ alkyl or alkenyl substituted at its terminus with a biodegradable group, such as —COOR¹³ where each R¹³ is independently alkyl (preferably C₁-C₄ alkyl such as methyl or ethyl).

In a preferred embodiment of the compound of formula (IC), R⁹ and R¹⁰ are each independently C₁₄-C₁₈ alkylene or C₁₄-C₁₈ alkenylene. In another preferred embodiment, the biodegradable group is —COOR¹³ where R¹³ is C₁-C₄ alkyl (such as methyl or ethyl).

The R′R¹R²N—(R)ₐ-Q-(R)_{b}— group can be any of the head groups described herein, including those shown in Table 1 below. In one preferred embodiment, R′R¹R²N—(R)ₐ-Q-(R)_{b}— is (CH₃)₂N—(CH₂)₃—C(O)O—, (CH₃)₂N—(CH₂)₂—NH—C(O)O—, (CH₃)₂N—(CH₂)₂—OC(O)—NH—, or (CH₃)₂N—(CH₂)₃—C(CH₃)═N—O—.

Yet another embodiment are intermediates of the formula:

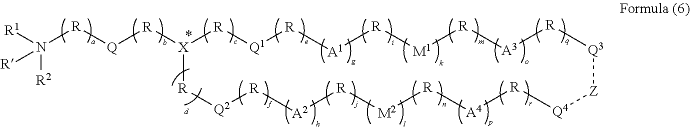

or a salt thereof (e.g., a pharmaceutically acceptable salt thereof),
wherein

X is N or P;

R′ is absent, hydrogen, or alkyl (e.g., C₁-C₄ alkyl);

R¹ and R² are each, independently, optionally substituted alkyl, alkenyl, alkynyl, cycloalkyl, cycloalkylalkyl, heterocycle or R¹⁰; or

R¹ and R², together with the nitrogen atom to which they are attached, form an optionally substituted heterocylic ring;

each occurrence of R is, independently, —(CR³R⁴)—;

each occurrence of R³ and R⁴ are, independently H, halogen, OH, alkyl, alkoxy, —NH₂, alkylamino, or dialkylamino (in one preferred embodiment, each occurrence of R³ and R⁴ are, independently H or alkyl);

or R³ and R⁴, together with the carbon atom to which they are directly attached, form a cycloalkyl group, wherein no more than three R groups in each chain attached to the atom X* are cycloalkyl (e.g., cyclopropyl);

each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups (preferably at most one R¹⁰ group);

Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;

Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;

Q³ and Q⁴ are each, independently, H, —(CR³R⁴)—, aryl, —OH, or a cholesterol moiety;

each occurrence of A¹, A², A³ and A⁴ is, independently, —(CR⁵R⁵—CR⁵═CR⁵)—;

each occurrence of R⁵ is, independently, H or alkyl;

M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—);

Z is absent, alkylene or —O—P(O)(OH)—O—;

each ------ attached to Z is an optional bond, such that when Z is absent, Q³ and Q⁴ are not directly covalently bound together;

a is 1, 2, 3, 4, 5 or 6;

b is 0, 1, 2, or 3;

c, d, e, f, i, j, m, n, q and r are each, independently, 0, 1, 2, 3, 4, 5, 6, 7, 8, 9, or 10;

g and h are each, independently, 0, 1 or 2;

k and l are each, independently, 0 or 1;

o and p are each, independently, 0, 1 or 2,

wherein

Q³ and Q⁴ are each, independently, separated from the tertiary atom marked with an asterisk (X*) by a chain of 8 or more atoms (e.g., 12 or 14 or more atoms).

In yet another embodiment, the cationic lipid is a compound selected from compounds of formulas 7-42:

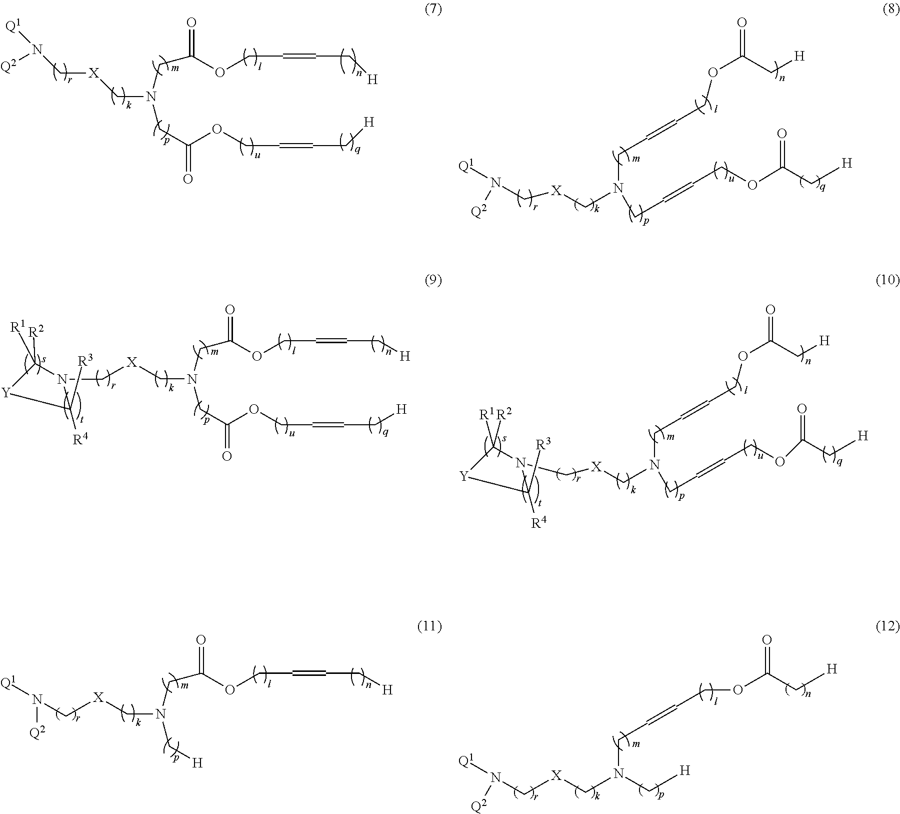

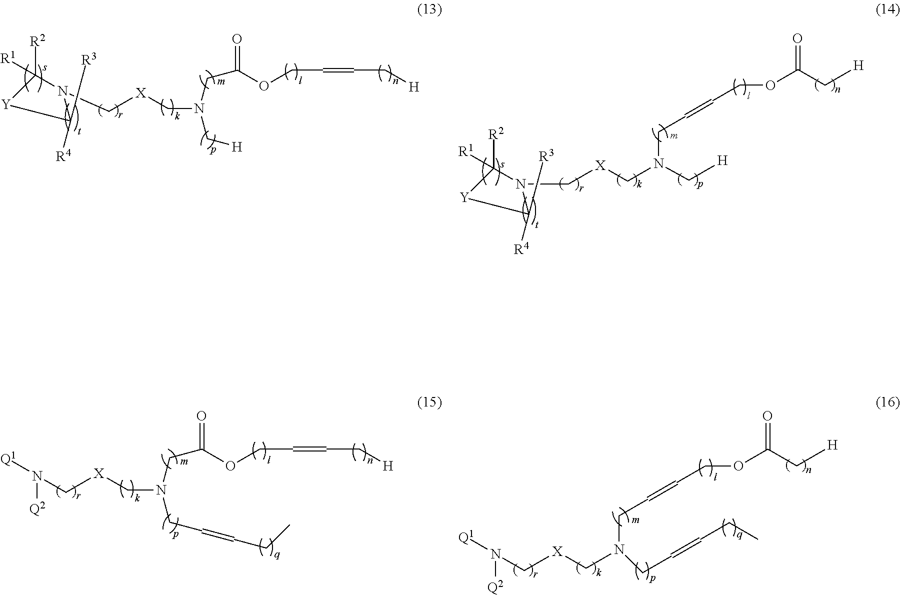

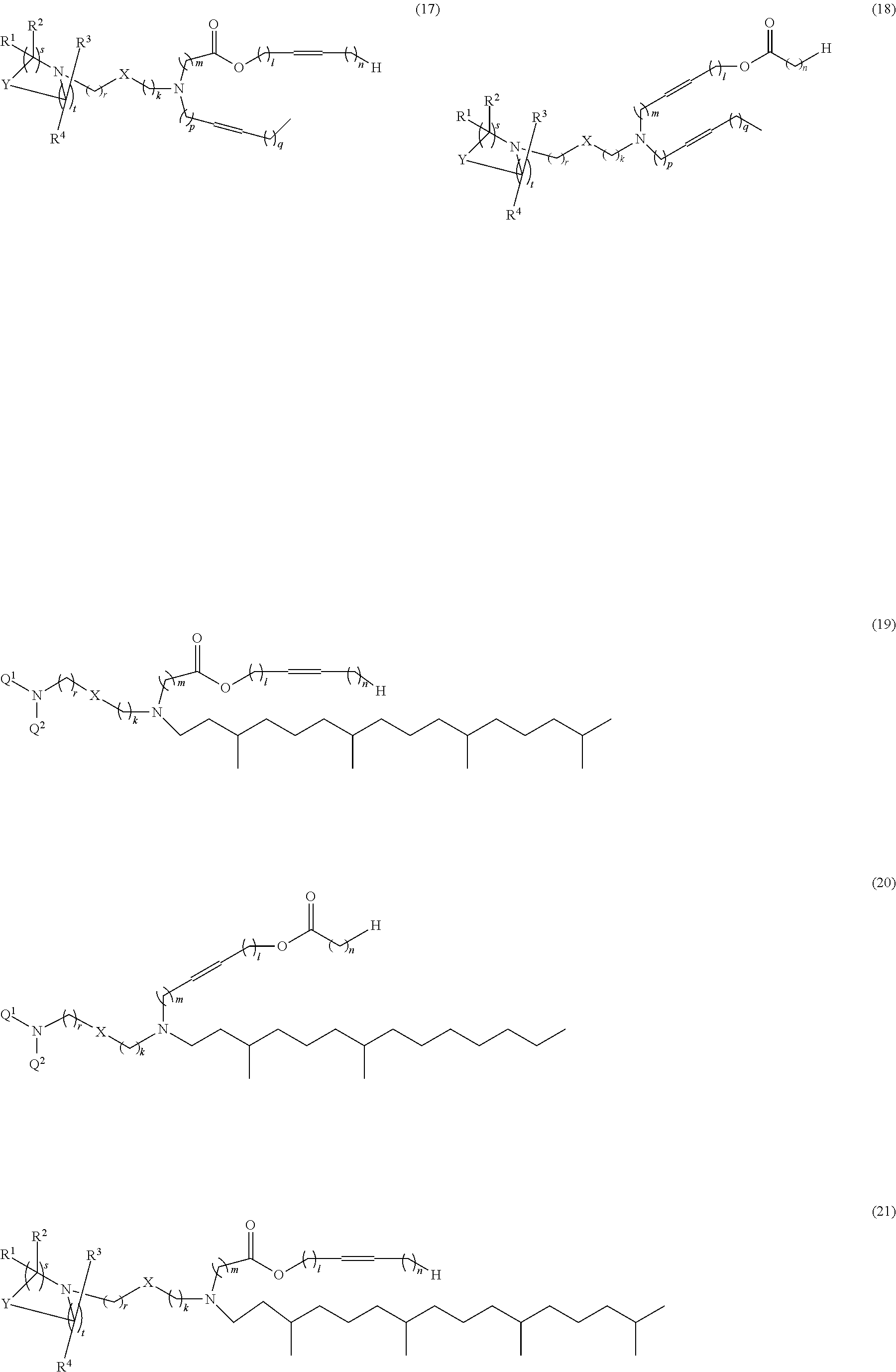

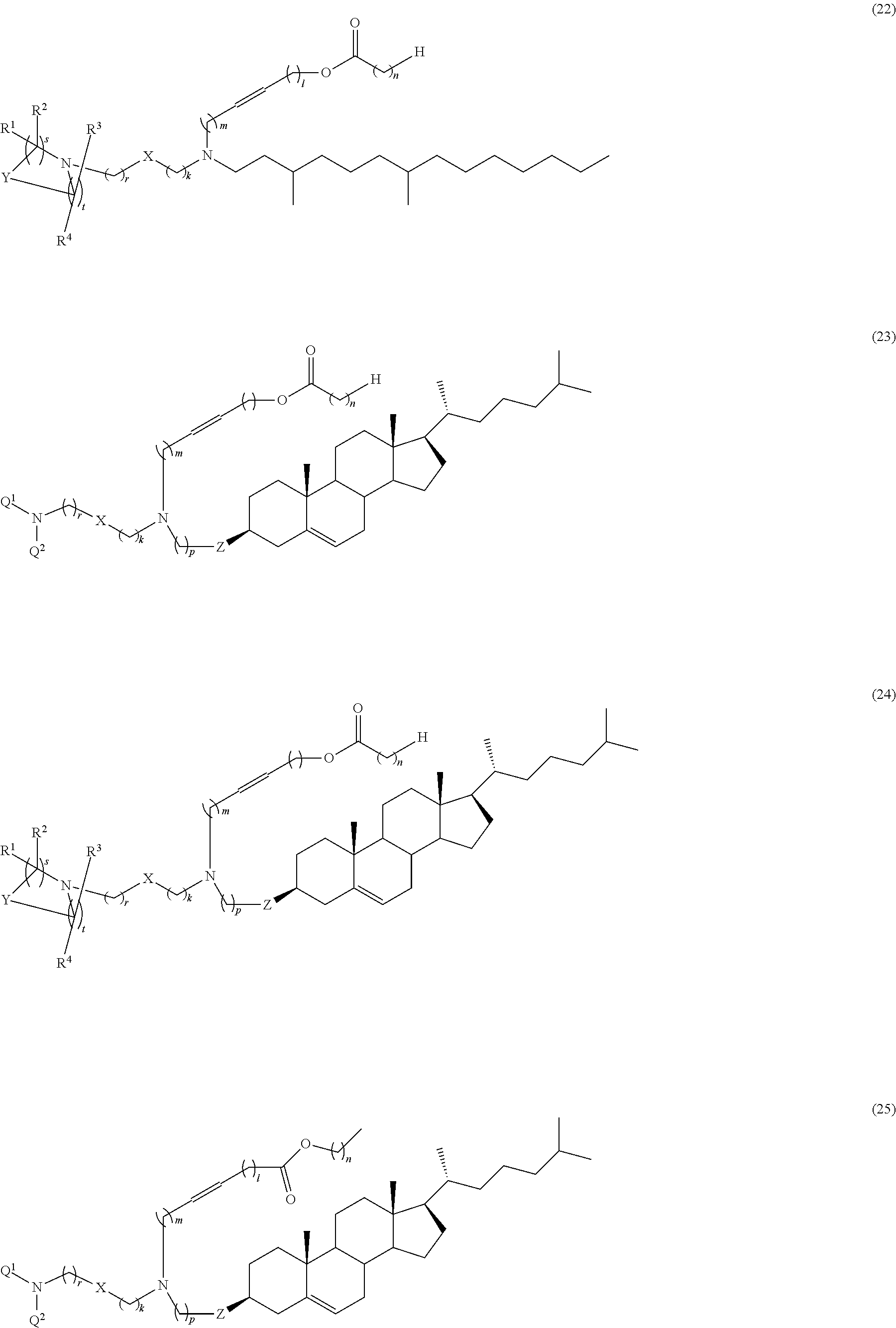

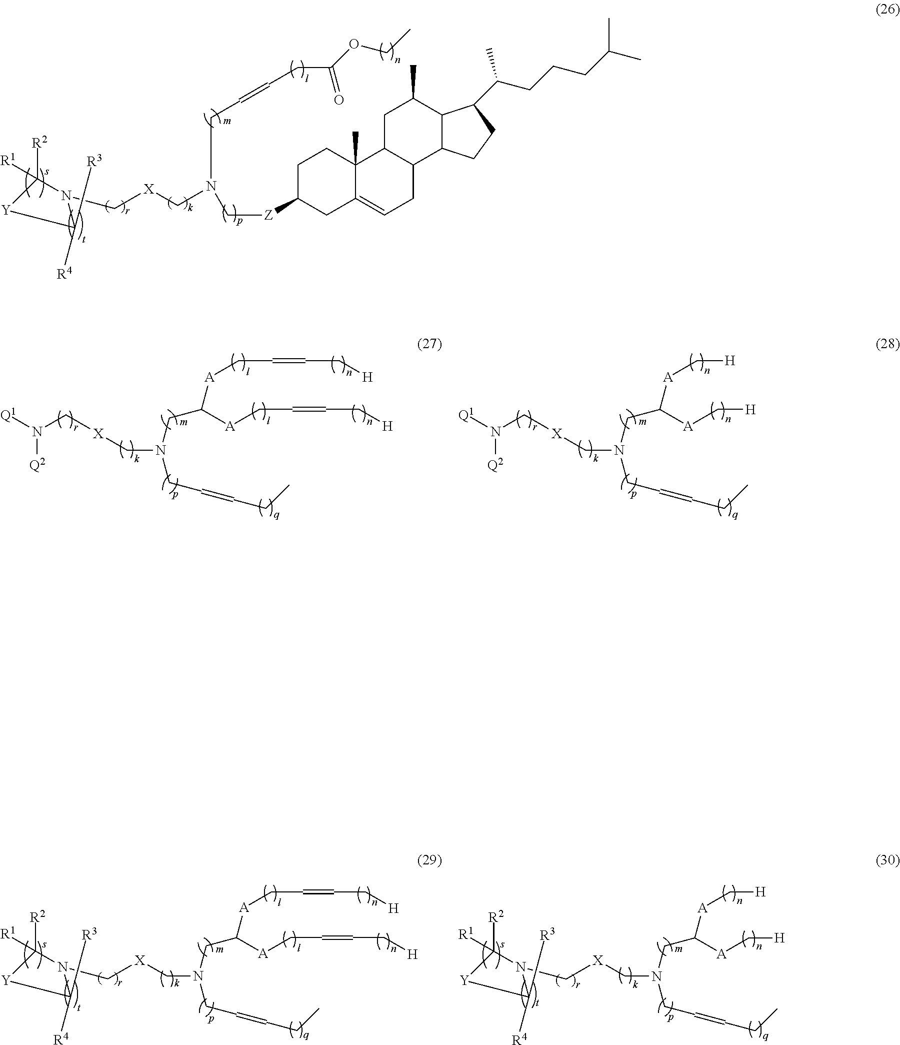

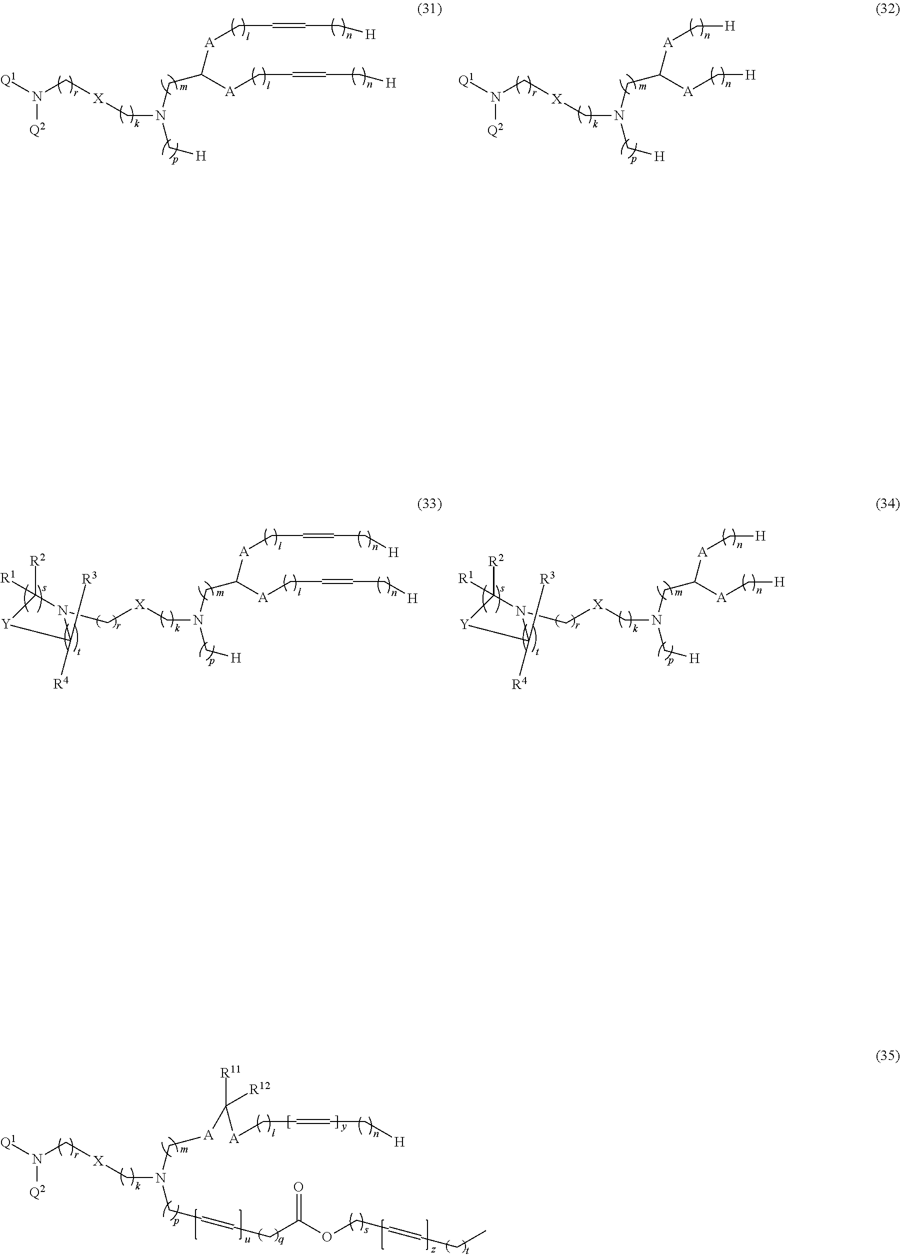

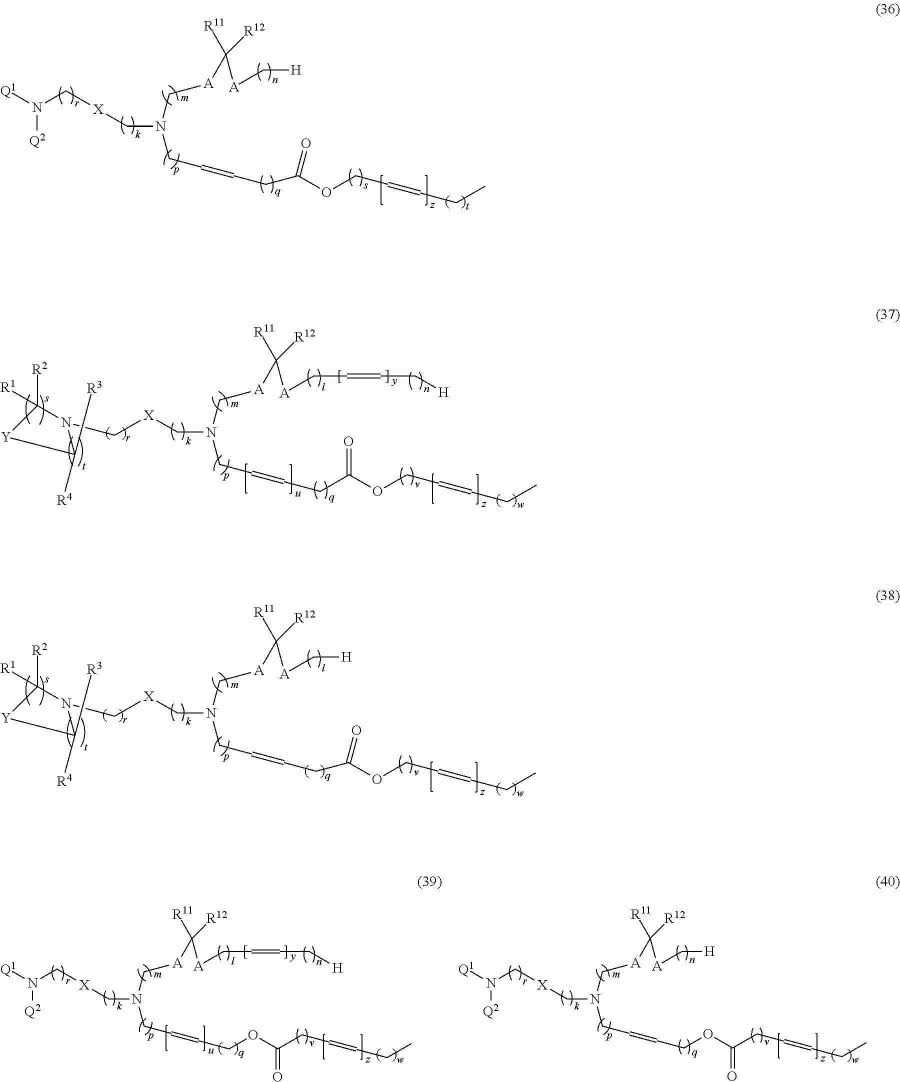

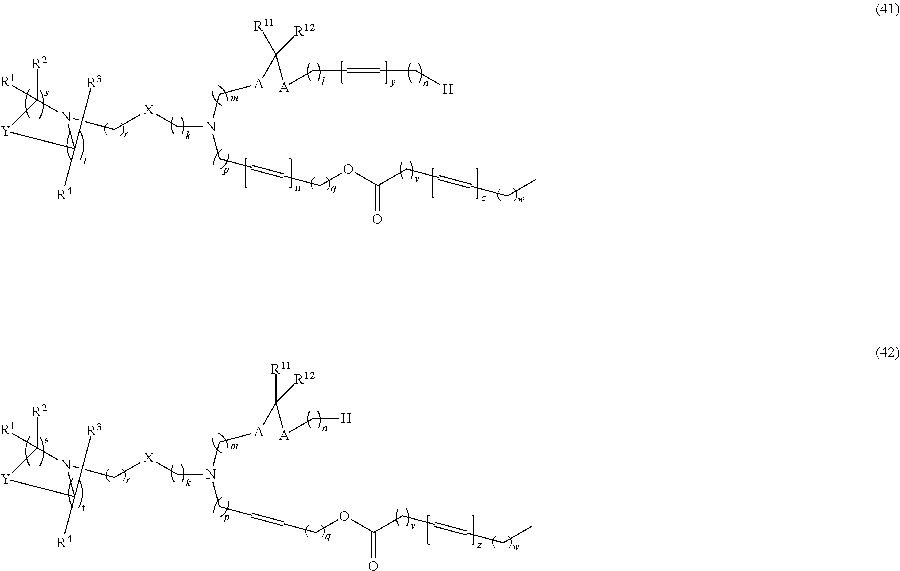

and salts (e.g., pharmaceutically acceptable salts) thereof,
wherein

each occurrence of X is, independently, O, S, N(R); CH₂, —CH═, —CH₂—CH₂—, —CH═CH—, —C≡C—, —OC(O)—, —C(O)O—, —OC(O)O—, —N(R)—C(O)—, —N(R)—C(O)O—, —N(R)—C(O)N(R′)—, —C(O)N(R′)—, —OC(O)N(R′)—, —C(O)S—, —S—S—, —SC(O)—, —N(R)—C(O)S—, or —SC(O)N(R′)—;

each occurrence of Y is, independently, C(R⁵)(R⁶), N(R′), O, S, —CH₂—CH₂—, —CH═CH—, or —C≡C—;

each occurrence of Z is, independently, O, S, N(R), CH₂, —CH═, —CH═CH—, —OC(O)—, —C(O)O—, —OC(O)O—, —N(R)—C(O)—, —N(R)—C(O)O—, —N(R)—C(O)N(R′)—, —C(O)N(R′)—, —OC(O)N(R′)—, —C(O)S—, —S—S—, —SC(O)—, —N(R)—C(O)S—, —SC(O)N(R′)—, —CH₂—CH₂—, —CH═CH—, or —C≡C—;

each occurrence of A is, independently, O or S;

each occurrence of k, l, m, n, p and q, v, w, and u is, independently, 0-20;

each occurrence of r is, independently, 0-10;

each occurrence of s and t is, independently, 0-6;

each occurrence of y and z is, independently, 0 or 1;

each occurrence of Q¹ and Q² is, independently, H, alkyl (e.g., Me, Et, Pr, iPr, Bu, iBu, tBu), substituted alkyl (e.g., alkoxyalkyl, fluoroalkyl such as perfluoroalkyl), aryl or substituted aryl;

each occurrence of R, R¹, R², R³, R⁴, R⁵, R⁶, R¹¹, R¹² and R′ is, independently, H, halogen (e.g., F), alkyl (e.g., Me, Et, Pr, iPr, Bu, iBu, and tBu), substituted alkyl (e.g., alkoxyalkyl and fluoroalkyl such as perfluoroalkyl), aryl or substituted aryl; and

wherein each hydrophobic group may, optionally, independently be further substituted by —OH, alkoxy, alkoxyalkyl, or a combination thereof.

In another embodiment, the present invention relates to a cationic lipid or a salt thereof having:

(i) a central nitrogen or phosphorous atom,

(ii) a nitrogen containing head group directly bound to the central nitrogen or phosphorous atom, and

(iii) two hydrophobic tails directly bound to the central nitrogen or phosphorous atom, each hydrophobic tail comprising a C₈ or greater aliphatic group (preferably a C₁₄ or greater aliphatic group) attached to the central nitrogen or phosphorous atom, where one or both of the aliphatic group(s) (a) is interrupted by a biodegradable group such that there is a chain of at least four carbon atoms between the biodegradable group and the central nitrogen or phosphorous atom, or (b) includes a biodegradable group at the terminal end of the hydrophobic tail. For instance, the biodegradable group is selected from —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, and —OC(O)O—.

In another aspect, the present invention relates to cationic lipids that include an acetal or ketal group (that provides a low pH sensitive chemical handle for degredation) and, optionally, one or more biodegradable groups.

3a) Cationic Lipids with an Acetal Head Group

In one embodiment of this invention, the cationic lipid is of Formula A:

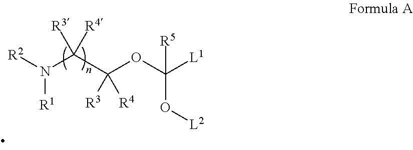

or a pharmaceutically acceptable salt or stereoisomer thereof, wherein:

n is 0-6 (e.g., n is 0, 1 or 2);

R¹ and R² are independently selected from H, (C₁-C₆)alkyl, heterocyclyl, and a polyamine, wherein said alkyl, heterocyclyl and polyamine are optionally substituted with one or more sub stituents selected from R′,

or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;

R³ is selected from H and (C₁-C₆)alkyl, wherein said alkyl is optionally substituted with one or more substituents selected from R′, or R³ can be taken together with R¹ to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;

each occurrence of R⁴, R^{3′} and R^{4′} is independently selected from H, (C₁-C₆)alkyl and O-alkyl, said alkyl is optionally substituted with one or more substituents selected from R′; or R^{3′} and R^{4′} when directly bound to the same carbon atom form an oxo (═O) group, cyclopropyl or cyclobutyl;

or R³ and R⁴ form an oxo (═O) group;

R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;

each occurrence of R′ is independently selected from halogen, R″, OR″, SR″, CN, CO₂R″ and CON(R″)₂;

each occurrence of R″ is selected from H and (C₁-C₆)alkyl, wherein said alkyl is optionally substituted with one or more substituents selected from halogen and OH;

L¹ is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more sub stituents selected from R′; and

L² is a C₄-C₂₂ alkyl or C₄-C₂₂ alkenyl, said alkyl or alkenyl is optionally interrupted by or terminated with one or more biodegradable groups; and said alkyl or alkenyl is optionally substituted with one or more sub stituents selected from R′;

with the proviso that the CR^{3′}R^{4′} group when present adjacent to the nitrogen atom in formula A is not a ketone (—C(O)—).

In another embodiment, the invention features a compound having Formula A, wherein:

L¹ and L² are

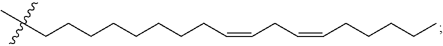 [SMILES: *CCCCCCCC/C=C\C/C=C\CCCCC]

and

all other variables are as defined in the first embodiment, or any pharmaceutically acceptable salt or stereoisomer thereof.

In another embodiment, L¹ is a C₄-C₂₂ alkyl or C₄-C₂₂ alkenyl interrupted by or terminated with one or more biodegradable groups. In yet another embodiment, L¹ is a C₄-C₂₂ alkyl or C₄-C₂₂ alkenyl interrupted or terminated with by one biodegradable group.

In another embodiment, L² is a C₄-C₂₂ alkyl or C₄-C₂₂ alkenyl interrupted by or terminated with one or more biodegradable groups. In yet another embodiment, L² is a C₄-C₂₂ alkyl or C₄-C₂₂ alkenyl interrupted by or terminated with one biodegradable group.

In another embodiment, each of L¹ and L² is, independently, a C₄-C₂₂ alkyl or C₄-C₂₂ alkenyl interrupted by or terminated with one or more biodegradable groups. In another embodiment, each of L¹ and L² is, independently, a C₄-C₂₂ alkyl or C₄-C₂₂ alkenyl interrupted by or terminated with one biodegradable group.

In another embodiment of this invention, the cationic lipids are illustrated by the Formula A:

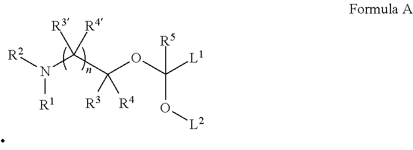

or a pharmaceutically acceptable salt or stereoisomer thereof, wherein:

n is 0, 1 or 2;

R¹ and R² are independently selected from H and (C₁-C₄)alkyl, wherein said alkyl is optionally substituted with one or more substituents selected from R′,

or R¹ and R² can be taken together with the nitrogen to which they are attached to form a monocyclic heterocycle which is optionally substituted with one or more substituents selected from R′;

R³ is selected from H and (C₁-C₄)alkyl, wherein said alkyl is optionally substituted with one or more substituents selected from R′, or R³ can be taken together with R¹ to form a monocyclic heterocycle which is optionally substituted with one or more substituents selected from R′, or R³ can be taken together with R⁴ to form cyclopropyl or cyclobutyl;

each occurrence of R⁴, R^{3′} and R^{4′} is independently selected from H and (C₁-C₄)alkyl, said alkyl is optionally substituted with one or more substituents selected from R′; or R^{3′} and R^{4′} when directly bound to a common carbon atom can form an oxo (═O) group, cyclopropyl or cyclobutyl;

R⁵ is selected from H and (C₁-C₄)alkyl, or R⁵ can be taken together with R¹ to form a monocyclic heterocycle which is optionally substituted with one or more substituents selected from R′;

R′ is independently selected from halogen, R″ and OR″;

R″ is selected from H and (C₁-C₄)alkyl, wherein said alkyl is optionally substituted with one or more substituents selected from halogen and OH;

L¹ is a C₄-C₂₂ alkyl or a C₄-C₂₂ alkenyl, said alkyl or alkenyl optionally interrupted by or terminated with one or more biodegradable groups; and

L² is a C₄-C₂₂ alkyl or a C₄-C₂₂ alkenyl, said alkyl or alkenyl optionally interrupted by or terminated with one or more biodegradable groups.

In another embodiment of this invention, the cationic lipids are illustrated by the Formula A:

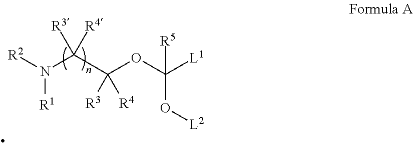

or a pharmaceutically acceptable salt or stereoisomer thereof, wherein:

n is 0, 1 or 2;

R¹ and R² are independently selected from H, methyl and ethyl, wherein said methyl and ethyl are optionally substituted with one or more sub stituents selected from R′, or

R¹ and R² can be taken together with the nitrogen to which they are attached to form a monocyclic heterocycle which is optionally substituted with one or more substituents selected from R¹;

R³ is selected from H, methyl and ethyl, wherein said methyl and ethyl are optionally substituted with one or more sub stituents selected from R′, or R³ can be taken together with R¹ to form a monocyclic heterocycle which is optionally substituted with one or more substituents selected from R′, or R³ can be taken together with R⁴ to form cyclopropyl;

each occurrence of R⁴, R^{3′} and R^{4′} is independently selected from H, methyl and ethyl, said methyl and ethyl are optionally substituted with one or more substituents selected from R′; or R^{3′} and R^{4′} when directly bound to a common carbon atom can form cyclopropyl;

R⁵ is selected from H, methyl and ethyl, or R⁵ can be taken together with R¹ to form a monocyclic heterocycle which is optionally substituted with one or more substituents selected from R′;

R′ is independently selected from OH and R″;

R″ is selected from H, methyl and ethyl, wherein said methyl and ethyl are optionally substituted with one or more substituents selected from halogen and OH;

L¹ is a C₄-C₂₂ alkyl or a C₄-C₂₂ alkenyl, said alkyl or alkenyl optionally interrupted by or terminated with one or more biodegradable groups; and

L² is a C₄-C₂₂ alkyl or a C₄-C₂₂ alkenyl, said alkyl or alkenyl optionally interrupted by or terminated with one or more biodegradable groups.

Yet another embodiment is a cationic lipid of formula B:

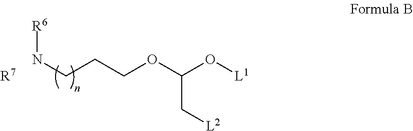

or a pharmaceutically acceptable salt or stereoisomer thereof, wherein

n is 0, 1, 2, 3, 4, or 5;

R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 sub stituents selected from R′; and

L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 sub stituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one preferred embodiment of the cationic lipid of formula B, R⁶ and R⁷ are methyl.

In another preferred embodiment of the cationic lipid of formula B, R⁶ and R⁷ together with the nitrogen atom adjacent to them form a 3 membered ring

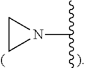 [SMILES: *N1CC1]

In one preferred embodiment of the cationic lipid of formula B, L¹ and L² are each independently C₄-C₂₂ alkenyl optionally substituted with 1-5 sub stituents selected from R′. In one more preferred embodiment, L¹ and L² are each independently unsubstituted C₄-C₂₂ alkenyl (e.g., C₁₆-C₂₀ alkenyl).

In another preferred embodiment, L¹ is a C₄-C₂₂ alkyl interrupted by or terminated with one or more biodegradable groups. In yet another preferred embodiment, L¹ is a C₄-C₂₂ alkyl interrupted by or terminated with one biodegradable group.

In another preferred embodiment, L² is a C₄-C₂₂ alkenyl interrupted by or terminated with one or more biodegradable groups. In yet another preferred embodiment, L² is a C₄-C₂₂ alkyl interrupted by or terminated with one biodegradable group.

In another preferred embodiment, each of L¹ and L² is, independently, a C₄-C₂₂ alkyl or C₄-C₂₂ alkenyl interrupted by or terminated with one or more biodegradable groups. In yet another preferred embodiment, each of L¹ and L² is, independently, a C₄-C₂₂ alkyl or C₄-C₂₂ alkenyl interrupted by or terminated with one biodegradable group.

Yet another embodiment is a cationic lipid of formula C:

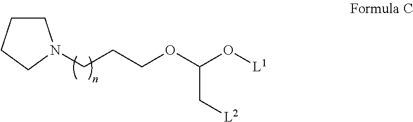

or a pharmaceutically acceptable salt or stereoisomer thereof, wherein

n is 0, 1, 2, 3, 4, or 5;

L¹ is a C₄-C₂₂ alkyl or C₄-C₂₂ alkenyl, said alkyl or alkenyl optionally has one or more biodegradable groups; each biodegradable group independently interrupts the alkyl or alkenyl group or is substituted at the terminus of the alkyl or alkenyl group, and said alkyl or alkenyl is optionally substituted with 1-5 sub stituents selected from R′; and

L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 sub stituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one preferred embodiment of the cationic lipid of formula C, one of L¹ and L² is a C₄-C₂₂ alkyl optionally substituted with 1-5 substituents selected from R′, and the other is a C₄-C₂₂ alkenyl optionally substituted with 1-5 sub stituents selected from R′.

In another preferred embodiment of the cationic lipid of formula C, one of L¹ and L² is a C₄-C₂₂ alkyl optionally interrupted by or terminated with one or more biodegradable groups, said alkyl is optionally substituted with 1-5 substituents selected from R′, and the other is a C₄-C₂₂ alkenyl optionally interrupted by or terminated with one or more biodegradable groups, and said alkenyl is optionally substituted with 1-5 substituents selected from R′.

In another embodiment, one of L¹ and L² is an unsubstituted C₄-C₂₂ alkyl, and the other is an unsubstituted C₄-C₂₂ alkenyl. For instance, in one embodiment, L¹ is an unsubstituted C₈-C₂₀ alkyl (e.g., C₁₄-C₁₈ alkyl) and L² is an unsubstituted C₁₄-C₂₂ alkenyl (e.g., C₁₆-C₂₀ alkenyl). In another embodiment, L¹ is an unsubstituted C₁₄-C₂₂ alkenyl (e.g., C₁₆-C₂₀ alkenyl) and L² is an unsubstituted C₈-C₂₀ alkyl (e.g., C₈-C₁₄ alkyl).

In another embodiment, one of L¹ and L² is an unsubstituted C₄-C₂₂ alkyl optionally interrupted by or terminated with one or more biodegradable groups; and the other is an unsubstituted C₄-C₂₂ alkenyl optionally interrupted by or terminated with one or more biodegradable groups.

For instance, in one embodiment, L¹ is an unsubstituted C₈-C₂₀ alkyl (e.g., C₁₄-C₁₈ alkyl) optionally interrupted by or terminated with one or more biodegradable groups, and L² is an unsubstituted C₁₄-C₂₂ alkenyl (e.g., C₁₆-C₂₀ alkenyl) optionally interrupted by or terminated with one or more biodegradable groups.

In another embodiment, L¹ is an unsubstituted C₁₄-C₂₂ alkenyl (e.g., C₁₆-C₂₀ alkenyl) optionally interrupted by or terminated with one or more biodegradable groups, and L² is an unsubstituted C₈-C₂₀ alkyl (e.g., C₈-C₁₄ alkyl) optionally interrupted by or terminated with one or more biodegradable groups.

Yet another embodiment is a cationic lipid of formula D:

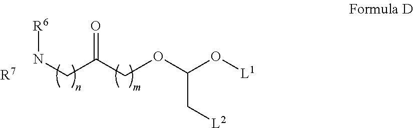

or a pharmaceutically acceptable salt or stereoisomer thereof, wherein

m is 0, 1, 2, or 3;

n is 0, 1, 2, 3, 4, or 5;

R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′; and

L² is a C₄-C₂₂ alkyl or C₄-C₂₂ alkenyl, said alkyl or alkenyl optionally interrupted by or terminated with one or more biodegradable groups; and said alkyl or alkenyl is optionally substituted with 1-5 substituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one embodiment of the cationic lipid of formula D, R⁶ and R⁷ are C₁-C₄ linear or branched alkyl.

In one preferred embodiment of the cationic lipid of formula D, R⁶ and R⁷ are methyl.

In another embodiment of the cationic lipid of formula D, R⁶ and R⁷ together with the nitrogen atom adjacent to them form a 3-6 membered ring. In one embodiment, R⁶ and R⁷ together with the nitrogen atom adjacent to them form a 3-membered ring

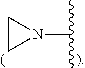 [SMILES: *N1CC1]

In one preferred embodiment, R⁶ and R⁷ together with the nitrogen atom adjacent to them form a 5-membered ring

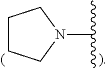 [SMILES: *N1CCCC1]

In one preferred embodiment of the cationic lipid of formula D, one of L¹ and L² is a C₄-C₂₂ alkyl optionally substituted with 1-5 substituents selected from R′, and the other is a C₄-C₂₂ alkenyl optionally substituted with 1-5 substituents selected from R′. In another embodiment, one of L¹ and L² is an unsubstituted C₄-C₂₂ alkyl, and the other is an unsubstituted C₄-C₂₂ alkenyl. For instance, in one embodiment, L¹ is an unsubstituted C₈-C₂₀ alkyl (e.g., C₁₄-C₁₈ alkyl) and L² is an unsubstituted C₁₄-C₂₂ alkenyl (e.g., C₁₆-C₂₀ alkenyl). In another embodiment, L¹ is an unsubstituted C₁₄-C₂₂ alkenyl (e.g., C₁₆-C₂₀ alkenyl) and L² is an unsubstituted C₈-C₂₀ alkyl (e.g., C₈-C₁₄ alkyl).

In another embodiment of the cationic lipid of formula D, one of L¹ and L² is a C₄-C₂₂ alkyl optionally interrupted by or terminated with one or more biodegradable groups, and said alkyl is optionally substituted with 1-5 substituents selected from R′, and the other is a C₄-C₂₂ alkenyl optionally interrupted by or terminated with one or more biodegradable groups, and said alkenyl is optionally substituted with 1-5 substituents selected from R′.

In yet another embodiment, one of L¹ and L² is an unsubstituted C₄-C₂₂ alkyl optionally interrupted by or terminated with one or more biodegradable groups; and the other is an unsubstituted C₄-C₂₂ alkenyl optionally interrupted by or terminated with one or more biodegradable groups.

For instance, in one embodiment, L¹ is an unsubstituted C₈-C₂₀ alkyl (e.g., C₁₄-C₁₈ alkyl) optionally interrupted by or terminated with one or more biodegradable groups; and L² is an unsubstituted C₁₄-C₂₂ alkenyl (e.g., C₁₆-C₂₀ alkenyl) optionally interrupted by or terminated with one or more biodegradable groups.

In another embodiment, L¹ is an unsubstituted C₁₄-C₂₂ alkenyl (e.g., C₁₆-C₂₀ alkenyl) optionally interrupted by or terminated with one or more biodegradable groups; and L² is an unsubstituted C₈-C₂₀ alkyl (e.g., C₈-C₁₄ alkyl) optionally interrupted by or terminated with one or more biodegradable groups.

Yet another embodiment is a cationic lipid of formula E:

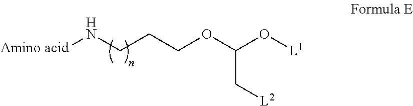

or a pharmaceutically acceptable salt or stereoisomer thereof, wherein

n is 0, 1, 2, 3, 4, or 5;

the group “amino acid” is an amino acid residue;

L¹ is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 sub stituents selected from R′; and

L² is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 sub stituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

The amino acid residue in formula E may have the formula —C(O)—C(R⁹)(NH₂), where R⁹ is an amino acid side chain.

Yet another embodiment is a cationic lipid of formula E′:

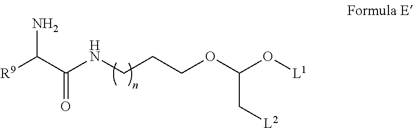

or a pharmaceutically acceptable salt thereof, wherein

n is 0, 1, 2, 3, 4, or 5;

R⁹ is an amino acid side chain;

L¹ is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 sub stituents selected from R′; and

L² is a C₄-C₂₂ alkyl or C₄-C₂₂ alkenyl, said alkyl or alkenyl optionally interrupted by or terminated with one or more biodegradable groups, and said alkyl or alkenyl is optionally substituted with 1-5 sub stituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

The “side chain” of an amino acid refers to the chemical moiety attached to the group containing the amino and carboxyl moieties. For example, many α-amino acids have the general formula

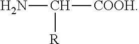

In one embodiment of the cationic lipid of Formula E′, R⁹ is an amino acid side chain of a naturally occurring amino acid residue of a naturally occurring amino acid optionally substituted with 1-5 R′. In another embodiment, R⁹ is an amino acid side chain of one of the standard 20 amino acids optionally substituted with 1-5 R′.

In another embodiment of the cationic lipid of Formula E′, R⁹ is an amino acid side chain of a naturally occurring amino acid and is not further substituted. In yet another embodiment, R⁹ is an amino acid side chain of one of the standard 20 amino acids and is not further substituted.

In one embodiment of the cationic lipid of formula E or E′, L¹ and L² are each independently C₄-C₂₂ alkenyl optionally interrupted by or terminated with one or more biodegradable groups, and said alkenyl is optionally substituted with 1-5 substituents selected from R′.

In one more preferred embodiment, L¹ and L² are each independently unsubstituted C₄-C₂₂ alkenyl (e.g., C₁₆-C₂₀ alkenyl) optionally interrupted by or terminated with one or more biodegradable groups.

In another embodiment of the cationic lipid of formula E or E′, one of L¹ and L² is a C₄-C₂₂ alkyl optionally interrupted by or terminated with one or more biodegradable groups, and said alkyl is optionally substituted with 1-5 substituents selected from R′, and the other is a C₄-C₂₂ alkenyl optionally interrupted by or terminated with one or more biodegradable groups, and said alkenyl is optionally substituted with 1-5 substituents selected from R′.

In another embodiment, one of L¹ and L² is an unsubstituted C₄-C₂₂ alkyl, optionally interrupted by or terminated with one or more biodegradable groups, and the other is an unsubstituted C₄-C₂₂ alkenyl optionally interrupted by or terminated with one or more biodegradable groups.

For instance, in one embodiment, L¹ is an unsubstituted C₈-C₂₀ alkyl (e.g., C₁₄-C₁₈ alkyl) optionally interrupted by or terminated with one or more biodegradable groups, and L² is an unsubstituted C₁₄-C₂₂ alkenyl (e.g., C₁₆-C₂₀ alkenyl) optionally interrupted by or terminated with one or more biodegradable groups.

In another embodiment, L¹ is an unsubstituted C₁₄-C₂₂ alkenyl (e.g., C₁₆-C₂₀ alkenyl) optionally interrupted by or terminated with one or more biodegradable groups, and L² is an unsubstituted C₈-C₂₀ alkyl (e.g., C₈-C₁₄ alkyl) optionally interrupted by or terminated with one or more biodegradable groups.

Examples of an amino acid side chain include those having a releasing functional group having a pKa from about 5 to about 7.5, or from about 6 to about 7. In general, a releasing functional group which is a weak base may exhibit a predominant neutral form at a local pH above pKa, and may exhibit a predominant ionic form at a local pH below pKa. A releasing functional group which is a weak acid may exhibit an ionic form at a local pH above pKa, and may exhibit a neutral form at a local pH below pKa. See, e.g., P. Heinrich Stahl, Handbook of Pharmaceutical Salts, (2002). Examples of a substituent on a side chain of an amino acid suitable for a releasable form of an amino acid lipid include, but are not limited to, releasing functional groups derived from 3,5-diiodo-tyrosine, 1-methylhistidine, 2-methylbutanoic acid, 2-o-anisylpropanoic acid, meso-tartaric acid, 4,6-dimethylpyrimidinamine, p-phthalic acid, creatinine, butanoic acid, N,N-dimethyl-1-naphthylamine, pentanoic acid, 4-methylpentanoic acid, N-methylaniline, 1,10-phenanthroline, 3-pyridinecarboxylic acid, hexanoic acid, propanoic acid, 4-aminobenzoic acid, 2-methylpropanoic acid, heptanoic acid, octanoic acid, cyclohexanecarboxylic acid, quinoline, 3-quinolinamine, 2-aminobenzoic acid, 4-pyridinecarboxylic acid, nonanoic acid, melamine, 8-quinolinol, trimethylacetic acid, 6-methoxyquinoline, 4-(methylamino)benzoic acid, p-methylaniline, 3-(methylamino)benzoic acid, malic acid, N-ethylaniline, 2-benzylpyridine, 3,6-dinitrophenol, N,N-dimethylaniline, 2,5-dimethylpiperazine, p-phenetidine, 5-methylquinoline, 2-phenylbenzimidazole, pyridine, picolinic acid, 3,5-diiodotyrosine, p-anisidine, 2-(methylamino)benzoic acid, 2-thiazolamine, glutaric acid, adipic acid, isoquinoline, itaconic acid, o-phthalic acid, benzimidazole, piperazine, heptanedioic acid, acridine, phenanthridine, succinic acid, methylsuccinic acid, 4-methylquinoline, 3-methylpyridine, 7-isoquinolinol, malonic acid, methylmalonic acid, 2-methylquinoline, 2-ethylpyridine, 2-methylpyridine, 4-methylpyridine, histamine, histidine, maleic acid, cis-1,2-cyclohexanediamine, 3,5-dimethylpyridine, 2-ethylbenzimidazole, 2-methylbenzimidazole, cacodylic acid, perimidine, citric acid, isocitric acid, 2,5-dimethylpyridine, papaverine, 6-hydroxy-4-methylpteridine, L-thyroxine, 3,4-dimethylpyridine, methoxypyridine, trans-1,2-cyclohexanediamine, 2,5-pyridinediamine, 1-1-methylhistidine, 1-3-methylhistidine, 2,3-dimethylpyridine, xanthopterin, 1,2-propanediamine, N,N-diethylaniline, alloxanic acid, 2,6-dimethylpyridine, L-carnosine, 2-pyridinamine, N-b-alanylhistidine, pilocarpine, 1-methylimidazol, 1H-imidazole, 2,4-dimethylpyridine, 4-nitrophenol, 2-nitrophenol, tyrosinamide, 5-hydroxyquinazoline, 1,1-cyclopropanedicarboxylic acid, 2,4,6-trimethylpyridine, veronal, 2,3-dichlorophenol, 1,2-ethanediamine, 1-isoquinolinamine, and combinations thereof. For example, examples of a substituted side chain of an amino acid suitable for a releasable form of an amino acid lipid include (1) 1-methylhistidine and (2) 3,5-diiodo-tyrosine.

Other examples of a substituted side chain of an amino acid suitable for a releasable form of an amino acid lipid include the following structures:

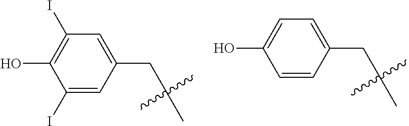 [SMILES: *Cc1cc(I)c(O)c(I)c1 ; *Cc1ccc(O)cc1]

In one embodiment, the amino acid side chain is basic. Examples of amino acids having a basic side chain include arginine (Arg), homoarginine (homoArg) (side chain —(CH₂)₄NH(C═NH)NH₂), norarginine (norArg) (side chain —(CH₂)₂NH(C═NH)NH₂), nor-norarginine (nornorArg) (side chain —(CH₂)NH(C═NH)NH₂), ornithine, lysine, homolysine, histidine, 1-methylhistidine, pyridylalanine (Pal), asparagine, N-ethylasparagine, glutamine, and 4-aminophenylalanine. The side chain of any of these amino acids may be used. In some embodiments, the amino acid side chain is that from cysteine or serine.

Examples of side chains include the following structures, as well as their salt forms:

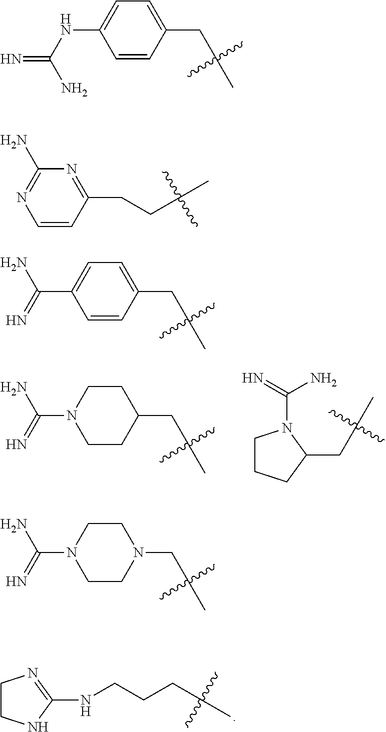

Yet another embodiment is a cationic lipid of formula F:

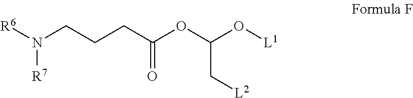

or a pharmaceutically acceptable salt or stereoisomer thereof, wherein

R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

each occurrence of R′ is independently selected from halogen, R″, OR″, SR″, CN, CO₂R″ and CON(R″)₂;

each occurrence of R″ is independently selected from H and (C₁-C₆)alkyl, wherein said alkyl is optionally substituted with one or more substituents selected from halogen and OH.

In one preferred embodiment of the cationic lipid of formula F, R⁶ and R⁷ are methyl.

In another preferred embodiment of the cationic lipid of formula F, R⁶ and R⁷ together with the nitrogen atom adjacent to them form a 3 membered ring

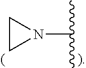 [SMILES: *N1CC1]

In one embodiment of the cationic lipid of formula F, L¹ and L² are each independently C₄-C₂₂ alkenyl optionally interrupted by or terminated with one or more biodegradable groups, and said alkenyl is optionally substituted with 1-5 substituents selected from R′.

In one more preferred embodiment, L¹ and L² are each independently unsubstituted C₄-C₂₂ alkenyl (e.g., C₁₆-C₂₀ alkenyl) optionally interrupted by or terminated with one or more biodegradable groups.

In another embodiment of the cationic lipid of formula F, one of L¹ and L² is a C₄-C₂₂ alkyl optionally interrupted by or terminated with one or more biodegradable groups, and said alkyl is optionally substituted with 1-5 substituents selected from R′, and the other is a C₄-C₂₂ alkenyl optionally interrupted by or terminated with one or more biodegradable groups, and said alkenyl is optionally substituted with 1-5 sub stituents selected from R′.

In another embodiment, one of L¹ and L² is an unsubstituted C₄-C₂₂ alkyl optionally interrupted by or terminated with one or more biodegradable groups, and the other is an unsubstituted C₄-C₂₂ alkenyl optionally interrupted by or terminated with one or more biodegradable groups.

For instance, in one embodiment, L¹ is an unsubstituted C₈-C₂₀ alkyl (e.g., C₁₄-C₁₈ alkyl) optionally interrupted by or terminated with one or more biodegradable groups, and L² is an unsubstituted C₁₄-C₂₂ alkenyl (e.g., C₁₆-C₂₀ alkenyl) optionally interrupted by or terminated with one or more biodegradable groups.

In another embodiment, L¹ is an unsubstituted C₁₄-C₂₂ alkenyl (e.g., C₁₆-C₂₀ alkenyl) optionally interrupted by or terminated with one or more biodegradable groups, and L² is an unsubstituted C₈-C₂₀ alkyl (e.g., C₈-C₁₄ alkyl) optionally interrupted by or terminated with one or more biodegradable groups.

Yet another embodiment is a cationic lipid of formula G:

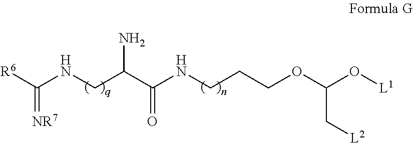

or a pharmaceutically acceptable salt or stereoisomer thereof, wherein

n is 0, 1, 2, 3, 4, or 5;

q is 1, 2, 3, or 4

R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl);

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

each occurrence of R′ is independently selected from halogen, R″, OR″, SR″, CN, CO₂R″ and CON(R″)₂;

each occurrence of R″ is independently selected from H and (C₁-C₆)alkyl, wherein said alkyl is optionally substituted with one or more substituents selected from halogen and OH.

In one embodiment of the cationic lipid of formula G, R⁶ and R⁷ are methyl.

In one embodiment of the cationic lipid of formula G′, L¹ and L² are each independently C₄-C₂₂ alkenyl optionally interrupted by or terminated with one or more biodegradable groups, and said alkenyl is optionally substituted with 1-5 substituents selected from R′.

In one more preferred embodiment, L¹ and L² are each independently unsubstituted C₄-C₂₂ alkenyl (e.g., C₁₆-C₂₀ alkenyl) optionally interrupted by or terminated with one or more biodegradable groups.

In another embodiment of the cationic lipid of formula G, one of L¹ and L² is a C₄-C₂₂ alkyl optionally interrupted by or terminated with one or more biodegradable groups, and said alkyl is optionally substituted with 1-5 substituents selected from R′, and the other is a C₄-C₂₂ alkenyl optionally interrupted by or terminated with one or more biodegradable groups, and said alkenyl is optionally substituted with 1-5 sub stituents selected from R′.

In another embodiment, one of L¹ and L² is an unsubstituted C₄-C₂₂ alkyl optionally interrupted by or terminated with one or more biodegradable groups, and the other is an unsubstituted C₄-C₂₂ alkenyl optionally interrupted by or terminated with one or more biodegradable groups.

For instance, in one embodiment, L¹ is an unsubstituted C₈-C₂₀ alkyl (e.g., C₁₄-C₁₈ alkyl), optionally interrupted by or terminated with one or more biodegradable groups, and L² is an unsubstituted C₁₄-C₂₂ alkenyl (e.g., C₁₆-C₂₀ alkenyl) optionally interrupted by or terminated with one or more biodegradable groups.

In another embodiment, L¹ is an unsubstituted C₁₄-C₂₂ alkenyl (e.g., C₁₆-C₂₀ alkenyl), optionally interrupted by or terminated with one or more biodegradable groups, and L² is an unsubstituted C₈-C₂₀ alkyl (e.g., C₈-C₁₄ alkyl) optionally interrupted by or terminated with one or more biodegradable groups.

In one embodiment of any of Formulas A-G shown above, each of L¹ and L² is interrupted by or terminated with one or more biodegradable groups. In one embodiment of any of Formulas A-G shown above, each of L¹ and L² is interrupted by or terminated with one biodegradable group.

3b) Cationic Lipids with Acetal and Biodegradable Tail Groups

In another embodiment of this invention, the cationic lipid is of Formula A1:

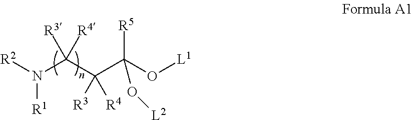

or a pharmaceutically acceptable salt or stereoisomer thereof, wherein:

n is 0-6 (e.g., n is 0, 1 or 2);

R¹ and R² are independently selected from H, (C₁-C₆)alkyl, heterocyclyl, and a polyamine, wherein said alkyl, heterocyclyl and polyamine are optionally substituted with one or more substituents selected from R′,

or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;

R³ is selected from H and (C₁-C₆)alkyl, wherein said alkyl is optionally substituted with one or more substituents selected from R′, or R³ can be taken together with R¹ to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′;

each occurrence of R⁴, R^{3′} and R^{4′} is independently selected from H, (C₁-C₆)alkyl and O-alkyl, said alkyl is optionally substituted with one or more substituents selected from R′; or R^{3′} and R^{4′} when directly bound to the same carbon atom form an oxo (═O) group, cyclopropyl or cyclobutyl;

or R³ and R⁴ form an oxo (═O) group;

R⁵ is selected from H and (C₁-C₆)alkyl; or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′;

R′ is independently selected from halogen, R″, OR″, SR″, CN, CO₂R″ and CON(R″)₂;

R″ is selected from H and (C₁-C₆)alkyl, wherein said alkyl is optionally substituted with one or more substituents selected from halogen and OH;

L¹ is a C₄-C₂₂ alkyl or C₄-C₂₂ alkenyl, said alkyl or alkenyl optionally interrupted by or terminated with one or more biodegradable groups, and said alkyl or alkenyl is optionally substituted with one or more sub stituents selected from R′; and

L² is a C₄-C₂₂ alkyl or C₄-C₂₂ alkenyl, said alkyl or alkenyl optionally interrupted by or terminated with one or more biodegradable groups, and said alkyl or alkenyl is optionally substituted with one or more sub stituents selected from R′;

wherein at last one of L¹ or L² is interrupted by or terminated with one or more biodegradable groups; and with the proviso that the CR^{3′}R^{4′} group when present adjacent to the nitrogen atom in formula A is not a ketone (—C(O)—).

In another embodiment, L¹ is a C₄-C₂₂ alkyl or C₄-C₂₂ alkenyl interrupted by or terminated with one or more biodegradable groups.

In another embodiment, L² is a C₄-C₂₂ alkyl or C₄-C₂₂ alkenyl interrupted by or terminated with one or more biodegradable groups.

In another embodiment, each of L¹ and L² is, independently, a C₄-C₂₂ alkyl or C₄-C₂₂ alkenyl interrupted by or terminated with one or more biodegradable groups. In yet another embodiment, each of L¹ and L² is, independently, a C₄-C₂₂ alkyl or C₄-C₂₂ alkenyl interrupted by or terminated with one biodegradable group.

In another embodiment of this invention, the cationic lipids are illustrated by the Formula A1:

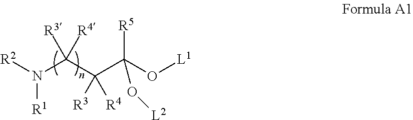

or a pharmaceutically acceptable salt or stereoisomer thereof, wherein:

n is 0, 1 or 2;

R¹ and R² are independently selected from H and (C₁-C₄)alkyl, wherein said alkyl is optionally substituted with one or more substituents selected from R′,

or R¹ and R² can be taken together with the nitrogen to which they are attached to form a monocyclic heterocycle which is optionally substituted with one or more substituents selected from R′;

R³ is selected from H and (C₁-C₄)alkyl, wherein said alkyl is optionally substituted with one or more substituents selected from R′, or R³ can be taken together with R¹ to form a monocyclic heterocycle which is optionally substituted with one or more substituents selected from R′, or R³ can be taken together with R⁴ to form cyclopropyl or cyclobutyl;

each occurrence of R⁴, R^{3′} and R^{4′} is independently selected from H and (C₁-C₄)alkyl, said alkyl is optionally substituted with one or more substituents selected from R′; or R^{3′} and R^{4′} when directly bound to a common carbon atom can form an oxo (═O) group, cyclopropyl or cyclobutyl;

R⁵ is selected from H and (C₁-C₄)alkyl, or R⁵ can be taken together with R¹ to form a monocyclic heterocycle which is optionally substituted with one or more substituents selected from R′;

R′ is independently selected from halogen, R″ and OR″;

R″ is selected from H and (C₁-C₄)alkyl, wherein said alkyl is optionally substituted with one or more substituents selected from halogen and OH;

L¹ is a C₄-C₂₂ alkyl or a C₄-C₂₂ alkenyl optionally interrupted by or terminated with one or more biodegradable groups; and

L² is a C₄-C₂₂ alkyl or a C₄-C₂₂ alkenyl optionally interrupted by or terminated with one or more biodegradable groups;

wherein at last one of L¹ or L² is interrupted by or terminated with one or more biodegradable groups.

In another embodiment of this invention, the cationic lipids are illustrated by the Formula A1:

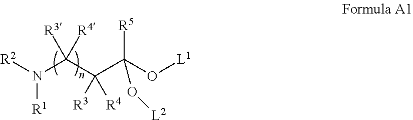

or a pharmaceutically acceptable salt or stereoisomer thereof, wherein:

n is 0, 1 or 2;

R¹ and R² are independently selected from H, methyl and ethyl, wherein said methyl and ethyl are optionally substituted with one or more sub stituents selected from R′, or

R¹ and R² can be taken together with the nitrogen to which they are attached to form a monocyclic heterocycle which is optionally substituted with one or more substituents selected from R¹;

R³ is selected from H, methyl and ethyl, wherein said methyl and ethyl are optionally substituted with one or more sub stituents selected from R′, or R³ can be taken together with R¹ to form a monocyclic heterocycle which is optionally substituted with one or more substituents selected from R′, or R³ can be taken together with R⁴ to form cyclopropyl;

each occurrence of R⁴, R^{3′} and R^{4′} is independently selected from H, methyl and ethyl, said methyl and ethyl are optionally substituted with one or more substituents selected from R′; or R^{3′} and R^{4′} when directly bound to a common carbon atom can form cyclopropyl;

R⁵ is selected from H, methyl and ethyl, or R⁵ can be taken together with R¹ to form a monocyclic heterocycle which is optionally substituted with one or more substituents selected from R′;

R′ is independently selected from OH and R″;

R″ is selected from H, methyl and ethyl, wherein said methyl and ethyl are optionally substituted with one or more substituents selected from halogen and OH;

L¹ is a C₄-C₂₂ alkyl or a C₄-C₂₂ alkenyl optionally interrupted by or terminated with one or more biodegradable groups; and

L² is a C₄-C₂₂ alkyl or a C₄-C₂₂ alkenyl optionally interrupted by or terminated with one or more biodegradable groups;

wherein at last one of L¹ or L² is interrupted by or terminated with one or more biodegradable groups.

Yet another embodiment is a cationic lipid of the formula B1:

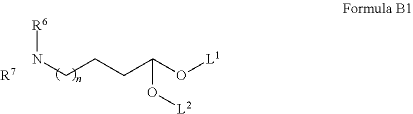

or a pharmaceutically acceptable salt or stereoisomer thereof, wherein

n is 0, 1, 2, 3, 4, or 5;

R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one embodiment of the cationic lipid of formula B1, R⁶ and R⁷ are methyl.

In another embodiment of the cationic lipid of formula B1, R⁶ and R⁷ together with the nitrogen atom adjacent to them form a 3 membered ring

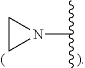 [SMILES: *N1CC1]

In one embodiment of the cationic lipid of formula B1, L¹ and L² are each independently C₄-C₂₂ alkenyl optionally substituted with 1-5 sub stituents selected from R′, with at least one of L¹ and L² interrupted by or terminated with a biodegradable group. In one more embodiment, L¹ and L² are each independently unsubstituted C₄-C₂₂ alkenyl (e.g., C₁₆-C₂₀ alkenyl), with at least one of L¹ and L² interrupted by or terminated with a biodegradable group.

In another embodiment, L¹ is a C₄-C₂₂ alkyl interrupted by or terminated with one or more biodegradable groups.

In another embodiment, L² is a C₄-C₂₂ alkenyl interrupted by or terminated with one or more biodegradable groups.

In another embodiment, each of L¹ and L² is, independently, a C₄-C₂₂ alkyl or C₄-C₂₂ alkenyl interrupted by or terminated with one or more biodegradable groups. In yet another embodiment, each of L¹ and L² is, independently, a C₄-C₂₂ alkyl or C₄-C₂₂ alkenyl interrupted by or terminated with one biodegradable groups.

Yet another embodiment is a cationic lipid of the formula C1:

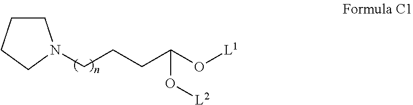

or a pharmaceutically acceptable salt or stereoisomer thereof, wherein

n is 0, 1, 2, 3, 4, or 5;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one embodiment of the cationic lipid of formula C1, one of L¹ and L² is a C₄-C₂₂ alkyl interrupted by or terminated with one or more biodegradable groups, and said alkyl is optionally substituted with 1-5 substituents selected from R′, and the other is a C₄-C₂₂ alkenyl interrupted by or terminated with one or more biodegradable groups, and said alkenyl is optionally substituted with 1-5 sub stituents selected from R′.

In another embodiment, one of L¹ and L² is an unsubstituted C₄-C₂₂ alkyl interrupted by or terminated with one or more biodegradable groups, and the other is an unsubstituted C₄-C₂₂ alkenyl interrupted by or terminated with one or more biodegradable groups.

For instance, in one embodiment, L¹ is an unsubstituted C₈-C₂₀ alkyl (e.g., C₁₄-C₁₈ alkyl) interrupted by or terminated with one or more biodegradable groups, and L² is an unsubstituted C₁₄-C₂₂ alkenyl (e.g., C₁₆-C₂₀ alkenyl) interrupted by or terminated with one or more biodegradable groups.

Yet another embodiment is a cationic lipid of the formula D1:

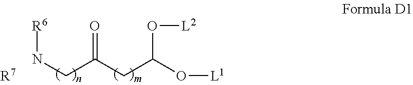

or a pharmaceutically acceptable salt or stereoisomer thereof, wherein

m is 0, 1, 2, or 3;

n is 0, 1, 2, 3, 4, or 5;

R⁶ and R⁷ are each independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

In one embodiment of the cationic lipid of formula D1, R⁶ and R⁷ are C₁-C₄ linear or branched alkyl.

In one embodiment of the cationic lipid of formula D1, R⁶ and R⁷ are methyl.

In another embodiment of the cationic lipid of formula D1, R⁶ and R⁷ together with the nitrogen atom adjacent to them form a 3-6 membered ring. In one embodiment, R⁶ and R⁷ together with the nitrogen atom adjacent to them form a 3-membered ring

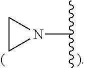 [SMILES: *N1CC1]

In one preferred embodiment, R⁶ and R⁷ together with the nitrogen atom adjacent to them form a 5-membered ring

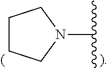 [SMILES: *N1CCCC1]

In one embodiment of the cationic lipid of formula D1, one of L¹ and L² is a C₄-C₂₂ alkyl interrupted by or terminated with one or more biodegradable groups, and said alkyl is optionally substituted with 1-5 substituents selected from R′, and the other is a C₄-C₂₂ alkenyl interrupted by or terminated with one or more biodegradable groups, and said alkenyl is optionally substituted with 1-5 substituents selected from R′.

In another embodiment, one of L¹ and L² is an unsubstituted C₄-C₂₂ alkyl interrupted by or terminated with one or more biodegradable groups, and the other is an unsubstituted C₄-C₂₂ alkenyl interrupted by or terminated with one or more biodegradable groups.

For instance, in one embodiment, L¹ is an unsubstituted C₈-C₂₀ alkyl (e.g., C₁₄-C₁₈ alkyl) interrupted by or terminated with one or more biodegradable groups, and L² is an unsubstituted C₁₄-C₂₂ alkenyl (e.g., C₁₆-C₂₀ alkenyl) interrupted by or terminated with one or more biodegradable groups.

Yet another embodiment is a cationic lipid of the formula E1:

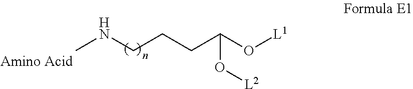

or a pharmaceutically acceptable salt or stereoisomer thereof, wherein

n is 0, 1, 2, 3, 4, or 5; the group “amino acid” is an amino acid residue;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

The amino acid residue in formula E may have the formula —C(O)—C(R⁹)(NH₂), where R⁹ is an amino acid side chain.

Yet another embodiment is a cationic lipid of the formula E1′:

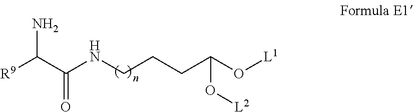

or a pharmaceutically acceptable salt thereof, wherein

n is 0, 1, 2, 3, 4, or 5;

R⁹ is an amino acid side chain;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂; and

each occurrence of R″ is independently selected from H and (C₁-C₆)alkyl, wherein said alkyl is optionally substituted with one or more substituents selected from halogen and OH.

The “side chain” of an amino acid refers to the chemical moiety attached to the group containing the amino and carboxyl moieties. For example, many α-amino acids have the general formula

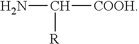

In one embodiment of the cationic lipid of Formula E1′, R⁹ is an amino acid side chain of a naturally occurring amino acid residue of a naturally occurring amino acid optionally substituted with 1-5 R′. In another embodiment, R⁹ is an amino acid side chain of one of the standard 20 amino acids optionally substituted with 1-5 R′.

In another embodiment of the cationic lipid of Formula E1′, R⁹ is an amino acid side chain of a naturally occurring amino acid and is not further substituted. In yet another embodiment, R⁹ is an amino acid side chain of one of the standard 20 amino acids and is not further substituted.

In one embodiment of the cationic lipid of formula E1′, one of L¹ and L² is a C₄-C₂₂ alkyl interrupted by or terminated with one or more biodegradable groups, and said alkyl is optionally substituted with 1-5 substituents selected from R′, and the other is a C₄-C₂₂ alkenyl interrupted by or terminated with one or more biodegradable groups, and said alkenyl is optionally substituted with 1-5 substituents selected from R′.

In another embodiment, one of L¹ and L² is an unsubstituted C₄-C₂₂ alkyl interrupted by or terminated with one or more biodegradable groups, and the other is an unsubstituted C₄-C₂₂ alkenyl interrupted by or terminated with one or more biodegradable groups.

For instance, in one embodiment, L¹ is an unsubstituted C₈-C₂₀ alkyl (e.g., C₁₄-C₁₈ alkyl) interrupted by or terminated with one or more biodegradable groups, and L² is an unsubstituted C₁₄-C₂₂ alkenyl (e.g., C₁₆-C₂₀ alkenyl) interrupted by or terminated with one or more biodegradable groups.

Examples of amino acid side chains include those described above.

Yet another embodiment is a cationic lipid of the formula F1:

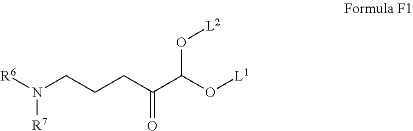

or a pharmaceutically acceptable salt or stereoisomer thereof, wherein

R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl); or R⁶ and R⁷ together with the nitrogen atom adjacent to them form a 3-6 membered ring;

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂;

each occurrence of R″ is independently selected from H and (C₁-C₆)alkyl, wherein said alkyl is optionally substituted with one or more substituents selected from halogen and OH.

In one embodiment of the cationic lipid of formula F1, R⁶ and R⁷ are methyl.

In another embodiment of the cationic lipid of formula F1, R⁶ and R⁷ together with the nitrogen atom adjacent to them form a 3 membered ring

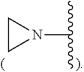 [SMILES: *N1CC1]

In one embodiment of the cationic lipid of formula F1, one of L¹ and L² is a C₄-C₂₂ alkyl interrupted by or terminated with one or more biodegradable groups, and said alkyl is optionally substituted with 1-5 substituents selected from R′, and the other is a C₄-C₂₂ alkenyl interrupted by or terminated with one or more biodegradable groups, and said alkenyl is optionally substituted with 1-5 sub stituents selected from R′.

In another embodiment, one of L¹ and L² is an unsubstituted C₄-C₂₂ alkyl interrupted by or terminated with one or more biodegradable groups, and the other is an unsubstituted C₄-C₂₂ alkenyl interrupted by or terminated with one or more biodegradable groups.

For instance, in one embodiment, L¹ is an unsubstituted C₈-C₂₀ alkyl (e.g., C₁₄-C₁₈ alkyl) interrupted by or terminated with one or more biodegradable groups, and L² is an unsubstituted C₁₄-C₂₂ alkenyl (e.g., C₁₆-C₂₀ alkenyl) interrupted by or terminated with one or more biodegradable groups.

Yet another embodiment is a cationic lipid of the formula G1:

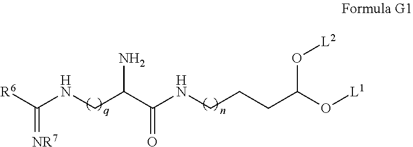

or a pharmaceutically acceptable salt or stereoisomer thereof, wherein

n is 0, 1, 2, 3, 4, or 5;

q is 1, 2, 3, or 4

R⁶ and R⁷ are independently (i) C₁-C₄ linear or branched alkyl (e.g., methyl or ethyl) optionally substituted with 1-4 R′, or (ii) C₃-C₈ cycloalkyl (e.g., C₃-C₆ cycloalkyl);

L¹ is a C₄-C₂₂ alkyl or C₄-C₂₂ alkenyl optionally interrupted by or terminated with one or more biodegradable groups, and said alkyl or alkenyl is optionally substituted with 1-5 substituents selected from R′; and

L² is a C₄-C₂₂ alkyl or C₄-C₂₂ alkenyl optionally interrupted by or terminated with one or more biodegradable groups, and said alkyl or alkenyl is optionally substituted with 1-5 substituents selected from R′;

wherein at last one of L¹ or L² is interrupted by or terminated with one or more biodegradable groups;

each occurrence of R′ is independently selected from halogen, R″, OR″, SR″, CN, CO₂R″ and CON(R″)₂;

each occurrence of R″ is independently selected from H and (C₁-C₆)alkyl, wherein said alkyl is optionally substituted with one or more substituents selected from halogen and OH.

In one embodiment of the cationic lipid of formula G1, R⁶ and R⁷ are methyl.

In one embodiment of the cationic lipid of formula G1, one of L¹ and L² is a C₄-C₂₂ alkyl interrupted by or terminated with one or more biodegradable groups, and said alkyl is optionally substituted with 1-5 substituents selected from R′, and the other is a C₄-C₂₂ alkenyl interrupted by or terminated with one or more biodegradable groups, and said alkenyl is optionally substituted with 1-5 sub stituents selected from R′.

In another embodiment, one of L¹ and L² is an unsubstituted C₄-C₂₂ alkyl interrupted by or terminated with one or more biodegradable groups, and the other is an unsubstituted C₄-C₂₂ alkenyl interrupted by or terminated with one or more biodegradable groups.

For instance, in one embodiment, L¹ is an unsubstituted C₈-C₂₀ alkyl (e.g., C₁₄-C₁₈ alkyl) interrupted by or terminated with one or more biodegradable groups, and L² is an unsubstituted C₁₄-C₂₂ alkenyl (e.g., C₁₆-C₂₀ alkenyl) interrupted by or terminated with one or more biodegradable groups.

Yet another embodiment is a lipid particle that includes a cationic lipid as described in any embodiment herein.

In one embodiment, the lipid particle includes a compound of any of formulas III-XXIV as described herein. In another embodiment, the lipid particle includes a compound of formula I or II as described herein. In another embodiment, the lipid particle includes a compound of formula IA, IB, IC or ID. In another embodiment, the lipid particle includes a compound of formula IIA, IIB, IIC or IID.

In a preferred embodiment, the lipid particle includes a neutral lipid, a lipid capable of reducing aggregation, a cationic lipid, and optionally, a sterol (e.g., cholesterol). Suitable neutral lipids include, but are not limited to, distearoylphosphatidylcholine (DSPC), dipalmitoylphosphatidylcholine (DPPC), POPC, DOPE, and SM. Suitable lipids capable of reducing aggregation include, but are not limited to, a PEG lipid, such as PEG-DMA, PEG-DMG, or a combination thereof.

The lipid particle may further include an active agent (e.g., a therapeutic agent). The active agent can be a nucleic acid such as a plasmid, an immunostimulatory oligonucleotide, an siRNA, an antisense oligonucleotide, a microRNA, an antagomir, an aptamer, or a ribozyme. In a preferred embodiment, the nucleic acid is a siRNA. In another preferred embodiment, the nucleic acid is a miRNA.

In another embodiment, the lipid particle includes a cationic lipid of the present invention, a neutral lipid and a sterol. The lipid particle may further include an active agent, such as a nucleic acid (e.g., an siRNA or miRNA).

The lipid particles described herein may be lipid nanoparticles.

Yet another embodiment of the invention is a pharmaceutical composition which includes a lipid particle of the present invention and a pharmaceutically acceptable carrier.

Yet another embodiment is a method of delivering a nucleic acid molecule in a subject comprising administering to the subject a lipid particle comprising the nucleic acid molecule and a cationic lipid (or a salt thereof), the cationic lipid having

(i) a central carbon or nitrogen atom,

(ii) an amino acid containing head group directly bound to the central carbon or nitrogen atom, and

(iii) two hydrophobic tails directly bound to the central carbon or nitrogen atom, each hydrophobic tail comprising a C₈ or greater aliphatic group (preferably a C₁₄ or greater aliphatic group) attached to the central carbon or nitrogen atom, where one or both of the aliphatic group(s) (a) is interrupted by a biodegradable group such that there is a chain of at least four carbon atoms between the biodegradable group and the central carbon or nitrogen atom, or (b) includes a biodegradable group at the terminal end of the hydrophobic tail.

Yet another embodiment is a method of delivering a nucleic acid molecule in a subject comprising administering to the subject a lipid particle comprising the nucleic acid molecule and a cationic lipid (or a salt thereof), the cationic lipid having

(i) a central nitrogen or phosphorous atom,

(ii) an amino acid containing head group directly bound to the central carbon or nitrogen atom, and

(iii) two hydrophobic tails directly bound to the central carbon or nitrogen atom, each hydrophobic tail comprising a C₈ or greater aliphatic group (preferably a C₁₄ or greater aliphatic group) attached to the central carbon or nitrogen atom, where one or both of the aliphatic group(s) (a) is interrupted by a biodegradable group such that there is a chain of at least four carbon atoms between the biodegradable group and the central carbon or nitrogen atom, or (b) includes a biodegradable group at the terminal end of the hydrophobic tail.

In another embodiment, the present invention relates to a method of delivering a nucleic acid molecule comprising administering a nucleic lipid particle comprising the nucleic acid molecule and a cationic lipid of the present invention. In one embodiment, the cationic lipid remains intact until delivery of the nucleic acid molecule after which cleavage of the hydrophobic tail occurs in vivo.

In one embodiment, the cationic lipid remains intact until delivery of the nucleic acid molecule after which cleavage of the hydrophobic tail occurs in vivo.

Yet another aspect is a method of modulating the expression of a target gene in a cell by providing to the cell a lipid particle of the present invention. The active agent can be a nucleic acid selected from a plasmid, an immunostimulatory oligonucleotide, an siRNA, an antisense oligonucleotide, a microRNA, an antagomir, an aptamer, and a ribozyme.

Yet another aspect is a method of treating a disease or disorder characterized by the overexpression of a polypeptide in a subject by providing to the subject a pharmaceutical composition of the present invention, wherein the active agent is a nucleic acid selected from an siRNA, a microRNA, and an antisense oligonucleotide, and wherein the siRNA, microRNA, or antisense oligonucleotide includes a polynucleotide that specifically binds to a polynucleotide that encodes the polypeptide, or a complement thereof.

Yet another aspect is a method of treating a disease or disorder characterized by underexpression of a polypeptide in a subject by providing to the subject a pharmaceutical composition of the present invention, wherein the active agent is a plasmid that encodes the polypeptide or a functional variant or fragment thereof.

Yet another aspect is a method of inducing an immune response in a subject by providing to the subject a pharmaceutical composition wherein the active agent is an immunostimulatory oligonucleotide.

Yet another aspect is a transfection agent that includes the composition or lipid particles described above, where the composition or lipid particles include a nucleic acid. The agent, when contacted with cells, can efficiently deliver nucleic acids to the cells. Yet another aspect is a method of delivering a nucleic acid to the interior of a cell, by obtaining or forming a composition or lipid particles described above, and contacting the composition or lipid particles with a cell.

### DETAILED DESCRIPTION

In one aspect, the present invention relates to a lipid particle that includes a neutral lipid, a lipid capable of reducing aggregation, an amino acid conjugate cationic lipid, and optionally a sterol. In certain embodiments, the lipid particle further includes an active agent (e.g., a therapeutic agent). Various exemplary embodiments of these lipids, lipid particles and compositions comprising the same, and their use to deliver therapeutic agents and modulate gene and protein expression are described in further detail below.

### The Cationic Lipid

### Amino Acid

In some aspects, amino acid lipids of this disclosure may provide delivery of a therapeutic agent in a releasable form. Releasable forms and compositions are designed to provide sufficient uptake of an agent by a cell to provide a therapeutic effect.

Releasable forms include amino acid lipids that bind and release an active agent. In some embodiments, release of the active agent may be provided by an acid-labile linker.

Examples of acid-labile linkers include linkers containing an orthoester group, a hydrazone, a cis-acetonyl, an acetal, a ketal, a silyl ether, a silazane, an imine, a citraconic anhydride, a maleic anhydride, a crown ether, an azacrown ether, a thiacrown ether, a dithiobenzyl group, a cis-aconitic acid, a cis-carboxylic alkatriene, methacrylic acid, and mixtures thereof.

Examples of acid-labile groups and linkers are given in for example, U.S. Pat. Nos. 7,098,032, 6,897,196, 6,426,086, 7,138,382, 5,563,250, and 5,505,931.

Releasable forms of compounds and compositions of this disclosure include molecules that bind an active agent and discharge a moiety that assists in release of the agent. In some embodiments, an amino acid lipid may include a group which releases a small molecule such as ethanol that assists in delivering an agent to a cell. An amino acid lipid may bind an active agent and, subsequent to contact with a cell, or subsequent to transport within a biological compartment having a local pH lower than physiological pH, be hydrolyzed in an acidic environment to release ethanol to assist in delivery of the agent. In some embodiments, a small molecule such as ethanol, which assists in delivery of the agent, may be bound to a lipid component.

In some embodiments, an amino acid lipid may be admixed with a compound that releases a small molecule such as ethanol to assists in delivering an agent to a cell.

Releasable forms of compounds and compositions of this disclosure include amino acid lipids which may bind an active agent and, subsequent to contact with a cell, or subsequent to transport within a biological compartment having a local pH lower than physiological pH, be modulated in an acidic environment into a cationic form to assist in release of the agent.

In some embodiments, an amino acid lipid may bind an active agent, and may be admixed with a compound that can be modulated in an acidic environment into a cationic form to assist in release of an active agent.

Examples of hydrolysable and modulatable groups are given in, for example, U.S. Pat. Nos. 6,849,272 and 6,200,599; as well as Z. H. Huang et al., “Bioresponsive liposomes and their use for macromolecular delivery,” in: G. Gregoriadis (ed.), Liposome Technology, 3rd ed. (CRC Press 2006).

In some embodiments, releasable forms of compounds and compositions of this disclosure include amino acid lipids which can bind an active agent, and may be admixed with a lipid or compound that can be modulated in an acidic environment into a neutral form to assist in release of an active agent. The acidic environment may be entered subsequent to contact with a cell, or subsequent to transport within a biological compartment having a local pH lower than physiological pH.

Examples of lipids which are modulatable from anionic to neutral forms include cholesteryl hemisuccinate (CHEMS) as described in U.S. Pat. Nos. 6,897,196, 6,426,086 and 7,108,863.

Examples of a substituted side chain (e.g., corresponding to R¹ in formula (I)) of an amino acid suitable for a releasable form of an amino acid lipid include a releasing functional group having a pKa from about 5 to about 7.5, or from about 6 to about 7. In general, a releasing functional group which is a weak base may exhibit a predominant neutral form at a local pH above pKa, and may exhibit a predominant ionic form at a local pH below pKa. A releasing functional group which is a weak acid may exhibit an ionic form at a local pH above pKa, and may exhibit a neutral form at a local pH below pKa. See, e.g., P. Heinrich Stahl, Handbook of Pharmaceutical Salts, (2002). Examples of a substituent on a side chain of an amino acid suitable for a releasable form of an amino acid lipid include, but are not limited to, releasing functional groups derived from 3,5-diiodo-tyrosine, 1-methylhistidine, 2-methylbutanoic acid, 2-o-anisylpropanoic acid, meso-tartaric acid, 4,6-dimethylpyrimidinamine, p-phthalic acid, creatinine, butanoic acid, N,N-dimethyl-1-naphthylamine, pentanoic acid, 4-methylpentanoic acid, N-methylaniline, 1,10-phenanthroline, 3-pyridinecarboxylic acid, hexanoic acid, propanoic acid, 4-aminobenzoic acid, 2-methylpropanoic acid, heptanoic acid, octanoic acid, cyclohexanecarboxylic acid, quinoline, 3-quinolinamine, 2-aminobenzoic acid, 4-pyridinecarboxylic acid, nonanoic acid, melamine, 8-quinolinol, trimethylacetic acid, 6-methoxyquinoline, 4-(methylamino)benzoic acid, p-methylaniline, 3-(methylamino)benzoic acid, malic acid, N-ethylaniline, 2-benzylpyridine, 3,6-dinitrophenol, N,N-dimethylaniline, 2,5-dimethylpiperazine, p-phenetidine, 5-methylquinoline, 2-phenylbenzimidazole, pyridine, picolinic acid, 3,5-diiodotyrosine, p-anisidine, 2-(methylamino)benzoic acid, 2-thiazolamine, glutaric acid, adipic acid, isoquinoline, itaconic acid, o-phthalic acid, benzimidazole, piperazine, heptanedioic acid, acridine, phenanthridine, succinic acid, methylsuccinic acid, 4-methylquinoline, 3-methylpyridine, 7-isoquinolinol, malonic acid, methylmalonic acid, 2-methylquinoline, 2-ethylpyridine, 2-methylpyridine, 4-methylpyridine, histamine, histidine, maleic acid, cis-1,2-cyclohexanediamine, 3,5-dimethylpyridine, 2-ethylbenzimidazole, 2-methylbenzimidazole, cacodylic acid, perimidine, citric acid, isocitric acid, 2,5-dimethylpyridine, papaverine, 6-hydroxy-4-methylpteridine, L-thyroxine, 3,4-dimethylpyridine, methoxypyridine, trans-1,2-cyclohexanediamine, 2,5-pyridinediamine, 1-1-methylhistidine, 1-3-methylhistidine, 2,3-dimethylpyridine, xanthopterin, 1,2-propanediamine, N,N-diethylaniline, alloxanic acid, 2,6-dimethylpyridine, L-carnosine, 2-pyridinamine, N-b-alanylhistidine, pilocarpine, 1-methylimidazol, 1H-imidazole, 2,4-dimethylpyridine, 4-nitrophenol, 2-nitrophenol, tyrosinamide, 5-hydroxyquinazoline, 1,1-cyclopropanedicarboxylic acid, 2,4,6-trimethylpyridine, veronal, 2,3-dichlorophenol, 1,2-ethanediamine, 1-isoquinolinamine, and combinations thereof.

In some embodiments, Xaa may have a side chain (e.g., corresponding to R¹ in formula (I)) containing a functional group having a pKa from 5 to 7.5. Examples of a substituted side chain of an amino acid suitable for a releasable form of an amino acid lipid include (1) 1-methylhistidine and (2) 3,5-diiodo-tyrosine.

Examples of a substituted side chain of an amino acid suitable for a releasable form of an amino acid lipid include the following structures:

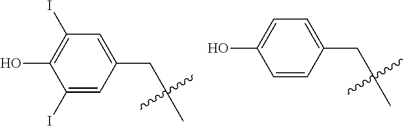 [SMILES: *Cc1cc(I)c(O)c(I)c1 ; *Cc1ccc(O)cc1]

In another embodiment, Xaa may have a side chain containing a functional group having a pKa from 5 to 7.5.

In one embodiment, Xaa has a basic side chain. Examples of amino acids having a basic side chain include arginine (Arg), homoarginine (homoArg) (side chain —(CH₂)₄NH(C═NH)NH₂), norarginine (norArg) (side chain —(CH₂)₂NH(C═NH)NH₂), nor-norarginine (nornorArg) (side chain —(CH₂)NH(C═NH)NH₂), ornithine, lysine, homolysine, histidine, 1-methylhistidine, pyridylalanine (Pal), asparagine, N-ethylasparagine, glutamine, and 4-aminophenylalanine, N-methylated versions thereof, and side chain modified derivatives thereof. In some embodiments, Xaa is selected from cysteine and serine.

As used herein, the term “homo,” when referring to an amino acid, means that an additional carbon is added to the side chain, while the term “nor,” when referring to an amino acid, means that a carbon is subtracted from the side chain. Thus, homolysine refers to side chain —(CH₂)₅NH₂.

Examples of Xaa side chains include the following structures, as well as their salt forms:

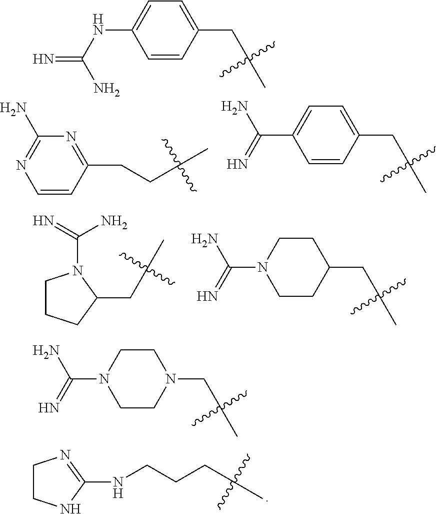

In one embodiment, Xaa is a residue of a naturally occurring amino acid. In another embodiment, Xaa is a peptide of one or more naturally occurring amino acids. In yet another embodiment, all the amino acids in the peptide Xaa are naturally occurring amino acids. For example, a naturally occurring amino acid having the formula NHR^{N}—CR¹R²—(C═O)OH would provide a residue of the formula —NR^{N}—CR¹R²—(C═O)—. In yet another embodiment, Xaa is one of the standard 20 amino acids. In yet another embodiment, Xaa is a peptide of one or more of the standard 20 amino acids. In yet another embodiment, all of the amino acids in the peptide Xaa are naturally occurring amino acids.

Lipids

In one embodiment, the cationic lipid is a compound of formula I-XXIV. In another embodiment, the cationic lipid is a compound of one of formulas III-XXIV. In one embodiment, the cationic lipid is a compound of formula I of formula II. In another embodiment, the cationic lipid is a compound of formula IA, IB, IC or ID. In another embodiment, the cationic lipid is a compound of formula IIA, IIB, IIC or IID. In another embodiment, the cationic lipid is a compound of formulas I-7. In another embodiment, the cationic lipid is a compound of formulas 8-18. In another embodiment, the cationic lipid is a compound of formulas 19-25.

In one embodiment, M¹ and M² are each, independently:

—OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—.

In another embodiment, M¹ and M² are each, independently:

—OC(O)—, —C(O)—O—, —C(R⁵)═N—, —N═C(R⁵)—, —C(R⁵)═N—O—, —O—N═C(R⁵)—, —O—C(O)O—, —C(O)N(R⁵)—, —N(R⁵)C(O)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—.

In yet another embodiment, M¹ and M² are each, independently:

—C(O)—O—, —OC(O)—, —C(R⁵)═N—, —C(R⁵)═N—O—, —O—C(O)O—, —C(O)N(R⁵)—, —C(O)S—, —C(S)O—, —OSi(R⁵)₂O—, —C(O)(CR³R⁴)C(O)O—, or —OC(O)(CR³R⁴)C(O)—.

In another embodiment, M¹ and M² are each —C(O)O— or —OC(O)—.

For cationic lipid compounds which contain an atom (e.g., a nitrogen atom) that carries a positive charge, the compound also contains a negatively charged counter ion. The counterion can be any anion, such as an organic or inorganic anion. Suitable examples of anions include, but are not limited to, tosylate, methanesulfonate, acetate, citrate, malonate, tartarate, succinate, benzoate, ascorbate, a-ketoglutarate, a-glycerophosphate, halide (e.g., chloride), sulfate, nitrate, bicarbonate, and carbonate. In one embodiment, the counterion is a halide (e.g., Cl).

In one embodiment each R is, independently, —(CR³R⁴)—, wherein R³ and R⁴ are each, independently, H or alkyl (e.g., C₁-C₄ alkyl). For example, in one embodiment each R is, independently, —(CHR⁴)—, wherein each R⁴ is, independently H or alkyl (e.g., C₁-C₄ alkyl). In another embodiment, each R is, independently, —CH₂—, —C(CH₃)₂— or —CH(iPr)— (where iPr is isopropyl). In another embodiment, each R is —CH₂—.

In another embodiment R⁵ is, in each case, hydrogen or methyl. For example, R⁵ can be, in each case, hydrogen.

In one embodiment, Q is absent, —C(O)O—, —OC(O)—, —C(O)N(R⁴)—, —N(R⁵)C(O)—, —S—S—, —OC(O)O—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—. In one embodiment, Q is —C(O)O—.

In one embodiment, Q¹ and Q² are each, independently, absent or —O—. For example, in one embodiment, Q¹ and Q² are each absent. In another embodiment, Q¹ and Q² are each —O—.

In one embodiment, the cationic lipid is a compound of subformula:

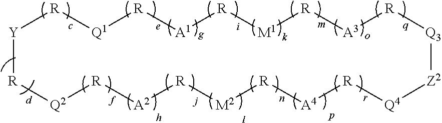

wherein

### Y is —C(O)-Xaa-Z—, —Z-Xaa-C(O)—, or

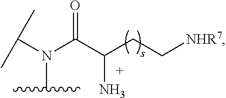

wherein Xaa and Z are defined with respect to formula (I) and R⁷ and s are defined with respect to formula (II); and

R, A¹, A², A³, A⁴, Q¹, Q², Q³, Q⁴, Z², c, d, e, f, g, h, i, j, k, l, m, n, o, p, q and r are as defined in any of the embodiments disclosed herein.

In additional embodiments of the compound of subformula shown above, one or more of the following applies:

(i) Q¹ and Q² are absent;

(ii) M¹ and M² are both —C(O)O—;

(iii) g and h are both 1;

(iv) g and h are both 0;

(v) c and e total 7;

(vi) d and f total 7;

(vii) c, e, and i total 7;

(viii) d, f and j total 7;

(ix) i and j are each 7;

(x) k and l are both 1;

(xi) m and n are both 0;

(xii) m and q total 1 or m and q total 2;

(xiii) m and l total 6;

(xiv) r and n total 6;

(xv) p and o are both 0;

(xvi) n and r total 2 or n and r total 1; and

(xvii) Q³ is H.

In certain embodiments, the biodegradable group present in the cationic lipid is selected from an ester (e.g., —C(O)O— or —OC(O)—), disulfide (—S—S—), oxime (e.g., —C(H)═N—O— or —O—N═C(H)—), —C(O)—O—, —OC(O)—, —C(R⁵)═N—, —N═C(R⁵)—, —C(R⁵)═N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, —OC(O)(CR³R⁴)C(O)—, or

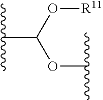

(wherein R¹¹ is a C₂-C₈ alkyl or alkenyl).

In one embodiment, the aliphatic group in one or both of the hydrophobic tails of the cationic lipid includes at least one carbon-carbon double bond.

In one embodiment, the cationic lipid is a compound of any one of Formulas I-64. The following disclosure represents various embodiments of the compounds described above, including one or more of the compounds of Formulas 1-64.

In one embodiment, M¹ and M² are each, independently:

—OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

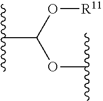

(wherein R¹¹ is a C₂-C₈ alkyl or alkenyl).

In another embodiment, M¹ and M² are each, independently: —OC(O)—, —C(O)—O—, —C(R⁵)═N—, —N═C(R⁵)—, —C(R⁵)═N—O—, —O—N═C(R⁵)—, —O—C(O)O—, —C(O)N(R⁵)—, —N(R⁵)C(O)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—.

In yet another embodiment, M¹ and M² are each, independently:

—C(O)—O—, —OC(O)—, —C(R⁵)═N—, —C(R⁵)═N—O—, —O—C(O)O—, —C(O)N(R⁵)—, —C(O)S—, —C(S)O—, —OSi(R⁵)₂O—, —C(O)(CR³R⁴)C(O)O—, or —OC(O)(CR³R⁴)C(O)—.

In another embodiment, M¹ and M² are each —C(O)O—.

In one embodiment, R¹ and R² are each, individually, optionally substituted alkyl, cycloalkyl, cycloalkylalkyl, or heterocycle. In one embodiment, R¹ is alkyl and R² is alkyl, cycloalkyl or cycloalkylalkyl. In one embodiment, R¹ and R² are each, individually, alkyl (e.g., C₁-C₄ alkyl, such as methyl, ethyl, or isopropyl). In one embodiment, R¹ and R² are both methyl. In another embodiment, R¹ and R², together with the nitrogen atom to which they are attached, form an optionally substituted heterocylic ring (e.g., N-methylpiperazinyl). In another embodiment, one of R¹ and R² is

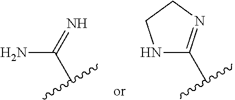

(e.g., R¹ is one of the two aforementioned groups and R² is hydrogen).

In one embodiment, R′ is hydrogen or alkyl. In another embodiment, R′ is hydrogen or methyl. In one embodiment, R′ is absent. In one embodiment, R′ is absent or methyl.

A suitable cholesterol moiety for the cationic lipids of the present invention (including compounds of formulas (I), (IA), (II) and (IIA)) has the formula:

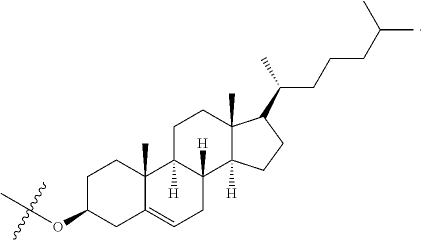

Additional embodiments include a cationic lipid having a head group, one or more hydrophobic tails, and a linker between the amino acid head group and the one or more tails. The head group can include an amine; for example an amine having a desired pKₐ. The pKₐ can be influenced by the structure of the lipid, particularly the nature of head group; e.g., the presence, absence, and location of functional groups such as anionic functional groups, hydrogen bond donor functional groups, hydrogen bond acceptor groups, hydrophobic groups (e.g., aliphatic groups), hydrophilic groups (e.g., hydroxyl or methoxy), or aryl groups. The head group amine can be a cationic amine; a primary, secondary, or tertiary amine; the head group can include one amine group (monoamine), two amine groups (diamine), three amine groups (triamine), or a larger number of amine groups, as in an oligoamine or polyamine. The head group can include a functional group that is less strongly basic than an amine, such as, for example, an imidazole, a pyridine, or a guanidinium group. The head group can be zwitterionic. Other head groups are suitable as well.

The one or more hydrophobic tails can include two hydrophobic chains, which may be the same or different. The tails can be aliphatic, for example, they can be composed of carbon and hydrogen, either saturated or unsaturated but without aromatic rings. The tails can be fatty acid tails. Some such groups include octanyl, nonanyl, decyl, lauryl, myristyl, palmityl, stearyl, α-linoleyl, stearidonyl, linoleyl, γ-linolenyl, arachadonyl, and oleyl. Other hydrophobic tails are suitable as well.

The linker can include, for example, a glyceride linker, an acyclic glyceride analog linker, or a cyclic linker (including a spiro linker, a bicyclic linker, and a polycyclic linker). The linker can include functional groups such as an ether, an ester, a phosphate, a phosphonate, a phosphorothioate, a sulfonate, a disulfide, an acetal, a ketal, an imine, a hydrazone, or an oxime. Other linkers and functional groups are suitable as well.

In one embodiment, the cationic lipid is a racemic mixture. In another embodiment, the cationic lipid is enriched in one diastereomer, e.g. the cationic lipid has at least 95%, at least 90%, at least 80% or at least 70% diastereomeric excess. In yet another embodiment, the cationic lipid is enriched in one enantiomer, e.g. the lipid has at least 95%, at least 90%, at least 80% or at least 70% enantiomer excess. In yet another embodiment, the cationic lipid is chirally pure, e.g. is a single optical isomer. In yet another embodiment, the cationic lipid is enriched for one optical isomer.

Where a double bond is present (e.g., a carbon-carbon double bond or carbon-nitrogen double bond), there can be isomerism in the configuration about the double bond (i.e. cis/trans or E/Z isomerism). Where the configuration of a double bond is illustrated in a chemical structure, it is understood that the corresponding isomer can also be present. The amount of isomer present can vary, depending on the relative stabilities of the isomers and the energy required to convert between the isomers. Accordingly, some double bonds are, for practical purposes, present in only a single configuration, whereas others (e.g., where the relative stabilities are similar and the energy of conversion low) may be present as inseparable equilibrium mixture of configurations.

In some cases, a double-bonded unsaturation can be replaced by a cyclic unsaturation. The cyclic unsaturation can be a cycloaliphatic unsaturation, e.g., a cyclopropyl, cyclobutyl, cyclopentyl, cyclohexyl, cycloheptyl, or cyclooctyl group. In some cases, the cyclic group can be a polycyclic group, e.g., a bicyclic group or tricyclic group. A bicyclic group can be bridged, fused, or have a spiro structure.

In some cases, a double bond moiety can be replaced by a cyclopropyl moiety, e.g.,

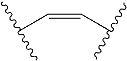 [SMILES: */C=C\*]

can be replaced by

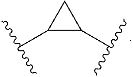 [SMILES: *C1CC1*]

For example, the moiety shown below has two carbon-carbon double bonds, each of which can independently be replaced by a cyclic moiety, e.g., a cyclopropyl moiety. Thus, substitutes for:

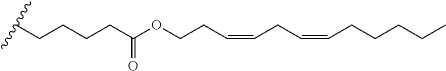 [SMILES: *CCCCC(=O)OCC/C=C\C/C=C\CCCCC]

can include:

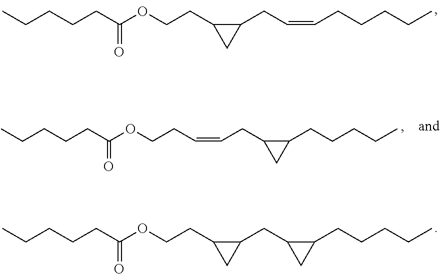

For further example, substitutes for

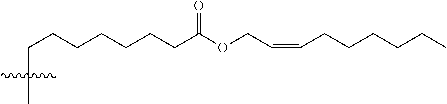 [SMILES: *CCCCCCCC(=O)OC/C=C\CCCCCC]

include:

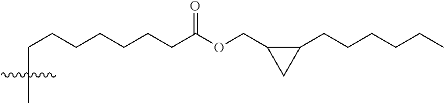 [SMILES: *CCCCCCCC(=O)OCC1CC1CCCCCC]

For further example, substitutes for

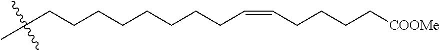 [SMILES: *CCCCCCCC/C=C\CCCCC(=O)OC]

include:

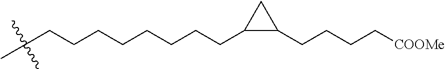 [SMILES: *CCCCCCCCC1(C)CC1CCCCC(=O)OC]

For further example, substitutes for

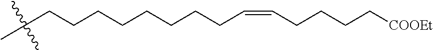 [SMILES: *CCCCCCCC/C=C\CCCCC(=O)OCC]

include:

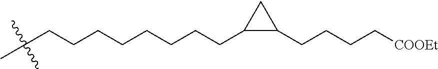 [SMILES: *CCCCCCCCC1(C)CC1CCCCC(=O)OCC]

The cationic lipid may include one or more biodegradable groups. The biodegradable group(s) include one or more bonds that may undergo bond breaking reactions in a biological environment, e.g., in an organism, organ, tissue, cell, or organelle. Functional groups that contain a biodegradable bond include, for example, esters, dithiols, and oximes. Biodegradation can be a factor that influences the clearance of the compound from the body when administered to a subject. Biodegredation can be measured in a cell based assay, where a formulation including a cationic lipid is exposed to cells, and samples are taken at various time points. The lipid fractions can be extracted from the cells and separated and analyzed by LC-MS. From the LC-MS data, rates of biodegradation (e.g., as t_{1/2} values) can be measured.

For example, compounds of the formula:

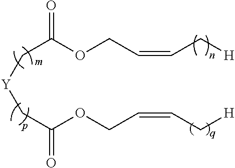

in which Y, m, n, p and q are as defined herein, includes an ester linkage in each aliphatic chain, which can undergo hydrolysis in a biological environment, for example, when exposed to, e.g., a lipase or an esterase. The structure of the compound, of course, influences the rate at which the compound undergoes biodegradation. Thus, a related compound such as

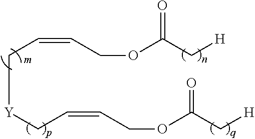

in which Y, m, n, p and q are as defined herein, would be expected to exhibit a different rate of biodegradation. Greater effects on that rate would be expected from changes in the structure of the compound at the site of hydrolysis. One modification that can influence the rate of hydrolysis, and thereby influence the rate of biodegradation and clearance from a subject's body, is to make the leaving group of the hydrolysis reaction have a primary, rather than secondary, alcohol.

For example, without wishing to be bound by theory, a compound of the formula:

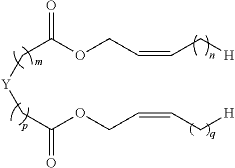

may be metabolized as shown in the scheme below.

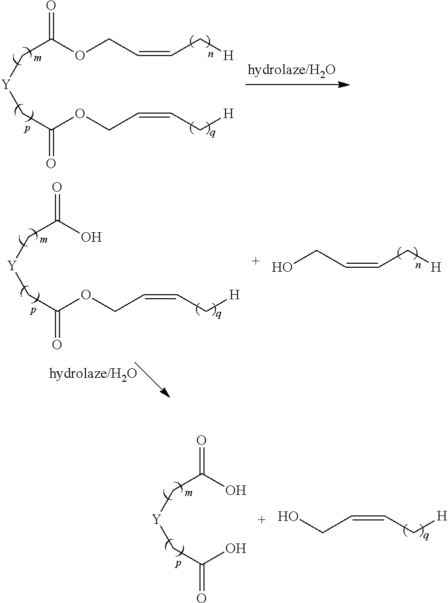

Some suitable hydrophobic tail groups include those depicted in Table 2A:

TABLE 2A

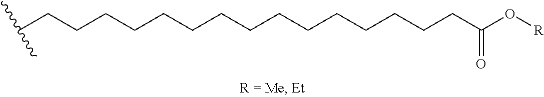

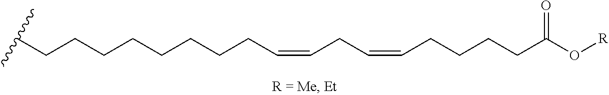

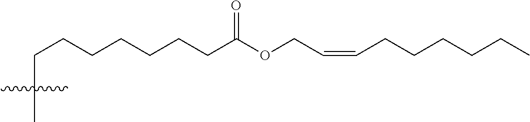 [SMILES: *CCCCCCCC(=O)OC/C=C\CCCCCC]

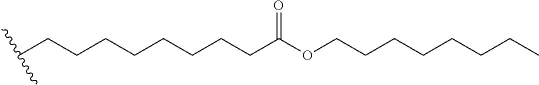 [SMILES: *CCCCCCCCC(=O)OCCCCCCCC]

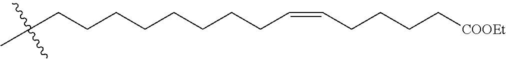 [SMILES: *CCCCCCCC/C=C\CCCCC(=O)OCC]

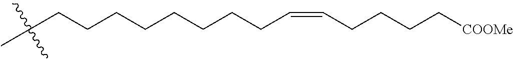 [SMILES: *CCCCCCCC/C=C\CCCCC(=O)OC]

Some additional suitable hydrophobic tail groups include those depicted in Table 2B. Each hydrophilic tail group may be attached, for example, to the central nitrogen or phosphorous atom in a compound of Formula (I)

TABLE 2B

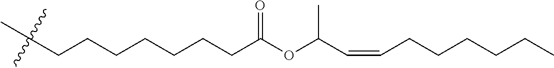 [SMILES: *CCCCCCCC(=O)OC(C)/C=C\CCCCCC]

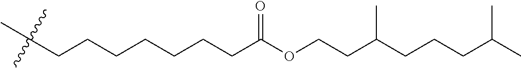 [SMILES: *CCCCCCCC(=O)OCCC(C)CCCC(C)C]

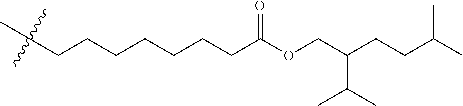 [SMILES: *CCCCCCCC(=O)OCC(CCC(C)C)C(C)C]

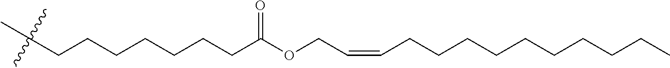 [SMILES: *CCCCCCCC(=O)OC/C=C\CCCCCCCCCC]

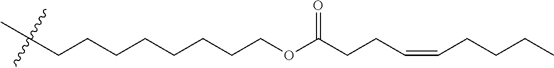 [SMILES: *CCCCCCCCOC(=O)CC/C=C\CCCC]

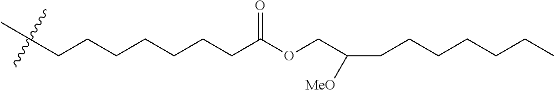 [SMILES: *CCCCCCCC(=O)OCC(CCCCCCC)OC]

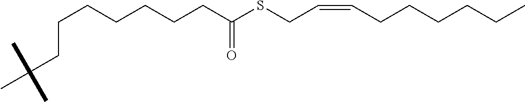

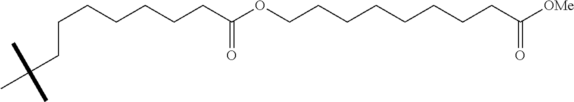

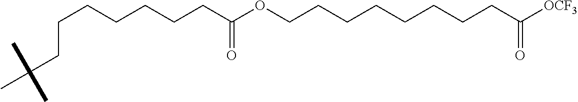

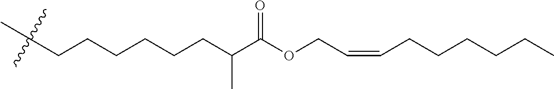 [SMILES: *CCCCCCC(C)C(=O)OC/C=C\CCCCCC]

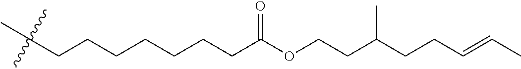 [SMILES: *CCCCCCCC(=O)OCCC(C)CC/C=C/C]

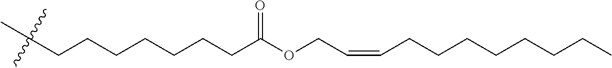 [SMILES: *CCCCCCCC(=O)OC/C=C\CCCCCCCC]

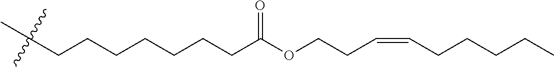 [SMILES: *CCCCCCCC(=O)OCC/C=C\CCCCC]

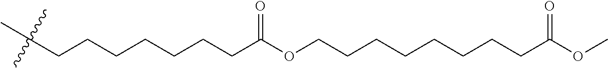 [SMILES: *CCCCCCCC(=O)OCCCCCCCCC(=O)OC]

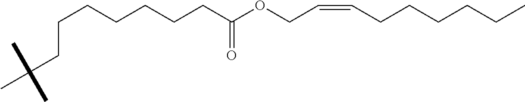

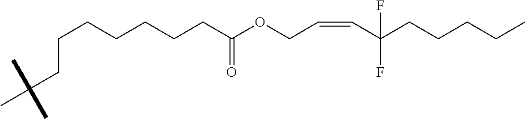

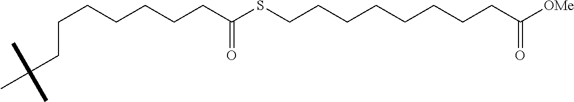

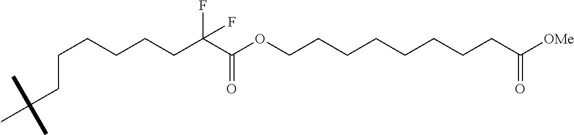

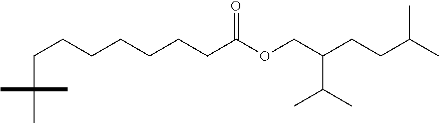

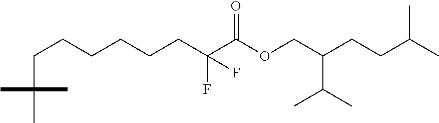

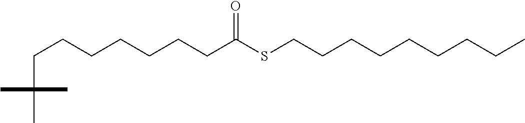

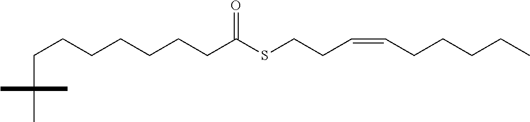

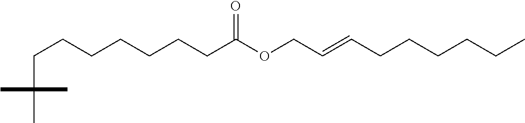

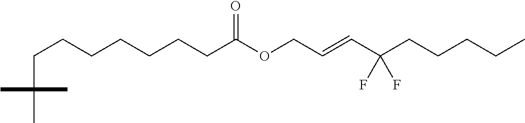

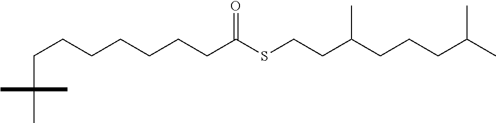

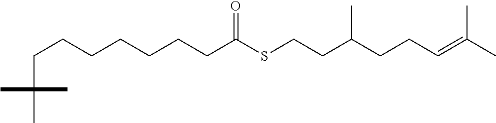

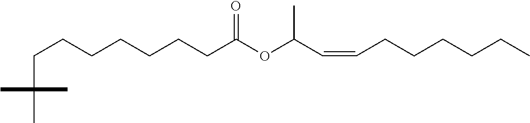

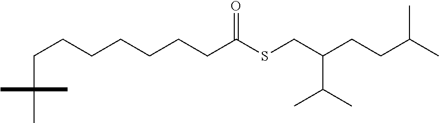

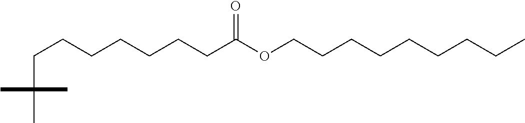

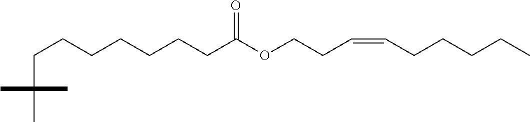

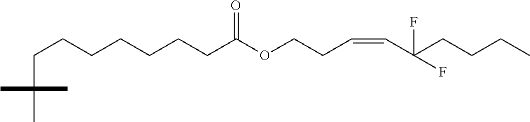

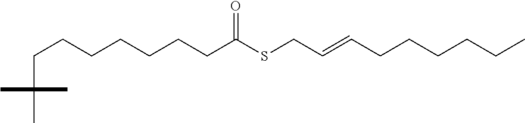

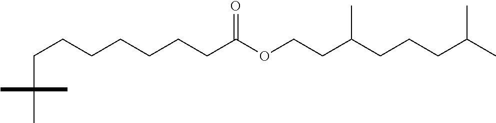

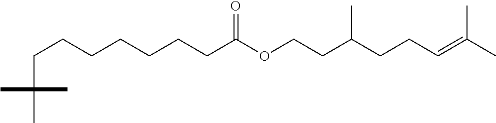

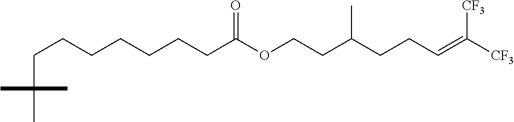

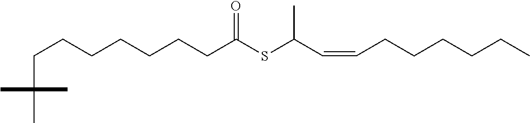

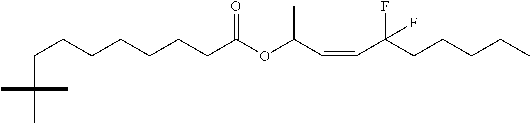

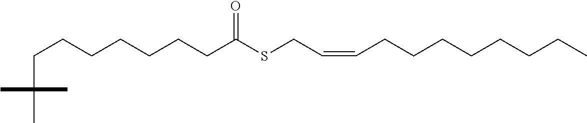

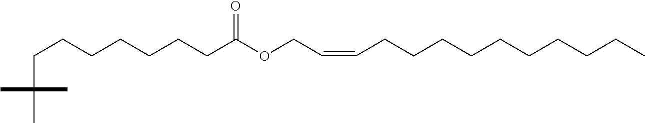

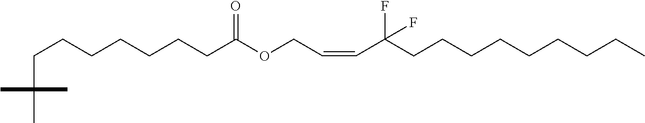

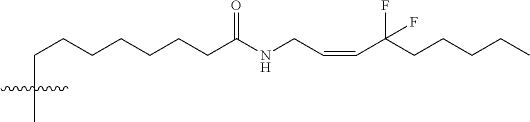 [SMILES: *CCCCCCCC(=O)NC/C=C\C(F)(F)CCCCC]

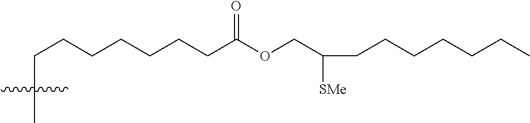 [SMILES: *CCCCCCCC(=O)OCC(CCCCCCC)SC]

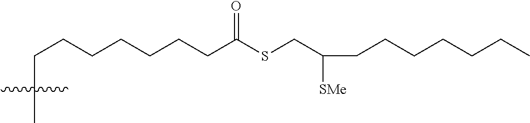 [SMILES: *CCCCCCCC(=O)SCC(CCCCCCC)SC]

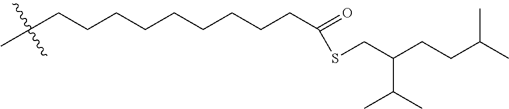 [SMILES: *CCCCCCCCCC(=O)SCC(CCC(C)C)C(C)C]

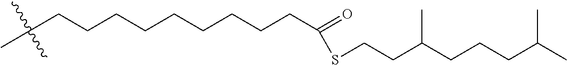 [SMILES: *CCCCCCCCCC(=O)SCCC(C)CCCC(C)C]

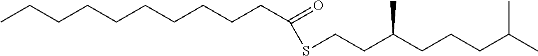 [SMILES: CCCCCCCCCCC(=O)SCC[C@@H](C)CCCC(C)C]

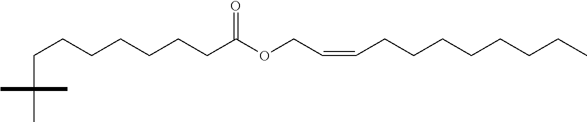

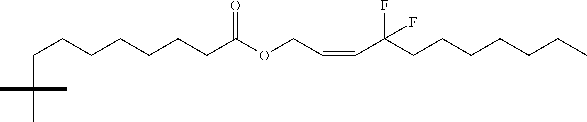

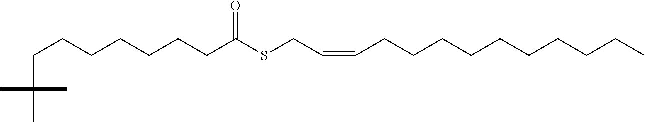

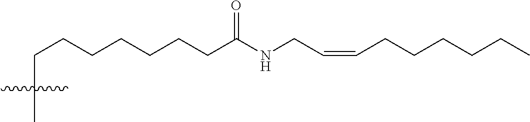 [SMILES: *CCCCCCCC(=O)NC/C=C\CCCCCC]

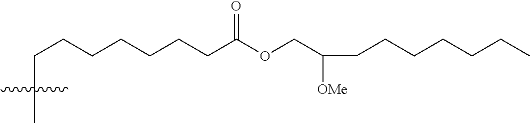 [SMILES: *CCCCCCCC(=O)OCC(CCCCCCC)OC]

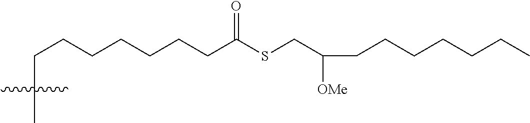 [SMILES: *CCCCCCCC(=O)SCC(CCCCCCC)OC]

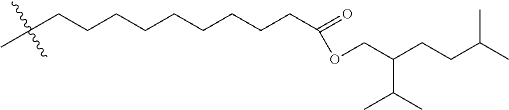 [SMILES: *CCCCCCCCCC(=O)OCC(CCC(C)C)C(C)C]

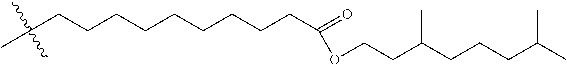 [SMILES: *CCCCCCCCCC(=O)OCCC(C)CCCC(C)C]

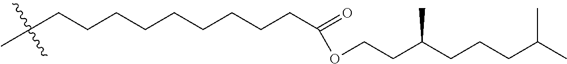 [SMILES: *CCCCCCCCCC(=O)OCC[C@@H](C)CCCC(C)C]

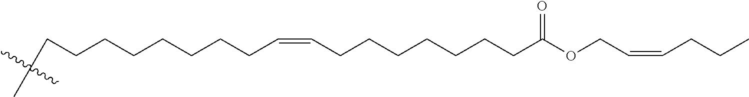 [SMILES: *CCCCCCCC/C=C\CCCCCCCC(=O)OC/C=C\CCC]

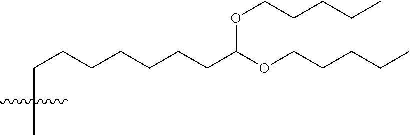 [SMILES: *CCCCCCCC(OCCCCC)OCCCCC]

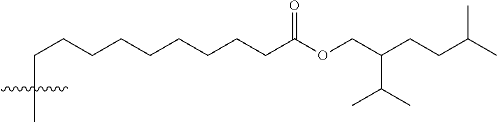 [SMILES: *CCCCCCCCCC(=O)OCC(CCC(C)C)C(C)C]

Other suitable tail groups (e.g., for a compound of Formula (I)) include those of the formula —R¹²-M¹-R¹³ where R¹² is a C₄-C₁₄ alkyl or C₄-C₁₄ alkenyl, M¹ is a biodegradable group as defined above, and R¹³ is a branched alkyl or alkenyl (e.g., a C₁₀-C₂₀ alkyl or C₁₀-C₂₀ alkenyl), such that (i) the chain length of —R¹²-M¹-R¹³ is at most 21 atoms (i.e., the total length of the tail from the first carbon after the tertiary carbon (marked with an asterisk) to a terminus of the tail is at most 21), and (ii) the group —R¹²-M¹-R¹³ has at least 20 carbon atoms (e.g., at least 21 or 22 carbon atoms).

In one preferred embodiment, the chain length of —R¹²-M¹-R¹³ is at most 21 (e.g., at most 20). For example, the chain length can be from about 17 to about 24 or from about 18 to about 20.

In one embodiment, the total carbon atom content of each tail (—R¹²-M¹-R¹³) is from about 17 to about 26. For example, the total carbon atom content can be from about 19 to about 26 or from about 21 to about 26.

In one embodiment, the tail has the formula:

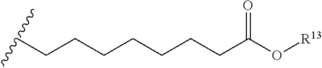

where R¹³ is an alkyl or alkenyl group having from about 13 to about 17 carbon atoms, and the total carbon length of the tail from the first carbon (the leftmost carbon atom above) to a terminus of the tail is at most 20. Preferably, the tail has from about 22 to about 26 carbon atoms. In one embodiment, the maximum length of R¹³ from its attachment point to the ester group of the compound is 12 carbon atoms (e.g., the maximum length can be 11 carbon atoms). In one preferred embodiment, the branch in the alkyl or alkenyl group is at the δ-position or later from the point of attachment of R¹³ to the ester group. Suitable R¹³ groups include, but are not limited to

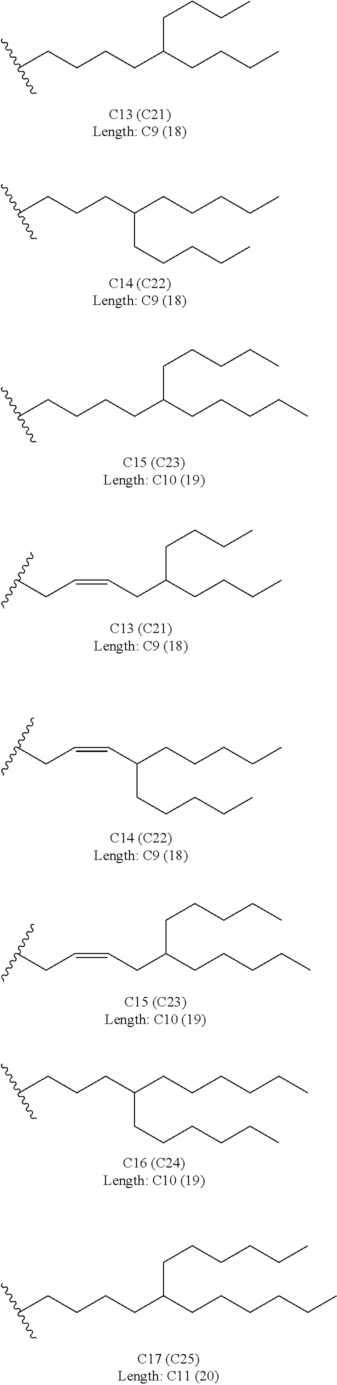

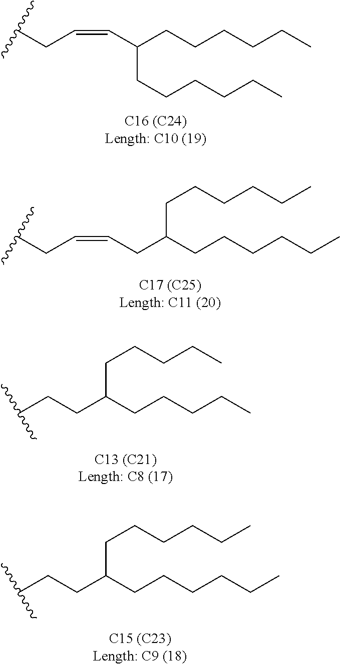 [SMILES: *C/C=C\C(CCCCCC)CCCCCC ; *C/C=C\CC(CCCCCC)CCCCCC ; *CCC(CCCCC)CCCCC ; *CCC(CCCCCC)CCCCCC]

For example, the cationic lipid can be

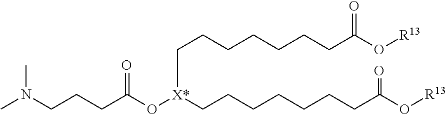

or a salt thereof (e.g., a pharmaceutically acceptable salt thereof), where X is N or P and R¹³ is selected from the groups mentioned above.

Another example is a tail of the formula

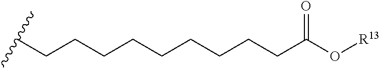

where R¹³ is an alkyl or alkenyl group having from about 13 to about 15 carbon atoms, and the total carbon length of the tail from the first carbon (i.e., the leftmost carbon atom, which is attached to a tertiary carbon) to a terminus of the tail is at most 20. Preferably, the tail has from about 24 to about 26 carbon atoms. In one embodiment, the maximum length of R¹³ from its attachment point to the ester group of the compound is 10 carbon atoms (e.g., the maximum length can be 9 carbon atoms). In one preferred embodiment, the branch in the alkyl or alkenyl group is at the δ-position or later from the point of attachment of R¹³ to the ester group. Suitable R¹³ groups include, but are not limited to

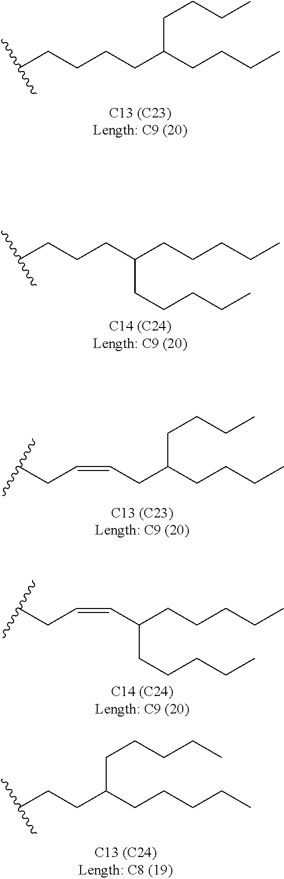

For example, the cationic lipid can be

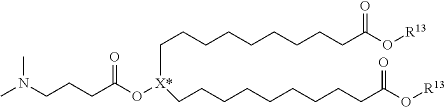

or a salt thereof (e.g., a pharmaceutically acceptable salt thereof), where X is N or P and R¹³ is selected from the groups above.

The R¹³ group may be derived from a natural product, such as dihydrocitgronellol, lavandulol, phytol, or dihydrophytol. In one embodiment, the R¹³ group in the tails above is a dihydrocitronellol group (either as a racemic group or a chirally pure group):

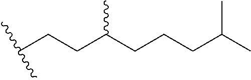 [SMILES: *CCC(C)CCCC(C)C]

For example, the cationic lipid having a dihydroitronellol group can be

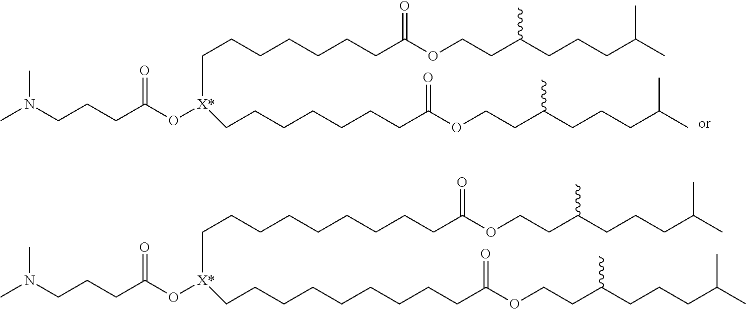

or a salt thereof.

In another embodiment, the R¹³ group in the tails above is a lavandulol group or a homolog of it as shown below:

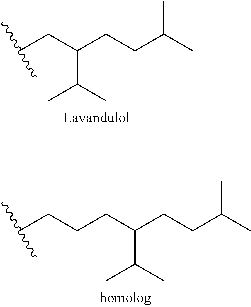

In another embodiment, the R¹³ group in the tails above is a phytol or dihydrophytol group:

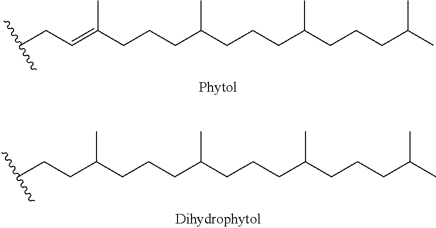 [SMILES: *C/C=C(\C)CCCC(C)CCCC(C)CCCC(C)C ; *CCC(C)CCCC(C)CCCC(C)CCCC(C)C]

For instance, the cationic lipid can be:

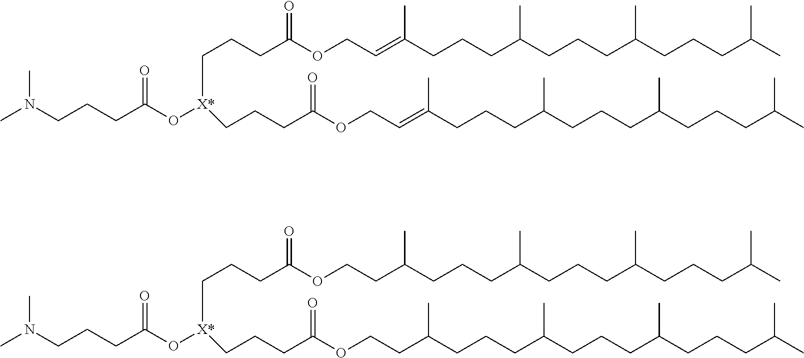

For instance, the cationic lipid can contain one or two tails shown above. For lipids containing two tails, the tails can be the same or different. In one preferred embodiment, the cationic lipid has two tails which are the same.

### Synthesis

In another aspect, the present invention relates to a method of preparing a compound of any of the formulas recited herein.

(1) The amino acid conjugate cationic lipids described herein may be prepared according to the synthetic procedures described in, e.g., U.S. Pat. No. 7,939,505 and International Publication No. WO 07/121,947 (both of which are incorporated by reference in their entirety) using the appropriately substituted starting materials. Suitable exemplary synthetic methods are illustrated in Schemes 1-9 below. The variables in the schemes below are the same as those variables at the same position in the corresponding formula recited above.

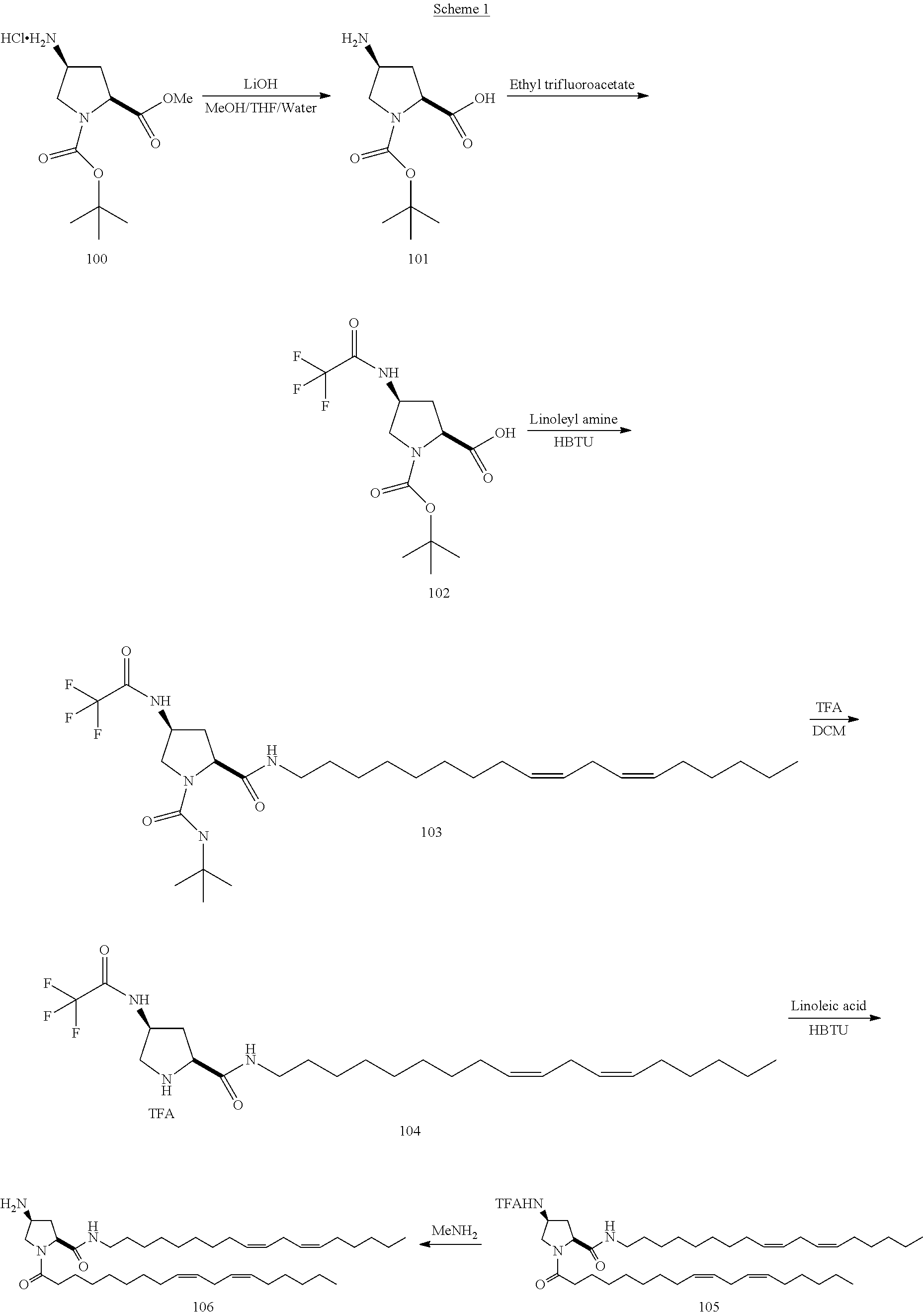

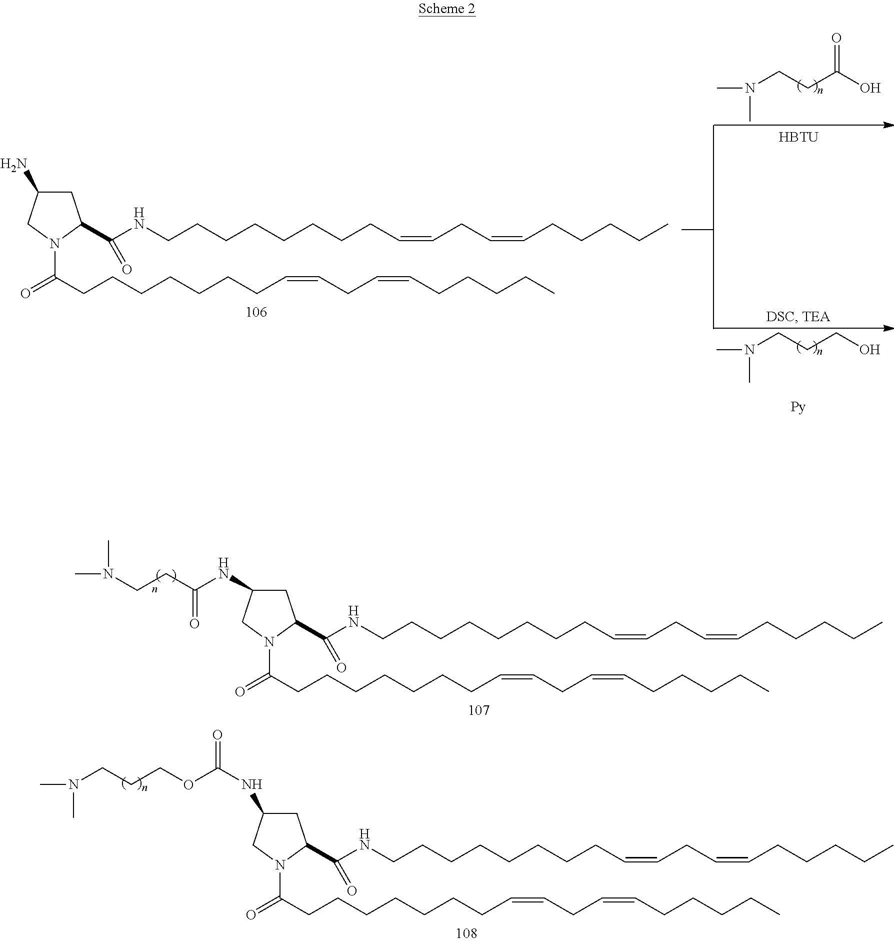

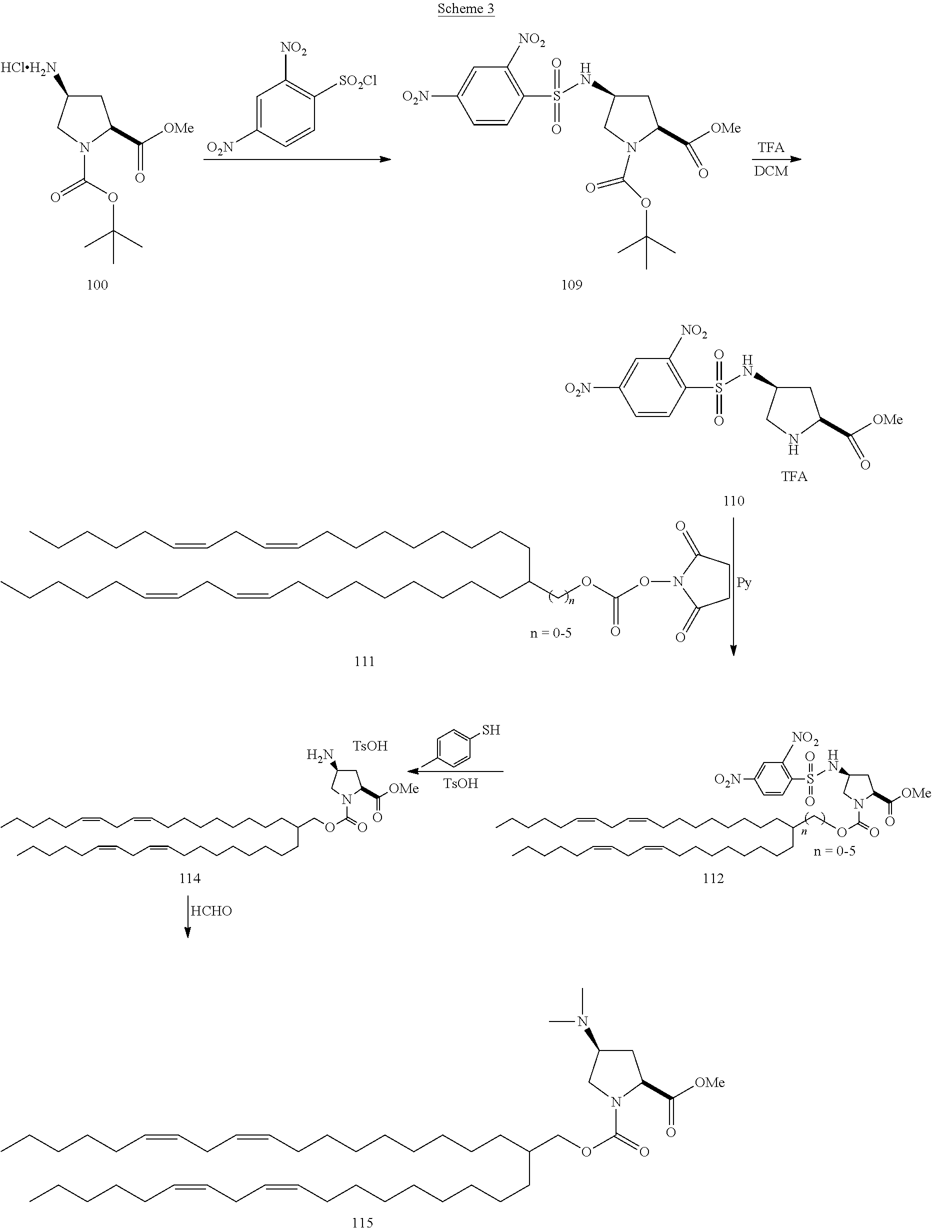

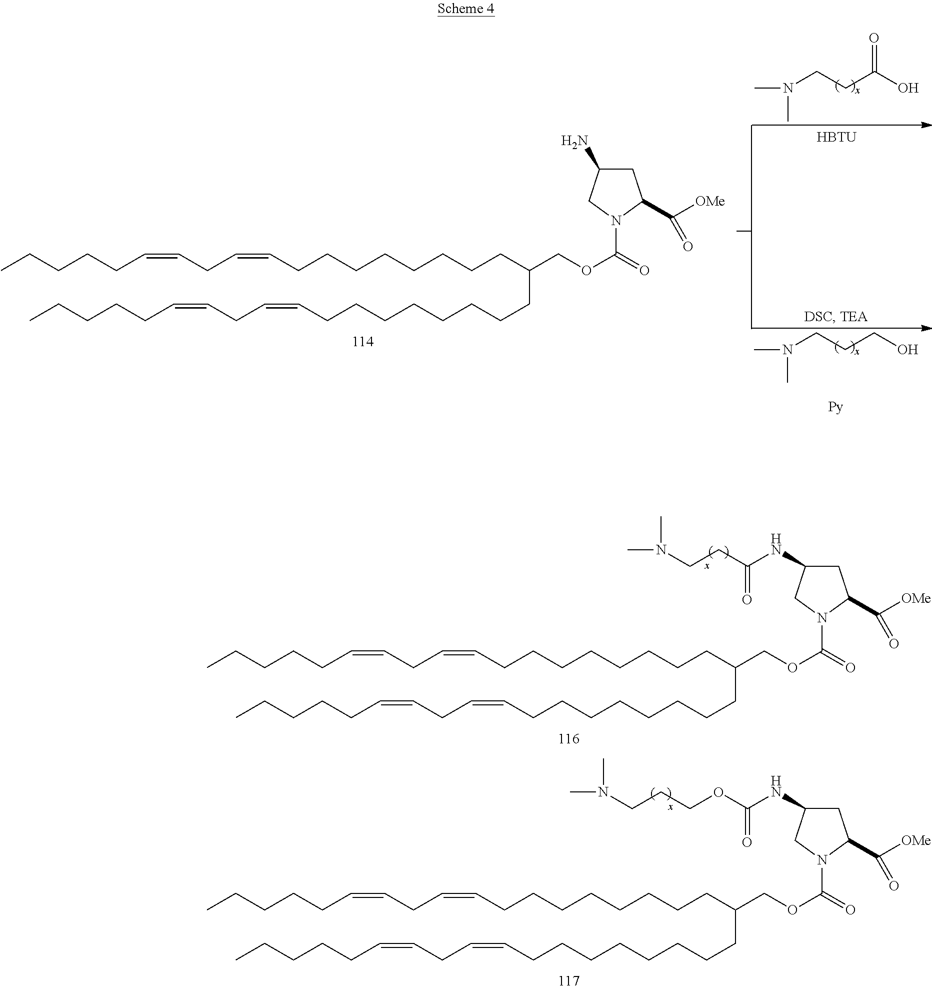

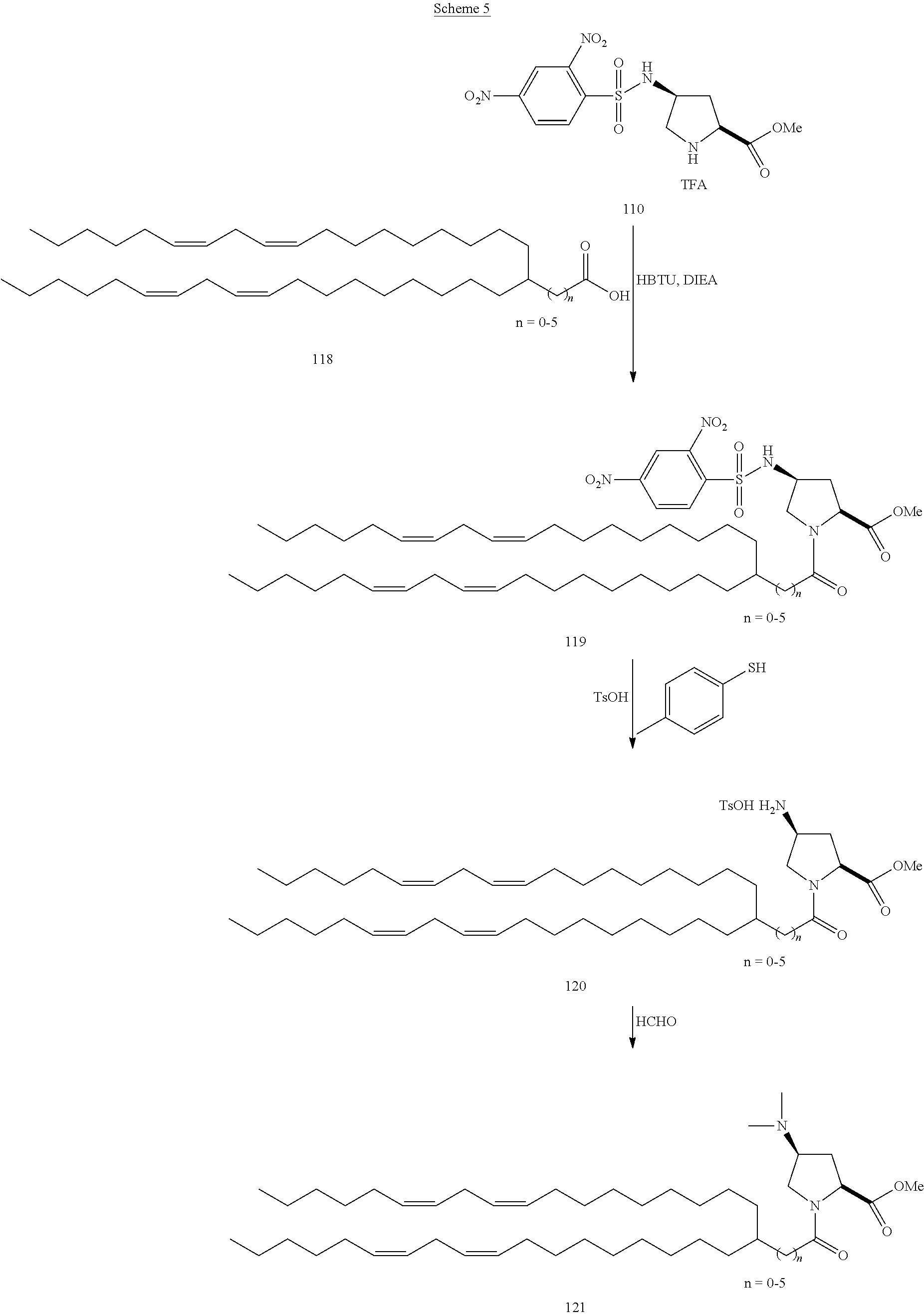

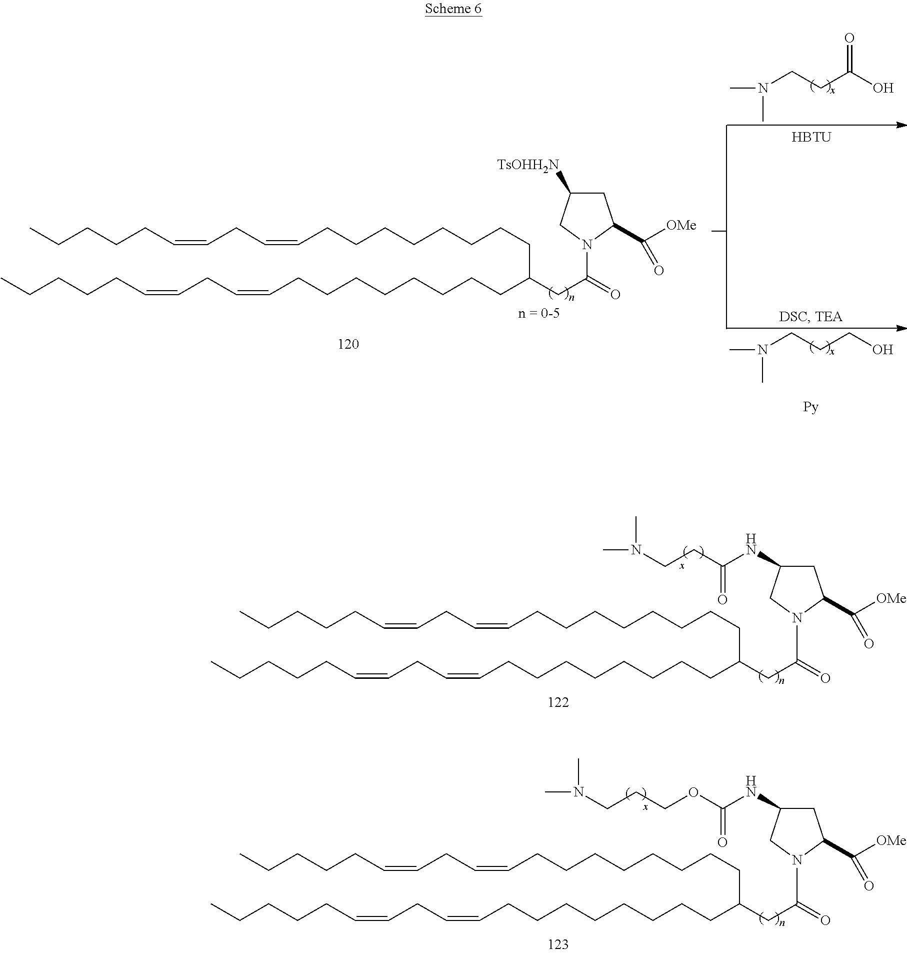

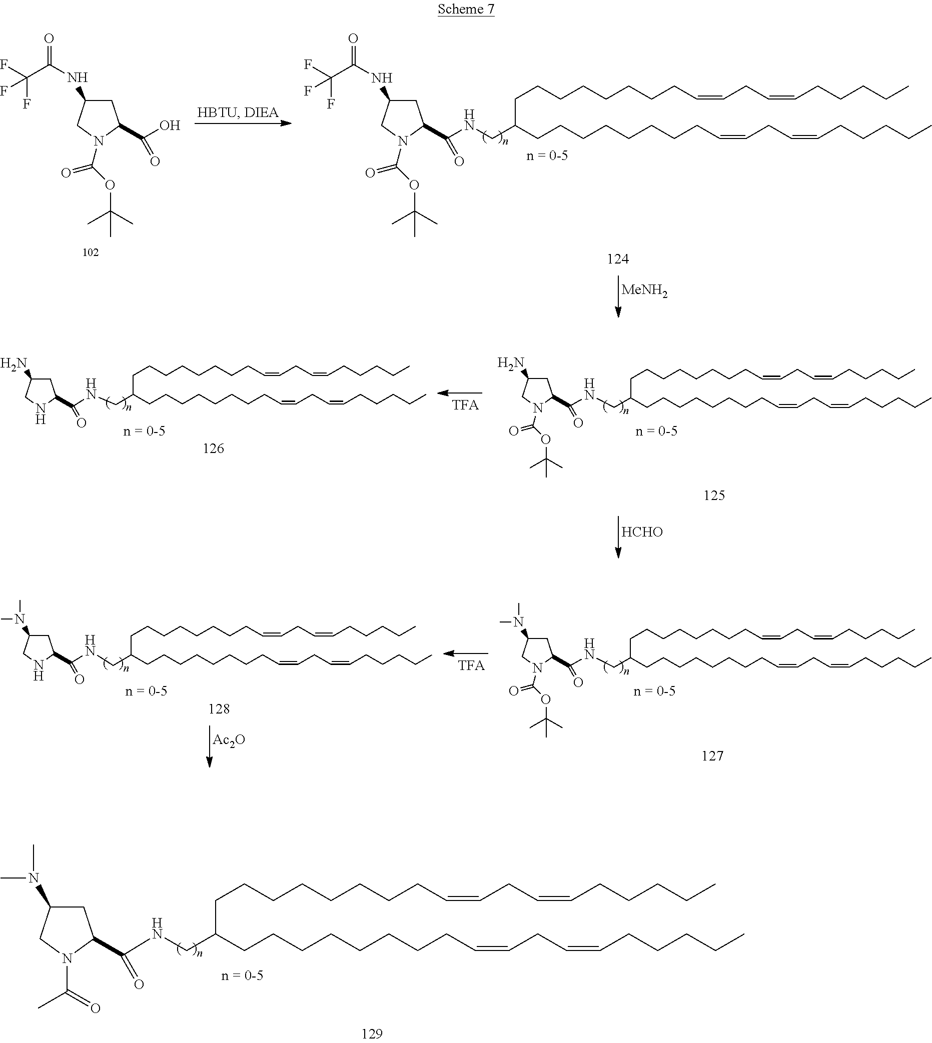

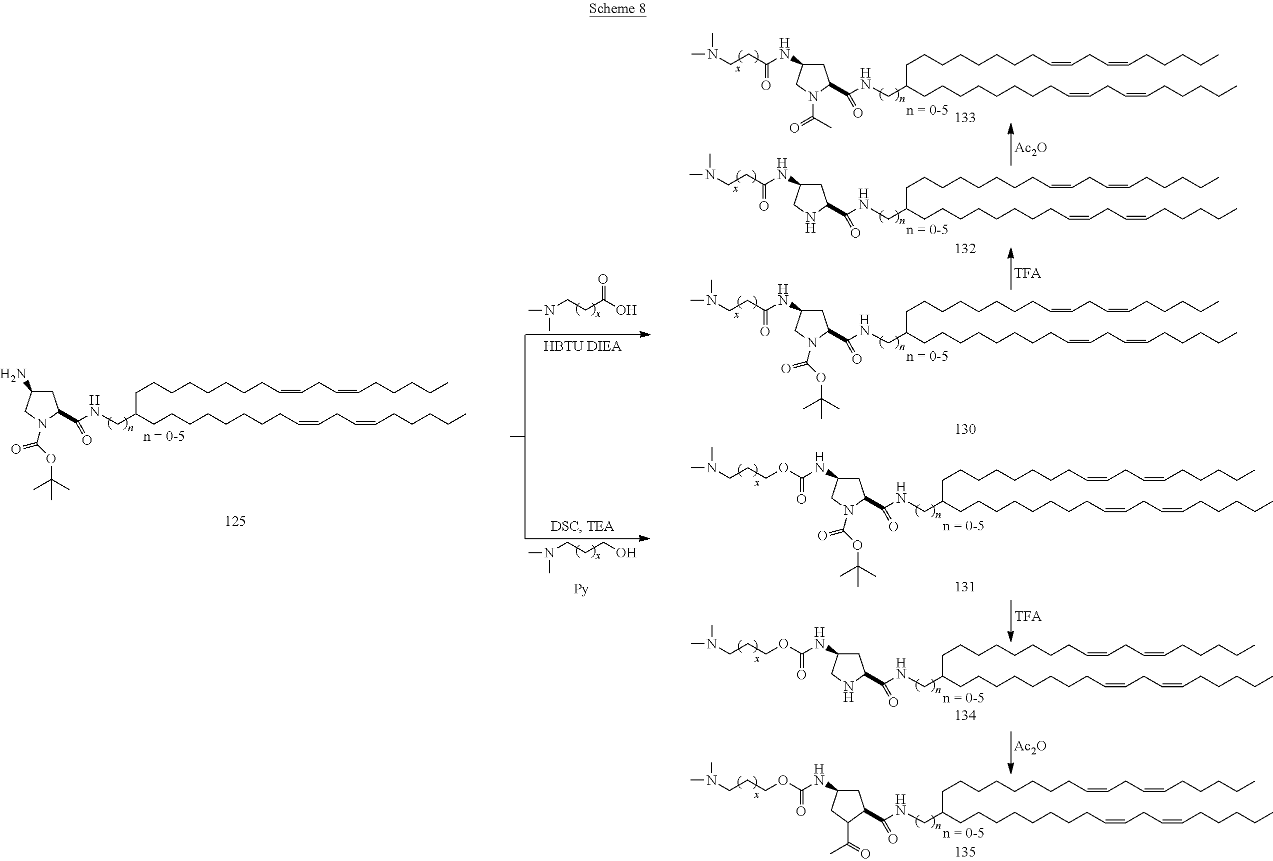

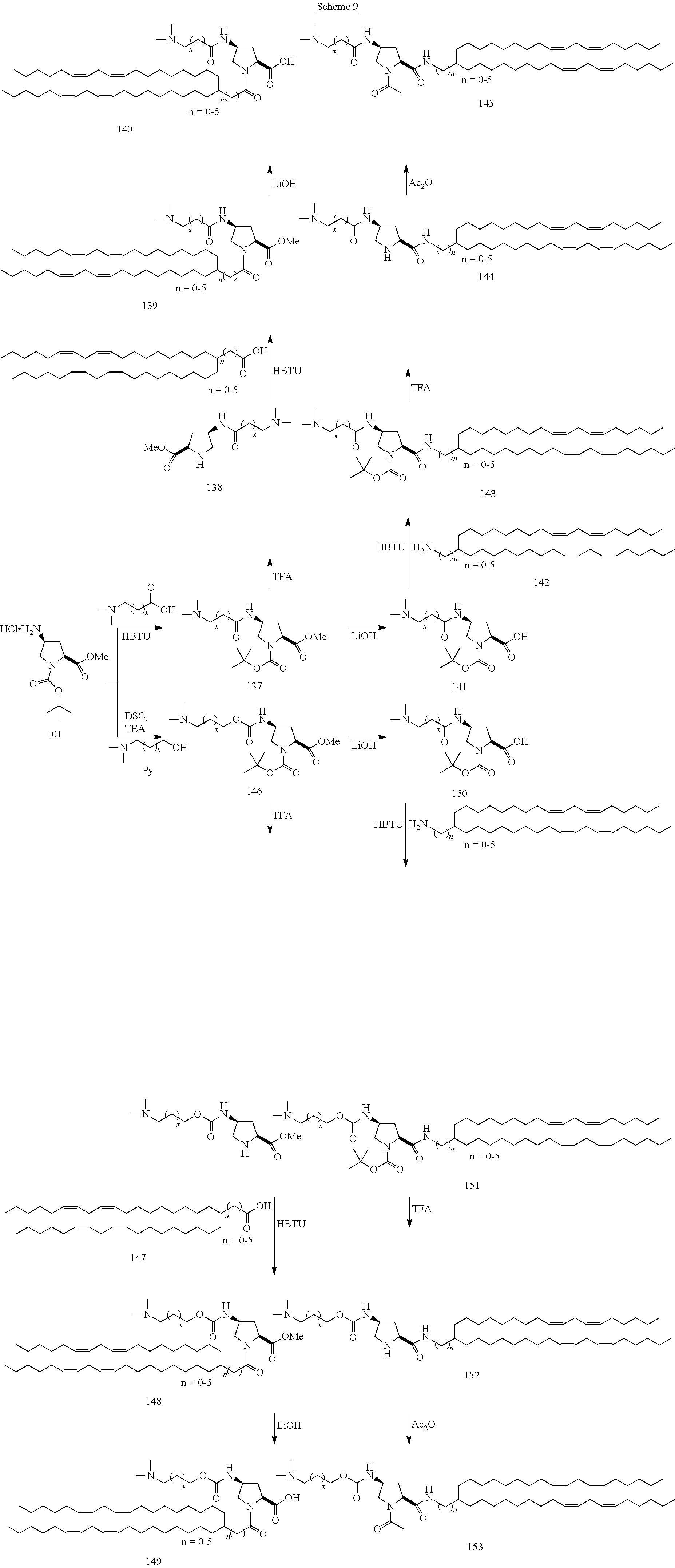

In another aspect, the present invention relates to a method of preparing a compound of Formula I-64. Suitable exemplary synthetic methods are illustrated in Schemes 10-13 shown below.

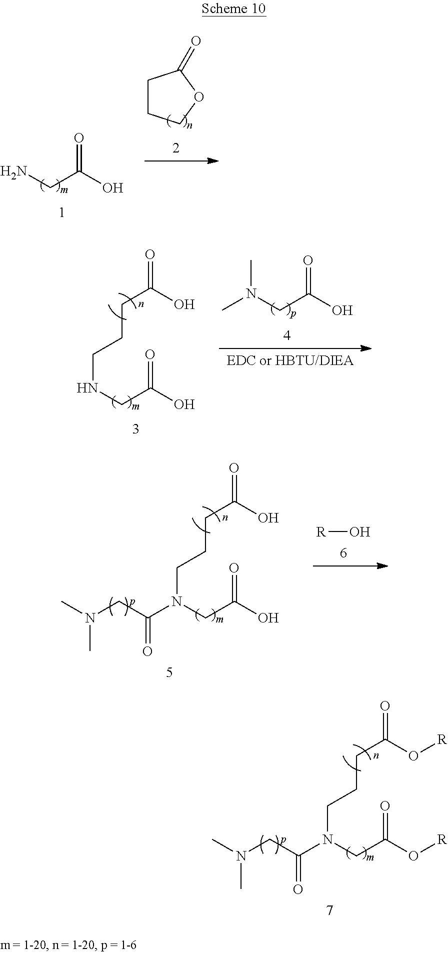

Variable R in the alcohol 6 (R—OH) may be selected accordingly to obtain the desired compound of formula 1-64.

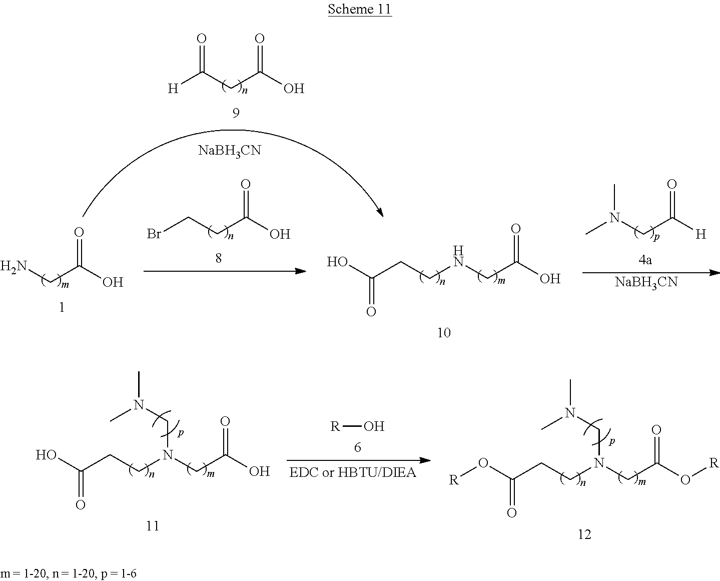

Variable R in alcohol 6 (R—OH) i may be selected accordingly to obtain the desired compound of formula 1-64.

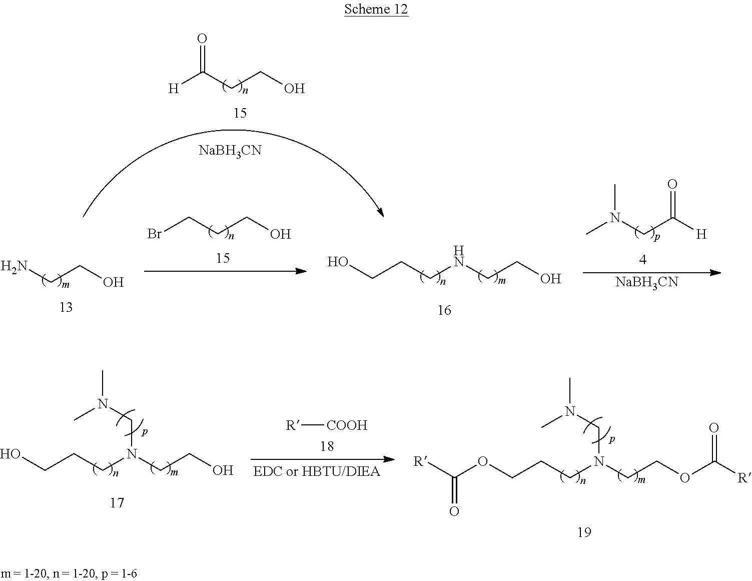

Variable R′ in carboxylic acid 18 (R′—COOH) may be selected accordingly to obtain the desired compound of formula 1-64.

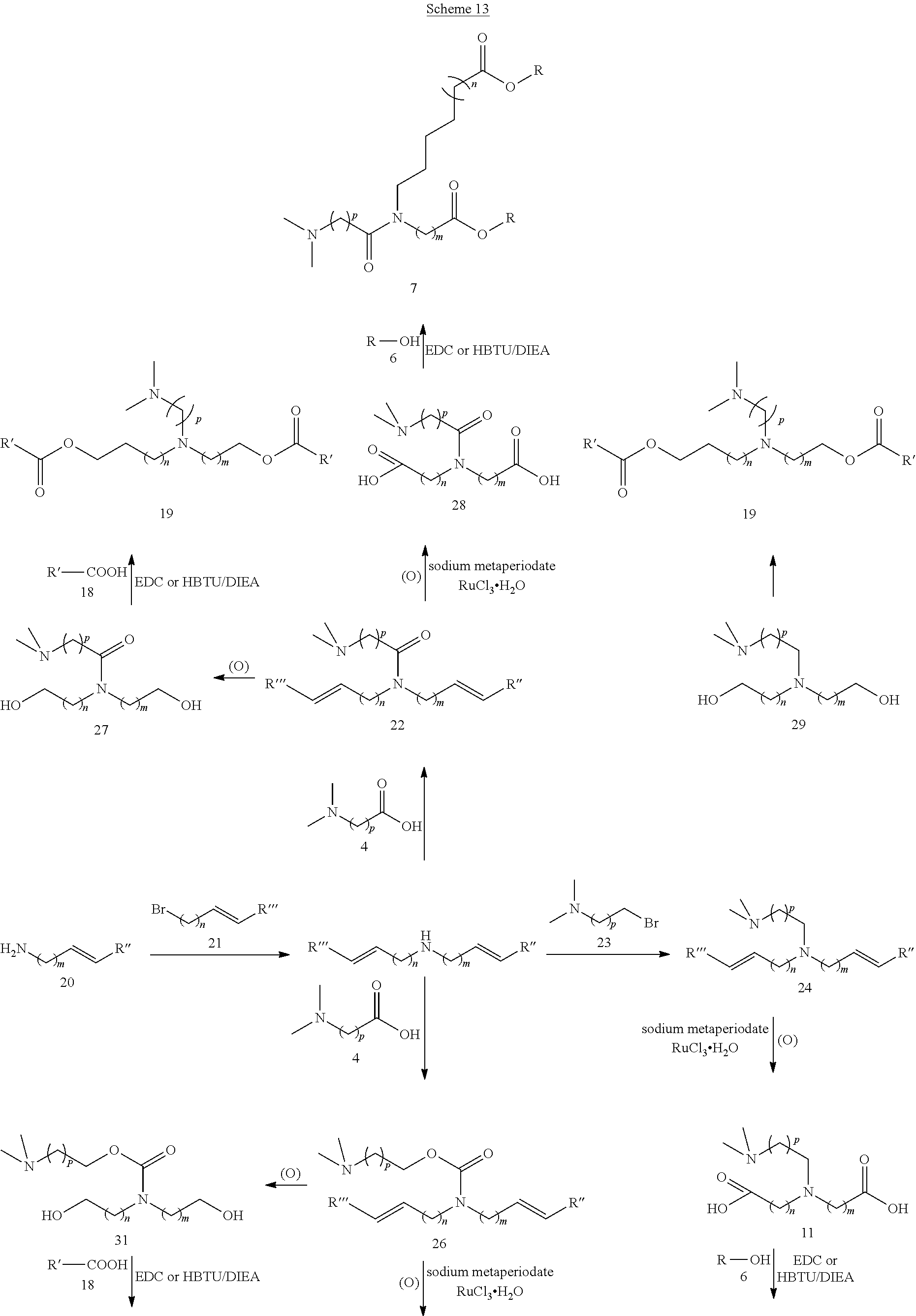

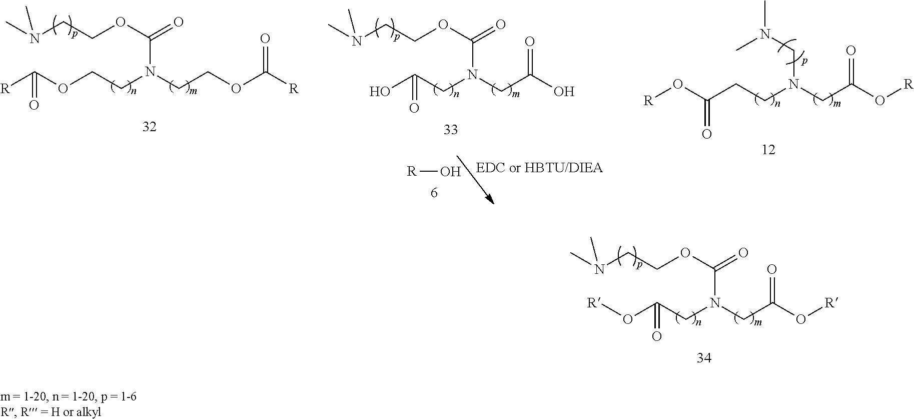

Variable R′ in carboxylic acid 18 (R′—COOH) may be selected accordingly to obtain the desired compound of formula 1-64.

Synthesis of the acetal containing cationic lipids may be a linear process starting with acetal/ketal formation followed by amine displacement of the alkyl bromide as shown in Scheme 14 below.

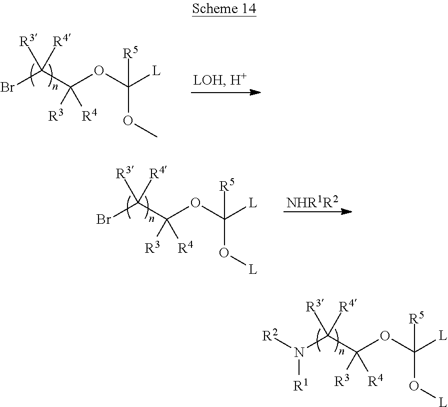

Primary amine containing acetals/ketals may be prepared by converting a phthalamide containing ethyl acetal/ketal to a lipid acetal/ketal and deprotecting it (i.e., remove of the phthalamide protecting group), as shown in Scheme 15 below.

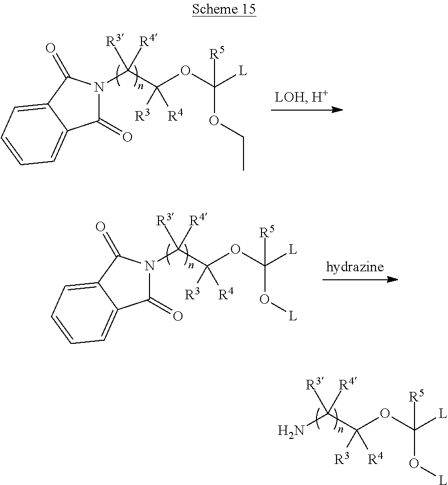

In some examples, as shown in Scheme 16, acetals/ketals may be prepared directly from an aldehyde/ketone by direct acetal/ketal formation. Deprotection generates secondary amine cationic lipids. Reductive amination gives tertiary amine cationic lipids.

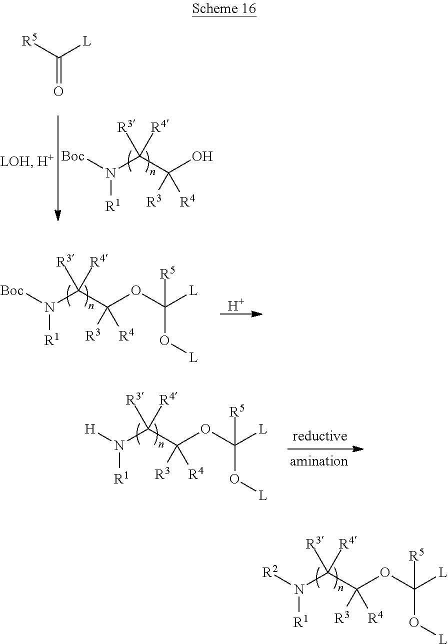

As shown in Scheme 17 below, geminally di-substituted cationic lipids may be prepared by protecting the starting aminoalchol with a phthalamide. Acetal/ketal formation is followed by deprotection with hydrazine.

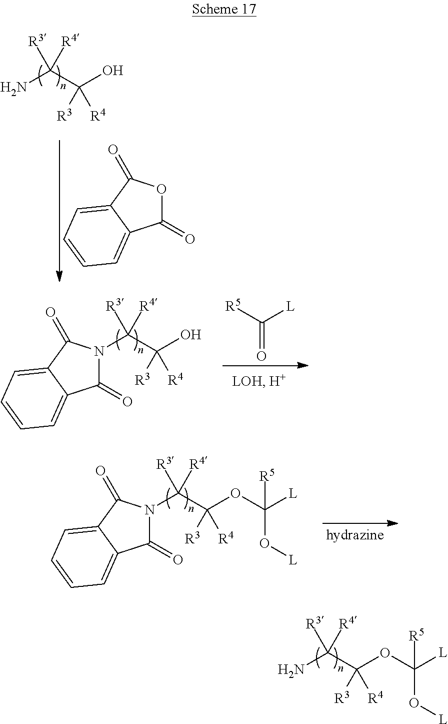

As shown in Scheme 18 below, cyclic ketals may be prepared by first protecting the free amine of an ethyl ketal followed by ketalization with the lipid alcohol. Deprotection of the amine gives the free secondary amine. Reductive amination provides tertiary amine cationic lipids.

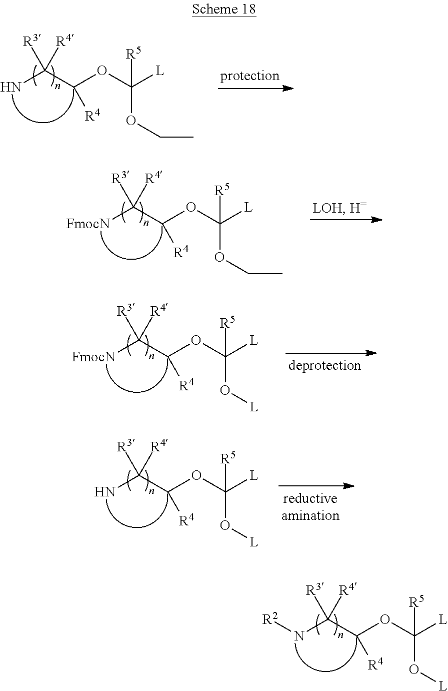

Scheme 19 is an extension of General Scheme 1 wherein the alkylating agent is a phthalamide protected primary amine. Deprotection of the amine with hydrazine affords a cationic lipid.

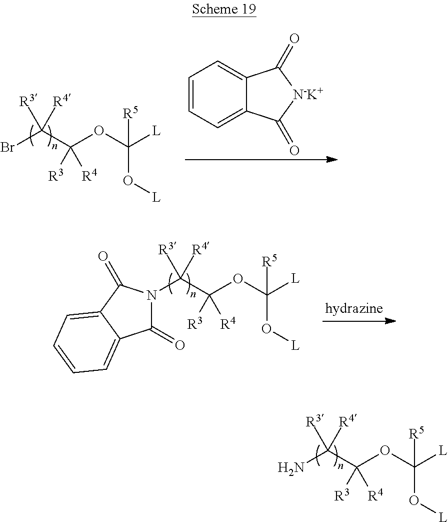

Scheme 20 outlines the preparation of mixed acetals. The mixed acetal may be prepared by converting an intermediate acetal to a mixed lipid acetal using TMSOTf/lutidine followed by addition of a lipid alcohol. Finally, the bromide may be displaced with an amine to provide the final lipid.

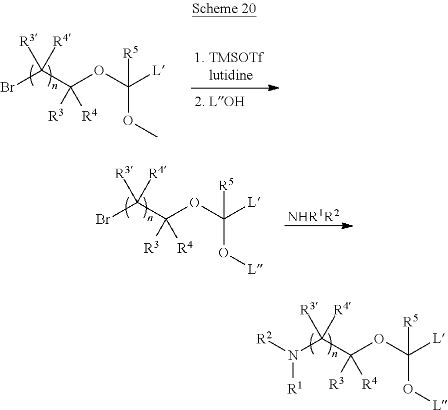

Scheme 21 is analogous to General Scheme 20, where the starting material is a phthalamide protected amine acetal. Mixed acetal formation followed by deprotection of the amine generates the final lipid compound.

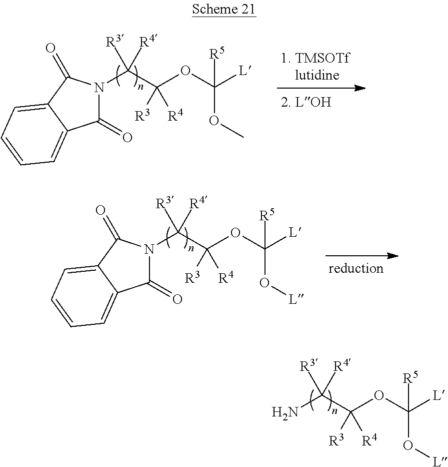

Lipid compounds of the present invention may also be prepared according to Schemes 22 and 23.

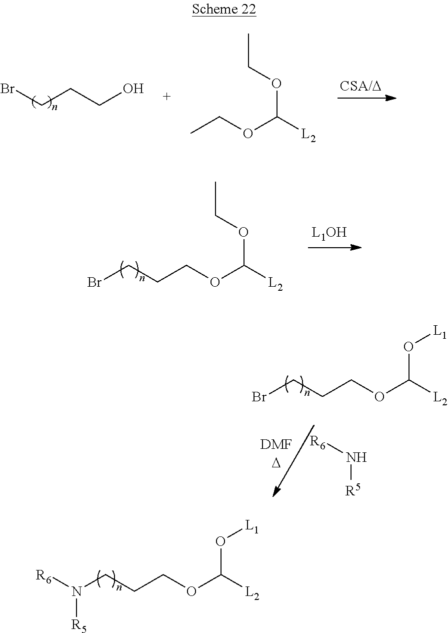

In Scheme 9, a bromoalcohol is reacted with a diethyl acetal to form an acetal intermediate. The acetal intermediate is then reacted with an alcohol of the formula L₁OH to yield a second acetal intermediate having two lipidic moieties. The second acetal intermediate is aminated by reaction with a compound of the formula NHR⁵R⁶ to yield the desired cationic lipid.

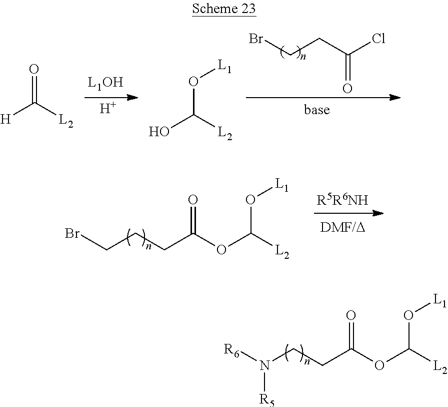

In Scheme 10, an aldehyde of the formula L₂OH is reacted with an alcohol of the formula L₁OH to form an ether intermediate. The ether intermediate is reacted with an acid chloride of the formula Br(CH₂)ₙ—CH₂C(O)Cl to form an ester intermediate, which is aminated with a compound of the formula NHR⁵R⁶ to yield the desired cationic lipid.

Examples of cationic lipids of the present invention include those shown in Tables 3-12 below, and salts thereof (including pharmaceutically acceptable salts thereof). The variables in Tables 3-12 below are the same as those variables at the same position in formulas I-XXIV above. For example, the variable Y in Table 3 can be —C(O)-Xaa-Z—, —Z-Xaa-C(O)—, or

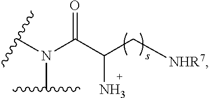

wherein Xaa and Z are defined with respect to formula (I) and R⁷ and s are defined with respect to formula (II).

TABLE 4

| 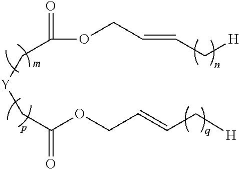 | | | |
|---|---|---|---|
| m | n | p | q |
| 1 | 12 | 1 | 12 |
| 2 | 11 | 2 | 11 |
| 3 | 10 | 3 | 10 |
| 4 | 9 | 4 | 9 |
| 5 | 8 | 5 | 8 |
| 6 | 7 | 6 | 7 |
| 7 | 6 | 7 | 6 |
| 8 | 5 | 8 | 5 |
| 9 | 4 | 9 | 4 |
| 10 | 3 | 10 | 3 |
| 11 | 2 | 11 | 2 |
| 12 | 1 | 12 | 1 |
| 1 | 12 | 2 | 11 |
| 2 | 11 | 3 | 10 |
| 3 | 10 | 4 | 9 |
| 4 | 9 | 5 | 8 |
| 5 | 8 | 6 | 7 |
| 6 | 7 | 7 | 6 |
| 7 | 6 | 8 | 5 |
| 8 | 5 | 9 | 4 |
| 9 | 4 | 10 | 3 |
| 10 | 3 | 11 | 2 |
| 11 | 2 | 12 | 1 |
| 12 | 1 | 1 | 12 |
| 1 | 12 | 3 | 10 |
| 2 | 11 | 4 | 9 |
| 3 | 10 | 5 | 8 |
| 4 | 9 | 6 | 7 |
| 5 | 8 | 7 | 6 |
| 6 | 7 | 8 | 5 |
| 7 | 6 | 9 | 4 |
| 8 | 5 | 10 | 3 |
| 9 | 4 | 11 | 2 |
| 10 | 3 | 12 | 1 |
| 11 | 2 | 2 | 11 |
| 12 | 1 | 4 | 9 |
| 1 | 12 | 4 | 9 |
| 2 | 11 | 5 | 8 |
| 3 | 10 | 6 | 7 |
| 4 | 9 | 7 | 6 |
| 5 | 8 | 8 | 5 |
| 6 | 7 | 9 | 4 |
| 7 | 6 | 10 | 3 |
| 8 | 5 | 11 | 2 |
| 9 | 4 | 12 | 1 |
| 10 | 3 | 2 | 11 |
| 11 | 2 | 3 | 10 |
| 12 | 1 | 4 | 9 |
| 1 | 12 | 5 | 8 |
| 2 | 11 | 6 | 7 |
| 3 | 10 | 7 | 6 |
| 4 | 9 | 8 | 5 |
| 5 | 8 | 9 | 4 |
| 6 | 7 | 10 | 3 |
| 7 | 6 | 11 | 2 |
| 8 | 5 | 12 | 1 |
| 9 | 4 | 2 | 11 |
| 10 | 3 | 3 | 10 |
| 11 | 2 | 4 | 9 |
| 12 | 1 | 5 | 8 |
| 1 | 12 | 6 | 7 |
| 2 | 11 | 7 | 6 |
| 3 | 10 | 8 | 5 |
| 4 | 9 | 9 | 4 |
| 5 | 8 | 10 | 3 |
| 6 | 7 | 11 | 2 |
| 7 | 6 | 12 | 1 |
| 8 | 5 | 2 | 11 |
| 9 | 4 | 3 | 10 |
| 10 | 3 | 4 | 9 |
| 11 | 2 | 5 | 8 |
| 12 | 1 | 6 | 7 |
| 1 | 12 | 7 | 6 |
| 2 | 11 | 8 | 5 |
| 3 | 10 | 9 | 4 |
| 4 | 9 | 8 | 5 |
| 5 | 8 | 9 | 4 |
| 6 | 7 | 10 | 3 |
| 7 | 6 | 11 | 2 |
| 8 | 5 | 12 | 1 |
| 9 | 4 | 2 | 11 |
| 10 | 3 | 3 | 10 |
| 11 | 2 | 4 | 9 |
| 12 | 1 | 5 | 8 |
| 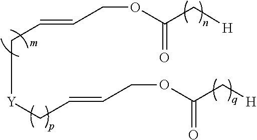 | | | |
| m | n | p | q |
| 12 | 1 | 12 | 1 |
| 11 | 2 | 11 | 2 |
| 10 | 3 | 10 | 3 |
| 9 | 4 | 9 | 4 |
| 8 | 5 | 8 | 5 |
| 7 | 6 | 7 | 6 |
| 6 | 7 | 6 | 7 |
| 5 | 8 | 5 | 8 |
| 4 | 9 | 4 | 9 |
| 3 | 10 | 3 | 10 |
| 2 | 11 | 2 | 11 |
| 1 | 12 | 1 | 12 |
| 12 | 1 | 11 | 2 |
| 11 | 2 | 10 | 3 |
| 10 | 3 | 9 | 4 |
| 9 | 4 | 8 | 5 |
| 8 | 5 | 7 | 6 |
| 7 | 6 | 6 | 7 |
| 6 | 7 | 5 | 8 |
| 5 | 8 | 4 | 9 |
| 4 | 9 | 3 | 10 |
| 3 | 10 | 2 | 11 |
| 2 | 11 | 1 | 12 |
| 1 | 12 | 12 | 1 |
| 12 | 1 | 10 | 3 |
| 11 | 2 | 9 | 4 |
| 10 | 3 | 8 | 5 |
| 9 | 4 | 7 | 6 |
| 8 | 5 | 6 | 7 |
| 7 | 6 | 5 | 8 |
| 6 | 7 | 4 | 9 |
| 5 | 8 | 3 | 10 |
| 4 | 9 | 2 | 11 |
| 3 | 10 | 1 | 12 |
| 2 | 11 | 11 | 2 |
| 1 | 12 | 10 | 3 |
| 12 | 1 | 9 | 4 |
| 11 | 2 | 8 | 5 |
| 10 | 3 | 7 | 6 |
| 9 | 4 | 6 | 7 |
| 8 | 5 | 5 | 8 |
| 7 | 6 | 4 | 9 |
| 6 | 7 | 3 | 10 |
| 5 | 8 | 2 | 11 |
| 4 | 9 | 1 | 12 |
| 3 | 10 | 11 | 2 |
| 2 | 11 | 10 | 3 |
| 1 | 12 | 11 | 2 |
| 12 | 1 | 8 | 5 |
| 11 | 2 | 7 | 6 |
| 10 | 3 | 6 | 7 |
| 9 | 4 | 5 | 8 |
| 8 | 5 | 4 | 9 |
| 7 | 6 | 3 | 10 |
| 6 | 7 | 2 | 11 |
| 5 | 8 | 1 | 12 |
| 4 | 9 | 11 | 2 |
| 3 | 10 | 10 | 3 |
| 2 | 11 | 11 | 2 |
| 1 | 12 | 12 | 1 |
| 12 | 1 | 7 | 6 |
| 11 | 2 | 6 | 7 |
| 10 | 3 | 5 | 8 |
| 9 | 4 | 4 | 9 |
| 8 | 5 | 3 | 10 |
| 7 | 6 | 2 | 11 |
| 6 | 7 | 1 | 12 |
| 5 | 8 | 11 | 2 |
| 4 | 9 | 10 | 3 |
| 3 | 10 | 11 | 2 |
| 2 | 11 | 12 | 1 |
| 1 | 12 | 1 | 12 |
| 12 | 1 | 6 | 7 |
| 11 | 2 | 5 | 8 |
| 10 | 3 | 4 | 9 |
| 9 | 4 | 3 | 10 |
| 8 | 5 | 2 | 11 |
| 7 | 6 | 1 | 12 |
| 6 | 7 | 11 | 2 |
| 5 | 8 | 10 | 3 |
| 4 | 9 | 11 | 2 |
| 3 | 10 | 12 | 1 |
| 2 | 11 | 1 | 12 |
| 1 | 12 | 2 | 11 |

TABLE 5

| 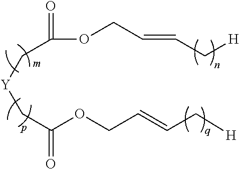 | | | |
|---|---|---|---|
| m | n | p | q |
| 1 | 12 | 1 | 12 |
| 2 | 11 | 2 | 11 |
| 3 | 10 | 3 | 10 |
| 4 | 9 | 4 | 9 |
| 5 | 8 | 5 | 8 |
| 6 | 7 | 6 | 7 |
| 7 | 6 | 7 | 6 |
| 8 | 5 | 8 | 5 |
| 9 | 4 | 9 | 4 |
| 10 | 3 | 10 | 3 |
| 11 | 2 | 11 | 2 |
| 12 | 1 | 12 | 1 |
| 1 | 12 | 2 | 11 |
| 2 | 11 | 3 | 10 |
| 3 | 10 | 4 | 9 |
| 4 | 9 | 5 | 8 |
| 5 | 8 | 6 | 7 |
| 6 | 7 | 7 | 6 |
| 7 | 6 | 8 | 5 |
| 8 | 5 | 9 | 4 |
| 9 | 4 | 10 | 3 |
| 10 | 3 | 11 | 2 |
| 11 | 2 | 12 | 1 |
| 12 | 1 | 1 | 12 |
| 1 | 12 | 3 | 10 |
| 2 | 11 | 4 | 9 |
| 3 | 10 | 5 | 8 |
| 4 | 9 | 6 | 7 |
| 5 | 8 | 7 | 6 |
| 6 | 7 | 8 | 5 |
| 7 | 6 | 9 | 4 |
| 8 | 5 | 10 | 3 |
| 9 | 4 | 11 | 2 |
| 10 | 3 | 12 | 1 |
| 11 | 2 | 2 | 11 |
| 12 | 1 | 4 | 9 |
| 1 | 12 | 4 | 9 |
| 2 | 11 | 5 | 8 |
| 3 | 10 | 6 | 7 |
| 4 | 9 | 7 | 6 |
| 5 | 8 | 8 | 5 |
| 6 | 7 | 9 | 4 |
| 7 | 6 | 10 | 3 |
| 8 | 5 | 11 | 2 |
| 9 | 4 | 12 | 1 |
| 10 | 3 | 2 | 11 |
| 11 | 2 | 3 | 10 |
| 12 | 1 | 4 | 9 |
| 1 | 12 | 5 | 8 |
| 2 | 11 | 6 | 7 |
| 3 | 10 | 7 | 6 |
| 4 | 9 | 8 | 5 |
| 5 | 8 | 9 | 4 |
| 6 | 7 | 10 | 3 |
| 7 | 6 | 11 | 2 |
| 8 | 5 | 12 | 1 |
| 9 | 4 | 2 | 11 |
| 10 | 3 | 3 | 10 |
| 11 | 2 | 4 | 9 |
| 12 | 1 | 5 | 8 |
| 1 | 12 | 6 | 7 |
| 2 | 11 | 7 | 6 |
| 3 | 10 | 8 | 5 |
| 4 | 9 | 9 | 4 |
| 5 | 8 | 10 | 3 |
| 6 | 7 | 11 | 2 |
| 7 | 6 | 12 | 1 |
| 8 | 5 | 2 | 11 |
| 9 | 4 | 3 | 10 |
| 10 | 3 | 4 | 9 |
| 11 | 2 | 5 | 8 |
| 12 | 1 | 6 | 7 |
| 1 | 12 | 7 | 6 |
| 2 | 11 | 8 | 5 |
| 3 | 10 | 9 | 4 |
| 4 | 9 | 8 | 5 |
| 5 | 8 | 9 | 4 |
| 6 | 7 | 10 | 3 |
| 7 | 6 | 11 | 2 |
| 8 | 5 | 12 | 1 |
| 9 | 4 | 2 | 11 |
| 10 | 3 | 3 | 10 |
| 11 | 2 | 4 | 9 |
| 12 | 1 | 5 | 8 |
| 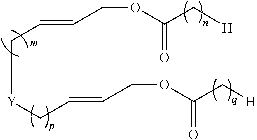 | | | |
| m | n | p | q |
| 12 | 1 | 12 | 1 |
| 11 | 2 | 11 | 2 |
| 10 | 3 | 10 | 3 |
| 9 | 4 | 9 | 4 |
| 8 | 5 | 8 | 5 |
| 7 | 6 | 7 | 6 |
| 6 | 7 | 6 | 7 |
| 5 | 8 | 5 | 8 |
| 4 | 9 | 4 | 9 |
| 3 | 10 | 3 | 10 |
| 2 | 11 | 2 | 11 |
| 1 | 12 | 1 | 12 |
| 12 | 1 | 11 | 2 |
| 11 | 2 | 10 | 3 |
| 10 | 3 | 9 | 4 |
| 9 | 4 | 8 | 5 |
| 8 | 5 | 7 | 6 |
| 7 | 6 | 6 | 7 |
| 6 | 7 | 5 | 8 |
| 5 | 8 | 4 | 9 |
| 4 | 9 | 3 | 10 |
| 3 | 10 | 2 | 11 |
| 2 | 11 | 1 | 12 |
| 1 | 12 | 12 | 1 |
| 12 | 1 | 10 | 3 |
| 11 | 2 | 9 | 4 |
| 10 | 3 | 8 | 5 |
| 9 | 4 | 7 | 6 |
| 8 | 5 | 6 | 7 |
| 7 | 6 | 5 | 8 |
| 6 | 7 | 4 | 9 |
| 5 | 8 | 3 | 10 |
| 4 | 9 | 2 | 11 |
| 3 | 10 | 1 | 12 |
| 2 | 11 | 11 | 2 |
| 1 | 12 | 10 | 3 |
| 12 | 1 | 9 | 4 |
| 11 | 2 | 8 | 5 |
| 10 | 3 | 7 | 6 |
| 9 | 4 | 6 | 7 |
| 8 | 5 | 5 | 8 |
| 7 | 6 | 4 | 9 |
| 6 | 7 | 3 | 10 |
| 5 | 8 | 2 | 11 |
| 4 | 9 | 1 | 12 |
| 3 | 10 | 11 | 2 |
| 2 | 11 | 10 | 3 |
| 1 | 12 | 11 | 2 |
| 12 | 1 | 8 | 5 |
| 11 | 2 | 7 | 6 |
| 10 | 3 | 6 | 7 |
| 9 | 4 | 5 | 8 |
| 8 | 5 | 4 | 9 |
| 7 | 6 | 3 | 10 |
| 6 | 7 | 2 | 11 |
| 5 | 8 | 1 | 12 |
| 4 | 9 | 11 | 2 |
| 3 | 10 | 10 | 3 |
| 2 | 11 | 11 | 2 |
| 1 | 12 | 12 | 1 |
| 12 | 1 | 7 | 6 |
| 11 | 2 | 6 | 7 |
| 10 | 3 | 5 | 8 |
| 9 | 4 | 4 | 9 |
| 8 | 5 | 3 | 10 |
| 7 | 6 | 2 | 11 |
| 6 | 7 | 1 | 12 |
| 5 | 8 | 11 | 2 |
| 4 | 9 | 10 | 3 |
| 3 | 10 | 11 | 2 |
| 2 | 11 | 12 | 1 |
| 1 | 12 | 1 | 12 |
| 12 | 1 | 6 | 7 |
| 11 | 2 | 5 | 8 |
| 10 | 3 | 4 | 9 |
| 9 | 4 | 3 | 10 |
| 8 | 5 | 2 | 11 |
| 7 | 6 | 1 | 12 |
| 6 | 7 | 11 | 2 |
| 5 | 8 | 10 | 3 |
| 4 | 9 | 11 | 2 |
| 3 | 10 | 12 | 1 |
| 2 | 11 | 1 | 12 |
| 1 | 12 | 2 | 11 |

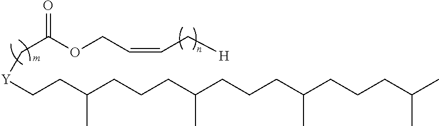

| m | N |
|---|---|
| 1 | 12 |
| 2 | 11 |
| 3 | 10 |
| 4 | 9 |
| 5 | 8 |
| 6 | 7 |
| 7 | 6 |
| 8 | 5 |
| 9 | 4 |
| 10 | 3 |
| 11 | 2 |
| 12 | 1 |

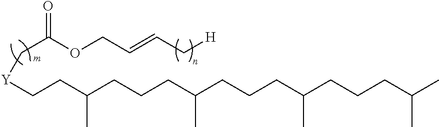

| | |
|---|---|
| 1 | 12 |
| 2 | 11 |
| 3 | 10 |
| 4 | 9 |
| 5 | 8 |
| 6 | 7 |
| 7 | 6 |
| 8 | 5 |
| 9 | 4 |
| 10 | 3 |
| 11 | 2 |
| 12 | 1 |

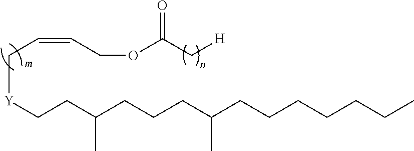

| m | n |
|---|---|
| 12 | 1 |
| 11 | 2 |
| 10 | 3 |
| 9 | 4 |
| 8 | 5 |
| 7 | 6 |
| 6 | 7 |
| 5 | 8 |
| 4 | 9 |
| 3 | 10 |
| 2 | 11 |
| 1 | 12 |

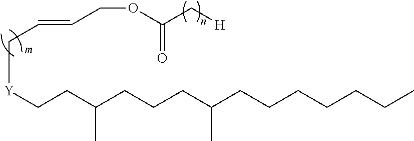

| | |
|---|---|
| 12 | 1 |
| 11 | 2 |
| 10 | 3 |
| 9 | 4 |
| 8 | 5 |
| 7 | 6 |
| 6 | 7 |
| 5 | 8 |
| 4 | 9 |
| 3 | 10 |
| 2 | 11 |
| 1 | 12 |

TABLE 12

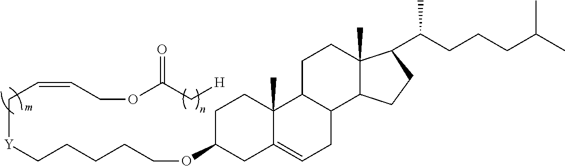

| m | n |
|---|---|
| 1 | 12 |
| 2 | 11 |
| 3 | 10 |
| 4 | 9 |
| 5 | 8 |
| 6 | 7 |
| 7 | 6 |
| 8 | 5 |
| 9 | 4 |
| 10 | 3 |
| 11 | 2 |
| 12 | 1 |

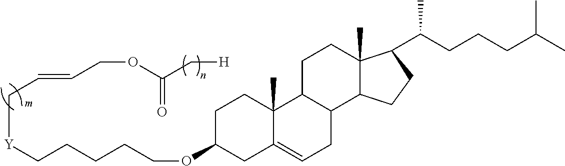

| | |
|---|---|
| 1 | 12 |
| 2 | 11 |
| 3 | 10 |
| 4 | 9 |
| 5 | 8 |
| 6 | 7 |
| 7 | 6 |
| 8 | 5 |
| 9 | 4 |
| 10 | 3 |
| 11 | 2 |
| 12 | 1 |

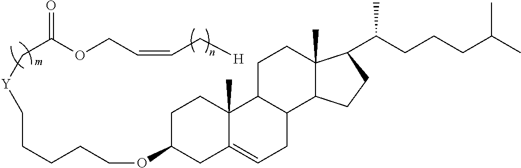

| m | n |
|---|---|
| 12 | 1 |
| 11 | 2 |
| 10 | 3 |
| 9 | 4 |
| 8 | 5 |
| 7 | 6 |
| 6 | 7 |
| 5 | 8 |
| 4 | 9 |
| 3 | 10 |
| 2 | 11 |
| 1 | 12 |

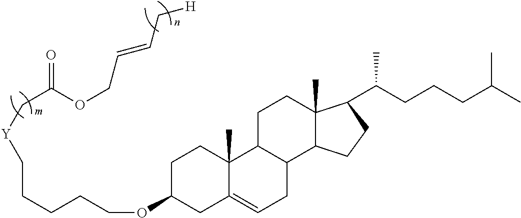

| | |
|---|---|
| 12 | 1 |
| 11 | 2 |
| 10 | 3 |
| 9 | 4 |
| 8 | 5 |
| 7 | 6 |
| 6 | 7 |
| 5 | 8 |
| 4 | 9 |
| 3 | 10 |
| 2 | 11 |
| 1 | 12 |
| 1 | 12 |

Additional examples of cationic lipids of the present invention include those shown in Table 13 below, and salts thereof (including pharmaceutically acceptable salts thereof). The variables in Table 13 below are the same as those variables at the same position in formulas I-25 above.

In one embodiment, the cationic lipid of the present invention is selected from the following compounds, and salts thereof (including pharmaceutically acceptable salts thereof):

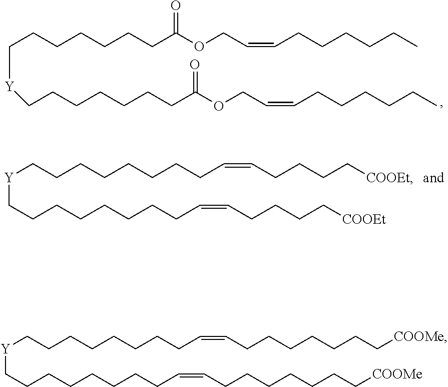

wherein Y is as defined above.

For example, in one embodiment, the cationic lipid of the present invention is selected from the following compounds:

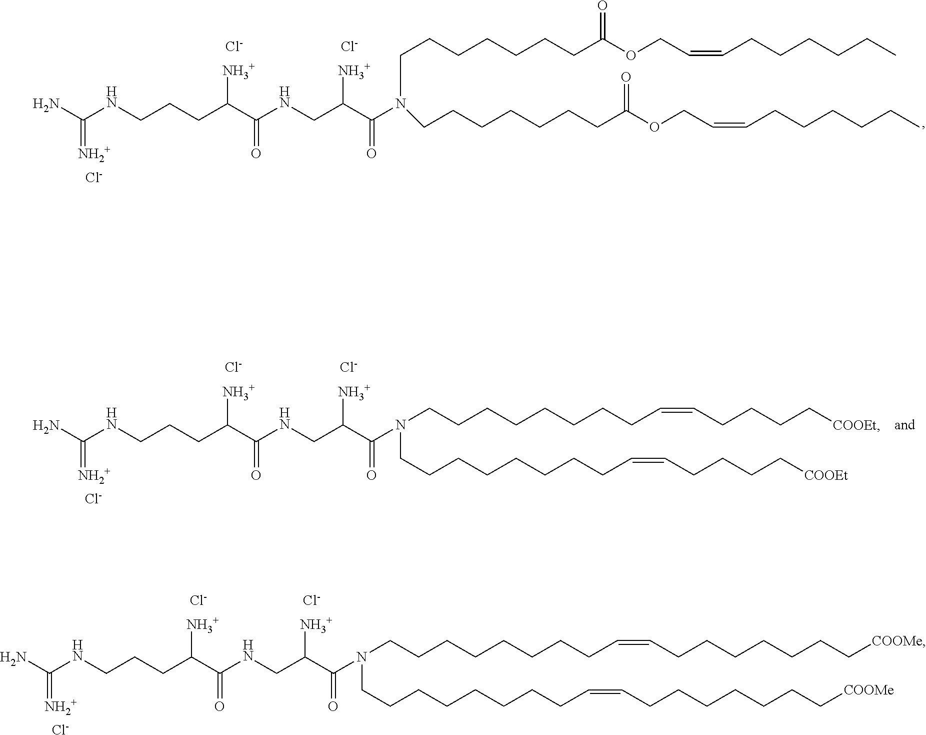

For example, in another embodiment, the cationic lipid of the present invention is selected from the following compounds:

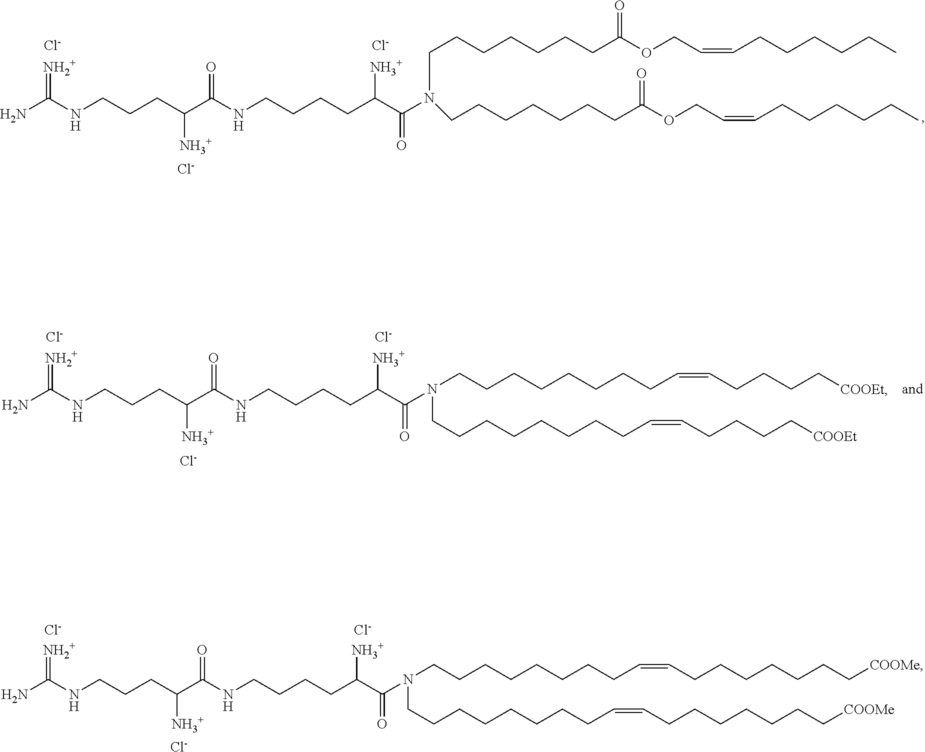

In a further embodiment, the cationic lipid of the present invention is selected from the following compounds, and salts thereof (including pharmaceutically acceptable salts thereof):

TABLE 13
Compound

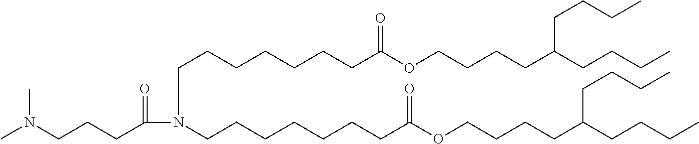 [SMILES: CCCCC(CCCC)CCCCOC(=O)CCCCCCCN(CCCCCCCC(=O)OCCCCC(CCCC)CCCC)C(=O)CCCN(C)C]

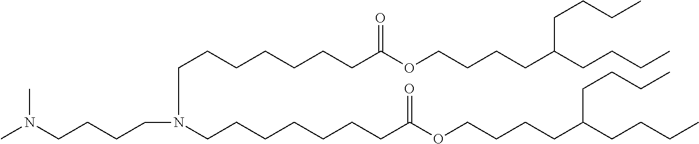 [SMILES: CCCCC(CCCC)CCCCOC(=O)CCCCCCCN(CCCCCCCC(=O)OCCCCC(CCCC)CCCC)CCCCN(C)C]

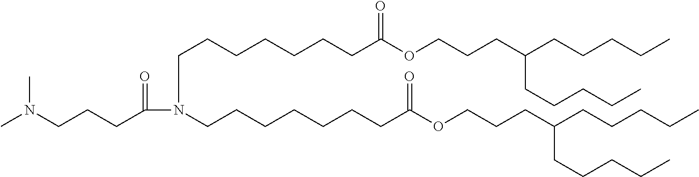 [SMILES: CCCCCC(CCCCC)CCCOC(=O)CCCCCCCN(CCCCCCCC(=O)OCCCC(CCCCC)CCCCC)C(=O)CCCN(C)C]

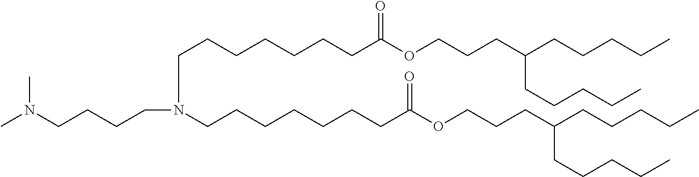 [SMILES: CCCCCC(CCCCC)CCCOC(=O)CCCCCCCN(CCCCCCCC(=O)OCCCC(CCCCC)CCCCC)CCCCN(C)C]

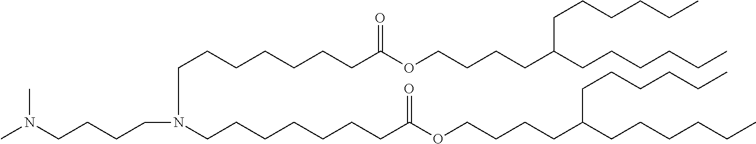 [SMILES: CCCCCCC(CCCCCC)CCCCOC(=O)CCCCCCCN(CCCCCCCC(=O)OCCCCC(CCCCCC)CCCCCC)CCCCN(C)C]

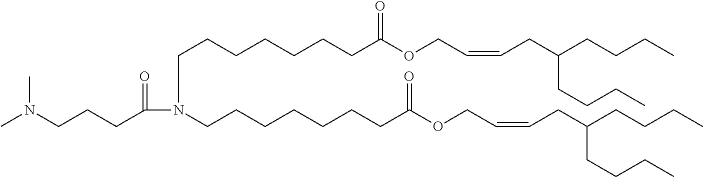 [SMILES: CCCCC(C/C=C\COC(=O)CCCCCCCN(CCCCCCCC(=O)OC/C=C\CC(CCCC)CCCC)C(=O)CCCN(C)C)CCCC]

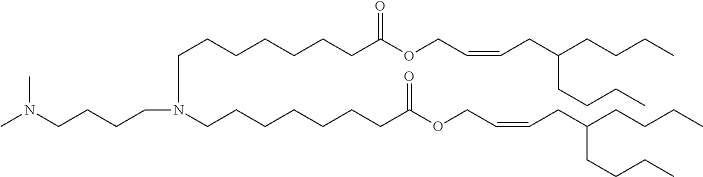 [SMILES: CCCCC(C/C=C\COC(=O)CCCCCCCN(CCCCCCCC(=O)OC/C=C\CC(CCCC)CCCC)CCCCN(C)C)CCCC]

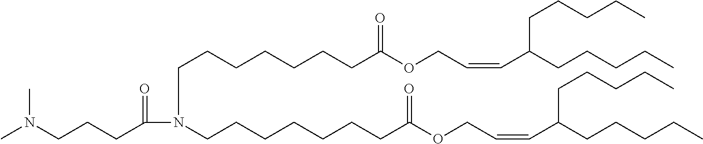 [SMILES: CCCCCC(/C=C\COC(=O)CCCCCCCN(CCCCCCCC(=O)OC/C=C\C(CCCCC)CCCCC)C(=O)CCCN(C)C)CCCCC]

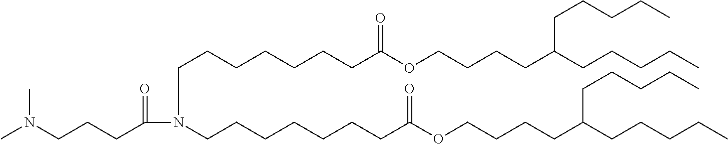 [SMILES: CCCCCC(CCCCC)CCCCOC(=O)CCCCCCCN(CCCCCCCC(=O)OCCCCC(CCCCC)CCCCC)C(=O)CCCN(C)C]

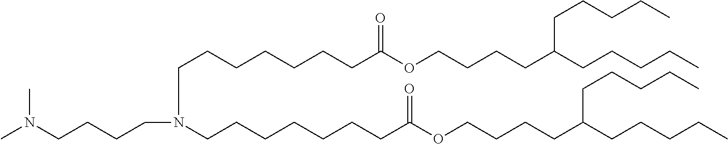 [SMILES: CCCCCC(CCCCC)CCCCOC(=O)CCCCCCCN(CCCCCCCC(=O)OCCCCC(CCCCC)CCCCC)CCCCN(C)C]

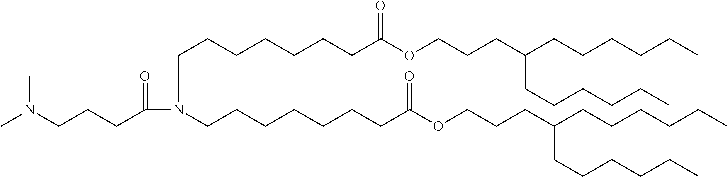 [SMILES: CCCCCCC(CCCCCC)CCCOC(=O)CCCCCCCN(CCCCCCCC(=O)OCCCC(CCCCCC)CCCCCC)C(=O)CCCN(C)C]

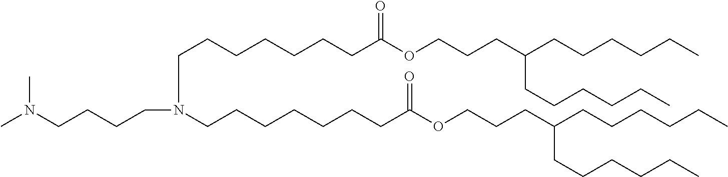 [SMILES: CCCCCCC(CCCCCC)CCCOC(=O)CCCCCCCN(CCCCCCCC(=O)OCCCC(CCCCCC)CCCCCC)CCCCN(C)C]

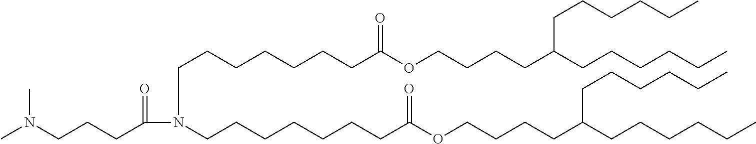 [SMILES: CCCCCCC(CCCCCC)CCCCOC(=O)CCCCCCCN(CCCCCCCC(=O)OCCCCC(CCCCCC)CCCCCC)C(=O)CCCN(C)C]

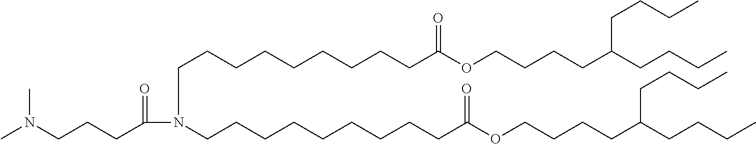 [SMILES: CCCCC(CCCC)CCCCOC(=O)CCCCCCCCCN(CCCCCCCCCC(=O)OCCCCC(CCCC)CCCC)C(=O)CCCN(C)C]

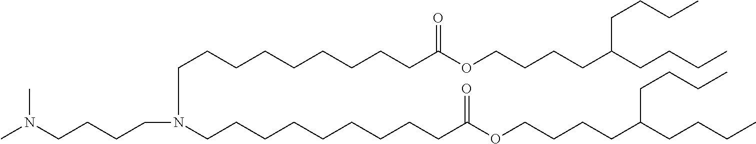 [SMILES: CCCCC(CCCC)CCCCOC(=O)CCCCCCCCCN(CCCCCCCCCC(=O)OCCCCC(CCCC)CCCC)CCCCN(C)C]

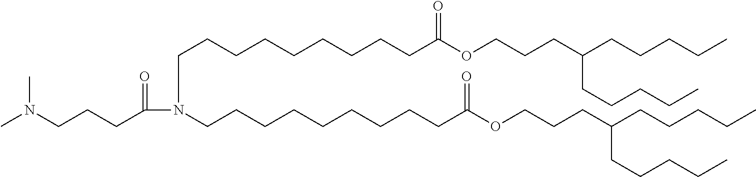 [SMILES: CCCCCC(CCCCC)CCCOC(=O)CCCCCCCCCN(CCCCCCCCCC(=O)OCCCC(CCCCC)CCCCC)C(=O)CCCN(C)C]

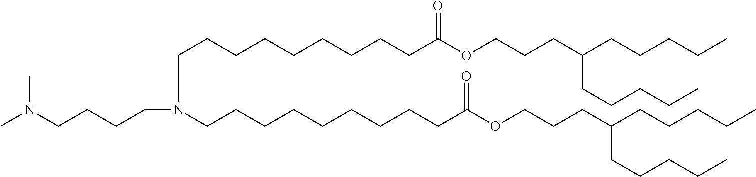 [SMILES: CCCCCC(CCCCC)CCCOC(=O)CCCCCCCCCN(CCCCCCCCCC(=O)OCCCC(CCCCC)CCCCC)CCCCN(C)C]

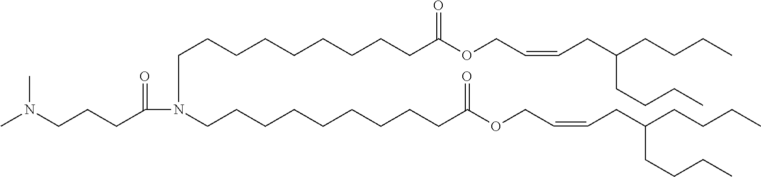 [SMILES: CCCCC(C/C=C\COC(=O)CCCCCCCCCN(CCCCCCCCCC(=O)OC/C=C\CC(CCCC)CCCC)C(=O)CCCN(C)C)CCCC]

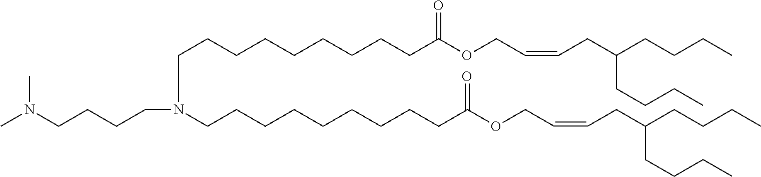 [SMILES: CCCCC(C/C=C\COC(=O)CCCCCCCCCN(CCCCCCCCCC(=O)OC/C=C\CC(CCCC)CCCC)CCCCN(C)C)CCCC]

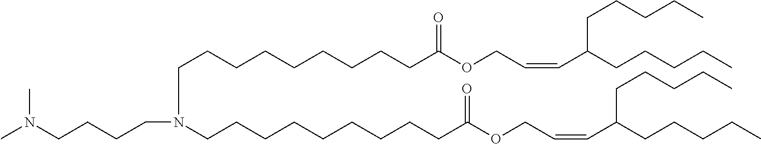 [SMILES: CCCCCC(/C=C\COC(=O)CCCCCCCCCN(CCCCCCCCCC(=O)OC/C=C\C(CCCCC)CCCCC)CCCCN(C)C)CCCCC]

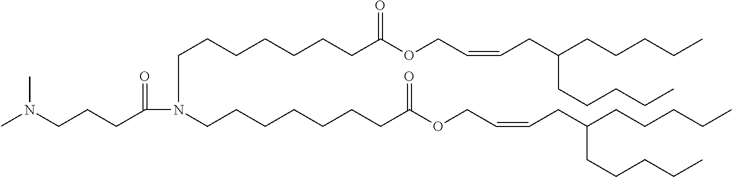 [SMILES: CCCCCC(C/C=C\COC(=O)CCCCCCCN(CCCCCCCC(=O)OC/C=C\CC(CCCCC)CCCCC)C(=O)CCCN(C)C)CCCCC]

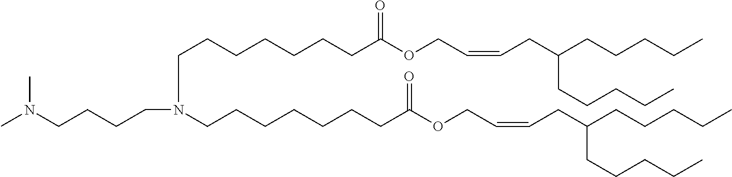 [SMILES: CCCCCC(C/C=C\COC(=O)CCCCCCCN(CCCCCCCC(=O)OC/C=C\CC(CCCCC)CCCCC)CCCCN(C)C)CCCCC]

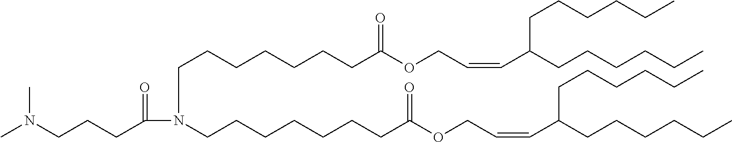 [SMILES: CCCCCCC(/C=C\COC(=O)CCCCCCCN(CCCCCCCC(=O)OC/C=C\C(CCCCCC)CCCCCC)C(=O)CCCN(C)C)CCCCCC]

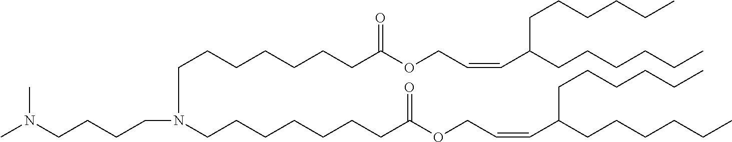 [SMILES: CCCCCCC(/C=C\COC(=O)CCCCCCCN(CCCCCCCC(=O)OC/C=C\C(CCCCCC)CCCCCC)CCCCN(C)C)CCCCCC]

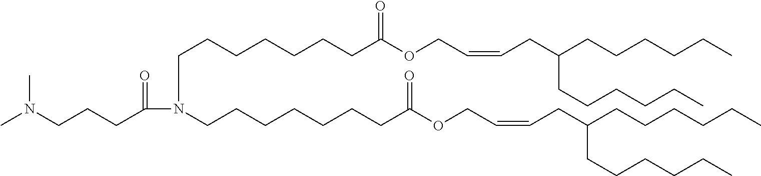 [SMILES: CCCCCCC(C/C=C\COC(=O)CCCCCCCN(CCCCCCCC(=O)OC/C=C\CC(CCCCCC)CCCCCC)C(=O)CCCN(C)C)CCCCCC]

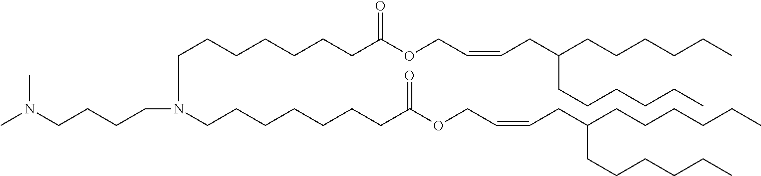 [SMILES: CCCCCCC(C/C=C\COC(=O)CCCCCCCN(CCCCCCCC(=O)OC/C=C\CC(CCCCCC)CCCCCC)CCCCN(C)C)CCCCCC]

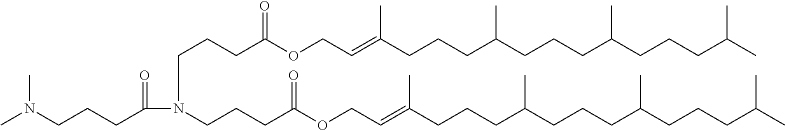 [SMILES: C/C(=C\COC(=O)CCCN(CCCC(=O)OC/C=C(\C)CCCC(C)CCCC(C)CCCC(C)C)C(=O)CCCN(C)C)CCCC(C)CCCC(C)CCCC(C)C]

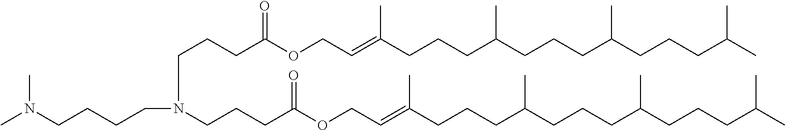 [SMILES: C/C(=C\COC(=O)CCCN(CCCCN(C)C)CCCC(=O)OC/C=C(\C)CCCC(C)CCCC(C)CCCC(C)C)CCCC(C)CCCC(C)CCCC(C)C]

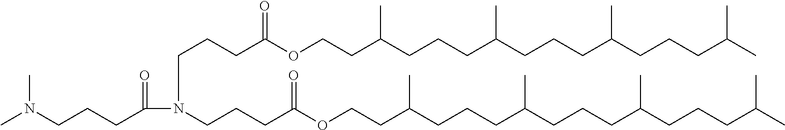 [SMILES: CC(C)CCCC(C)CCCC(C)CCCC(C)CCOC(=O)CCCN(CCCC(=O)OCCC(C)CCCC(C)CCCC(C)CCCC(C)C)C(=O)CCCN(C)C]

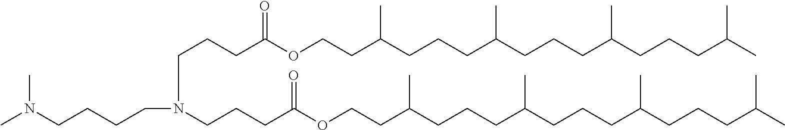 [SMILES: CC(C)CCCC(C)CCCC(C)CCCC(C)CCOC(=O)CCCN(CCCCN(C)C)CCCC(=O)OCCC(C)CCCC(C)CCCC(C)CCCC(C)C]

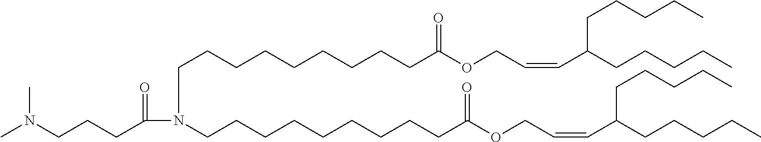 [SMILES: CCCCCC(/C=C\COC(=O)CCCCCCCCCN(CCCCCCCCCC(=O)OC/C=C\C(CCCCC)CCCCC)C(=O)CCCN(C)C)CCCCC]

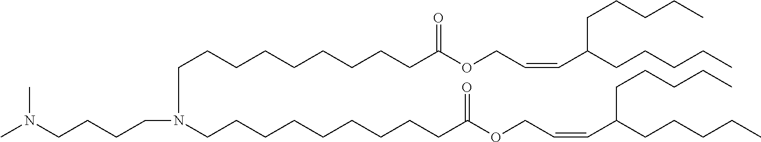 [SMILES: CCCCCC(/C=C\COC(=O)CCCCCCCCCN(CCCCCCCCCC(=O)OC/C=C\C(CCCCC)CCCCC)CCCCN(C)C)CCCCC]

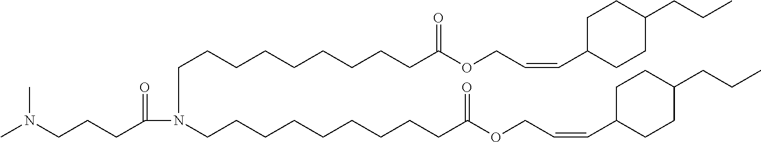 [SMILES: CCCC1CCC(/C=C\COC(=O)CCCCCCCCCN(CCCCCCCCCC(=O)OC/C=C\C2CCC(CCC)CC2)C(=O)CCCN(C)C)CC1]

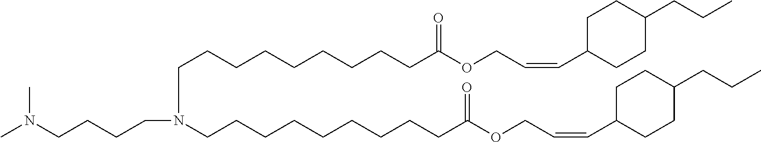 [SMILES: CCCC1CCC(/C=C\COC(=O)CCCCCCCCCN(CCCCCCCCCC(=O)OC/C=C\C2CCC(CCC)CC2)CCCCN(C)C)CC1]

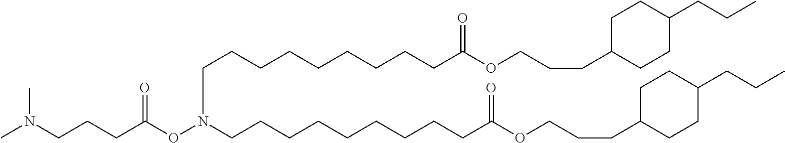 [SMILES: CCCC1CCC(CCCOC(=O)CCCCCCCCCN(CCCCCCCCCC(=O)OCCCC2CCC(CCC)CC2)OC(=O)CCCN(C)C)CC1]

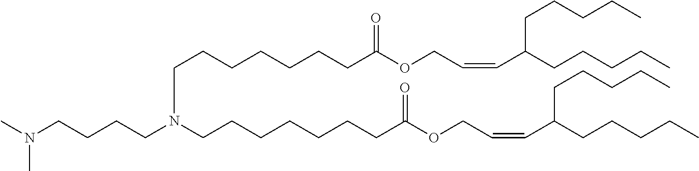 [SMILES: CCCCCC(/C=C\COC(=O)CCCCCCCN(CCCCCCCC(=O)OC/C=C\C(CCCCC)CCCCC)CCCCN(C)C)CCCCC]

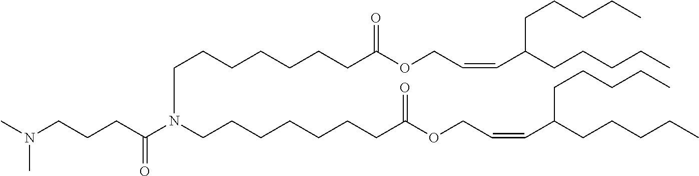 [SMILES: CCCCCC(/C=C\COC(=O)CCCCCCCN(CCCCCCCC(=O)OC/C=C\C(CCCCC)CCCCC)C(=O)CCCN(C)C)CCCCC]

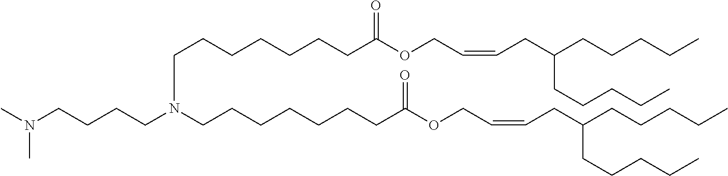 [SMILES: CCCCCC(C/C=C\COC(=O)CCCCCCCN(CCCCCCCC(=O)OC/C=C\CC(CCCCC)CCCCC)CCCCN(C)C)CCCCC]

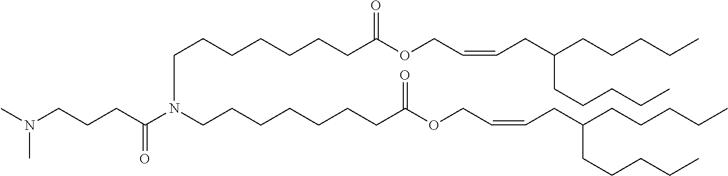 [SMILES: CCCCCC(C/C=C\COC(=O)CCCCCCCN(CCCCCCCC(=O)OC/C=C\CC(CCCCC)CCCCC)C(=O)CCCN(C)C)CCCCC]

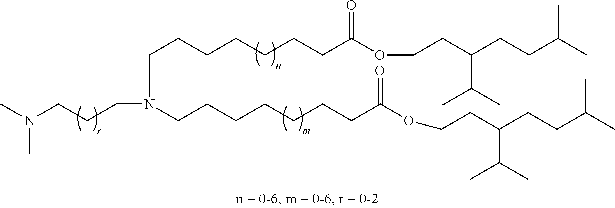

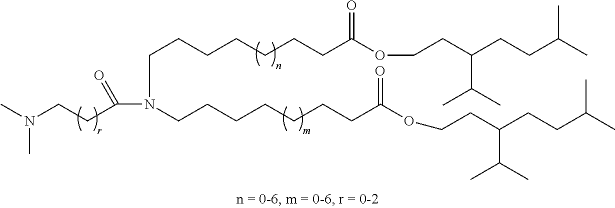

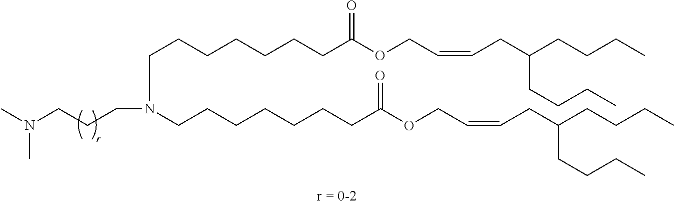

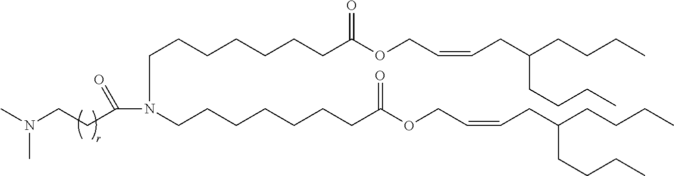

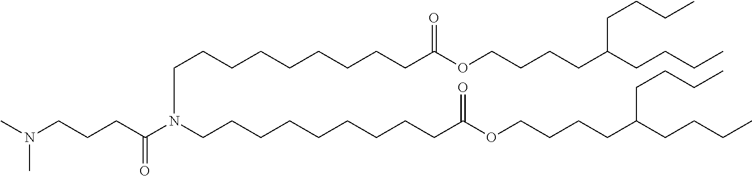 [SMILES: CCCCC(CCCC)CCCCOC(=O)CCCCCCCCCN(CCCCCCCCCC(=O)OCCCCC(CCCC)CCCC)C(=O)CCCN(C)C]

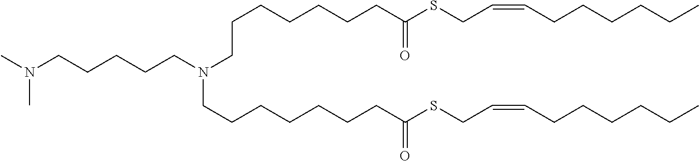 [SMILES: CCCCCC/C=C\CSC(=O)CCCCCCCN(CCCCCCCC(=O)SC/C=C\CCCCCC)CCCCCN(C)C]

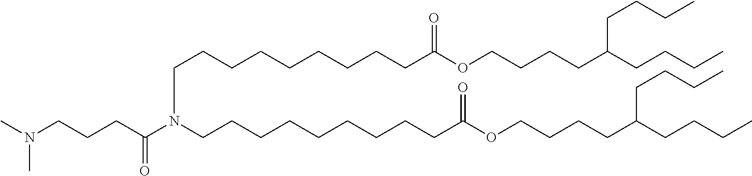 [SMILES: CCCCC(CCCC)CCCCOC(=O)CCCCCCCCCN(CCCCCCCCCC(=O)OCCCCC(CCCC)CCCC)C(=O)CCCN(C)C]

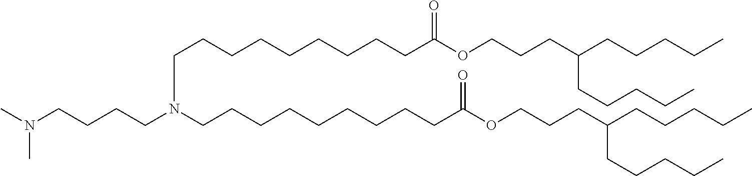 [SMILES: CCCCCC(CCCCC)CCCOC(=O)CCCCCCCCCN(CCCCCCCCCC(=O)OCCCC(CCCCC)CCCCC)CCCCN(C)C]

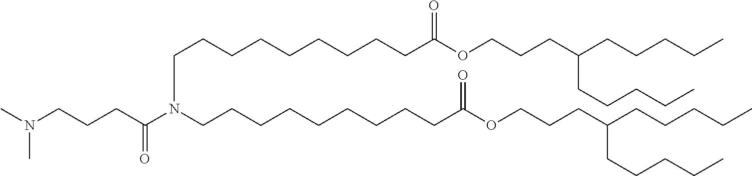 [SMILES: CCCCCC(CCCCC)CCCOC(=O)CCCCCCCCCN(CCCCCCCCCC(=O)OCCCC(CCCCC)CCCCC)C(=O)CCCN(C)C]

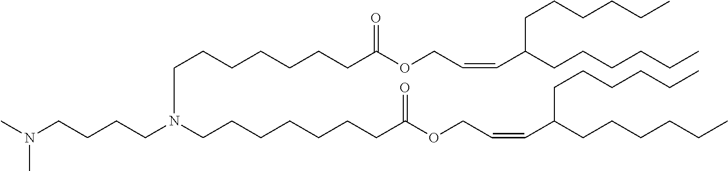 [SMILES: CCCCCCC(/C=C\COC(=O)CCCCCCCN(CCCCCCCC(=O)OC/C=C\C(CCCCCC)CCCCCC)CCCCN(C)C)CCCCCC]

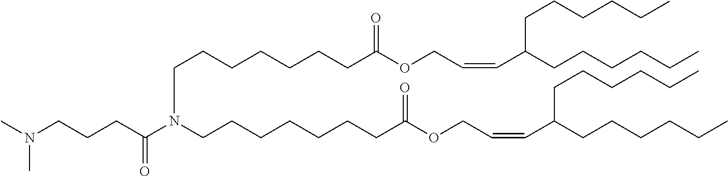 [SMILES: CCCCCCC(/C=C\COC(=O)CCCCCCCN(CCCCCCCC(=O)OC/C=C\C(CCCCCC)CCCCCC)C(=O)CCCN(C)C)CCCCCC]

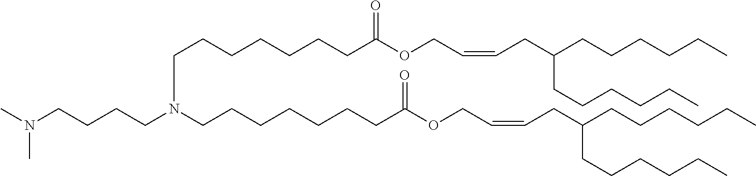 [SMILES: CCCCCCC(C/C=C\COC(=O)CCCCCCCN(CCCCCCCC(=O)OC/C=C\CC(CCCCCC)CCCCCC)CCCCN(C)C)CCCCCC]

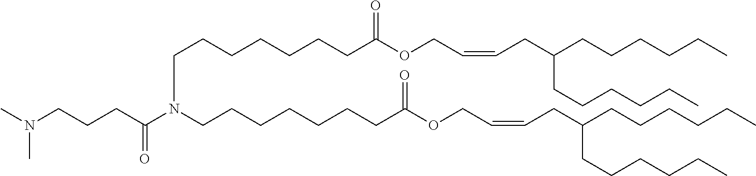 [SMILES: CCCCCCC(C/C=C\COC(=O)CCCCCCCN(CCCCCCCC(=O)OC/C=C\CC(CCCCCC)CCCCCC)C(=O)CCCN(C)C)CCCCCC]

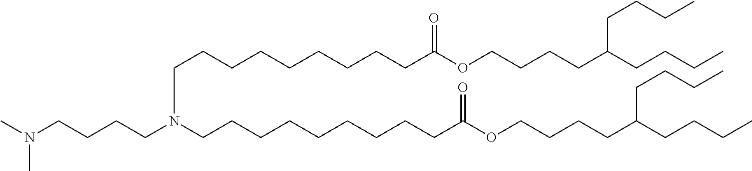 [SMILES: CCCCC(CCCC)CCCCOC(=O)CCCCCCCCCN(CCCCCCCCCC(=O)OCCCCC(CCCC)CCCC)CCCCN(C)C]

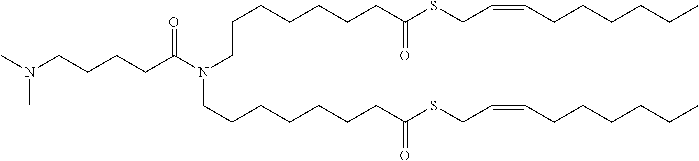 [SMILES: CCCCCC/C=C\CSC(=O)CCCCCCCN(CCCCCCCC(=O)SC/C=C\CCCCCC)C(=O)CCCCN(C)C]

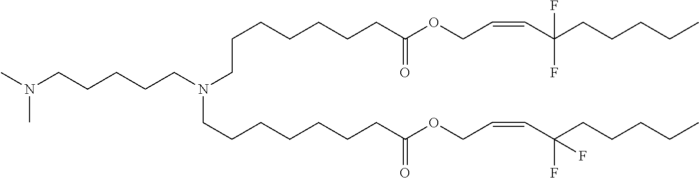 [SMILES: CCCCCC(F)(F)/C=C\COC(=O)CCCCCCCN(CCCCCCCC(=O)OC/C=C\C(F)(F)CCCCC)CCCCCN(C)C]

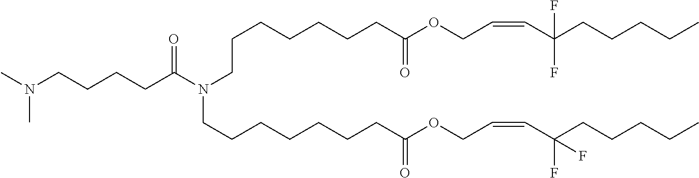 [SMILES: CCCCCC(F)(F)/C=C\COC(=O)CCCCCCCN(CCCCCCCC(=O)OC/C=C\C(F)(F)CCCCC)C(=O)CCCCN(C)C]

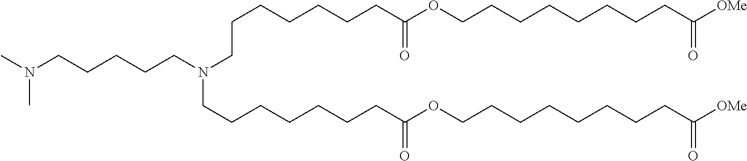 [SMILES: COC(=O)CCCCCCCCOC(=O)CCCCCCCN(CCCCCCCC(=O)OCCCCCCCCC(=O)OC)CCCCCN(C)C]

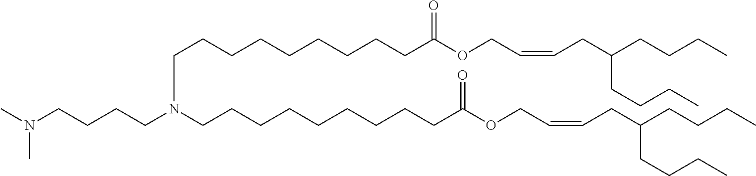 [SMILES: CCCCC(C/C=C\COC(=O)CCCCCCCCCN(CCCCCCCCCC(=O)OC/C=C\CC(CCCC)CCCC)CCCCN(C)C)CCCC]

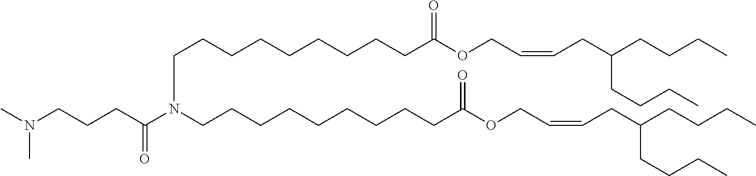 [SMILES: CCCCC(C/C=C\COC(=O)CCCCCCCCCN(CCCCCCCCCC(=O)OC/C=C\CC(CCCC)CCCC)C(=O)CCCN(C)C)CCCC]

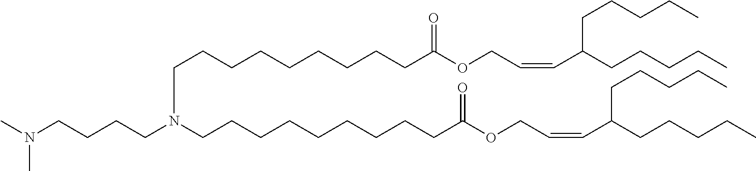 [SMILES: CCCCCC(/C=C\COC(=O)CCCCCCCCCN(CCCCCCCCCC(=O)OC/C=C\C(CCCCC)CCCCC)CCCCN(C)C)CCCCC]

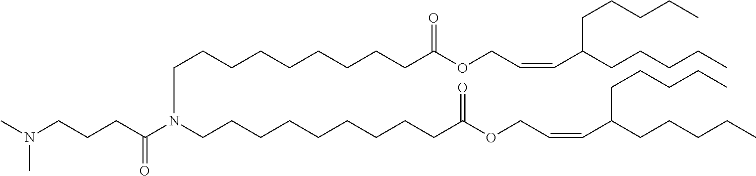 [SMILES: CCCCCC(/C=C\COC(=O)CCCCCCCCCN(CCCCCCCCCC(=O)OC/C=C\C(CCCCC)CCCCC)C(=O)CCCN(C)C)CCCCC]

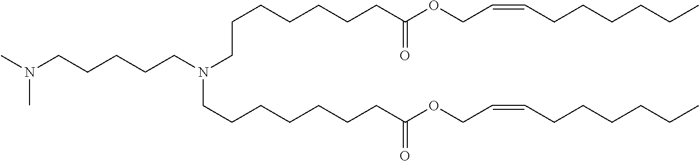 [SMILES: CCCCCC/C=C\COC(=O)CCCCCCCN(CCCCCCCC(=O)OC/C=C\CCCCCC)CCCCCN(C)C]

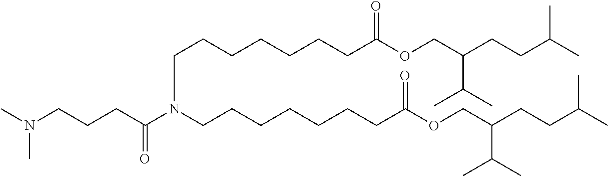 [SMILES: CC(C)CCC(COC(=O)CCCCCCCN(CCCCCCCC(=O)OCC(CCC(C)C)C(C)C)C(=O)CCCN(C)C)C(C)C]

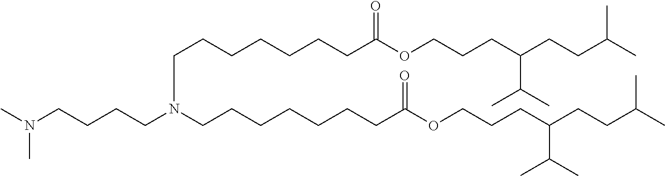 [SMILES: CC(C)CCC(CCCOC(=O)CCCCCCCN(CCCCCCCC(=O)OCCCC(CCC(C)C)C(C)C)CCCCN(C)C)C(C)C]

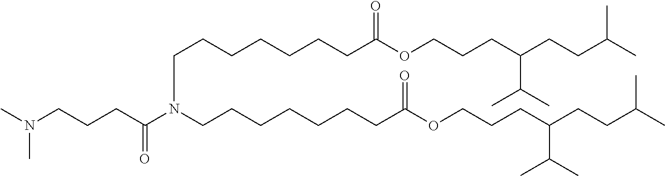 [SMILES: CC(C)CCC(CCCOC(=O)CCCCCCCN(CCCCCCCC(=O)OCCCC(CCC(C)C)C(C)C)C(=O)CCCN(C)C)C(C)C]

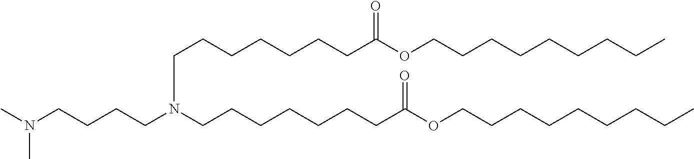 [SMILES: CCCCCCCCCOC(=O)CCCCCCCN(CCCCCCCC(=O)OCCCCCCCCC)CCCCN(C)C]

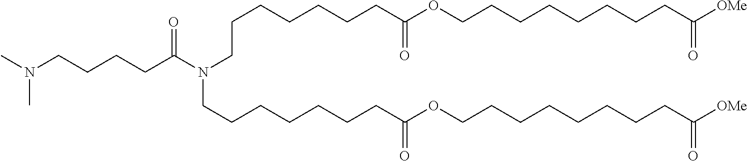 [SMILES: COC(=O)CCCCCCCCOC(=O)CCCCCCCN(CCCCCCCC(=O)OCCCCCCCCC(=O)OC)C(=O)CCCCN(C)C]

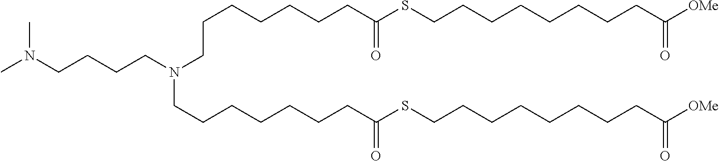 [SMILES: COC(=O)CCCCCCCCSC(=O)CCCCCCCN(CCCCCCCC(=O)SCCCCCCCCC(=O)OC)CCCCN(C)C]

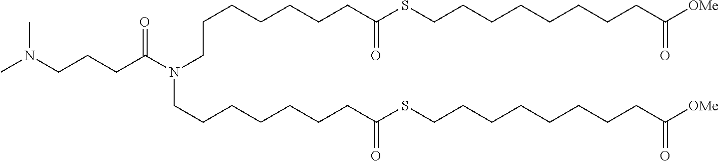 [SMILES: COC(=O)CCCCCCCCSC(=O)CCCCCCCN(CCCCCCCC(=O)SCCCCCCCCC(=O)OC)C(=O)CCCN(C)C]

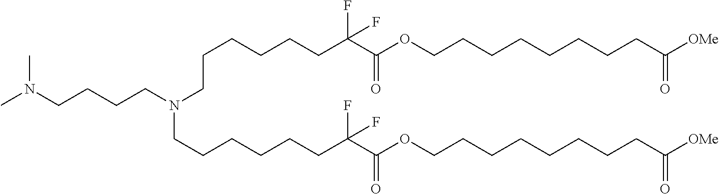 [SMILES: COC(=O)CCCCCCCCOC(=O)C(F)(F)CCCCCCN(CCCCCCC(F)(F)C(=O)OCCCCCCCCC(=O)OC)CCCCN(C)C]

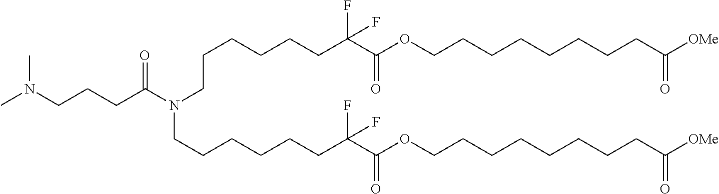 [SMILES: COC(=O)CCCCCCCCOC(=O)C(F)(F)CCCCCCN(CCCCCCC(F)(F)C(=O)OCCCCCCCCC(=O)OC)C(=O)CCCN(C)C]

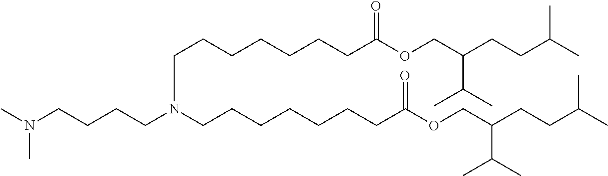 [SMILES: CC(C)CCC(COC(=O)CCCCCCCN(CCCCCCCC(=O)OCC(CCC(C)C)C(C)C)CCCCN(C)C)C(C)C]

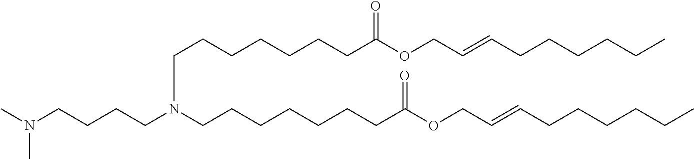 [SMILES: CCCCCC/C=C/COC(=O)CCCCCCCN(CCCCCCCC(=O)OC/C=C/CCCCCC)CCCCN(C)C]

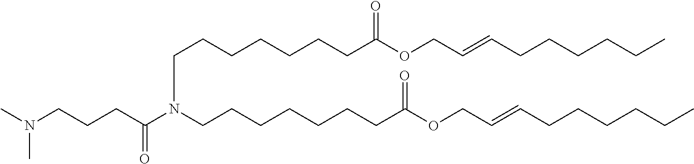 [SMILES: CCCCCC/C=C/COC(=O)CCCCCCCN(CCCCCCCC(=O)OC/C=C/CCCCCC)C(=O)CCCN(C)C]

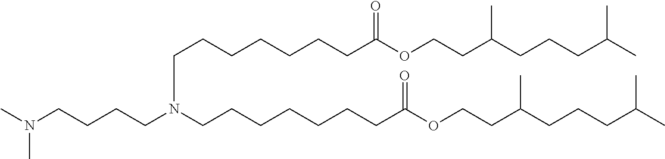 [SMILES: CC(C)CCCC(C)CCOC(=O)CCCCCCCN(CCCCCCCC(=O)OCCC(C)CCCC(C)C)CCCCN(C)C]

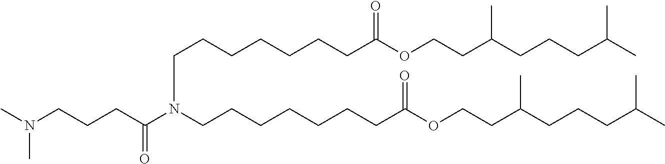 [SMILES: CC(C)CCCC(C)CCOC(=O)CCCCCCCN(CCCCCCCC(=O)OCCC(C)CCCC(C)C)C(=O)CCCN(C)C]

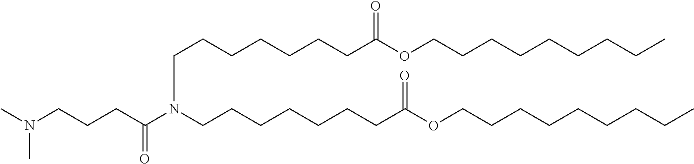 [SMILES: CCCCCCCCCOC(=O)CCCCCCCN(CCCCCCCC(=O)OCCCCCCCCC)C(=O)CCCN(C)C]

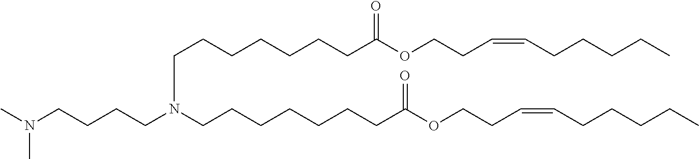 [SMILES: CCCCC/C=C\CCOC(=O)CCCCCCCN(CCCCCCCC(=O)OCC/C=C\CCCCC)CCCCN(C)C]

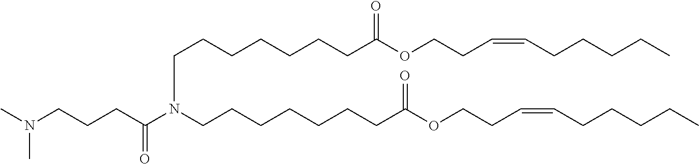 [SMILES: CCCCC/C=C\CCOC(=O)CCCCCCCN(CCCCCCCC(=O)OCC/C=C\CCCCC)C(=O)CCCN(C)C]

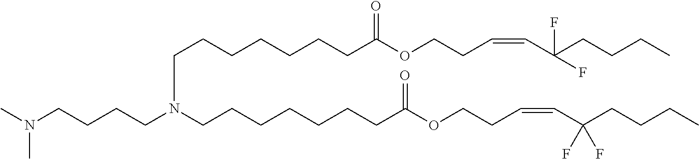 [SMILES: CCCCC(F)(F)/C=C\CCOC(=O)CCCCCCCN(CCCCCCCC(=O)OCC/C=C\C(F)(F)CCCC)CCCCN(C)C]

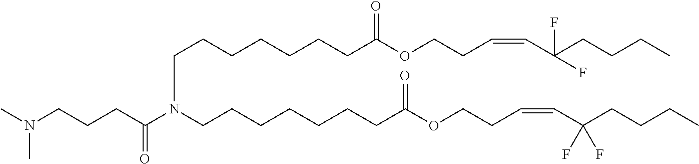 [SMILES: CCCCC(F)(F)/C=C\CCOC(=O)CCCCCCCN(CCCCCCCC(=O)OCC/C=C\C(F)(F)CCCC)C(=O)CCCN(C)C]

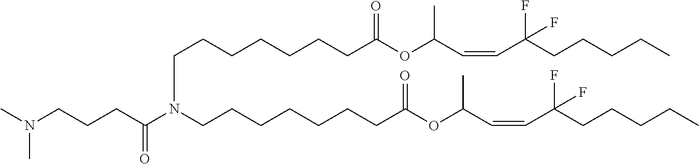 [SMILES: CCCCCC(F)(F)/C=C\C(C)OC(=O)CCCCCCCN(CCCCCCCC(=O)OC(C)/C=C\C(F)(F)CCCCC)C(=O)CCCN(C)C]

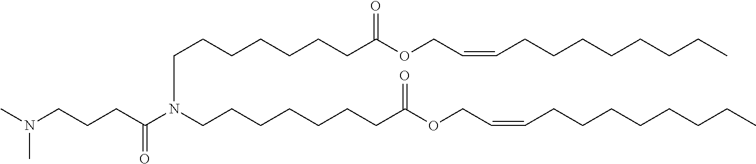 [SMILES: CCCCCCCC/C=C\COC(=O)CCCCCCCN(CCCCCCCC(=O)OC/C=C\CCCCCCCC)C(=O)CCCN(C)C]

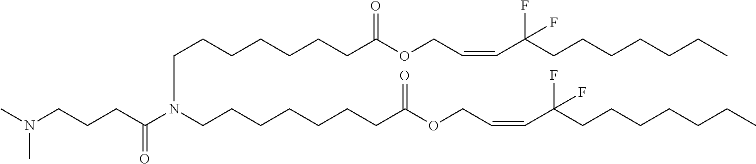 [SMILES: CCCCCCCC(F)(F)/C=C\COC(=O)CCCCCCCN(CCCCCCCC(=O)OC/C=C\C(F)(F)CCCCCCC)C(=O)CCCN(C)C]

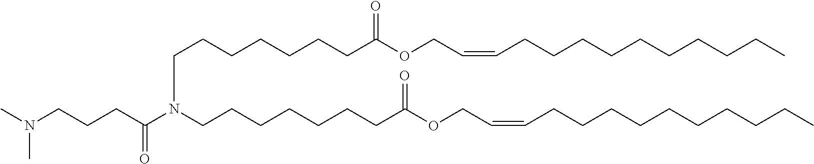 [SMILES: CCCCCCCCCC/C=C\COC(=O)CCCCCCCN(CCCCCCCC(=O)OC/C=C\CCCCCCCCCC)C(=O)CCCN(C)C]

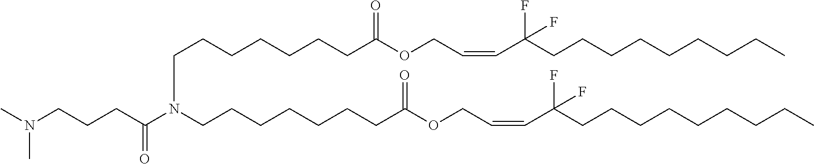 [SMILES: CCCCCCCCCC(F)(F)/C=C\COC(=O)CCCCCCCN(CCCCCCCC(=O)OC/C=C\C(F)(F)CCCCCCCCC)C(=O)CCCN(C)C]

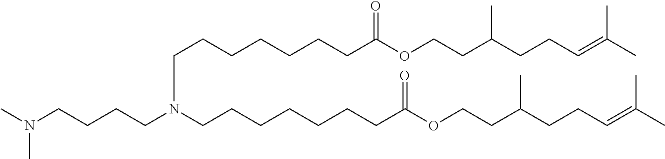 [SMILES: CC(C)=CCCC(C)CCOC(=O)CCCCCCCN(CCCCCCCC(=O)OCCC(C)CCC=C(C)C)CCCCN(C)C]

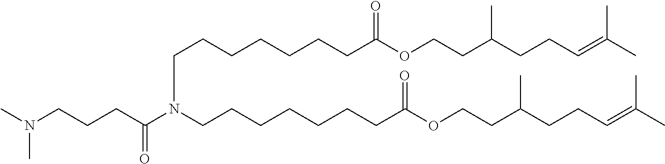 [SMILES: CC(C)=CCCC(C)CCOC(=O)CCCCCCCN(CCCCCCCC(=O)OCCC(C)CCC=C(C)C)C(=O)CCCN(C)C]

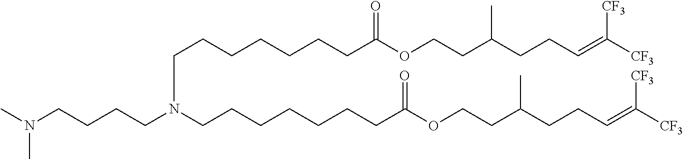 [SMILES: CC(CCC=C(C(F)(F)F)C(F)(F)F)CCOC(=O)CCCCCCCN(CCCCCCCC(=O)OCCC(C)CCC=C(C(F)(F)F)C(F)(F)F)CCCCN(C)C]

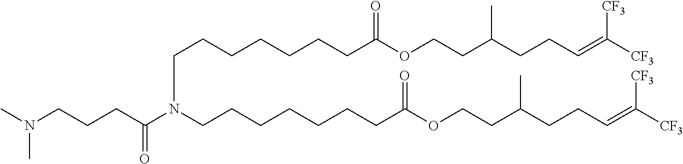 [SMILES: CC(CCC=C(C(F)(F)F)C(F)(F)F)CCOC(=O)CCCCCCCN(CCCCCCCC(=O)OCCC(C)CCC=C(C(F)(F)F)C(F)(F)F)C(=O)CCCN(C)C]

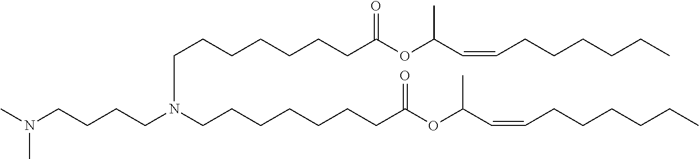 [SMILES: CCCCCC/C=C\C(C)OC(=O)CCCCCCCN(CCCCCCCC(=O)OC(C)/C=C\CCCCCC)CCCCN(C)C]

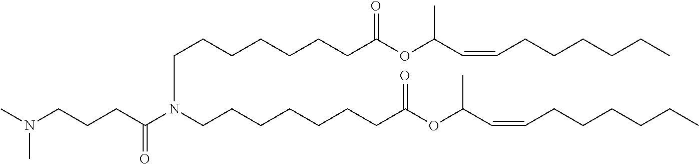 [SMILES: CCCCCC/C=C\C(C)OC(=O)CCCCCCCN(CCCCCCCC(=O)OC(C)/C=C\CCCCCC)C(=O)CCCN(C)C]

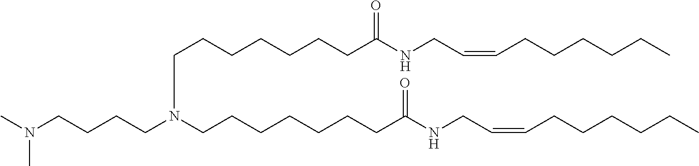 [SMILES: CCCCCC/C=C\CNC(=O)CCCCCCCN(CCCCCCCC(=O)NC/C=C\CCCCCC)CCCCN(C)C]

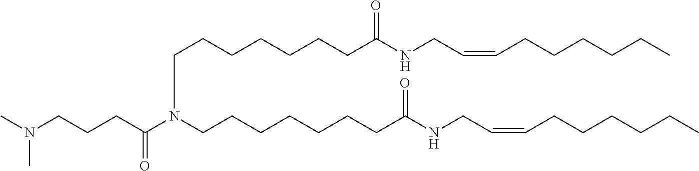 [SMILES: CCCCCC/C=C\CNC(=O)CCCCCCCN(CCCCCCCC(=O)NC/C=C\CCCCCC)C(=O)CCCN(C)C]

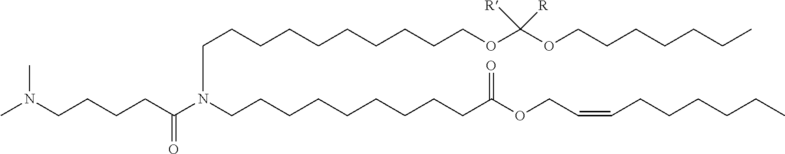

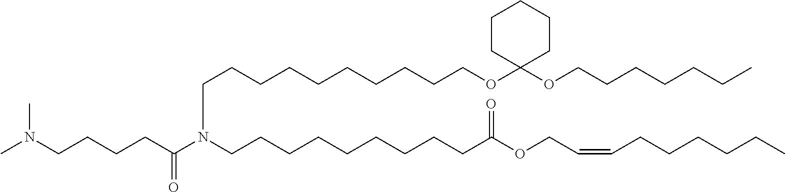 [SMILES: CCCCCC/C=C\COC(=O)CCCCCCCCCN(CCCCCCCCCCOC1(OCCCCCCC)CCCCC1)C(=O)CCCCN(C)C]

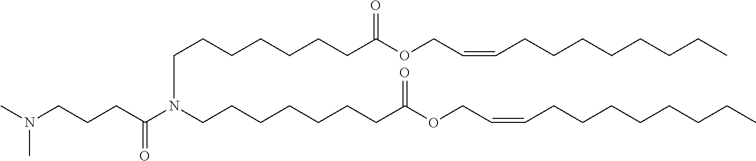 [SMILES: CCCCCCCC/C=C\COC(=O)CCCCCCCN(CCCCCCCC(=O)OC/C=C\CCCCCCCC)C(=O)CCCN(C)C]

In another embodiment, the cationic lipid of the present invention is selected from the following compounds, and salts thereof (including pharmaceutically acceptable salts thereof):

TABLE 14
Compound

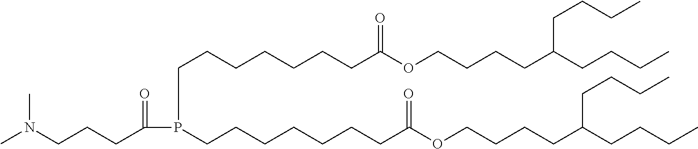 [SMILES: CCCCC(CCCC)CCCCOC(=O)CCCCCCCP(CCCCCCCC(=O)OCCCCC(CCCC)CCCC)C(=O)CCCN(C)C]

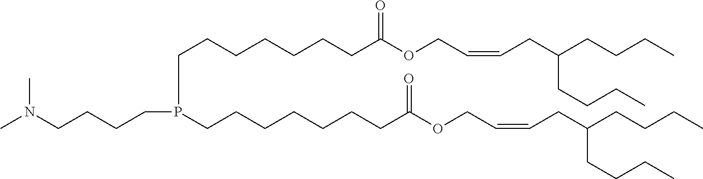 [SMILES: CCCCC(C/C=C\COC(=O)CCCCCCCP(CCCCCCCC(=O)OC/C=C\CC(CCCC)CCCC)CCCCN(C)C)CCCC]

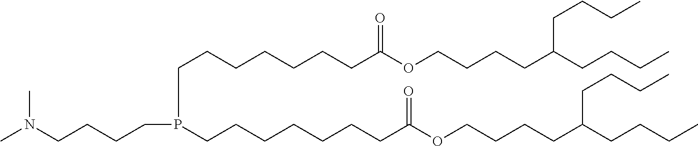 [SMILES: CCCCC(CCCC)CCCCOC(=O)CCCCCCCP(CCCCCCCC(=O)OCCCCC(CCCC)CCCC)CCCCN(C)C]

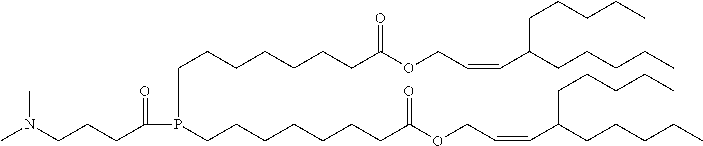 [SMILES: CCCCCC(/C=C\COC(=O)CCCCCCCP(CCCCCCCC(=O)OC/C=C\C(CCCCC)CCCCC)C(=O)CCCN(C)C)CCCCC]

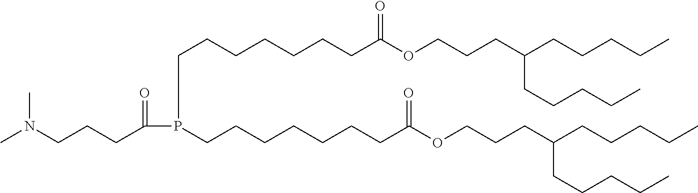 [SMILES: CCCCCC(CCCCC)CCCOC(=O)CCCCCCCP(CCCCCCCC(=O)OCCCC(CCCCC)CCCCC)C(=O)CCCN(C)C]

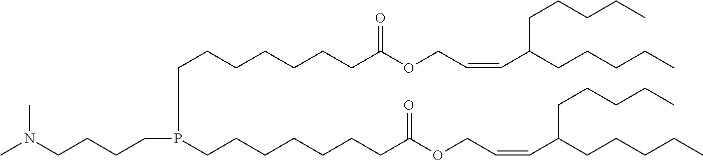 [SMILES: CCCCCC(/C=C\COC(=O)CCCCCCCP(CCCCCCCC(=O)OC/C=C\C(CCCCC)CCCCC)CCCCN(C)C)CCCCC]

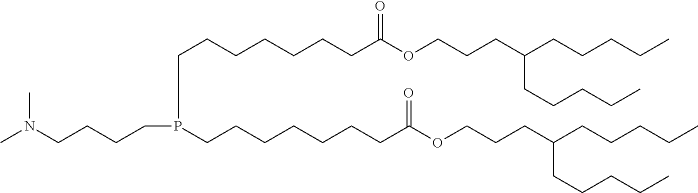 [SMILES: CCCCCC(CCCCC)CCCOC(=O)CCCCCCCP(CCCCCCCC(=O)OCCCC(CCCCC)CCCCC)CCCCN(C)C]

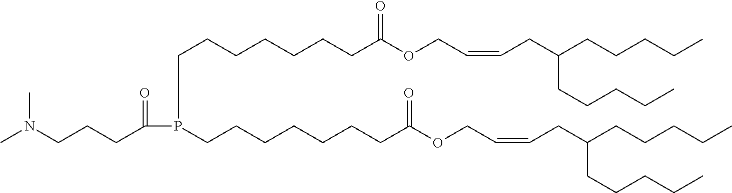 [SMILES: CCCCCC(C/C=C\COC(=O)CCCCCCCP(CCCCCCCC(=O)OC/C=C\CC(CCCCC)CCCCC)C(=O)CCCN(C)C)CCCCC]

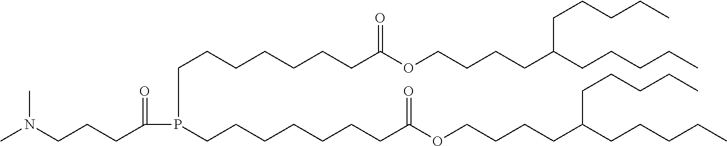 [SMILES: CCCCCC(CCCCC)CCCCOC(=O)CCCCCCCP(CCCCCCCC(=O)OCCCCC(CCCCC)CCCCC)C(=O)CCCN(C)C]

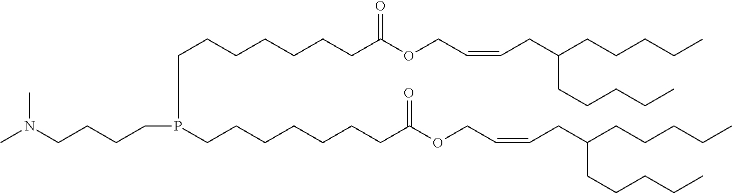 [SMILES: CCCCCC(C/C=C\COC(=O)CCCCCCCP(CCCCCCCC(=O)OC/C=C\CC(CCCCC)CCCCC)CCCCN(C)C)CCCCC]

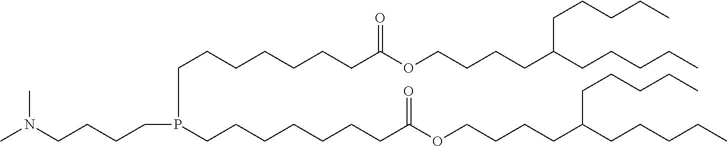 [SMILES: CCCCCC(CCCCC)CCCCOC(=O)CCCCCCCP(CCCCCCCC(=O)OCCCCC(CCCCC)CCCCC)CCCCN(C)C]

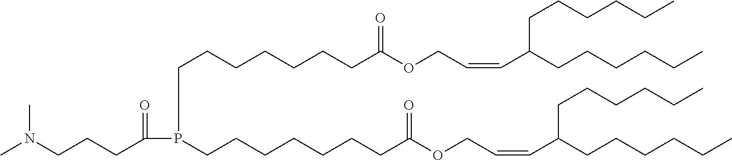 [SMILES: CCCCCCC(/C=C\COC(=O)CCCCCCCP(CCCCCCCC(=O)OC/C=C\C(CCCCCC)CCCCCC)C(=O)CCCN(C)C)CCCCCC]

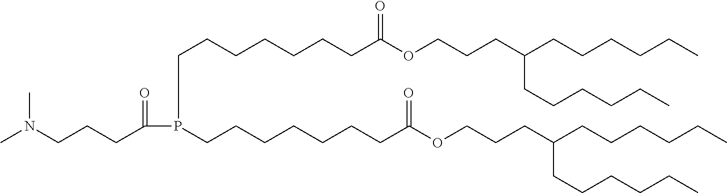 [SMILES: CCCCCCC(CCCCCC)CCCOC(=O)CCCCCCCP(CCCCCCCC(=O)OCCCC(CCCCCC)CCCCCC)C(=O)CCCN(C)C]

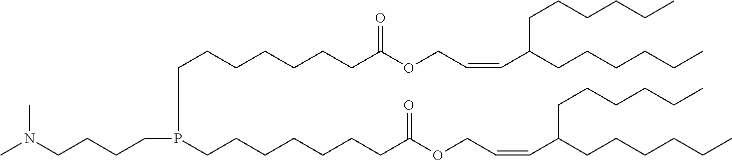 [SMILES: CCCCCCC(/C=C\COC(=O)CCCCCCCP(CCCCCCCC(=O)OC/C=C\C(CCCCCC)CCCCCC)CCCCN(C)C)CCCCCC]

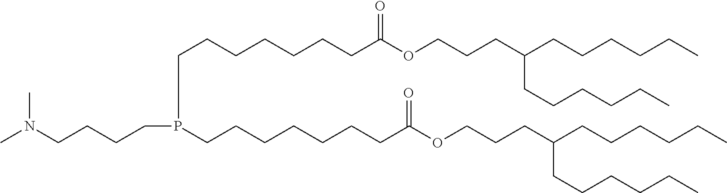 [SMILES: CCCCCCC(CCCCCC)CCCOC(=O)CCCCCCCP(CCCCCCCC(=O)OCCCC(CCCCCC)CCCCCC)CCCCN(C)C]

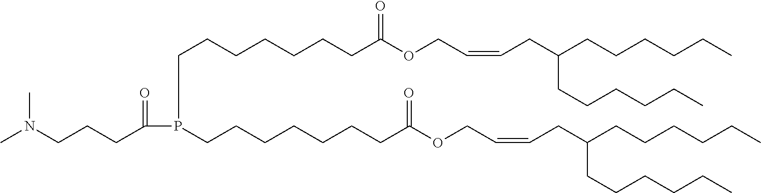 [SMILES: CCCCCCC(C/C=C\COC(=O)CCCCCCCP(CCCCCCCC(=O)OC/C=C\CC(CCCCCC)CCCCCC)C(=O)CCCN(C)C)CCCCCC]

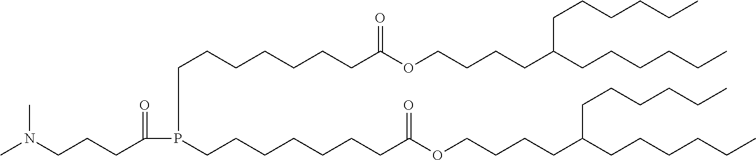 [SMILES: CCCCCCC(CCCCCC)CCCCOC(=O)CCCCCCCP(CCCCCCCC(=O)OCCCCC(CCCCCC)CCCCCC)C(=O)CCCN(C)C]

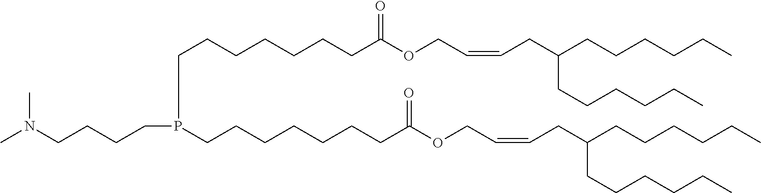 [SMILES: CCCCCCC(C/C=C\COC(=O)CCCCCCCP(CCCCCCCC(=O)OC/C=C\CC(CCCCCC)CCCCCC)CCCCN(C)C)CCCCCC]

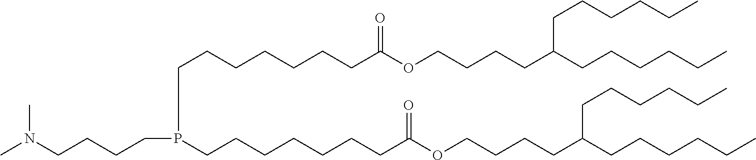 [SMILES: CCCCCCC(CCCCCC)CCCCOC(=O)CCCCCCCP(CCCCCCCC(=O)OCCCCC(CCCCCC)CCCCCC)CCCCN(C)C]

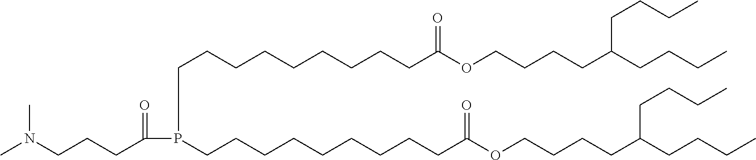 [SMILES: CCCCC(CCCC)CCCCOC(=O)CCCCCCCCCP(CCCCCCCCCC(=O)OCCCCC(CCCC)CCCC)C(=O)CCCN(C)C]

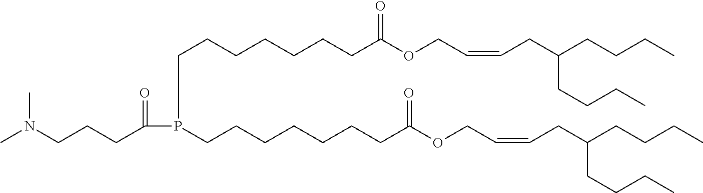 [SMILES: CCCCC(C/C=C\COC(=O)CCCCCCCP(CCCCCCCC(=O)OC/C=C\CC(CCCC)CCCC)C(=O)CCCN(C)C)CCCC]

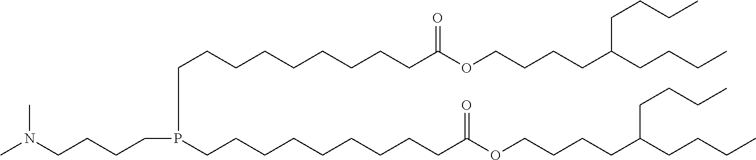 [SMILES: CCCCC(CCCC)CCCCOC(=O)CCCCCCCCCP(CCCCCCCCCC(=O)OCCCCC(CCCC)CCCC)CCCCN(C)C]

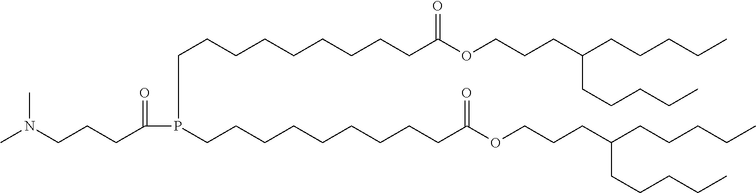 [SMILES: CCCCCC(CCCCC)CCCOC(=O)CCCCCCCCCP(CCCCCCCCCC(=O)OCCCC(CCCCC)CCCCC)C(=O)CCCN(C)C]

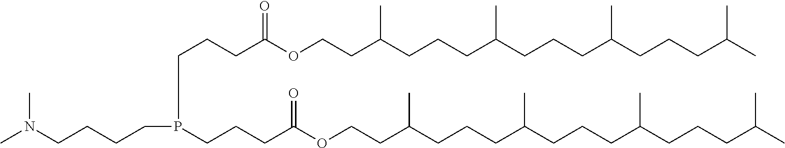 [SMILES: CC(C)CCCC(C)CCCC(C)CCCC(C)CCOC(=O)CCCP(CCCCN(C)C)CCCC(=O)OCCC(C)CCCC(C)CCCC(C)CCCC(C)C]

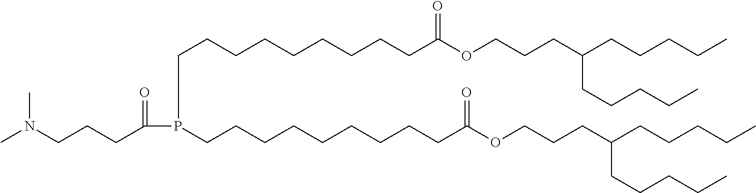 [SMILES: CCCCCC(CCCCC)CCCOC(=O)CCCCCCCCCP(CCCCCCCCCC(=O)OCCCC(CCCCC)CCCCC)C(=O)CCCN(C)C]

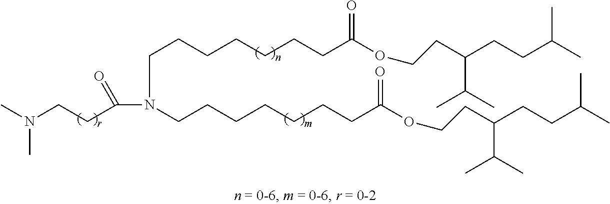

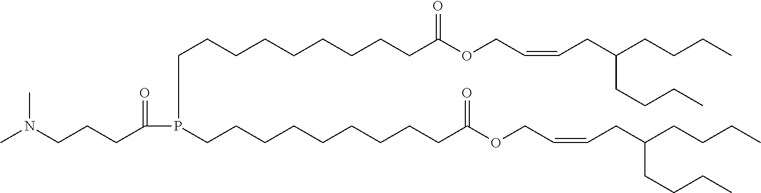 [SMILES: CCCCC(C/C=C\COC(=O)CCCCCCCCCP(CCCCCCCCCC(=O)OC/C=C\CC(CCCC)CCCC)C(=O)CCCN(C)C)CCCC]

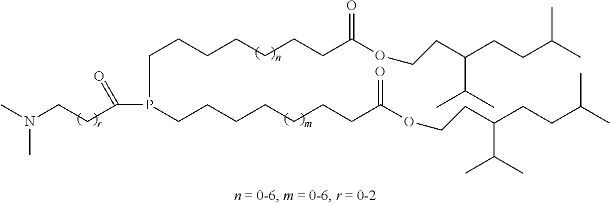

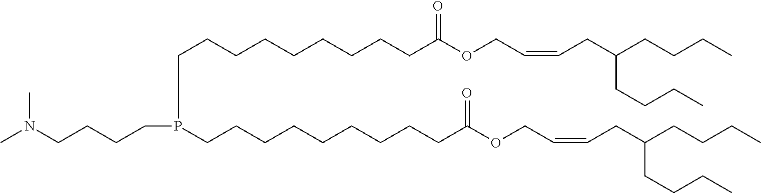 [SMILES: CCCCC(C/C=C\COC(=O)CCCCCCCCCP(CCCCCCCCCC(=O)OC/C=C\CC(CCCC)CCCC)CCCCN(C)C)CCCC]

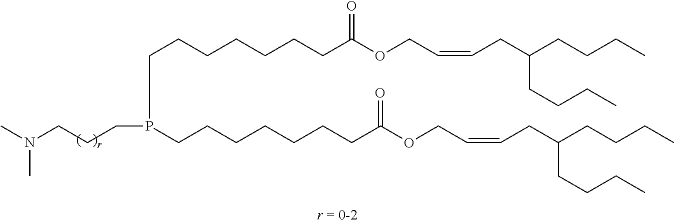

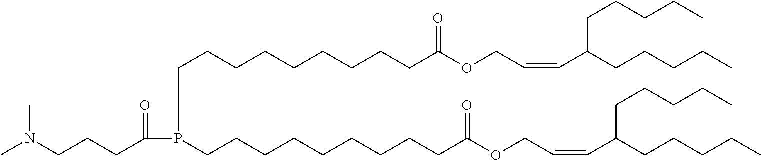 [SMILES: CCCCCC(/C=C\COC(=O)CCCCCCCCCP(CCCCCCCCCC(=O)OC/C=C\C(CCCCC)CCCCC)C(=O)CCCN(C)C)CCCCC]

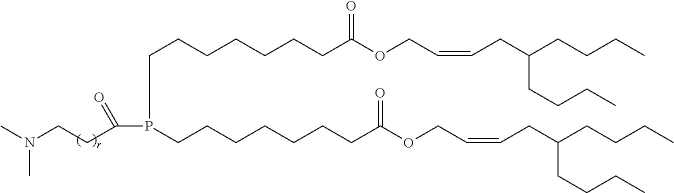

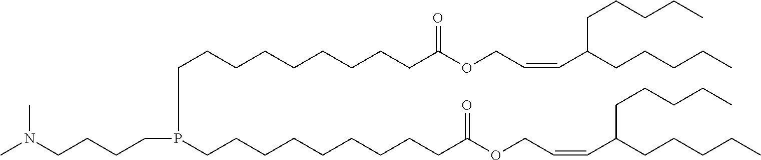 [SMILES: CCCCCC(/C=C\COC(=O)CCCCCCCCCP(CCCCCCCCCC(=O)OC/C=C\C(CCCCC)CCCCC)CCCCN(C)C)CCCCC]

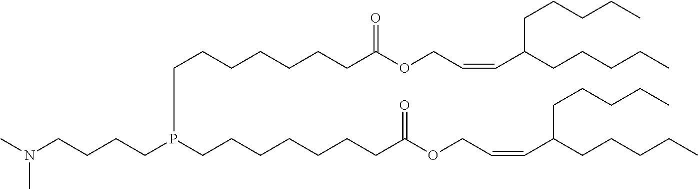 [SMILES: CCCCCC(/C=C\COC(=O)CCCCCCCP(CCCCCCCC(=O)OC/C=C\C(CCCCC)CCCCC)CCCCN(C)C)CCCCC]

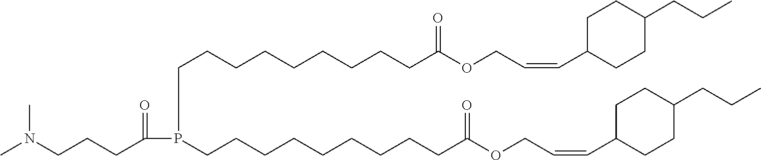 [SMILES: CCCC1CCC(/C=C\COC(=O)CCCCCCCCCP(CCCCCCCCCC(=O)OC/C=C\C2CCC(CCC)CC2)C(=O)CCCN(C)C)CC1]

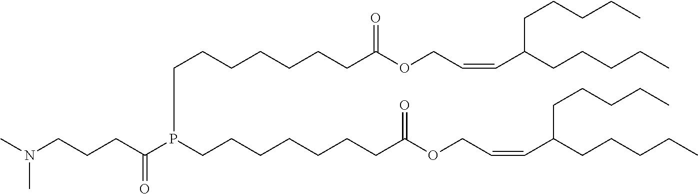 [SMILES: CCCCCC(/C=C\COC(=O)CCCCCCCP(CCCCCCCC(=O)OC/C=C\C(CCCCC)CCCCC)C(=O)CCCN(C)C)CCCCC]

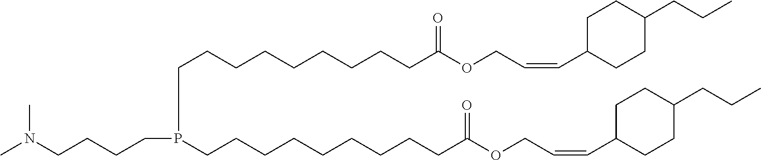 [SMILES: CCCC1CCC(/C=C\COC(=O)CCCCCCCCCP(CCCCCCCCCC(=O)OC/C=C\C2CCC(CCC)CC2)CCCCN(C)C)CC1]

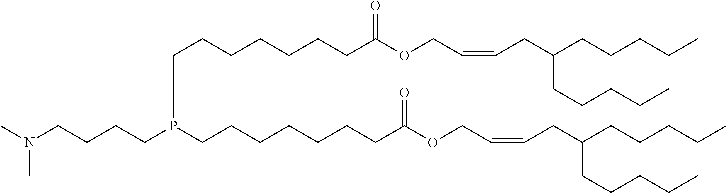 [SMILES: CCCCCC(C/C=C\COC(=O)CCCCCCCP(CCCCCCCC(=O)OC/C=C\CC(CCCCC)CCCCC)CCCCN(C)C)CCCCC]

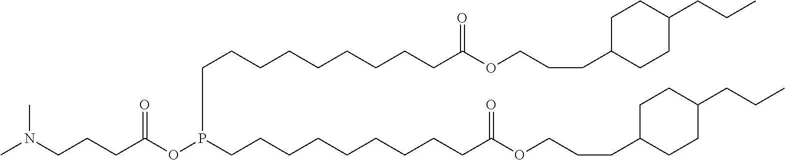 [SMILES: CCCC1CCC(CCCOC(=O)CCCCCCCCCP(CCCCCCCCCC(=O)OCCCC2CCC(CCC)CC2)OC(=O)CCCN(C)C)CC1]

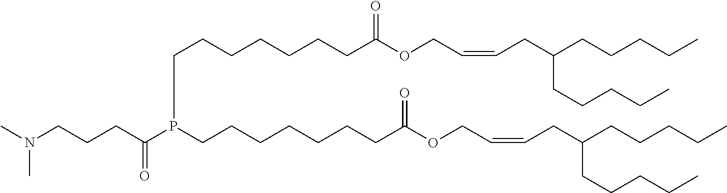 [SMILES: CCCCCC(C/C=C\COC(=O)CCCCCCCP(CCCCCCCC(=O)OC/C=C\CC(CCCCC)CCCCC)C(=O)CCCN(C)C)CCCCC]

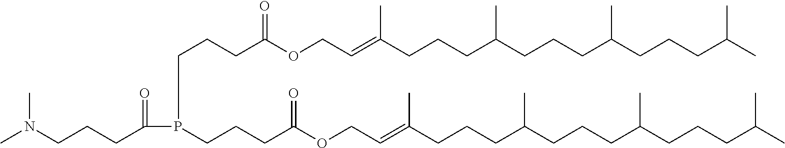 [SMILES: C/C(=C\COC(=O)CCCP(CCCC(=O)OC/C=C(\C)CCCC(C)CCCC(C)CCCC(C)C)C(=O)CCCN(C)C)CCCC(C)CCCC(C)CCCC(C)C]

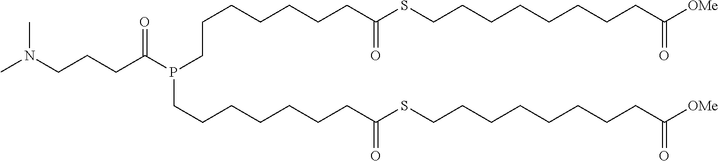 [SMILES: COC(=O)CCCCCCCCSC(=O)CCCCCCCP(CCCCCCCC(=O)SCCCCCCCCC(=O)OC)C(=O)CCCN(C)C]

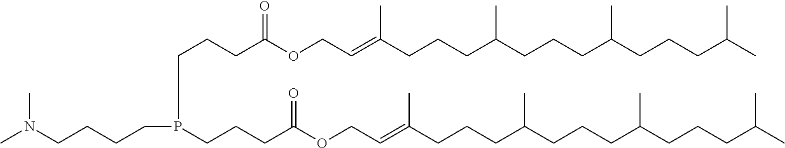 [SMILES: C/C(=C\COC(=O)CCCP(CCCCN(C)C)CCCC(=O)OC/C=C(\C)CCCC(C)CCCC(C)CCCC(C)C)CCCC(C)CCCC(C)CCCC(C)C]

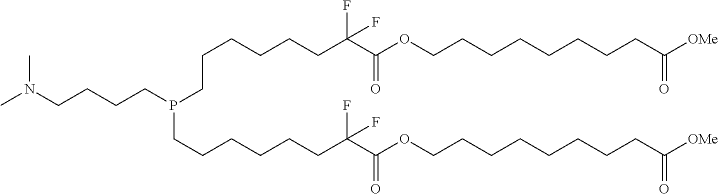 [SMILES: COC(=O)CCCCCCCCOC(=O)C(F)(F)CCCCCCP(CCCCCCC(F)(F)C(=O)OCCCCCCCCC(=O)OC)CCCCN(C)C]

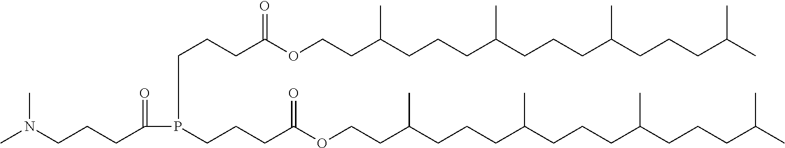 [SMILES: CC(C)CCCC(C)CCCC(C)CCCC(C)CCOC(=O)CCCP(CCCC(=O)OCCC(C)CCCC(C)CCCC(C)CCCC(C)C)C(=O)CCCN(C)C]

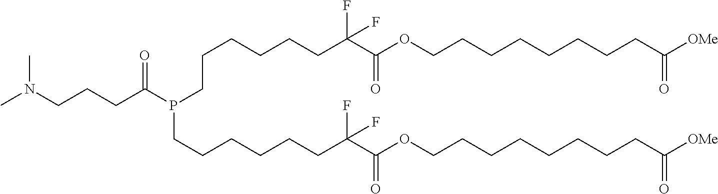 [SMILES: COC(=O)CCCCCCCCOC(=O)C(F)(F)CCCCCCP(CCCCCCC(F)(F)C(=O)OCCCCCCCCC(=O)OC)C(=O)CCCN(C)C]

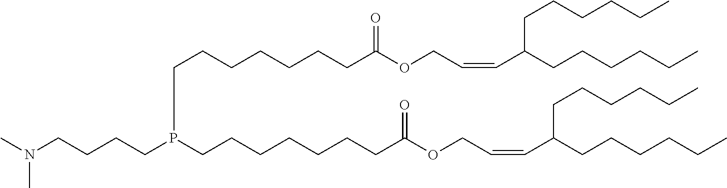 [SMILES: CCCCCCC(/C=C\COC(=O)CCCCCCCP(CCCCCCCC(=O)OC/C=C\C(CCCCCC)CCCCCC)CCCCN(C)C)CCCCCC]

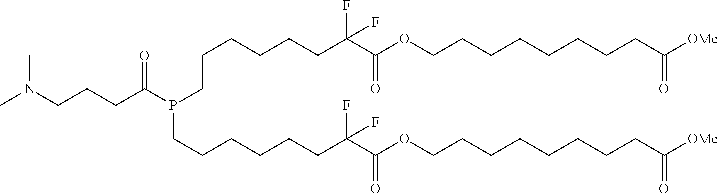 [SMILES: COC(=O)CCCCCCCCOC(=O)C(F)(F)CCCCCCP(CCCCCCC(F)(F)C(=O)OCCCCCCCCC(=O)OC)C(=O)CCCN(C)C]

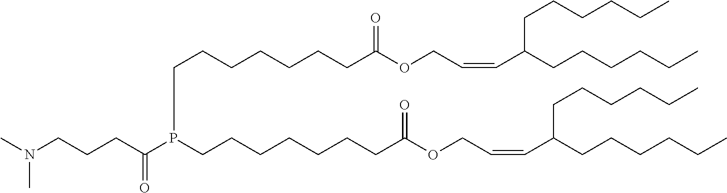 [SMILES: CCCCCCC(/C=C\COC(=O)CCCCCCCP(CCCCCCCC(=O)OC/C=C\C(CCCCCC)CCCCCC)C(=O)CCCN(C)C)CCCCCC]

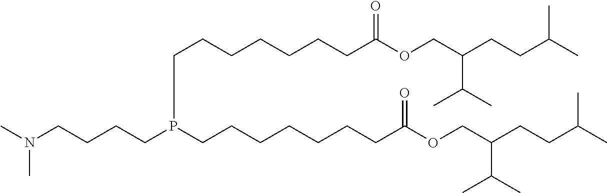 [SMILES: CC(C)CCC(COC(=O)CCCCCCCP(CCCCCCCC(=O)OCC(CCC(C)C)C(C)C)CCCCN(C)C)C(C)C]

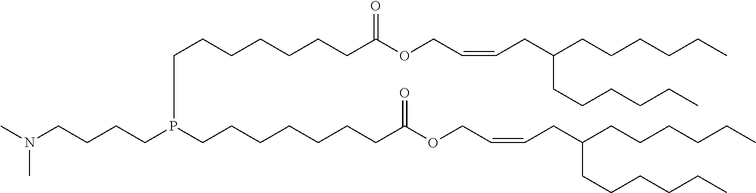 [SMILES: CCCCCCC(C/C=C\COC(=O)CCCCCCCP(CCCCCCCC(=O)OC/C=C\CC(CCCCCC)CCCCCC)CCCCN(C)C)CCCCCC]

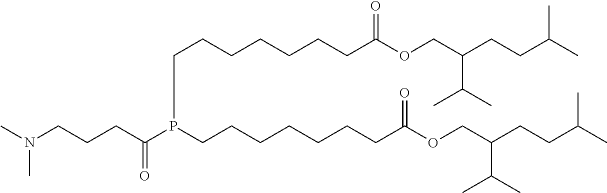 [SMILES: CC(C)CCC(COC(=O)CCCCCCCP(CCCCCCCC(=O)OCC(CCC(C)C)C(C)C)C(=O)CCCN(C)C)C(C)C]

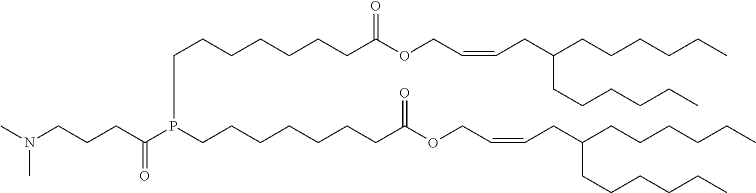 [SMILES: CCCCCCC(C/C=C\COC(=O)CCCCCCCP(CCCCCCCC(=O)OC/C=C\CC(CCCCCC)CCCCCC)C(=O)CCCN(C)C)CCCCCC]

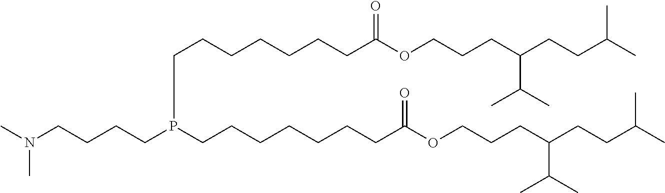 [SMILES: CC(C)CCC(CCCOC(=O)CCCCCCCP(CCCCCCCC(=O)OCCCC(CCC(C)C)C(C)C)CCCCN(C)C)C(C)C]

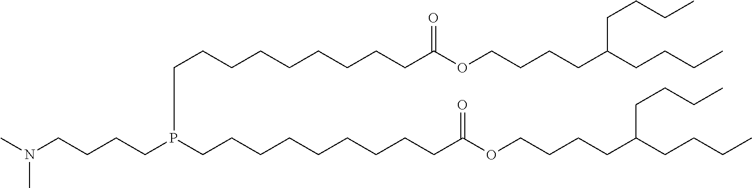 [SMILES: CCCCC(CCCC)CCCCOC(=O)CCCCCCCCCP(CCCCCCCCCC(=O)OCCCCC(CCCC)CCCC)CCCCN(C)C]

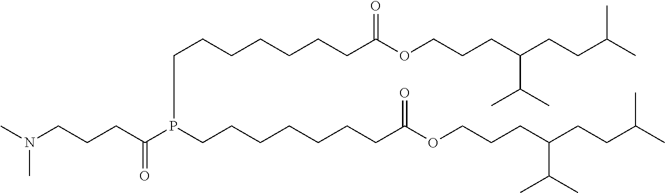 [SMILES: CC(C)CCC(CCCOC(=O)CCCCCCCP(CCCCCCCC(=O)OCCCC(CCC(C)C)C(C)C)C(=O)CCCN(C)C)C(C)C]

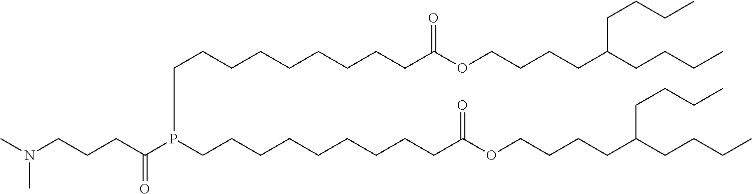 [SMILES: CCCCC(CCCC)CCCCOC(=O)CCCCCCCCCP(CCCCCCCCCC(=O)OCCCCC(CCCC)CCCC)C(=O)CCCN(C)C]

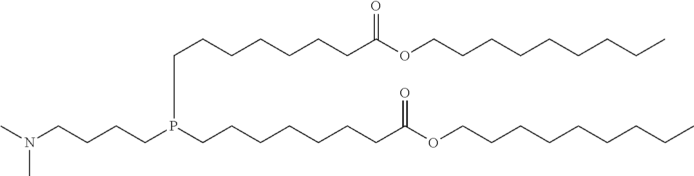 [SMILES: CCCCCCCCCOC(=O)CCCCCCCP(CCCCCCCC(=O)OCCCCCCCCC)CCCCN(C)C]

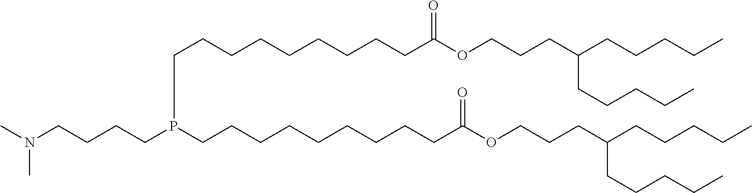 [SMILES: CCCCCC(CCCCC)CCCOC(=O)CCCCCCCCCP(CCCCCCCCCC(=O)OCCCC(CCCCC)CCCCC)CCCCN(C)C]

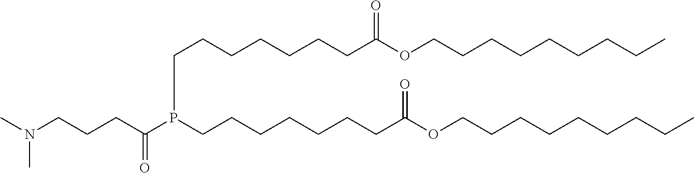 [SMILES: CCCCCCCCCOC(=O)CCCCCCCP(CCCCCCCC(=O)OCCCCCCCCC)C(=O)CCCN(C)C]

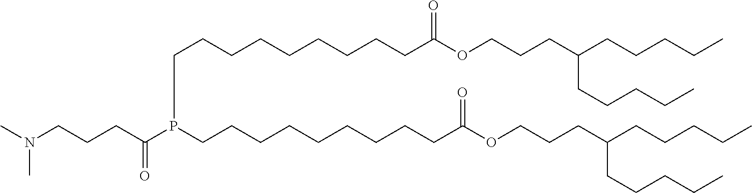 [SMILES: CCCCCC(CCCCC)CCCOC(=O)CCCCCCCCCP(CCCCCCCCCC(=O)OCCCC(CCCCC)CCCCC)C(=O)CCCN(C)C]

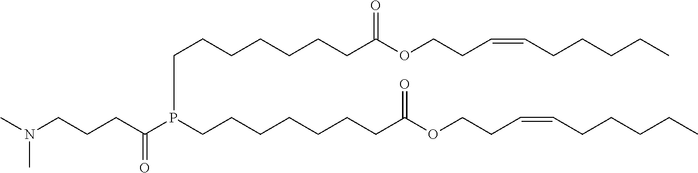 [SMILES: CCCCC/C=C\CCOC(=O)CCCCCCCP(CCCCCCCC(=O)OCC/C=C\CCCCC)C(=O)CCCN(C)C]

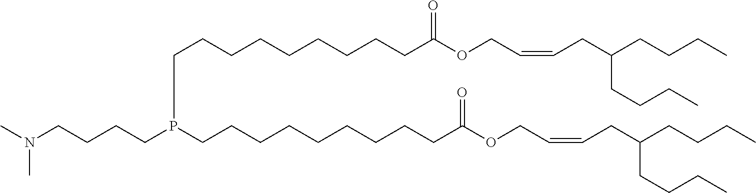 [SMILES: CCCCC(C/C=C\COC(=O)CCCCCCCCCP(CCCCCCCCCC(=O)OC/C=C\CC(CCCC)CCCC)CCCCN(C)C)CCCC]

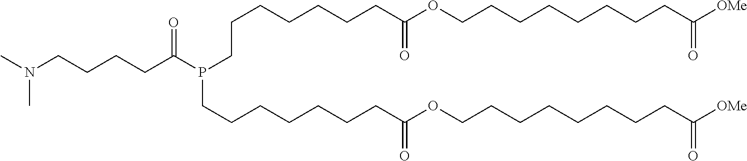 [SMILES: COC(=O)CCCCCCCCOC(=O)CCCCCCCP(CCCCCCCC(=O)OCCCCCCCCC(=O)OC)C(=O)CCCCN(C)C]

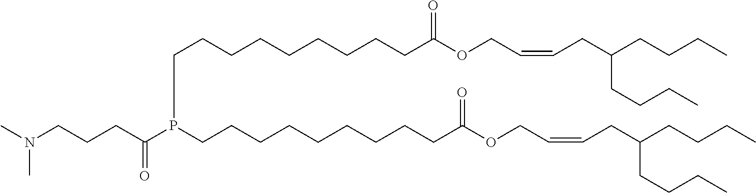 [SMILES: CCCCC(C/C=C\COC(=O)CCCCCCCCCP(CCCCCCCCCC(=O)OC/C=C\CC(CCCC)CCCC)C(=O)CCCN(C)C)CCCC]

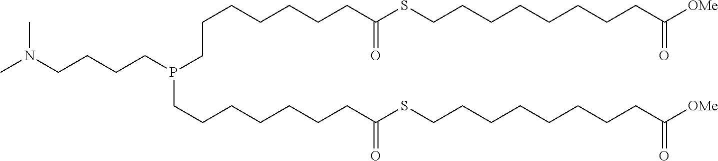 [SMILES: COC(=O)CCCCCCCCSC(=O)CCCCCCCP(CCCCCCCC(=O)SCCCCCCCCC(=O)OC)CCCCN(C)C]

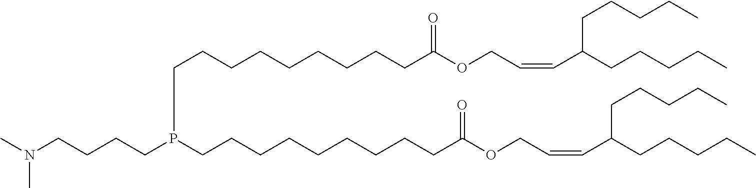 [SMILES: CCCCCC(/C=C\COC(=O)CCCCCCCCCP(CCCCCCCCCC(=O)OC/C=C\C(CCCCC)CCCCC)CCCCN(C)C)CCCCC]

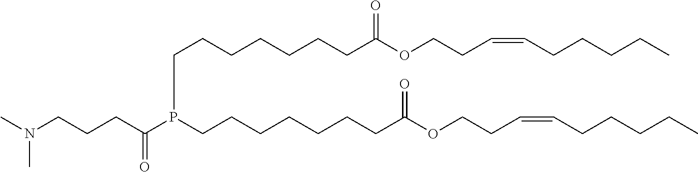 [SMILES: CCCCC/C=C\CCOC(=O)CCCCCCCP(CCCCCCCC(=O)OCC/C=C\CCCCC)C(=O)CCCN(C)C]

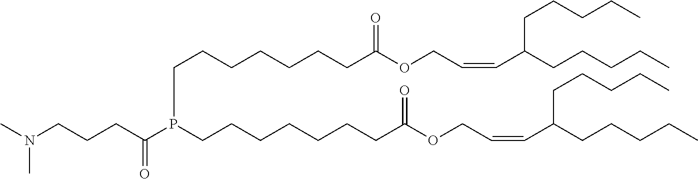 [SMILES: CCCCCC(/C=C\COC(=O)CCCCCCCP(CCCCCCCC(=O)OC/C=C\C(CCCCC)CCCCC)C(=O)CCCN(C)C)CCCCC]

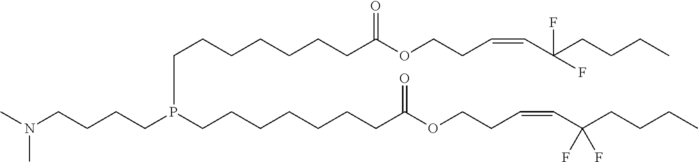 [SMILES: CCCCC(F)(F)/C=C\CCOC(=O)CCCCCCCP(CCCCCCCC(=O)OCC/C=C\C(F)(F)CCCC)CCCCN(C)C]

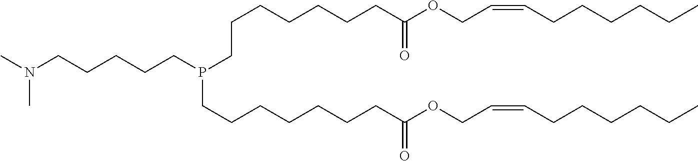 [SMILES: CCCCCC/C=C\COC(=O)CCCCCCCP(CCCCCCCC(=O)OC/C=C\CCCCCC)CCCCCN(C)C]

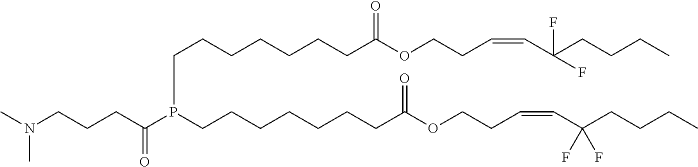 [SMILES: CCCCC(F)(F)/C=C\CCOC(=O)CCCCCCCP(CCCCCCCC(=O)OCC/C=C\C(F)(F)CCCC)C(=O)CCCN(C)C]

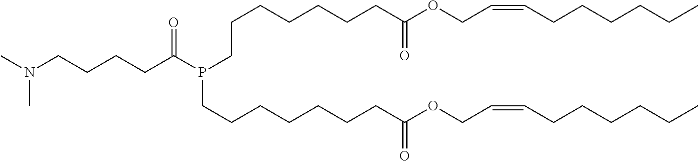 [SMILES: CCCCCC/C=C\COC(=O)CCCCCCCP(CCCCCCCC(=O)OC/C=C\CCCCCC)C(=O)CCCCN(C)C]

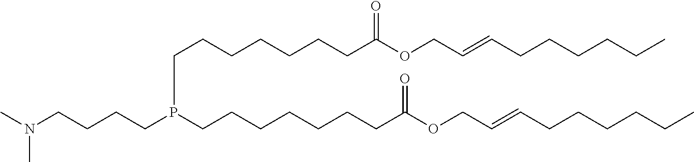 [SMILES: CCCCCC/C=C/COC(=O)CCCCCCCP(CCCCCCCC(=O)OC/C=C/CCCCCC)CCCCN(C)C]

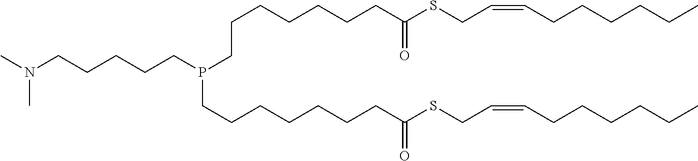 [SMILES: CCCCCC/C=C\CSC(=O)CCCCCCCP(CCCCCCCC(=O)SC/C=C\CCCCCC)CCCCCN(C)C]

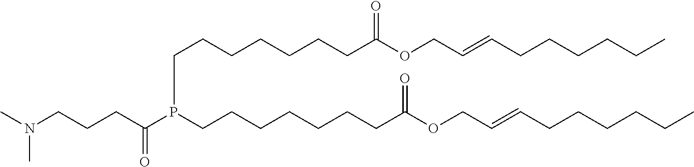 [SMILES: CCCCCC/C=C/COC(=O)CCCCCCCP(CCCCCCCC(=O)OC/C=C/CCCCCC)C(=O)CCCN(C)C]

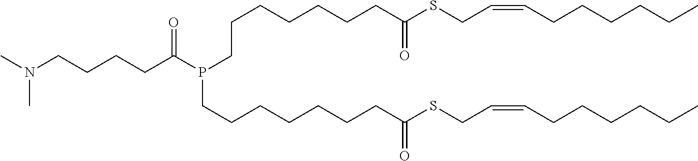 [SMILES: CCCCCC/C=C\CSC(=O)CCCCCCCP(CCCCCCCC(=O)SC/C=C\CCCCCC)C(=O)CCCCN(C)C]

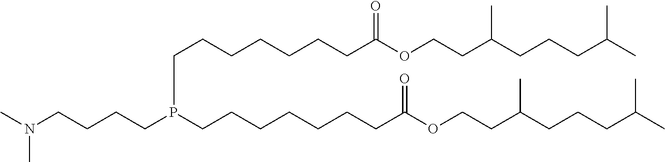 [SMILES: CC(C)CCCC(C)CCOC(=O)CCCCCCCP(CCCCCCCC(=O)OCCC(C)CCCC(C)C)CCCCN(C)C]

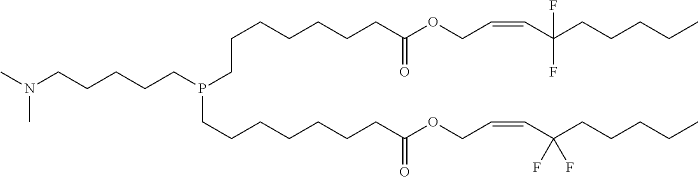 [SMILES: CCCCCC(F)(F)/C=C\COC(=O)CCCCCCCP(CCCCCCCC(=O)OC/C=C\C(F)(F)CCCCC)CCCCCN(C)C]

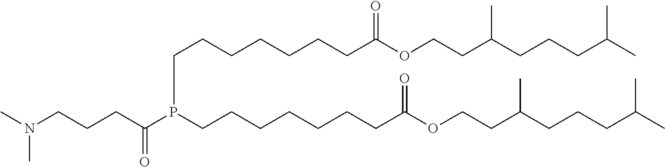 [SMILES: CC(C)CCCC(C)CCOC(=O)CCCCCCCP(CCCCCCCC(=O)OCCC(C)CCCC(C)C)C(=O)CCCN(C)C]

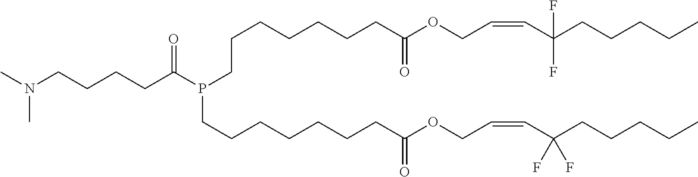 [SMILES: CCCCCC(F)(F)/C=C\COC(=O)CCCCCCCP(CCCCCCCC(=O)OC/C=C\C(F)(F)CCCCC)C(=O)CCCCN(C)C]

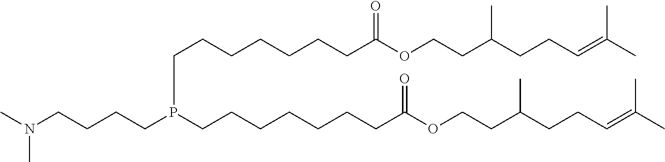 [SMILES: CC(C)=CCCC(C)CCOC(=O)CCCCCCCP(CCCCCCCC(=O)OCCC(C)CCC=C(C)C)CCCCN(C)C]

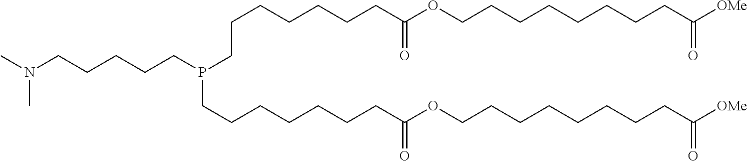 [SMILES: COC(=O)CCCCCCCCOC(=O)CCCCCCCP(CCCCCCCC(=O)OCCCCCCCCC(=O)OC)CCCCCN(C)C]

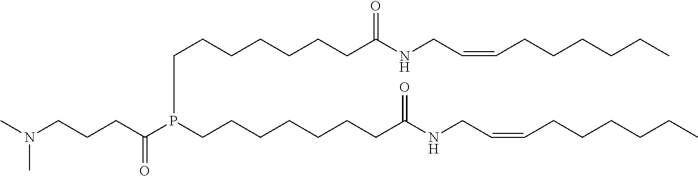 [SMILES: CCCCCC/C=C\CNC(=O)CCCCCCCP(CCCCCCCC(=O)NC/C=C\CCCCCC)C(=O)CCCN(C)C]

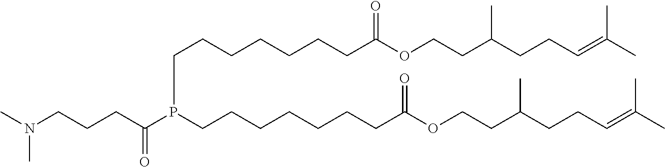 [SMILES: CC(C)=CCCC(C)CCOC(=O)CCCCCCCP(CCCCCCCC(=O)OCCC(C)CCC=C(C)C)C(=O)CCCN(C)C]

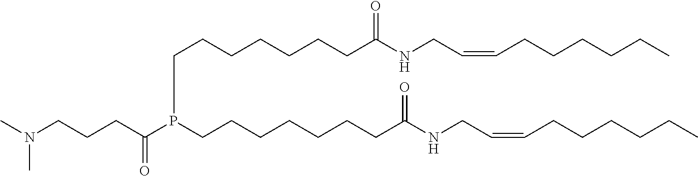 [SMILES: CCCCCC/C=C\CNC(=O)CCCCCCCP(CCCCCCCC(=O)NC/C=C\CCCCCC)C(=O)CCCN(C)C]

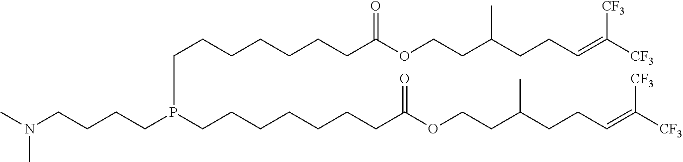 [SMILES: CC(CCC=C(C(F)(F)F)C(F)(F)F)CCOC(=O)CCCCCCCP(CCCCCCCC(=O)OCCC(C)CCC=C(C(F)(F)F)C(F)(F)F)CCCCN(C)C]

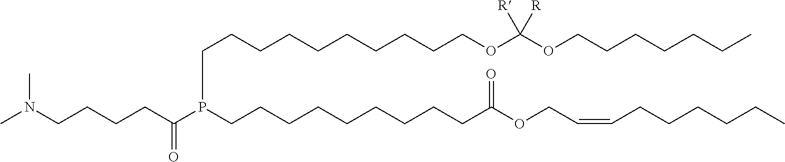

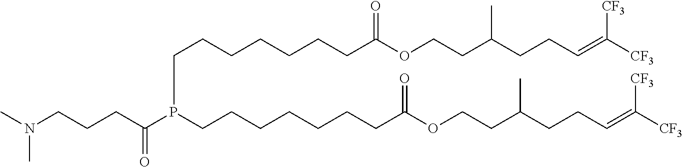 [SMILES: CC(CCC=C(C(F)(F)F)C(F)(F)F)CCOC(=O)CCCCCCCP(CCCCCCCC(=O)OCCC(C)CCC=C(C(F)(F)F)C(F)(F)F)C(=O)CCCN(C)C]

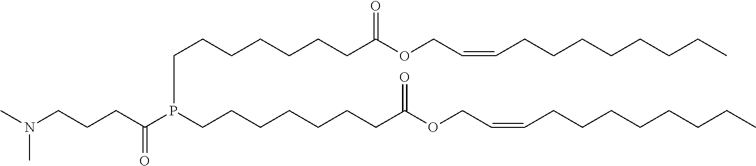 [SMILES: CCCCCCCC/C=C\COC(=O)CCCCCCCP(CCCCCCCC(=O)OC/C=C\CCCCCCCC)C(=O)CCCN(C)C]

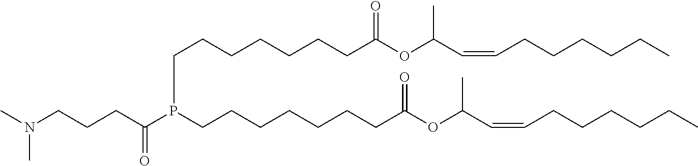 [SMILES: CCCCCC/C=C\C(C)OC(=O)CCCCCCCP(CCCCCCCC(=O)OC(C)/C=C\CCCCCC)C(=O)CCCN(C)C]

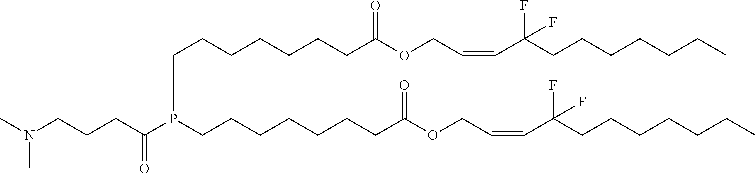 [SMILES: CCCCCCCC(F)(F)/C=C\COC(=O)CCCCCCCP(CCCCCCCC(=O)OC/C=C\C(F)(F)CCCCCCC)C(=O)CCCN(C)C]

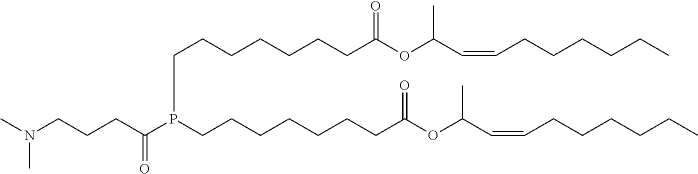 [SMILES: CCCCCC/C=C\C(C)OC(=O)CCCCCCCP(CCCCCCCC(=O)OC(C)/C=C\CCCCCC)C(=O)CCCN(C)C]

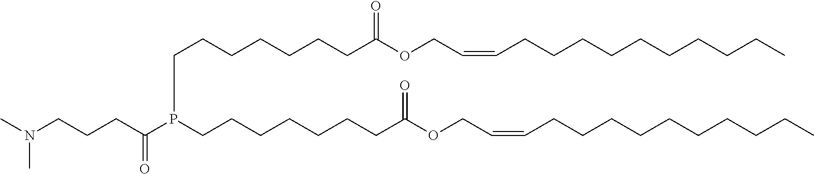 [SMILES: CCCCCCCCCC/C=C\COC(=O)CCCCCCCP(CCCCCCCC(=O)OC/C=C\CCCCCCCCCC)C(=O)CCCN(C)C]

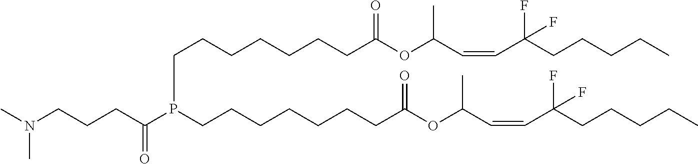 [SMILES: CCCCCC(F)(F)/C=C\C(C)OC(=O)CCCCCCCP(CCCCCCCC(=O)OC(C)/C=C\C(F)(F)CCCCC)C(=O)CCCN(C)C]

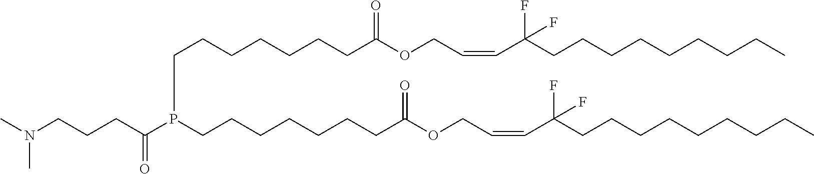 [SMILES: CCCCCCCCCC(F)(F)/C=C\COC(=O)CCCCCCCP(CCCCCCCC(=O)OC/C=C\C(F)(F)CCCCCCCCC)C(=O)CCCN(C)C]

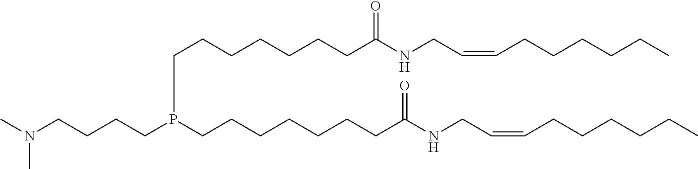 [SMILES: CCCCCC/C=C\CNC(=O)CCCCCCCP(CCCCCCCC(=O)NC/C=C\CCCCCC)CCCCN(C)C]

The following embodiments are directed to the acetal containing cationic lipids described herein.

In an embodiment of Formula A or A1, n is 0.

In an embodiment of Formula A or A1, n is 1.

In an embodiment of Formula A or A1, n is 2.

In an embodiment of Formula A or A1, R¹ and R² are independently selected from H and (C₁-C₆)alkyl, wherein said alkyl is optionally substituted with one or more substituents selected from R′, or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′.

In an embodiment of Formula A or A1, R¹ and R² are independently selected from H, methyl, ethyl and propyl, wherein said methyl, ethyl and propyl are optionally substituted with one or more substituents selected from R′, or R¹ and R² can be taken together with the nitrogen to which they are attached to form a monocyclic heterocycle with 3-7 (e.g., 4-7) members optionally containing, in addition to the nitrogen, one or two additional heteroatoms selected from N, O and S, said monocyclic heterocycle is optionally substituted with one or more substituents selected from R′.

In an embodiment of Formula A or A1, R¹ and R² are independently selected from H, methyl, ethyl and propyl.

In an embodiment of Formula A or A1, R¹ and R² are independently selected from H and methyl.

In an embodiment of Formula A or A1, R¹ and R² are both methyl.

In an embodiment of Formula A or A1, R³ is selected from H, methyl, ethyl and propyl, wherein said methyl, ethyl and propyl are optionally substituted with one or more substituents selected from R′, or R³ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′.

In an embodiment of Formula A or A1, R³ is selected from H, methyl, ethyl and propyl, wherein said methyl, ethyl and propyl are optionally substituted with one or more substituents selected from R¹, or R³ can be taken together with R¹ to form a monocyclic heterocycle which is optionally substituted with one or more substituents selected from R′, or R³ can be taken together with R⁴ to form cyclopropyl or cyclobutyl.

In an embodiment of Formula A or A1, R³ is selected from H, methyl, ethyl and propyl.

In an embodiment of Formula A or A1, R³ is selected from H, methyl and ethyl.

In an embodiment of Formula A or A1, R³ is methyl.

In an embodiment of Formula A or A1, R³ is H.

In an embodiment of Formula A or A1, R⁴ is selected from H, methyl, ethyl and propyl.

In an embodiment of Formula A or A1, R⁴ is selected from H and methyl.

In an embodiment of Formula A or A1, R⁴ is methyl.

In an embodiment of Formula A or A1, R⁴ is H.

In an embodiment of Formula A or A1, each R^{3′} is independently selected from H, methyl, ethyl and propyl.

In an embodiment of Formula A or A1, each R^{3′} is independently selected from H, methyl and ethyl.

In an embodiment of Formula A or A1, each R^{3′} is methyl.

In an embodiment of Formula A or A1, each R^{3′} is H.

In an embodiment of Formula A or A1, each R^{4′} is independently selected from H, methyl, ethyl and propyl.

In an embodiment of Formula A or A1, each R^{4′} is independently selected from H, methyl and ethyl.

In an embodiment of Formula A or A1, each R^{4′} is methyl.

In an embodiment of Formula A, each R^{4′} is H.

In an embodiment of Formula A or A1, R⁵ is selected from H, methyl, ethyl and propyl, wherein said methyl, ethyl and propyl are optionally substituted with one or more substituents selected from R′, or R⁵ can be taken together with R¹ to form a monocyclic heterocycle with 4-7 members optionally containing, in addition to the nitrogen, one or two additional heteroatoms selected from N, O and S, said monocyclic heterocycle is optionally substituted with one or more substituents selected from R′.

In an embodiment of Formula A or A1, R⁵ is selected from H, methyl, ethyl and propyl, wherein said methyl, ethyl and propyl are optionally substituted with one or more substituents selected from R′, or R⁵ can be taken together with R¹ to form a monocyclic heterocycle which is optionally substituted with one or more substituents selected from R′.

In an embodiment of Formula A or A1, R⁵ is selected from H, methyl, ethyl and propyl.

In an embodiment of Formula A or A1, R⁵ is selected from H and methyl.

In an embodiment of Formula A or A1, R⁵ is methyl.

In an embodiment of Formula A or A1, R⁵ is H.

In an embodiment of Formula A or A1, each R′ is OH or R″.

In an embodiment of Formula A or A1, each R′ is R″.

In an embodiment of Formula A or A1, R″ is selected from H, methyl, ethyl and propyl, wherein said methyl, ethyl and propyl are optionally substituted with one or more OH.

In an embodiment of Formula A or A1, R″ is selected from H, methyl and ethyl wherein said methyl and ethyl are optionally substituted with one or more OH.

In an embodiment of Formula A, L¹ is selected from C₄-C₂₂ alkyl and C₄-C₂₂ alkenyl, which are optionally substituted with halogen and OH.

In an embodiment of Formula A, L¹ is selected from C₄-C₂₂ alkyl and C₄-C₂₂ alkenyl.

In an embodiment of Formula A, L¹ is selected from C₆-C₁₈ alkyl and C₆-C₁₈ alkenyl.

In an embodiment of Formula A, L² is a C₄-C₂₄ alkenyl, which is optionally substituted with halogen and OH.

In an embodiment of Formula A, L² is a C₄-C₂₄ alkenyl.

In an embodiment of Formula A, L² is C₁₋₈ alkenyl.

In an embodiment of Formula A, L² is

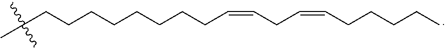 [SMILES: *CCCCCCCC/C=C\C/C=C\CCCCC]

In an embodiment of Formula A, L¹ and L² are

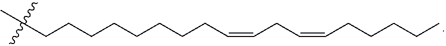 [SMILES: *CCCCCCCC/C=C\C/C=C\CCCCC]

In an embodiment of Formula A or A1, “heterocyclyl” is pyrrolidine, piperidine, morpholine, imidazole or piperazine.

In an embodiment of Formula A or A1, “monocyclic heterocycle” is pyrrolidine, piperidine, morpholine, imidazole or piperazine.

In an embodiment of Formula A or A1, “monocyclic heterocycle” is pyrrolidine or piperidine.

In an embodiment of Formula A or A1, “polyamine” is putrescine, cadaverine, spermidine or spermine.

Specific cationic lipids include:

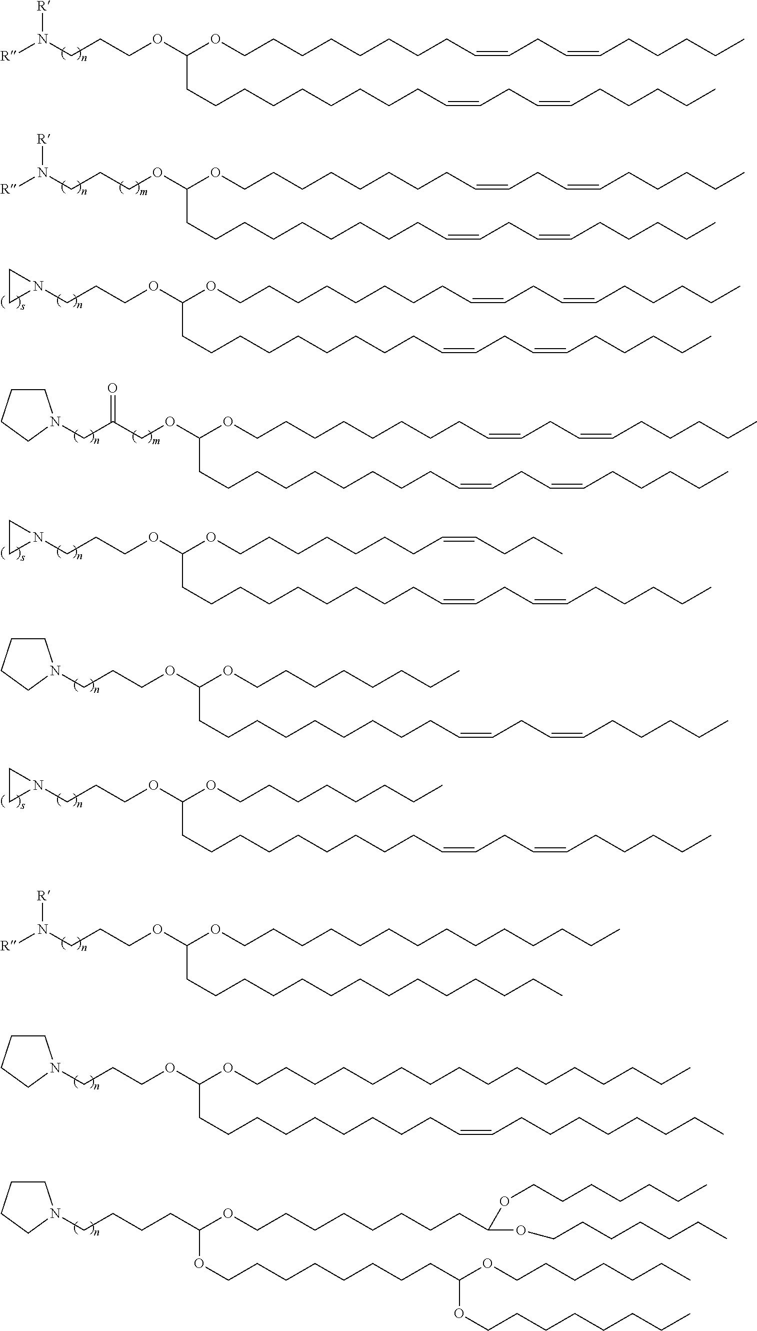

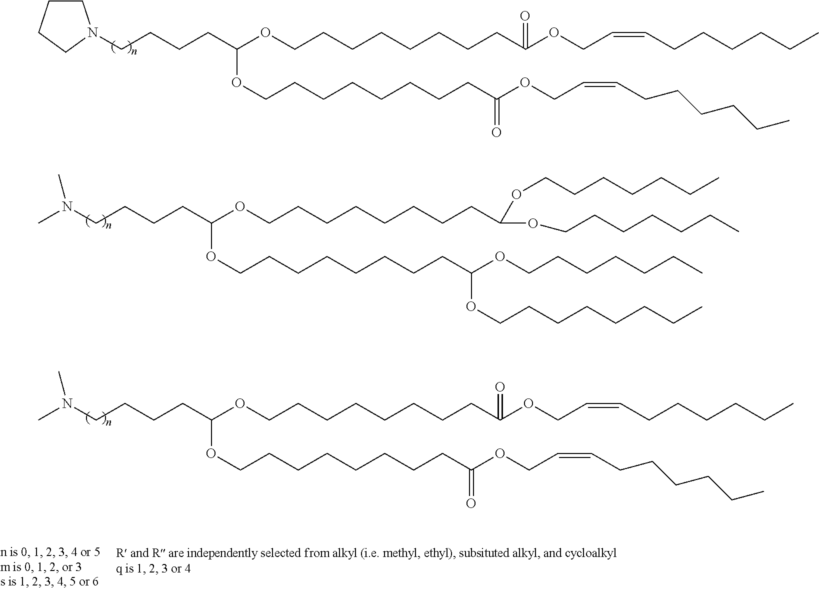

or any pharmaceutically acceptable salt or stereoisomer thereof.

Cationic lipids include those having alternative fatty acid groups and other dialkylamino groups than those shown, including those in which the alkyl substituents are different (e.g., N-ethyl-N-methylamino-, and N-propyl-N-ethylamino-).

In certain embodiments, the cationic lipids have at least one protonatable or deprotonatable group, such that the lipid is positively charged at a pH at or below physiological pH (e.g. pH 7.4), and neutral at a second pH, preferably at or above physiological pH. Such lipids are also referred to as cationic lipids. It will, of course, be understood that the addition or removal of protons as a function of pH is an equilibrium process, and that the reference to a charged or a neutral lipid refers to the nature of the predominant species and does not require that all of the lipid be present in the charged or neutral form. The lipids can have more than one protonatable or deprotonatable group, or can be zwiterrionic.

In certain embodiments, protonatable lipids (i.e., cationic lipids) have a pKₐ of the protonatable group in the range of about 4 to about 11. For example, the lipids can have a pKₐ of about 4 to about 7, e.g., from about 5 to about 7, such as from about 5.5 to about 6.8, when incorporated into lipid particles. Such lipids may be cationic at a lower pH formulation stage, while particles will be largely (though not completely) surface neutralized at physiological pH around pH 7.4.

In particular embodiments, the lipids are charged lipids. As used herein, the term “charged lipid” includes, but is not limited to, those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine).

Included in the instant invention is the free form of the cationic lipids described herein, as well as pharmaceutically acceptable salts and stereoisomers thereof. The cationic lipid can be a protonated salt of the amine cationic lipid. The term “free form” refers to the amine cationic lipids in non-salt form. The free form may be regenerated by treating the salt with a suitable dilute aqueous base solution such as dilute aqueous NaOH, potassium carbonate, ammonia and sodium bicarbonate.

The pharmaceutically acceptable salts of the instant cationic lipids can be synthesized from the cationic lipids of this invention which contain a basic or acidic moiety by conventional chemical methods. Generally, the salts of the basic cationic lipids are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the cationic lipids of this invention include non-toxic salts of the cationic lipids of this invention as formed by reacting a basic instant cationic lipids with an inorganic or organic acid. For example, non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, and trifluoroacetic (TFA).

When the cationic lipids of the present invention are acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, and zinc. In one embodiment, the base is selected from ammonium, calcium, magnesium, potassium and sodium. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, and tromethamine.

It will also be noted that the cationic lipids of the present invention may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

One or more additional cationic lipids, which carry a net positive charge at about physiological pH, in addition to those specifically described above, may also be included in the lipid particles and compositions described herein. Such cationic lipids include, but are not limited to N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL).

### The Other Lipid Components

The lipid particles and compositions described herein may also include one or more neutral lipids. Neutral lipids, when present, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. In one embodiment, the neutral lipid component is a lipid having two acyl groups (e.g., diacylphosphatidylcholine and diacylphosphatidylethanolamine). In one embodiment, the neutral lipid contains saturated fatty acids with carbon chain lengths in the range of C₁₀ to C₂₀. In another embodiment, the neutral lipid includes mono or diunsaturated fatty acids with carbon chain lengths in the range of C₁₀ to C₂₀. Suitable neutral lipids include, but are not limited to, DSPC, DPPC, POPC, DOPE, DSPC, and SM.

The lipid particles and compositions described herein may also include one or more lipids capable of reducing aggregation. Examples of lipids that reduce aggregation of particles during formation include polyethylene glycol (PEG)-modified lipids (PEG lipids, such as PEG-DMG and PEG-DMA), monosialoganglioside Gm1, and polyamide oligomers (“PAO”) such as (described in U.S. Pat. No. 6,320,017, which is incorporated by reference in its entirety). Suitable PEG lipids include, but are not limited to, PEG-modified phosphatidylethanolamine and phosphatidic acid, PEG-ceramide conjugates (e.g., PEG-CerC14 or PEG-CerC20) (such as those described in U.S. Pat. No. 5,820,873, incorporated herein by reference), PEG-modified dialkylamines and PEG-modified 1,2-diacyloxypropan-3-amines, PEG-modified diacylglycerols and dialkylglycerols, mPEG (mw2000)-diastearoylphosphatidylethanolamine (PEG-DSPE).

The lipid particles and compositions may include a sterol, such as cholesterol.

### Lipid Particles

In a further aspect, the present invent relates to lipid particles that include one or more of the cationic lipids described herein. In one embodiment, the lipid particle includes one or more compounds of formula I-VII.

Lipid particles include, but are not limited to, liposomes. As used herein, a liposome is a structure having lipid-containing membranes enclosing an aqueous interior.

Another embodiment is a nucleic acid-lipid particle (e.g., a SNALP) comprising a cationic lipid of the present invention, a non-cationic lipid (such as a neutral lipid), optionally a PEG-lipid conjugate (such as the lipids for reducing aggregation of lipid particles discussed herein), and a nucleic acid. As used herein, the term “SNALP” refers to a stable nucleic acid-lipid particle. A SNALP represents a particle made from lipids, wherein the nucleic acid (e.g., an interfering RNA) is encapsulated within the lipids. In certain instances, SNALPs are useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a SNALP as set forth in International Publication No. WO 00/03683, the disclosure of which is herein incorporated by reference in its entirety.

For example, the lipid particle may include a cationic lipid, a fusion-promoting lipid (e.g., DPPC), a neutral lipid, cholesterol, and a PEG-modified lipid. In one embodiment, the lipid particle includes the above lipid mixture in molar ratios of about 20-70% cationic lipid: 0.1-50% fusion promoting lipid: 5-45% neutral lipid: 20-55% cholesterol: 0.5-15% PEG-modified lipid.

In another embodiment of the lipid particle, the cationic lipid is present in a mole percentage of about 20% and about 60%; the neutral lipid is present in a mole percentage of about 5% to about 25%; the sterol is present in a mole percentage of about 25% to about 55%; and the PEG lipid is PEG-DMA, PEG-DMG, or a combination thereof, and is present in a mole percentage of about 0.5% to about 15%.

In particular embodiments, the molar lipid ratio, with regard to mol % cationic lipid/DSPC/Chol/PEG-DMG or PEG-DMA) is approximately 40/10/40/10, 35/15/40/10 or 52/13/30/5. This mixture may be further combined with a fusion-promoting lipid in a molar ratio of 0.1-50%, 0.1-50%, 0.5-50%, 1-50%, 5%-45%, 10%-40%, or 15%-35%. In other words, when a 40/10/40/10 mixture of lipid/DSPC/Chol/PEG-DMG or PEG-DMA is combined with a fusion-promoting peptide in a molar ratio of 50%, the resulting lipid particles can have a total molar ratio of (mol % cationic lipid/DSPC/Chol/PEG-DMG or PEG-DMA/fusion-promoting peptide) 20/5/20/5/50. In another embodiment, the neutral lipid, DSPC, in these compositions is replaced with POPC, DPPC, DOPE or SM.

In one embodiment, the lipid particles comprise a cationic lipid of the present invention, a neutral lipid, a sterol and a PEG-modified lipid. In one embodiment, the lipid particles include from about 25% to about 75% on a molar basis of cationic lipid, e.g., from about 35 to about 65%, from about 45 to about 65%, about 60%, about 57.5%, about 57.1%, about 50% or about 40% on a molar basis. In one embodiment, the lipid particles include from about 0% to about 15% on a molar basis of the neutral lipid, e.g., from about 3 to about 12%, from about 5 to about 10%, about 15%, about 10%, about 7.5%, about 7.1% or about 0% on a molar basis. In one embodiment, the neutral lipid is DPPC. In one embodiment, the neutral lipid is DSPC. In one embodiment, the formulation includes from about 5% to about 50% on a molar basis of the sterol, e.g., about 15 to about 45%, about 20 to about 40%, about 48%, about 40%, about 38.5%, about 35%, about 34.4%, about 31.5% or about 31% on a molar basis. In one embodiment, the sterol is cholesterol.

The lipid particles described herein may further include one or more therapeutic agents. In a preferred embodiment, the lipid particles include a nucleic acid (e.g., an oligonucleotide), such as siRNA or miRNA.

In one embodiment, the lipid particles include from about 0.1% to about 20% on a molar basis of the PEG-modified lipid, e.g., about 0.5 to about 10%, about 0.5 to about 5%, about 10%, about 5%, about 3.5%, about 1.5%, about 0.5%, or about 0.3% on a molar basis. In one embodiment, the PEG-modified lipid is PEG-DMG. In one embodiment, the PEG-modified lipid is PEG-c-DMA. In one embodiment, the lipid particles include 25-75% of cationic lipid, 0.5-15% of the neutral lipid, 5-50% of the sterol, and 0.5-20% of the PEG-modified lipid on a molar basis.

In one embodiment, the lipid particles include 35-65% of cationic lipid, 3-12% of the neutral lipid, 15-45% of the sterol, and 0.5-10% of the PEG-modified lipid on a molar basis. In one embodiment, the lipid particles include 45-65% of cationic lipid, 5-10% of the neutral lipid, 25-40% of the sterol, and 0.5-5% of the PEG-modified lipid on a molar basis. In one embodiment, the PEG modified lipid comprises a PEG molecule of an average molecular weight of 2,000 Da. In one embodiment, the PEG modified lipid is PEG-distyryl glycerol (PEG-DSG).

In one embodiment, the ratio of lipid:siRNA is at least about 0.5:1, at least about 1:1, at least about 2:1, at least about 3:1, at least about 4:1, at least about 5:1, at least about 6:1, at least about 7:1, at least about 11:1 or at least about 33:1. In one embodiment, the ratio of lipid:siRNA ratio is between about 1:1 to about 35:1, about 3:1 to about 15:1, about 4:1 to about 15:1, or about 5:1 to about 13:1. In one embodiment, the ratio of lipid:siRNA ratio is between about 0.5:1 to about 12:1.

In one embodiment, the lipid particles are nanoparticles. In additional embodiments, the lipid particles have a mean diameter size of from about 50 nm to about 300 nm, such as from about 50 nm to about 250 nm, for example, from about 50 nm to about 200 nm.

In one embodiment, a lipid particle containing a cationic lipid of any of the embodiments described herein has an in vivo half life (t_{1/2}) (e.g., in the liver, spleen or plasma) of less than about 3 hours, such as less than about 2.5 hours, less than about 2 hours, less than about 1.5 hours, less than about 1 hour, less than about 0.5 hour or less than about 0.25 hours.

In another embodiment, a lipid particle containing a cationic lipid of any of the embodiments described herein has an in vivo half life (t_{1/2}) (e.g., in the liver, spleen or plasma) of less than about 10% (e.g., less than about 7.5%, less than about 5%, less than about 2.5%) of that for the same cationic lipid without the biodegradable group or groups.

### Additional Components

The lipid particles and compositions described herein can further include one or more antioxidants. The antioxidant stabilizes the lipid particle and prevents, decreases, and/or inhibits degradation of the cationic lipid and/or active agent present in the lipid particles. The antioxidant can be a hydrophilic antioxidant, a lipophilic antioxidant, a metal chelator, a primary antioxidant, a secondary antioxidant, salts thereof, and mixtures thereof. In certain embodiments, the antioxidant comprises a metal chelator such as EDTA or salts thereof, alone or in combination with one, two, three, four, five, six, seven, eight, or more additional antioxidants such as primary antioxidants, secondary antioxidants, or other metal chelators. In one preferred embodiment, the antioxidant comprises a metal chelator such as EDTA or salts thereof in a mixture with one or more primary antioxidants and/or secondary antioxidants. For example, the antioxidant may comprise a mixture of EDTA or a salt thereof, a primary antioxidant such as a-tocopherol or a salt thereof, and a secondary antioxidant such as ascorbyl palmitate or a salt thereof. In one embodiment, the antioxidant comprises at least about 100 mM citrate or a salt thereof. Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTPA), 2,3-dimercapto-1-propanesulfonic acid (DMPS), dimercaptosuccinic acid (DMSA), α-lipoic acid, salicylaldehyde isonicotinoyl hydrazone (SIH), hexyl thioethylamine hydrochloride (HTA), desferrioxamine, salts thereof, and mixtures thereof. Additional hydrophilic antioxidants include ascorbic acid, cysteine, glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-teri-butyl-4-hydroxyanisole and 3-tert-butyl-4-hydroxyanisole); butylhydroxytoluene (BHT); tert-butylhydroquinone (TBHQ); ascorbyl palmitate; rc-propyl gallate; salts thereof; and mixtures thereof. Suitable antioxidants and formulations containing such antioxidants are described in International Publication No. WO 2011/066651, which is hereby incorporated by reference.

In another embodiment, the lipid particles or compositions contain the antioxidant EDTA (or a salt thereof), the antioxidant citrate (or a salt thereof), or EDTA (or a salt thereof) in combination with one or more (e.g., a mixture of) primary and/or secondary antioxidants such as α-tocopherol (or a salt thereof) and/or ascorbyl palmitate (or a salt thereof).

In one embodiment, the antioxidant is present in an amount sufficient to prevent, inhibit, or reduce the degradation of the cationic lipid present in the lipid particle. For example, the antioxidant may be present at a concentration of at least about or about 0.1 mM, 0.5 mM, 1 mM, 10 mM, 100 mM, 500 mM, 1M, 2M, or 5M, or from about 0.1 mM to about 1M, from about 0.1 mM to about 500 mM, from about 0.1 mM to about 250 mM, or from about 0.1 mM to about 100 mM.

The lipid particles and compositions described herein can further include an apolipoprotein. As used herein, the term “apolipoprotein” or “lipoprotein” refers to apolipoproteins known to those of skill in the art and variants and fragments thereof and to apolipoprotein agonists, analogues or fragments thereof described below.

In a preferred embodiment, the active agent is a nucleic acid, such as a siRNA. For example, the active agent can be a nucleic acid encoded with a product of interest, including but not limited to, RNA, antisense oligonucleotide, an antagomir, a DNA, a plasmid, a ribosomal RNA (rRNA), a micro RNA (miRNA) (e.g., a miRNA which is single stranded and 17-25 nucleotides in length), transfer RNA (tRNA), a small interfering RNA (siRNA), small nuclear RNA (snRNA), antigens, fragments thereof, proteins, peptides, vaccines and small molecules or mixtures thereof. In one more preferred embodiment, the nucleic acid is an oligonucleotide (e.g., 15-50 nucleotides in length (or 15-30 or 20-30 nucleotides in length)). An siRNA can have, for instance, a duplex region that is 16-30 nucleotides long. In another embodiment, the nucleic acid is an immunostimulatory oligonucleotide, decoy oligonucleotide, supermir, miRNA mimic, or miRNA inhibitor. A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression.

The nucleic acid that is present in a lipid-nucleic acid particle can be in any form. The nucleic acid can, for example, be single-stranded DNA or RNA, or double-stranded DNA or RNA, or DNA-RNA hybrids. Non-limiting examples of double-stranded RNA include siRNA. Single-stranded nucleic acids include, e.g., antisense oligonucleotides, ribozymes, microRNA, and triplex-forming oligonucleotides. The lipid particles of the present invention can also deliver nucleic acids which are conjugated to one or more ligands.

### Pharmaceutical Compositions

The lipid particles, particularly when associated with a therapeutic agent, may be formulated as a pharmaceutical composition, e.g., which further comprises a pharmaceutically acceptable diluent, excipient, or carrier, such as physiological saline or phosphate buffer.

The resulting pharmaceutical preparations may be sterilized by conventional, well known sterilization techniques. The aqueous solutions can then be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, and tonicity adjusting agents, for example, sodium acetate, sodium lactate, sodium chloride, potassium chloride, and calcium chloride. Additionally, the lipidic suspension may include lipid-protective agents which protect lipids against free-radical and lipid-peroxidative damages on storage. Lipophilic free-radical quenchers, such as α-tocopherol and water-soluble iron-specific chelators, such as ferrioxamine, are suitable.

The concentration of lipid particle or lipid-nucleic acid particle in the pharmaceutical formulations can vary, for example, from less than about 0.01%, to at or at least about 0.05-5% to as much as 10 to 30% by weight.

### Methods of Manufacture

Methods of making cationic lipids, lipid particles containing them, and pharmaceutical compositions containing the cationic lipids and/or lipid particles are described in, for example, International Publication Nos. WO 2010/054406, WO 2010/054401, WO 2010/054405, WO 2010/054384, WO 2010/042877, WO 2010/129709, WO 2009/086558, and WO 2008/042973, and U.S. Patent Publication Nos. 2004/0142025, 2006/0051405 and 2007/0042031, each of which is incorporated by reference in its entirety.

For example, in one embodiment, a solution of one or more lipids (including a cationic lipid of any of the embodiments described herein) in an organic solution (e.g., ethanol) is prepared. Similarly, a solution of one or more active (therapeutic) agents (such as, for example an siRNA molecule or a 1:1 molar mixture of two siRNA molecules) in an aqueous buffered (e.g., citrate buffer) solution is prepared. The two solutions are mixed and diluted to form a colloidal suspension of siRNA lipid particles. In one embodiment, the siRNA lipid particles have an average particle size of about 80-90 nm. In further embodiments, the dispersion may be filtered through 0.45/2 micron filters, concentrated and diafiltered by tangential flow filtration.

### DEFINITIONS

As used herein, the term “cationic lipid” includes those lipids having one or two fatty acid or fatty aliphatic chains and an amino acid containing head group that may be protonated to form a cationic lipid at physiological pH. In some embodiments, a cationic lipid is referred to as an “amino acid conjugate cationic lipid.”

As used herein, the term “SNALP” refers to a stable nucleic acid-lipid particle. A SNALP represents a particle made from lipids (e.g., a cationic lipid, a non-cationic lipid, and optionally a conjugated lipid that prevents aggregation of the particle), wherein the nucleic acid (e.g., an interfering RNA) is encapsulated within the lipid. In certain instances, SNALP are extremely useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a SNALP as set forth in PCT Publication No. WO 00/03683, the disclosure of which is herein incorporated by reference in its entirety for all purposes.

A subject or patient in whom administration of the complex is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds, i.e., for veterinary medical use.

Many of the chemical groups recited in the generic formulas above are written in a particular order (for example, —OC(O)—). It is intended that the chemical group is to be incorporated into the generic formula in the order presented unless indicated otherwise. For example, a generic formula of the form —(R)ᵢ-(M¹)ₖ-(R)ₘ— where M¹ is —C(O)O— and k is 1 refers to —(R)ᵢ—C(O)O—(R)ₘ— unless specified otherwise. It is to be understood that when a chemical group is written in a particular order, the reverse order is also contemplated unless otherwise specified. For example, in a generic formula —(R)ᵢ-(M¹)ₖ-(R)ₘ— where M¹ is defined as —C(O)NH— (i.e., —(R)ᵢ—C(O)—NH—(R)ₘ—), the compound where M¹ is —NHC(O)— (i.e., —(R)ᵢ—NHC(O)—(R)ₘ—) is also contemplated unless otherwise specified.

The “side chain” of an amino acid refers to the chemical moiety attached to the group containing the amino and carboxyl moieties. For example, many α-amino acids have the general formula

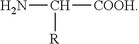

R in this formula is the side chain. In one embodiment, R is not hydrogen.

As used herein, the term “biodegradable group” refers to a group that include one or more bonds that may undergo bond breaking reactions in a biological environment, e.g., in an organism, organ, tissue, cell, or organelle. For example, the biodegradable group may be metabolizable by the body of a mammal, such as a human (e.g., by hydrolysis). Some groups that contain a biodegradable bond include, for example, but are not limited to esters, dithiols, and oximes. Non-limiting examples of biodegradable groups are —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—.

As used herein, an “aliphatic” group is a non-aromatic group in which carbon atoms are linked into chains, and is either saturated or unsaturated.

The terms “alkyl” and “alkylene” refer to a straight or branched chain saturated hydrocarbon moiety. In one embodiment, the alkyl group is a straight chain saturated hydrocarbon. Unless otherwise specified, the “alkyl” or “alkylene” group contains from 1 to 24 carbon atoms. Representative saturated straight chain alkyl groups include methyl, ethyl, n-propyl, n-butyl, n-pentyl, and n-hexyl. Representative saturated branched alkyl groups include isopropyl, sec-butyl, isobutyl, tert-butyl, and isopentyl.

The term “alkenyl” refers to a straight or branched chain hydrocarbon moiety having one or more carbon-carbon double bonds. In one embodiment, the alkenyl group contains 1, 2, or 3 double bonds and is otherwise saturated. Unless otherwise specified, the “alkenyl” group contains from 2 to 24 carbon atoms. Alkenyl groups include both cis and trans isomers. Representative straight chain and branched alkenyl groups include ethylenyl, propylenyl, 1-butenyl, 2-butenyl, isobutylenyl, 1-pentenyl, 2-pentenyl, 3-methyl-1-butenyl, 2-methyl-2-butenyl, and 2,3-dimethyl-2-butenyl.

The term “alkynyl” refers to a straight or branched chain hydrocarbon moiety having one or more carbon-carbon triple bonds. Unless otherwise specified, the “alkynyl” group contains from 2 to 24 carbon atoms. Representative straight chain and branched alkynyl groups include acetylenyl, propynyl, 1-butynyl, 2-butynyl, 1-pentynyl, 2-pentynyl, and 3-methyl-1-butynyl.

The term “acyl” refers to a carbonyl group substituted with hydrogen, alkyl, partially saturated or fully saturated cycloalkyl, partially saturated or fully saturated heterocycle, aryl, or heteroaryl. For example, acyl groups include groups such as (C₁-C₂₀)alkanoyl (e.g., formyl, acetyl, propionyl, butyryl, valeryl, caproyl, and t-butylacetyl), (C₃-C₂₀)cycloalkylcarbonyl (e.g., cyclopropylcarbonyl, cyclobutylcarbonyl, cyclopentylcarbonyl, and cyclohexylcarbonyl), heterocyclic carbonyl (e.g., pyrrolidinylcarbonyl, pyrrolid-2-one-5-carbonyl, piperidinylcarbonyl, piperazinylcarbonyl, and tetrahydrofuranylcarbonyl), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, and benzo[b]thiophenyl-2-carbonyl).

The term “aryl” refers to an aromatic monocyclic, bicyclic, or tricyclic hydrocarbon ring system. Unless otherwise specified, the “aryl” group contains from 6 to 14 carbon atoms. Examples of aryl moieties include, but are not limited to, phenyl, naphthyl, anthracenyl, and pyrenyl.

The terms “cycloalkyl” and “cycloalkylene” refer to a saturated monocyclic or bicyclic hydrocarbon moiety such as cyclopropyl, cyclobutyl, cyclopentyl, and cyclohexyl. Unless otherwise specified, the “cycloalkyl” or “cycloalkylene” group contains from 3 to 10 carbon atoms.

The term “cycloalkylalkyl” refers to a cycloalkyl group bound to an alkyl group, where the alkyl group is bound to the rest of the molecule.

The term “heterocycle” (or “heterocyclyl”) refers to a non-aromatic 5- to 8-membered monocyclic, or 7- to 12-membered bicyclic, or 11- to 14-membered tricyclic ring system which is either saturated or unsaturated, and which contains from 1 to 3 heteroatoms if monocyclic, 1-6 heteroatoms if bicyclic, or 1-9 heteroatoms if tricyclic, independently selected from nitrogen, oxygen and sulfur, and wherein the nitrogen and sulfur heteroatoms may be optionally oxidized, and the nitrogen heteroatom may be optionally quaternized. For instance, the heterocycle may be a cycloalkoxy group. The heterocycle may be attached to the rest of the molecule via any heteroatom or carbon atom in the heterocycle. Heterocycles include, but are not limited to, morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, and tetrahydrothiopyranyl.

The term “heteroaryl” refers to an aromatic 5-8 membered monocyclic, 7-12 membered bicyclic, or 11-14 membered tricyclic ring system having 1-3 heteroatoms if monocyclic, 1-6 heteroatoms if bicyclic, or 1-9 heteroatoms if tricyclic, where the heteroatoms are selected from O, N, or S (e.g., carbon atoms and 1-3, 1-6, or 1-9 heteroatoms of N, O, or S if monocyclic, bicyclic, or tricyclic, respectively). The heteroaryl groups herein described may also contain fused rings that share a common carbon-carbon bond.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, dialkylamino, and cyclic amino compounds.

The term “halogen” or “halo” refers to fluoro, chloro, bromo and iodo.

The terms “alkylamine” and “dialkylamine” refer to —NH(alkyl) and —N(alkyl)₂ radicals respectively.

The term “alkylphosphate” refers to —O—P(Q′)(Q″)—O—R, wherein Q′ and Q″ are each independently O, S, N(R)₂, optionally substituted alkyl or alkoxy; and R is optionally substituted alkyl, ω-aminoalkyl or ω-(substituted)aminoalkyl.

The term “alkylphosphorothioate” refers to an alkylphosphate wherein at least one of Q′ or Q″ is S.

The term “alkylphosphonate” refers to an alkylphosphate wherein at least one of Q′ or Q″ is alkyl.

The term “hydroxyalkyl” refers to —O-alkyl radical.

The term “alkylheterocycle” refers to an alkyl where at least one methylene has been replaced by a heterocycle.

The term “ω-aminoalkyl” refers to -alkyl-NH₂ radical. And the term “ω-(substituted)aminoalkyl refers to an ω-aminoalkyl wherein at least one of the H on N has been replaced with alkyl.

The term “ω-phosphoalkyl” refers to -alkyl-O—P(Q′)(Q″)—O—R, wherein Q′ and Q″ are each independently O or S and R optionally substituted alkyl.

The term “ω-thiophosphoalkyl refers to ω-phosphoalkyl wherein at least one of Q′ or Q″ is S.

The following abbreviations may be used in this application:

DSPC: distearoylphosphatidylcholine; DPPC: 1,2-Dipalmitoyl-sn-glycero-3-phosphocholine; POPC: 1-palmitoyl-2-oleoyl-sn-phosphatidylcholine; DOPE: 1,2-dileoyl-sn-3-phosphoethanolamine; PEG-DMG generally refers to 1,2-dimyristoyl-sn-glycerol-methoxy polyethylene glycol (e.g., PEG 2000); TBDPSC1: tert-Butylchlorodiphenylsilane; DMAP: dimethylaminopyridine; NMO: N-methylmorpholin-N-oxide; LiHDMS: lithium bis(trimethylsilyl)amide; HMPA: hexamethylphosphoramide; EDC: 1-ethyl-3-(3-dimethylaminopropyl)carbodiimide; DIPEA: diisopropylethylamine; DCM: dichloromethane; TEA: triethylamine; TBAF: tetrabutylammonium fluoride

Methods to prepare various organic groups and protective groups are known in the art and their use and modification is generally within the ability of one of skill in the art (see, for example, Green, T. W. et. al., Protective Groups in Organic Synthesis (1999); Stanley R. Sandler and Wolf Karo, Organic Functional Group Preparations (1989); Greg T. Hermanson, Bioconjugate Techniques (1996); and Leroy G. Wade, Compendium Of Organic Synthetic Methods (1980)). Briefly, protecting groups are any group that reduces or eliminates unwanted reactivity of a functional group. A protecting group can be added to a functional group to mask its reactivity during certain reactions and then removed to reveal the original functional group. In some embodiments an “alcohol protecting group” is used. An “alcohol protecting group” is any group which decreases or eliminates unwanted reactivity of an alcohol functional group. Protecting groups can be added and removed using techniques well known in the art.

The compounds may be prepared by at least one of the techniques described herein or known organic synthesis techniques.

### EXAMPLES

### Example 1

### FVII In Vivo Evaluation Using the Cationic Lipid Derived Liposomes

C57BL/6 mice (Charles River Labs, MA) receive either saline or siRNA in desired formulations via tail vein injection at a volume of 0.01 mL/g. At various time points post-administration, animals are anesthesized by isofluorane inhalation and blood is collected into serum separator tubes by retro orbital bleed. Serum levels of Factor VII protein are determined in samples using a chromogenic assay (Coaset Factor VII, DiaPharma Group, OH or Biophen FVII, Aniara Corporation, OH) according to manufacturer protocols. A standard curve is generated using serum collected from saline treated animals. In experiments where liver mRNA levels are assessed, at various time points post-administration, animals are sacrificed and livers are harvested and snap frozen in liquid nitrogen. Frozen liver tissue is ground into powder. Tissue lysates are prepared and liver mRNA levels of Factor VII and apoB are determined using a branched DNA assay (QuantiGene Assay, Panomics, CA).

### Example 2

### Determination of Efficacy of Lipid Particle Formulations Containing Various Cationic Lipids Using an In Vivo Rodent Factor VII Silencing Model

Factor VII (FVII), a prominent protein in the coagulation cascade, is synthesized in the liver (hepatocytes) and secreted into the plasma. FVII levels in plasma can be determined by a simple, plate-based colorimetric assay. As such, FVII represents a convenient model for determining siRNA-mediated downregulation of hepatocyte-derived proteins, as well as monitoring plasma concentrations and tissue distribution of the nucleic acid lipid particles and siRNA, such as the siRNA shown in Table 19.

TABLE 19

| | | SEQ | |
|---|---|---|---|
| Duplex | Sequence 5′-3′ | ID NO: | Target |
| AD-1661 | GGAfUfCAfUfCfUfCAAGfUfCfUf | | FVII |
| | UAfCdTsdT | | |
| | GfUAAGAfCfUfUGAGAfUGAfUfC | | |
| | fCdTsdT | | |

Lower case is 2′OMe modification and Nf is a 2′F modified nucleobase, dT is deoxythymidine, s is phosphothioate

The cationic lipids described herein are used to formulate liposomes containing the AD-1661 duplex using an in-line mixing method, as described in International Publication No. WO 2010/088537, which is incorporated by reference in its entirety. Lipid particles are formulated using the following molar ratio: 50% Cationic lipid/10% distearoylphosphatidylcholine (DSPC)/38.5% Cholesterol/1.5% PEG-DMG (1-(monomethoxy-polyethyleneglycol)-2,3-dimyristoylglycerol, with an average PEG molecular weight of 2000).

C57BL/6 mice (Charles River Labs, MA) receive either saline or formulated siRNA via tail vein injection. At various time points after administration, serum samples are collected by retroorbital bleed. Serum levels of Factor VII protein are determined in samples using a chromogenic assay (Biophen FVII, Aniara Corporation, OH). To determine liver mRNA levels of Factor VII, animals are sacrificed and livers are harvested and snap frozen in liquid nitrogen. Tissue lysates are prepared from the frozen tissues and liver mRNA levels of Factor VII are quantified using a branched DNA assay (QuantiGene Assay, Panomics, CA).

FVII activity is evaluated in FVII siRNA-treated animals at 48 hours after intravenous (bolus) injection in C57BL/6 mice. FVII is measured using a commercially available kit for determining protein levels in serum or tissue, following the manufacturer's instructions at a microplate scale. FVII reduction is determined against untreated control mice, and the results are expressed as % Residual FVII. Two dose levels (0.05 and 0.005 mg/kg FVII siRNA) are used in the screen of each novel liposome composition.

### Example 3

### siRNA Formulation Using Preformed Vesicles

Cationic lipid containing particles are made using the preformed vesicle method. Cationic lipid, DSPC, cholesterol and PEG-lipid are solubilized in ethanol at a molar ratio of 40/10/40/10, respectively. The lipid mixture is added to an aqueous buffer (50 mM citrate, pH 4) with mixing to a final ethanol and lipid concentration of 30% (vol/vol) and 6.1 mg/mL respectively and allowed to equilibrate at room temperature for 2 min before extrusion. The hydrated lipids are extruded through two stacked 80 nm pore-sized filters (Nuclepore) at 22° C. using a Lipex Extruder (Northern Lipids, Vancouver, BC) until a vesicle diameter of 70-90 nm, as determined by Nicomp analysis, is obtained. This generally requires 1-3 passes. For some cationic lipid mixtures which do not form small vesicles hydrating the lipid mixture with a lower pH buffer (50 mM citrate, pH 3) to protonate the phosphate group on the DSPC headgroup helps form stable 70-90 nm vesicles.

The FVII siRNA (solubilised in a 50 mM citrate, pH 4 aqueous solution containing 30% ethanol) is added to the vesicles, pre-equilibrated to 35° C., at a rate of ˜5 mL/min with mixing. After a final target siRNA/lipid ratio of 0.06 (wt/wt) is achieved, the mixture is incubated for a further 30 minutes at 35° C. to allow vesicle re-organization and encapsulation of the FVII siRNA. The ethanol is then removed and the external buffer replaced with PBS (155 mM NaCl, 3 mM Na2HPO4, 1 mM KH2PO4, pH 7.5) by either dialysis or tangential flow diafiltration. The final encapsulated siRNA-to-lipid ratio is determined after removal of unencapsulated siRNA using size-exclusion spin columns or ion exchange spin columns.

### Example 4

### In Vivo Determination of Efficacy of Lipid Formulations

Test formulations were prepared using the following in-line mixing method:

### General Protocol for the in-Line Mixing Method

Individual and separate stock solutions are prepared—one containing lipid and the other siRNA. Lipid stock containing lipid A, DSPC, cholesterol and PEG lipid is prepared by solubilized in 90% ethanol. The remaining 10% is low pH citrate buffer. The concentration of the lipid stock is 4 mg/mL. The pH of this citrate buffer can range between pH 3-5, depending on the type of fusogenic lipid employed. The siRNA is also solubilized in citrate buffer at a concentration of 4 mg/mL. For small scale, 5 mL of each stock solution is prepared.

Stock solutions are completely clear and lipids must be completely solubilized before combining with siRNA. Therefore stock solutions may be heated to completely solubilize the lipids. The siRNAs used in the process may be unmodified oligonucleotides or modified and may be conjugated with lipophilic moieties such as cholesterol.

The individual stocks are combined by pumping each solution to a T-junction. A dual-head Watson-Marlow pump is used to simultaneously control the start and stop of the two streams. A 1.6 mm polypropylene tubing is further downsized to a 0.8 mm tubing in order to increase the linear flow rate. The polypropylene line (ID=0.8 mm) are attached to either side of a T-junction. The polypropylene T has a linear edge of 1.6 mm for a resultant volume of 4.1 mm. Each of the large ends (1.6 mm) of polypropylene line is placed into test tubes containing either solubilized lipid stock or solubilized siRNA. After the T-junction a single tubing is placed where the combined stream will emit. The tubing is then extending into a container with 2× volume of PBS. The PBS is rapidly stirring. The flow rate for the pump is at a setting of 300 rpm or 110 mL/min. Ethanol is removed and exchanged for PBS by dialysis. The lipid formulations are then concentrated using centrifugation or diafiltration to an appropriate working concentration.

Test formulations are initially assessed for their FVII knockdown in female 7-9 week old, 15-25g, female C57B1/6 mice at 0.1, 0.3, 1.0 and 5.0 mg/kg with 3 mice per treatment group. All studies include animals receiving either phosphate-buffered saline (PBS, Control group) or a benchmark formulation. Formulations are diluted to the appropriate concentration in PBS immediately prior to testing. Mice are weighed and the appropriate dosing volumes calculated (10 μl/g body weight). Test and benchmark formulations as well as PBS (for Control animals) are administered intravenously via the lateral tail vein. Animals are anesthetised 24 hours later with an intraperitoneal injection of Ketamine/Xylazine and 500-700 μl of blood is collected by cardiac puncture into serum separator tubes (BD Microtainer). Blood is centrifuged at 2,000×g for 10 minutes at 15° C. and serum is collected and stored at −70° C. until analysis. Serum samples are thawed at 37° C. for 30 minutes, diluted in PBS and aliquoted into 96-well assay plates. Factor VII levels are assessed using a chromogenic assay (Biophen FVII kit, Hyphen BioMed) according to manufacturer's instructions and absorbance is measured in a microplate reader equipped with a 405 nm wavelength filter. Plasma FVII levels are quantified and ED₅₀s (dose resulting in a 50% reduction in plasma FVII levels compared to control animals) calculated using a standard curve generated from a pooled sample of serum from Control animals. Those formulations of interest showing high levels of FVII knockdown (ED₅₀<<0.1 mg/kg) are re-tested in independent studies at a lower dose range to confirm potency and establish ED₅₀ levels.

### Example 5

### Utility

### Lipid Nanoparticle (LNP) Compositions

The following lipid nanoparticle compositions (LNPs) of the instant invention are useful for the delivery of oligonucleotides, specifically siRNA and miRNA:

### Cationic Lipid/Cholesterol/PEG-DMG 56.6/38/5.4;

### Cationic Lipid/Cholesterol/PEG-DMG 60/38/2;

### Cationic Lipid/Cholesterol/PEG-DMG 67.3/29/3.7;

### Cationic Lipid/Cholesterol/PEG-DMG 49.3/47/3.7;

### Cationic Lipid/Cholesterol/PEG-DMG 50.3/44.3/5.4;

### Cationic Lipid/Cholesterol/PEG-C-DMA/DSPC 40/48/2/10; and

### Cationic Lipid/Cholesterol/PEG-DMG/DSPC 40/48/2/10.

### LNP Process Description:

The Lipid Nano-Particles (LNP) can be prepared by an impinging jet process. The particles can be formed by mixing lipids dissolved in alcohol with siRNA dissolved in a citrate buffer. In one embodiment, the mixing ratio of lipids to siRNA are targeted at 45-55% lipid and 65-45% siRNA.

For example, the lipid solution may contain a cationic lipid of the instant invention, a helper lipid (cholesterol), PEG (e.g. PEG-C-DMA, PEG-DMG) lipid, and DSPC at a concentration of 5-15 mg mL with a target of 9-12 mg/mL in an alcohol (for example ethanol).

In one embodiment, the ratio of the lipids has a mole percent range of 25-98 for the cationic lipid with a target of 35-65, the helper lipid has a mole percent range from 0-75 with a target of 30-50, the PEG lipid has a mole percent range from 1-15 with a target of 1-6, and the DSPC has a mole percent range of 0-15 with a target of 0-12. The siRNA solution contains one or more siRNA sequences at a concentration range from 0.3 to 1.0 mg mL with a target of 0.3-0.9 mg/mL in a sodium citrate buffered salt solution with pH in the range of 3.5-5. The two liquids are heated to a temperature in the range of 15-40° C., targeting 30-40° C., and then mixed in an impinging jet mixer instantly forming the LNP. The teeID has a range from 0.25 to 1.0 mm and a total flow rate from 10-600 mL/min. The combination of flow rate and tubing ID has effect of controlling the particle size of the LNPs between 30 and 200 nm. The solution is then mixed with a buffered solution at a higher pH with a mixing ratio in the range of 1:1 to 1:3 vol:vol but targeting 1:2 vol:vol. This buffered solution is at a temperature in the range of 15-40° C., targeting 30-40° C. The mixed LNPs are held from 30 minutes to 2 hrs prior to an anion exchange filtration step. The temperature during incubating is in the range of 15-40° C., targeting 30-40° C. After incubating the solution is filtered through a 0.8 μm filter containing an anion exchange separation step. This process uses tubing IDs ranging from 1 mm ID to 5 mm ID and a flow rate from 10 to 2000 mL/min. The LNPs are concentrated and diafiltered via an ultrafiltration process where the alcohol is removed and the citrate buffer is exchanged for the final buffer solution such as phosphate buffered saline. The ultrafiltration process uses a tangential flow filtration format (TFF). This process uses a membrane nominal molecular weight cutoff range from 30-500 KD. The membrane format can be hollow fiber or flat sheet cassette. The TFF processes with the proper molecular weight cutoff retains the LNP in the retentate and the filtrate or permeate contains the alcohol/citrate buffer/final buffer wastes. The TFF process is a multiple step process with an initial concentration to a siRNA concentration of 1-3 mg/mL. Following concentration, the LNPs solution is diafiltered against the final buffer for 10-20 volumes to remove the alcohol and perform buffer exchange. The material is then concentrated an additional 1-3 fold. The final steps of the LNP process are to sterile filter the concentrated LNP solution and vial the product.

### Analytical Procedure:

1) siRNA Concentration

The siRNA duplex concentrations are determined by Strong Anion-Exchange High-Performance Liquid Chromatography (SAX-HPLC) using Waters 2695 Alliance system (Water Corporation, Milford Mass.) with a 2996 PDA detector. The LNPs, otherwise referred to as RNAi Delivery Vehicles (RDVs), are treated with 0.5% Triton X-100 to free total siRNA and analyzed by SAX separation using a Dionex BioLC DNAPac PA 200 (4×250 mm) column with UV detection at 254 nm. Mobile phase is composed of A: 25 mM NaClO₄, 10 mM Tris, 20% EtOH, pH 7.0 and B: 250 mM NaClO₄, 10 mM Tris, 20% EtOH, pH 7.0 with liner gradient from 0-15 min and flow rate of 1 ml/min. The siRNA amount is determined by comparing to the siRNA standard curve.

### 2) Encapsulation Rate

Fluorescence reagent SYBR Gold is employed for RNA quantitation to monitor the encapsulation rate of RDVs. RDVs with or without Triton X-100 are used to determine the free siRNA and total siRNA amount. The assay is performed using a SpectraMax M5e microplate spectrophotometer from Molecular Devices (Sunnyvale, Calif.). Samples are excited at 485 run and fluorescence emission was measured at 530 nm. The siRNA amount is determined by comparing to the siRNA standard curve.

Encapsulation rate=(1−free siRN A/total siRNA)*100%

### 3) Particle Size and Polydispersitv

RDVs containing 1 μg siRNA are diluted to a final volume of 3 ml with 1×PBS. The particle size and polydispersity of the samples is measured by a dynamic light scattering method using ZetaPALS instrument (Brookhaven Instruments Corporation, Holtsville, N.Y.). The scattered intensity is measured with He—Ne laser at 25° C. with a scattering angle of 90°.

### 4) Zeta Potential Analysis

RDVs containing 1 μg siRNA are diluted to a final volume of 2 ml with 1 mM Tris buffer (pH 7.4). Electrophoretic mobility of samples is determined using ZetaPALS instrument (Brookhaven Instruments Corporation. Holtsville, N.Y.) with electrode and He—Ne laser as a light source. The Smoluchowski limit is assumed in the calculation of zeta potentials.

### 5) Lipid Analysis

Individual lipid concentrations are determined by Reverse Phase High-Performance Liquid Chromatography (RP-HPLC) using Waters 2695 Alliance system (Water Corporation. Milford Mass.) with a Corona charged aerosol detector (CAD) (ESA Biosciences, Inc, Chelmsford, Mass.). Individual lipids in RDVs are analyzed using an Agilent Zorbax SB-Cl 8 (50×4.6 mm, 1.8 μm particle size) column with CAD at 60° C. The mobile phase is composed of A: 0.1% TFA in H₂O and B: 0.1% TFA in IPA. The gradient changes from 60% mobile phase A and 40% mobile phase B from time 0 to 40% mobile phase A and 60% mobile phase B at 1.00 min; 40% mobile phase A and 60% mobile phase B from 1.00 to 5.00 min: 40% mobile phase A and 60% mobile phase B from 5.00 min to 25% mobile phase A and 75% mobile phase B at 10.00 min; 25% mobile phase A and 75% mobile phase B from 10.00 min to 5% mobile phase A and 95% mobile phase B at 15.00 min; and 5% mobile phase A and 95% mobile phase B from 15.00 to 60% mobile phase A and 40% mobile phase B at 20.00 min with flow rate of 1 ml/min. The individual lipid concentration is determined by comparing to the standard curve with all the lipid components in the RDVs with a quadratic curve fit. The molar percentage of each lipid is calculated based on its molecular weight.

Utilizing the above described LNP process, specific LNPs with the following ratios are identified:

### Nominal Composition:

### Cationic Lipid/Cholesterol/PEG-DMG 60/38/2

### Cationic Lipid/Cholesterol/PEG-DMG 67.3/29/3.7.

Luc siRNA

(SEQ.ID.NO.: 1)
5′-iB-AUAAGGCUAUGAAGAGAUATT-iB 3′
(SEQ.ID.NO.: 2)
3′-UUUAUUCCGAUACUUCUCUAU-5′

AUGC—Ribose
iB—Inverted deoxy abasic
UC—2′ Fluoro
AGT—2′ Deoxy
AGU—2′ OCH₃

### Nominal Composition

### Cationic Lipid/Cholesterol/PEG-DMG 60/38/2

### Cationic Lipid/Cholesterol/PEG-DMG/DSPC 40/48/2/10

### Cationic Lipid/Cholesterol/PEG-DMG/DSPC 58/30/2/10

ApoB siRNA

(SEQ ID NO.: 3)
5′-iB-CUUUAACAAUUCCUGAAAUTsT-iB-3′
(SEQ ID NO.: 4)
3′-UsUGAAAUUGUUAAGGACUsUsUsA-5′

AUGC—Ribose
iB—Inverted deoxy abasic
UC—2′ Fluoro
AGT—2′ Deoxy
AGU—2′ OCHj
UsA—phosphorothioate linkage

Oligonucleotide synthesis is well known in the art. (See US Patent Publication Nos. 2006/0083780, 2006/0240554, 2008/0020058, 2009/0263407 and 009/0285881 and International Publication Nos. WO 2009/086558, WO 2009/127060, WO 2009/132131, WO 2010/042877, WO 2010/054384, WO 2010/054401, WO 2010/054405 and WO 2010/054406).

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-113. (canceled)

114. A compound of formula (I): 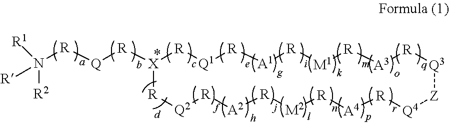
or a salt thereof, wherein
X is N or P;
R′ is absent, hydrogen, or alkyl;
with respect to R¹ and R²,
(i) R¹ and R² are each, independently, optionally substituted alkyl, alkenyl, alkynyl, cycloalkyl, cycloalkylalkyl, heterocycle or R¹⁰;
(ii) R¹ and R², together with the nitrogen atom to which they are attached, form an optionally substituted heterocylic ring; or
(iii) one of R¹ and R² is optionally substituted alkyl, alkenyl, alkynyl, cycloalkyl, cycloalkylalkyl, or heterocycle, and the other forms a 4-10 member heterocyclic ring or heteroaryl with (a) the adjacent nitrogen atom and (b) the (R)ₐ group adjacent to the nitrogen atom;
each occurrence of R is, independently, —(CR³R⁴)—;
each occurrence of R³ and R⁴ are, independently H, halogen, OH, alkyl, alkoxy, —NH₂, alkylamino, or dialkylamino;
or R³ and R⁴, together with the carbon atom to which they are directly attached, form a cycloalkyl group, wherein no more than three R groups in each chain attached to the atom X* are cycloalkyl;
each occurrence of R¹⁰ is independently selected from PEG and polymers based on poly(oxazoline), poly(ethylene oxide), poly(vinyl alcohol), poly(glycerol), poly(N-vinylpyrrolidone), poly[N-(2-hydroxypropyl)methacrylamide] and poly(amino acid)s, wherein (i) the PEG or polymer is linear or branched, (ii) the PEG or polymer is polymerized by n subunits, (iii) n is a number-averaged degree of polymerization between 10 and 200 units, and (iv) wherein the compound of formula has at most two R¹⁰ groups;
Q is absent or is —O—, —NH—, —S—, —C(O)O—, —OC(O)—, —C(O)N(R⁴)—, —N(R⁵)C(O)—, —S—S—, —OC(O)O—, —O—N═C(R⁵)—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—;
Q¹ and Q² are each, independently, absent, —O—, —S—, —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, or —OC(O)O—;
Q³ and Q⁴ are each, independently, H, —(CR³R⁴)—, aryl, or a cholesterol moiety;
each occurrence of A¹, A², A³ and A⁴ is, independently, —(CR⁵R⁵—CR⁵═CR⁵)—;
each occurrence of R⁵ is, independently, H or alkyl;
M¹ and M² are each, independently, a biodegradable group (e.g., —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—);
Z is absent, alkylene or —O—P(O)(OH)—O—;
each ------ attached to Z is an optional bond, such that when Z is absent, Q³ and Q⁴ are not directly covalently bound together;
a is 1, 2, 3, 4, 5 or 6;
b is 0, 1, 2, or 3;
c, d, e, f, i, j, m, n, q and r are each, independently, 0, 1, 2, 3, 4, 5, 6, 7, 8, 9, or 10;
g and h are each, independently, 0, 1 or 2;
k and l are each, independently, 0 or 1, where at least one of k and l is 1; and
o and p are each, independently, 0, 1 or 2,
wherein
Q³ and Q⁴ are each, independently, separated from the tertiary atom marked with an asterisk (X*) by a chain of 8 or more atoms.

115. The compound of claim 114, wherein M¹ and M² are each, independently: —OC(O)—, —C(O)O—, —SC(O)—, —C(O)S—, —OC(S)—, —C(S)O—, —S—S—, —C(R⁵)═N—, —N═C(R⁵)—, —C(R⁵)═N—O—, —O—N═C(R⁵)—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, or —OC(O)(CR³R⁴)C(O)—.

116. The compound of claim 115, wherein M¹ and M² are each, independently: —C(O)—O—, —OC(O)—, —C(R⁵)═N—, —C(R⁵)═N—O—, —O—C(O)O—, —C(O)N(R⁵)—, —C(O)S, —C(S)O—, —OSi(R⁵)₂O—, —C(O)(CR³R⁴)C(O)O—, or —OC(O)(CR³R⁴)C(O)—.

117. The compound of claim 115, wherein M¹ and M² are each —C(O)O—.

118. The compound of claim 114, wherein R¹ and R² are each alkyl.

119. The compound of claim 118, wherein R¹ and R² are each methyl.

120. The compound of claim 114, wherein Q is absent, —C(O)O—, —OC(O)—, —C(O)N(R⁴)—, —N(R⁵)C(O)—, —S—S—, —OC(O)O—, —C(R⁵)═N—O—, —OC(O)N(R⁵)—, —N(R⁵)C(O)N(R⁵)—, —N(R⁵)C(O)O—, —C(O)S—, —C(S)O— or —C(R⁵)═N—O—C(O)—.

121. The compound of claim 114, wherein Q is absent.

122. The compound of claim 114, wherein each instance of R is, independently, —CH₂—, —C(CH₃)₂— or —CH(iPr)—

123. The compound of claim 114, wherein Q¹ and Q² are each, independently, absent or —O—.

124. The compound of claim 114, wherein a is 2, 3, or 4 and b is 0.

125. The compound of claim 114, wherein a carbon atom alpha or beta to a biodegradable group is substituted with one or two alkyl groups or a spirocyclic group.

126. The compound of claim 114, wherein one or more of the following applies:
(i) Q¹ and Q² are absent;
(ii) M¹ and M² are both —C(O)O—;
(iii) g and h are both 1;
(iv) g and h are both 0;
(v) c and e total 7;
(vi) d and f total 7;
(vii) c, e and i total 7;
(viii) d, f and j total 7;
(ix) i and j are each 7;
(x) k and l are both 1;
(xi) m and n are both 0;
(xii) m and q total 1 or m and q total 2;
(xiii) m and l total 6;
(xiv) r and n total 6;
(xv) p and o are both 0;
(xvi) n and r total 2 or n and r total 1; and
(xvii) Q³ is H.

127. A compound selected from:
Compound 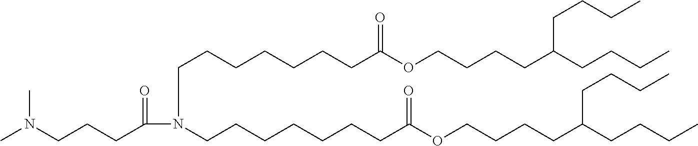 [SMILES: CCCCC(CCCC)CCCCOC(=O)CCCCCCCN(CCCCCCCC(=O)OCCCCC(CCCC)CCCC)C(=O)CCCN(C)C] 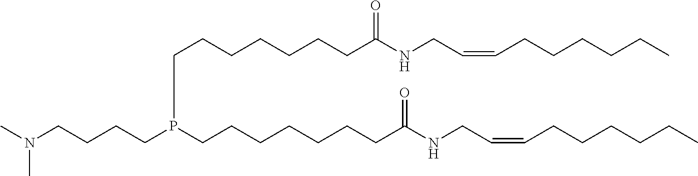 [SMILES: CCCCCC/C=C\CNC(=O)CCCCCCCP(CCCCCCCC(=O)NC/C=C\CCCCCC)CCCCN(C)C] 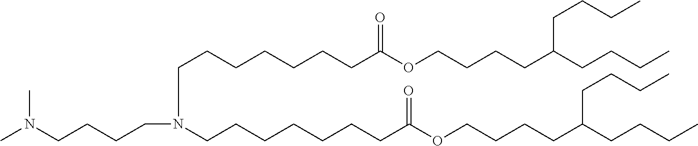 [SMILES: CCCCC(CCCC)CCCCOC(=O)CCCCCCCN(CCCCCCCC(=O)OCCCCC(CCCC)CCCC)CCCCN(C)C] 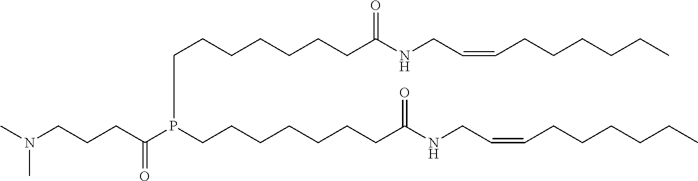 [SMILES: CCCCCC/C=C\CNC(=O)CCCCCCCP(CCCCCCCC(=O)NC/C=C\CCCCCC)C(=O)CCCN(C)C] 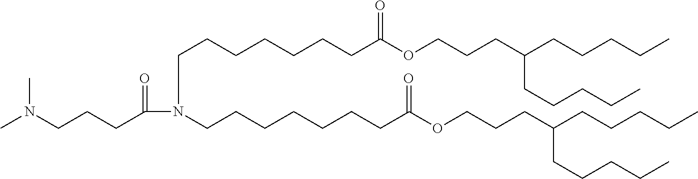 [SMILES: CCCCCC(CCCCC)CCCOC(=O)CCCCCCCN(CCCCCCCC(=O)OCCCC(CCCCC)CCCCC)C(=O)CCCN(C)C] 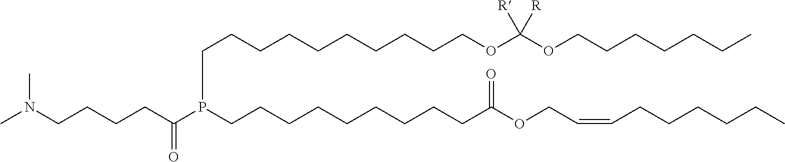 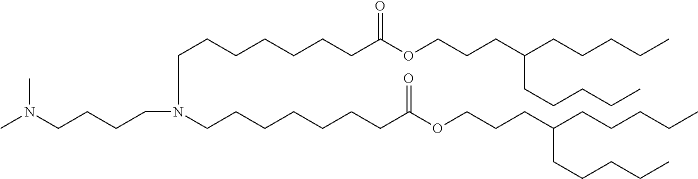 [SMILES: CCCCCC(CCCCC)CCCOC(=O)CCCCCCCN(CCCCCCCC(=O)OCCCC(CCCCC)CCCCC)CCCCN(C)C] 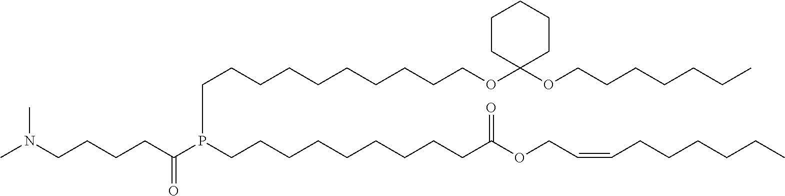 [SMILES: CCCCCC/C=C\COC(=O)CCCCCCCCCP(CCCCCCCCCCOC1(OCCCCCCC)CCCCC1)C(=O)CCCCN(C)C] 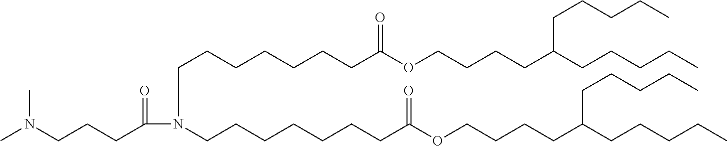 [SMILES: CCCCCC(CCCCC)CCCCOC(=O)CCCCCCCN(CCCCCCCC(=O)OCCCCC(CCCCC)CCCCC)C(=O)CCCN(C)C] 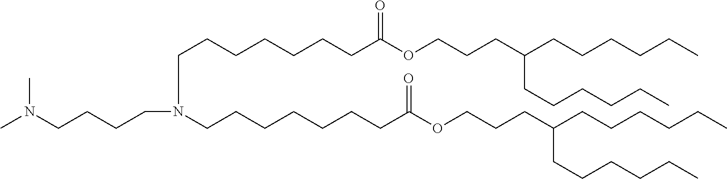 [SMILES: CCCCCCC(CCCCCC)CCCOC(=O)CCCCCCCN(CCCCCCCC(=O)OCCCC(CCCCCC)CCCCCC)CCCCN(C)C] 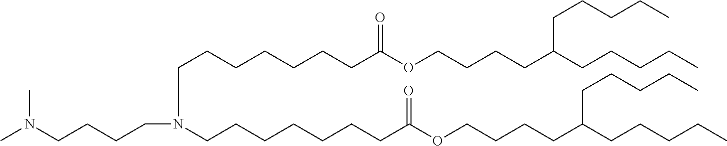 [SMILES: CCCCCC(CCCCC)CCCCOC(=O)CCCCCCCN(CCCCCCCC(=O)OCCCCC(CCCCC)CCCCC)CCCCN(C)C] 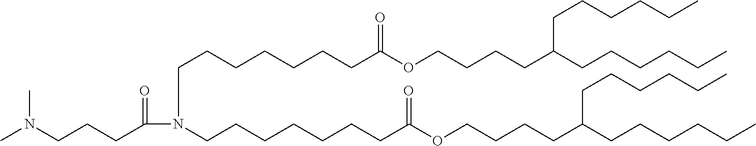 [SMILES: CCCCCCC(CCCCCC)CCCCOC(=O)CCCCCCCN(CCCCCCCC(=O)OCCCCC(CCCCCC)CCCCCC)C(=O)CCCN(C)C] 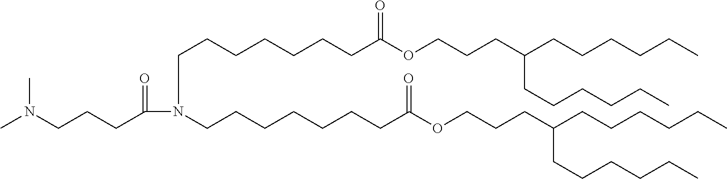 [SMILES: CCCCCCC(CCCCCC)CCCOC(=O)CCCCCCCN(CCCCCCCC(=O)OCCCC(CCCCCC)CCCCCC)C(=O)CCCN(C)C] 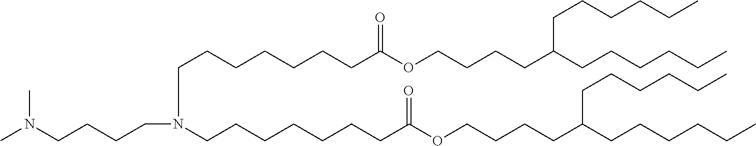 [SMILES: CCCCCCC(CCCCCC)CCCCOC(=O)CCCCCCCN(CCCCCCCC(=O)OCCCCC(CCCCCC)CCCCCC)CCCCN(C)C] 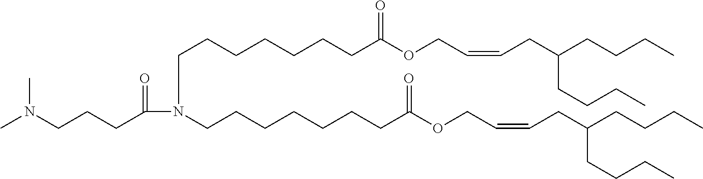 [SMILES: CCCCC(C/C=C\COC(=O)CCCCCCCN(CCCCCCCC(=O)OC/C=C\CC(CCCC)CCCC)C(=O)CCCN(C)C)CCCC] 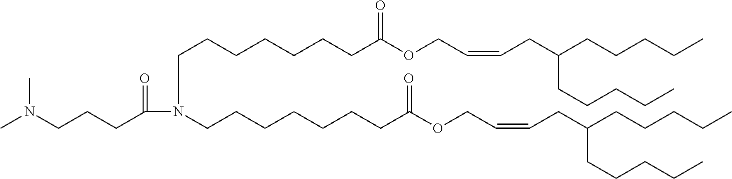 [SMILES: CCCCCC(C/C=C\COC(=O)CCCCCCCN(CCCCCCCC(=O)OC/C=C\CC(CCCCC)CCCCC)C(=O)CCCN(C)C)CCCCC] 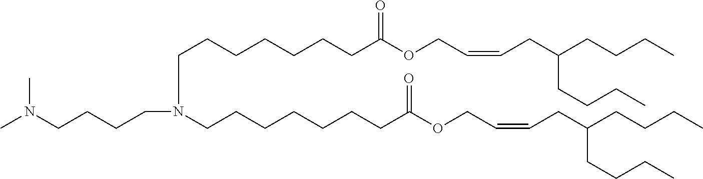 [SMILES: CCCCC(C/C=C\COC(=O)CCCCCCCN(CCCCCCCC(=O)OC/C=C\CC(CCCC)CCCC)CCCCN(C)C)CCCC] 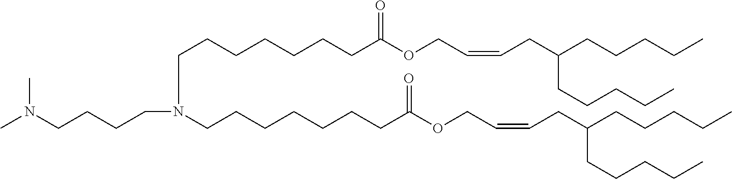 [SMILES: CCCCCC(C/C=C\COC(=O)CCCCCCCN(CCCCCCCC(=O)OC/C=C\CC(CCCCC)CCCCC)CCCCN(C)C)CCCCC] 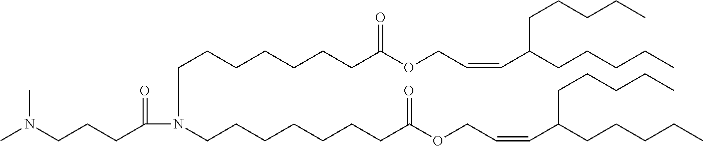 [SMILES: CCCCCC(/C=C\COC(=O)CCCCCCCN(CCCCCCCC(=O)OC/C=C\C(CCCCC)CCCCC)C(=O)CCCN(C)C)CCCCC] 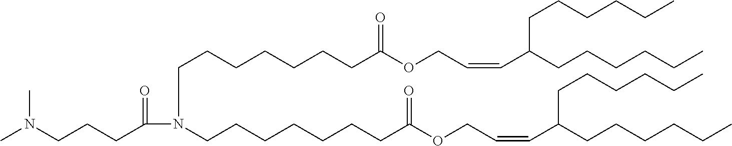 [SMILES: CCCCCCC(/C=C\COC(=O)CCCCCCCN(CCCCCCCC(=O)OC/C=C\C(CCCCCC)CCCCCC)C(=O)CCCN(C)C)CCCCCC] 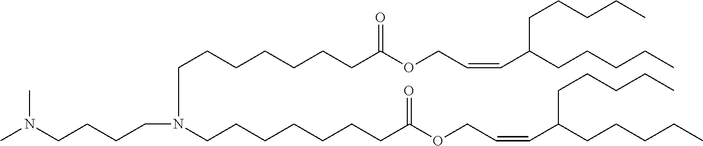 [SMILES: CCCCCC(/C=C\COC(=O)CCCCCCCN(CCCCCCCC(=O)OC/C=C\C(CCCCC)CCCCC)CCCCN(C)C)CCCCC] 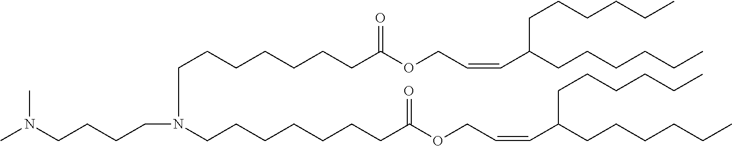 [SMILES: CCCCCCC(/C=C\COC(=O)CCCCCCCN(CCCCCCCC(=O)OC/C=C\C(CCCCCC)CCCCCC)CCCCN(C)C)CCCCCC] 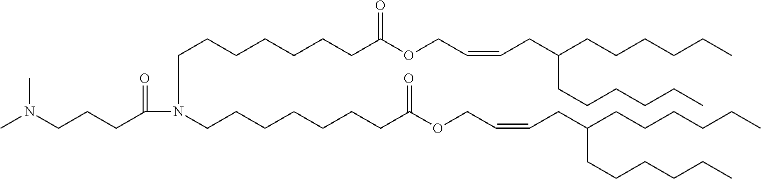 [SMILES: CCCCCCC(C/C=C\COC(=O)CCCCCCCN(CCCCCCCC(=O)OC/C=C\CC(CCCCCC)CCCCCC)C(=O)CCCN(C)C)CCCCCC] 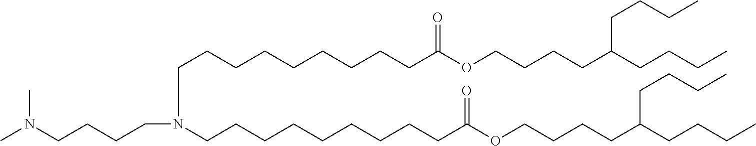 [SMILES: CCCCC(CCCC)CCCCOC(=O)CCCCCCCCCN(CCCCCCCCCC(=O)OCCCCC(CCCC)CCCC)CCCCN(C)C] 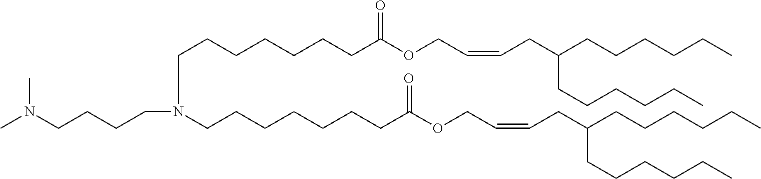 [SMILES: CCCCCCC(C/C=C\COC(=O)CCCCCCCN(CCCCCCCC(=O)OC/C=C\CC(CCCCCC)CCCCCC)CCCCN(C)C)CCCCCC] 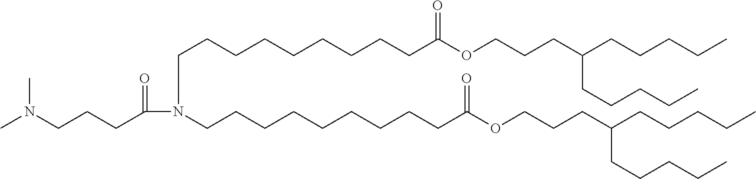 [SMILES: CCCCCC(CCCCC)CCCOC(=O)CCCCCCCCCN(CCCCCCCCCC(=O)OCCCC(CCCCC)CCCCC)C(=O)CCCN(C)C] 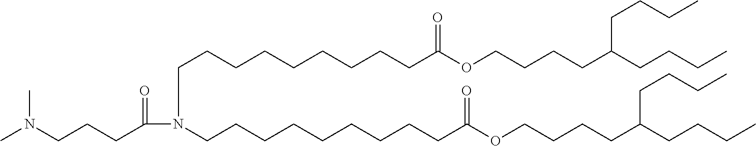 [SMILES: CCCCC(CCCC)CCCCOC(=O)CCCCCCCCCN(CCCCCCCCCC(=O)OCCCCC(CCCC)CCCC)C(=O)CCCN(C)C] 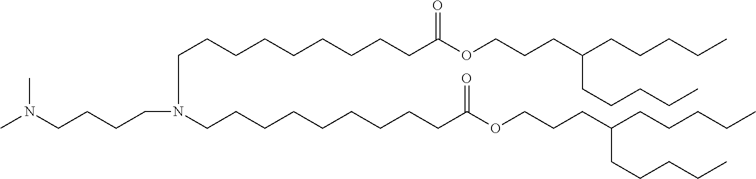 [SMILES: CCCCCC(CCCCC)CCCOC(=O)CCCCCCCCCN(CCCCCCCCCC(=O)OCCCC(CCCCC)CCCCC)CCCCN(C)C] 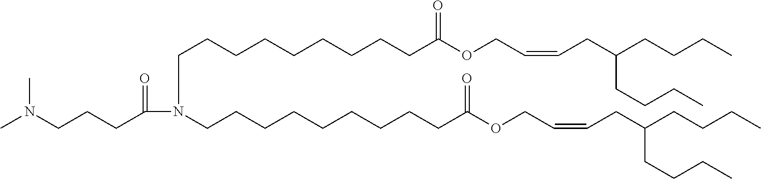 [SMILES: CCCCC(C/C=C\COC(=O)CCCCCCCCCN(CCCCCCCCCC(=O)OC/C=C\CC(CCCC)CCCC)C(=O)CCCN(C)C)CCCC] 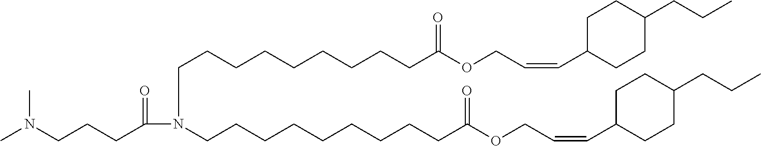 [SMILES: CCCC1CCC(/C=C\COC(=O)CCCCCCCCCN(CCCCCCCCCC(=O)OC/C=C\C2CCC(CCC)CC2)C(=O)CCCN(C)C)CC1] 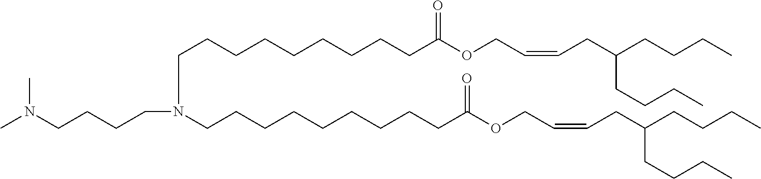 [SMILES: CCCCC(C/C=C\COC(=O)CCCCCCCCCN(CCCCCCCCCC(=O)OC/C=C\CC(CCCC)CCCC)CCCCN(C)C)CCCC] 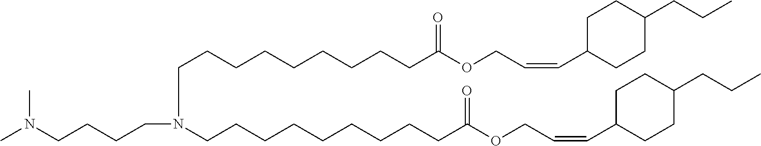 [SMILES: CCCC1CCC(/C=C\COC(=O)CCCCCCCCCN(CCCCCCCCCC(=O)OC/C=C\C2CCC(CCC)CC2)CCCCN(C)C)CC1] 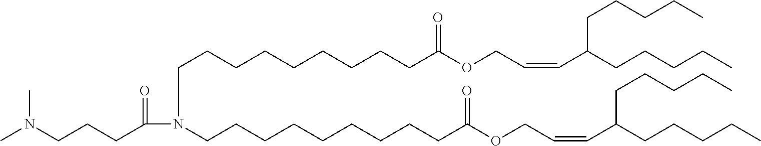 [SMILES: CCCCCC(/C=C\COC(=O)CCCCCCCCCN(CCCCCCCCCC(=O)OC/C=C\C(CCCCC)CCCCC)C(=O)CCCN(C)C)CCCCC] 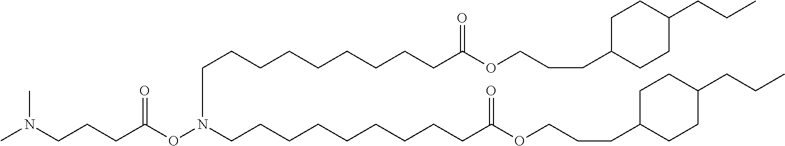 [SMILES: CCCC1CCC(CCCOC(=O)CCCCCCCCCN(CCCCCCCCCC(=O)OCCCC2CCC(CCC)CC2)OC(=O)CCCN(C)C)CC1] 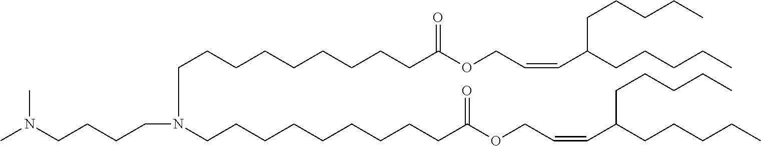 [SMILES: CCCCCC(/C=C\COC(=O)CCCCCCCCCN(CCCCCCCCCC(=O)OC/C=C\C(CCCCC)CCCCC)CCCCN(C)C)CCCCC] 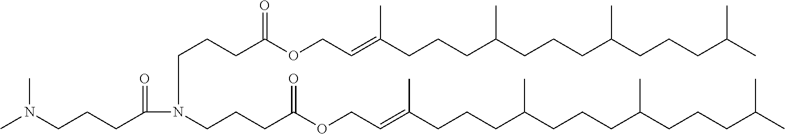 [SMILES: C/C(=C\COC(=O)CCCN(CCCC(=O)OC/C=C(\C)CCCC(C)CCCC(C)CCCC(C)C)C(=O)CCCN(C)C)CCCC(C)CCCC(C)CCCC(C)C] 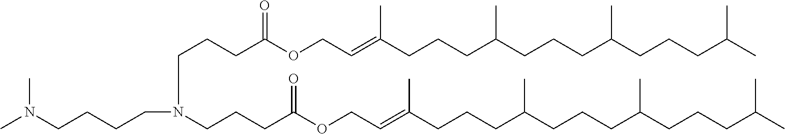 [SMILES: C/C(=C\COC(=O)CCCN(CCCCN(C)C)CCCC(=O)OC/C=C(\C)CCCC(C)CCCC(C)CCCC(C)C)CCCC(C)CCCC(C)CCCC(C)C] 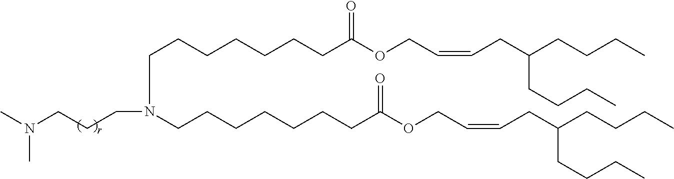 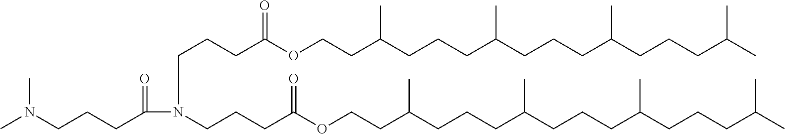 [SMILES: CC(C)CCCC(C)CCCC(C)CCCC(C)CCOC(=O)CCCN(CCCC(=O)OCCC(C)CCCC(C)CCCC(C)CCCC(C)C)C(=O)CCCN(C)C] 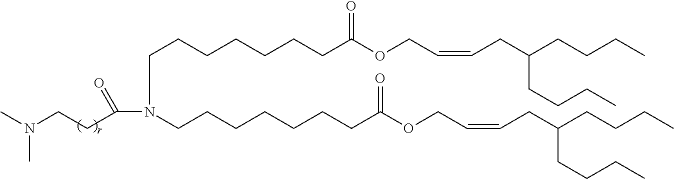 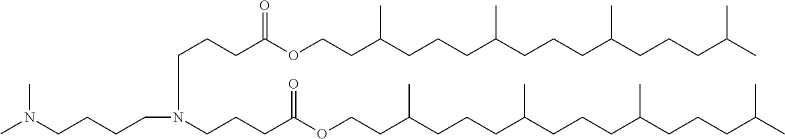 [SMILES: CC(C)CCCC(C)CCCC(C)CCCC(C)CCOC(=O)CCCN(CCCCN(C)C)CCCC(=O)OCCC(C)CCCC(C)CCCC(C)CCCC(C)C] 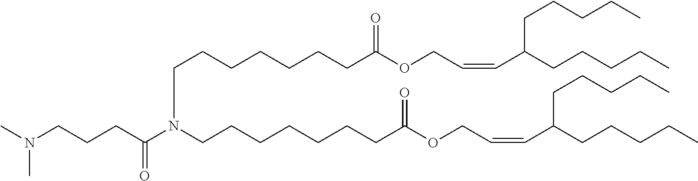 [SMILES: CCCCCC(/C=C\COC(=O)CCCCCCCN(CCCCCCCC(=O)OC/C=C\C(CCCCC)CCCCC)C(=O)CCCN(C)C)CCCCC] 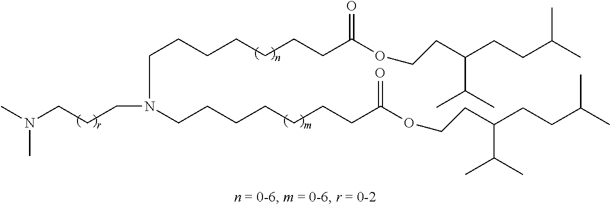 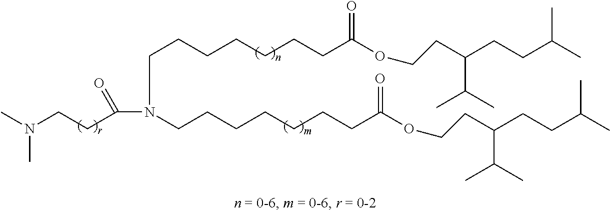 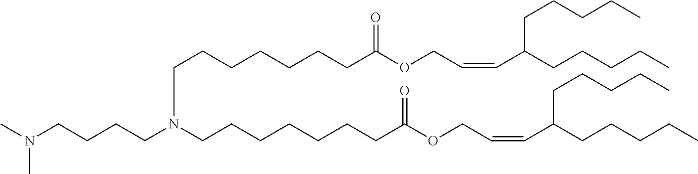 [SMILES: CCCCCC(/C=C\COC(=O)CCCCCCCN(CCCCCCCC(=O)OC/C=C\C(CCCCC)CCCCC)CCCCN(C)C)CCCCC] 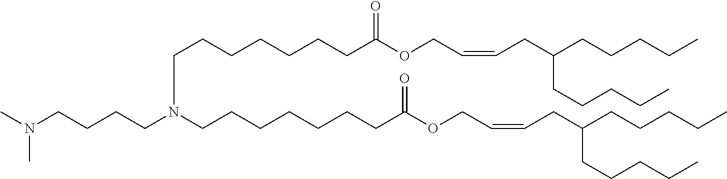 [SMILES: CCCCCC(C/C=C\COC(=O)CCCCCCCN(CCCCCCCC(=O)OC/C=C\CC(CCCCC)CCCCC)CCCCN(C)C)CCCCC] 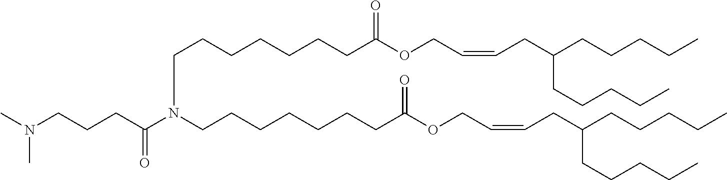 [SMILES: CCCCCC(C/C=C\COC(=O)CCCCCCCN(CCCCCCCC(=O)OC/C=C\CC(CCCCC)CCCCC)C(=O)CCCN(C)C)CCCCC] 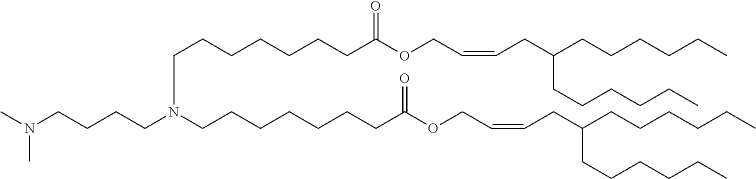 [SMILES: CCCCCCC(C/C=C\COC(=O)CCCCCCCN(CCCCCCCC(=O)OC/C=C\CC(CCCCCC)CCCCCC)CCCCN(C)C)CCCCCC] 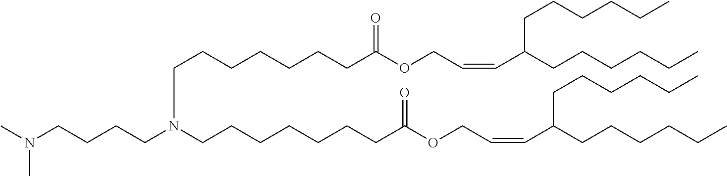 [SMILES: CCCCCCC(/C=C\COC(=O)CCCCCCCN(CCCCCCCC(=O)OC/C=C\C(CCCCCC)CCCCCC)CCCCN(C)C)CCCCCC] 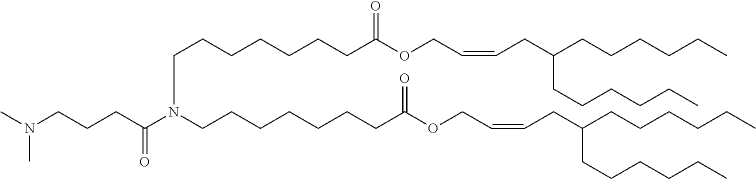 [SMILES: CCCCCCC(C/C=C\COC(=O)CCCCCCCN(CCCCCCCC(=O)OC/C=C\CC(CCCCCC)CCCCCC)C(=O)CCCN(C)C)CCCCCC] 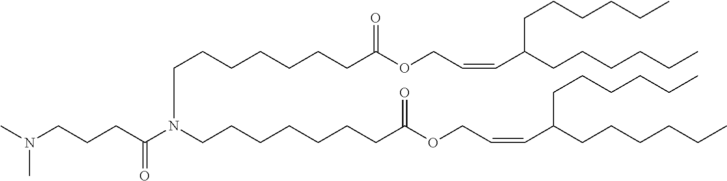 [SMILES: CCCCCCC(/C=C\COC(=O)CCCCCCCN(CCCCCCCC(=O)OC/C=C\C(CCCCCC)CCCCCC)C(=O)CCCN(C)C)CCCCCC] 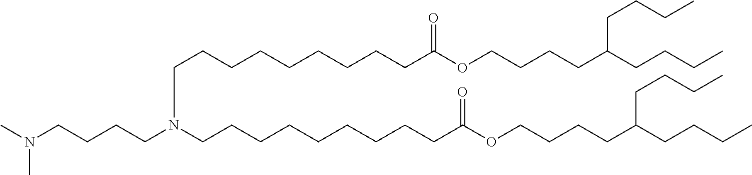 [SMILES: CCCCC(CCCC)CCCCOC(=O)CCCCCCCCCN(CCCCCCCCCC(=O)OCCCCC(CCCC)CCCC)CCCCN(C)C] 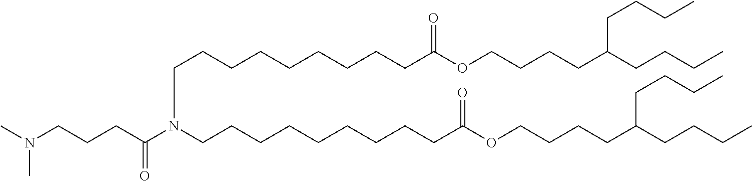 [SMILES: CCCCC(CCCC)CCCCOC(=O)CCCCCCCCCN(CCCCCCCCCC(=O)OCCCCC(CCCC)CCCC)C(=O)CCCN(C)C] 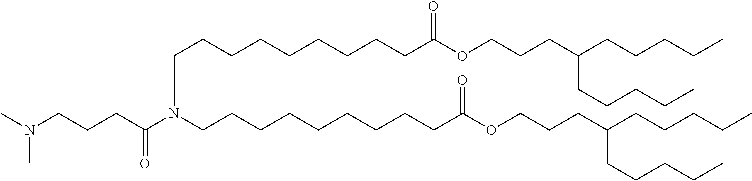 [SMILES: CCCCCC(CCCCC)CCCOC(=O)CCCCCCCCCN(CCCCCCCCCC(=O)OCCCC(CCCCC)CCCCC)C(=O)CCCN(C)C] 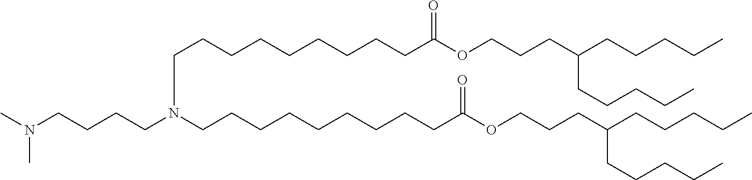 [SMILES: CCCCCC(CCCCC)CCCOC(=O)CCCCCCCCCN(CCCCCCCCCC(=O)OCCCC(CCCCC)CCCCC)CCCCN(C)C] 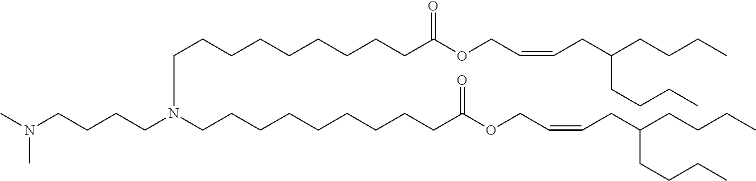 [SMILES: CCCCC(C/C=C\COC(=O)CCCCCCCCCN(CCCCCCCCCC(=O)OC/C=C\CC(CCCC)CCCC)CCCCN(C)C)CCCC] 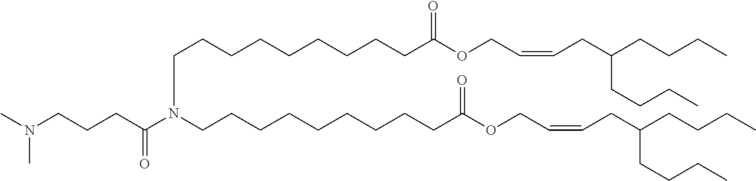 [SMILES: CCCCC(C/C=C\COC(=O)CCCCCCCCCN(CCCCCCCCCC(=O)OC/C=C\CC(CCCC)CCCC)C(=O)CCCN(C)C)CCCC] 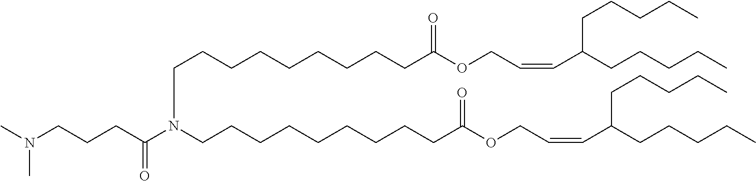 [SMILES: CCCCCC(/C=C\COC(=O)CCCCCCCCCN(CCCCCCCCCC(=O)OC/C=C\C(CCCCC)CCCCC)C(=O)CCCN(C)C)CCCCC] 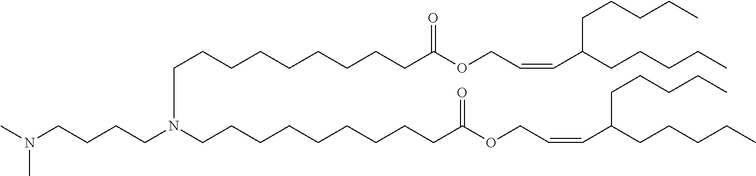 [SMILES: CCCCCC(/C=C\COC(=O)CCCCCCCCCN(CCCCCCCCCC(=O)OC/C=C\C(CCCCC)CCCCC)CCCCN(C)C)CCCCC] 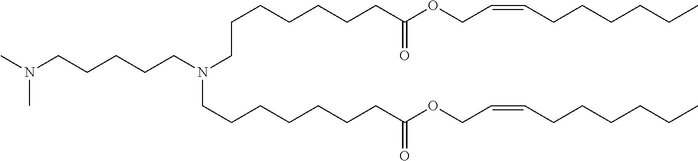 [SMILES: CCCCCC/C=C\COC(=O)CCCCCCCN(CCCCCCCC(=O)OC/C=C\CCCCCC)CCCCCN(C)C] 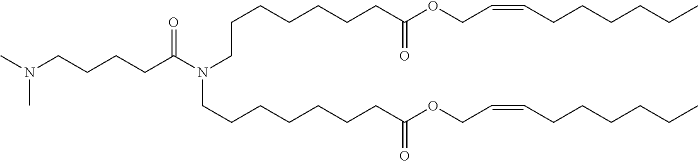 [SMILES: CCCCCC/C=C\COC(=O)CCCCCCCN(CCCCCCCC(=O)OC/C=C\CCCCCC)C(=O)CCCCN(C)C] 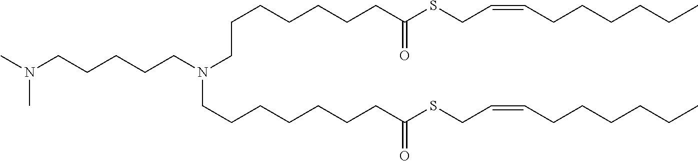 [SMILES: CCCCCC/C=C\CSC(=O)CCCCCCCN(CCCCCCCC(=O)SC/C=C\CCCCCC)CCCCCN(C)C] 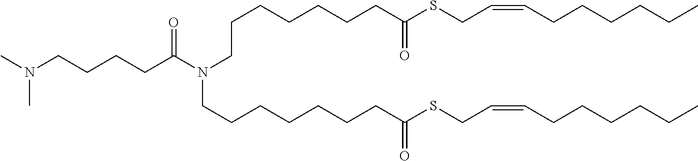 [SMILES: CCCCCC/C=C\CSC(=O)CCCCCCCN(CCCCCCCC(=O)SC/C=C\CCCCCC)C(=O)CCCCN(C)C] 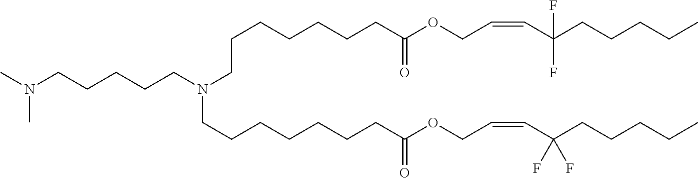 [SMILES: CCCCCC(F)(F)/C=C\COC(=O)CCCCCCCN(CCCCCCCC(=O)OC/C=C\C(F)(F)CCCCC)CCCCCN(C)C] 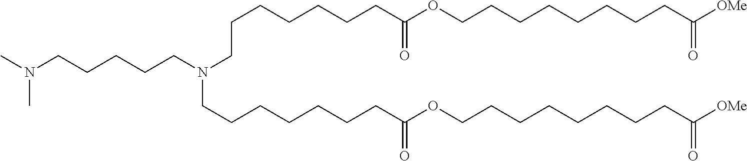 [SMILES: COC(=O)CCCCCCCCOC(=O)CCCCCCCN(CCCCCCCC(=O)OCCCCCCCCC(=O)OC)CCCCCN(C)C] 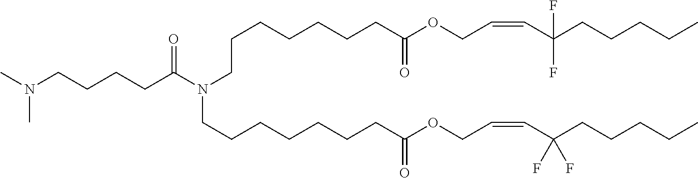 [SMILES: CCCCCC(F)(F)/C=C\COC(=O)CCCCCCCN(CCCCCCCC(=O)OC/C=C\C(F)(F)CCCCC)C(=O)CCCCN(C)C] 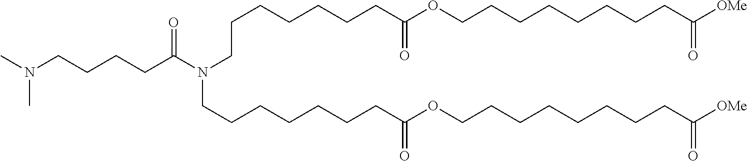 [SMILES: COC(=O)CCCCCCCCOC(=O)CCCCCCCN(CCCCCCCC(=O)OCCCCCCCCC(=O)OC)C(=O)CCCCN(C)C] 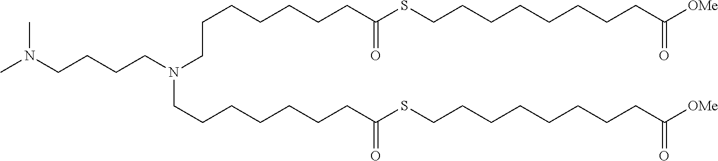 [SMILES: COC(=O)CCCCCCCCSC(=O)CCCCCCCN(CCCCCCCC(=O)SCCCCCCCCC(=O)OC)CCCCN(C)C] 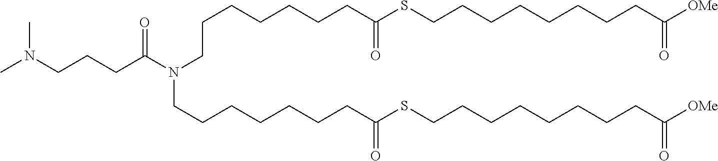 [SMILES: COC(=O)CCCCCCCCSC(=O)CCCCCCCN(CCCCCCCC(=O)SCCCCCCCCC(=O)OC)C(=O)CCCN(C)C] 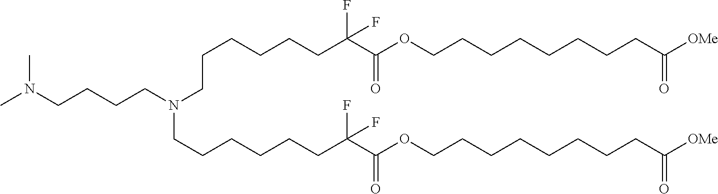 [SMILES: COC(=O)CCCCCCCCOC(=O)C(F)(F)CCCCCCN(CCCCCCC(F)(F)C(=O)OCCCCCCCCC(=O)OC)CCCCN(C)C] 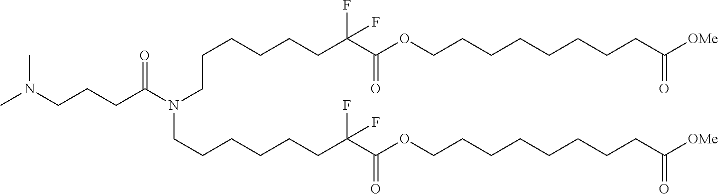 [SMILES: COC(=O)CCCCCCCCOC(=O)C(F)(F)CCCCCCN(CCCCCCC(F)(F)C(=O)OCCCCCCCCC(=O)OC)C(=O)CCCN(C)C] 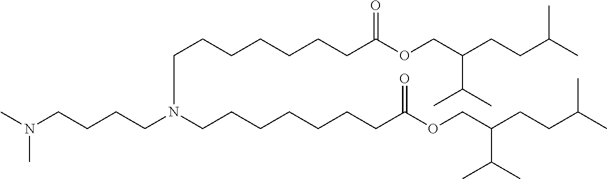 [SMILES: CC(C)CCC(COC(=O)CCCCCCCN(CCCCCCCC(=O)OCC(CCC(C)C)C(C)C)CCCCN(C)C)C(C)C] 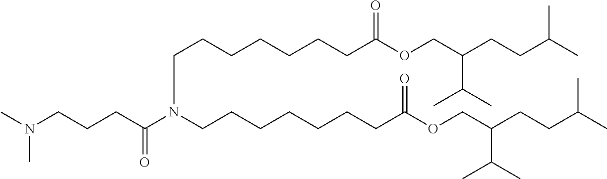 [SMILES: CC(C)CCC(COC(=O)CCCCCCCN(CCCCCCCC(=O)OCC(CCC(C)C)C(C)C)C(=O)CCCN(C)C)C(C)C] 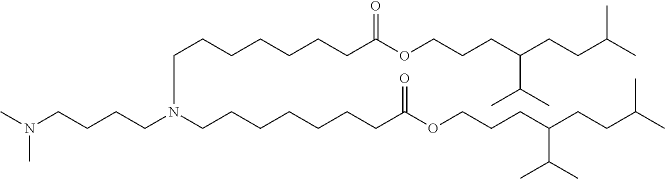 [SMILES: CC(C)CCC(CCCOC(=O)CCCCCCCN(CCCCCCCC(=O)OCCCC(CCC(C)C)C(C)C)CCCCN(C)C)C(C)C] 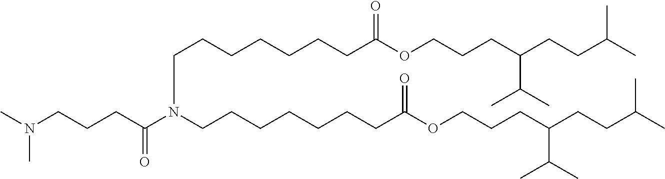 [SMILES: CC(C)CCC(CCCOC(=O)CCCCCCCN(CCCCCCCC(=O)OCCCC(CCC(C)C)C(C)C)C(=O)CCCN(C)C)C(C)C] 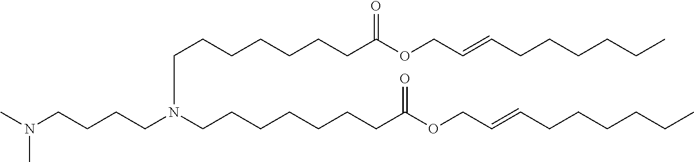 [SMILES: CCCCCC/C=C/COC(=O)CCCCCCCN(CCCCCCCC(=O)OC/C=C/CCCCCC)CCCCN(C)C] 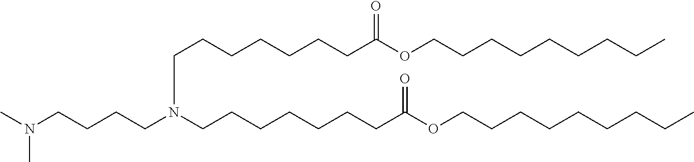 [SMILES: CCCCCCCCCOC(=O)CCCCCCCN(CCCCCCCC(=O)OCCCCCCCCC)CCCCN(C)C] 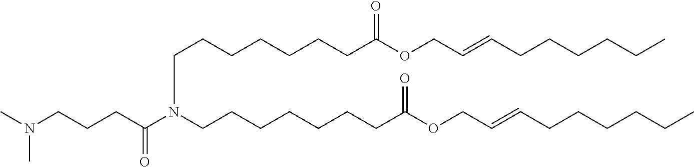 [SMILES: CCCCCC/C=C/COC(=O)CCCCCCCN(CCCCCCCC(=O)OC/C=C/CCCCCC)C(=O)CCCN(C)C] 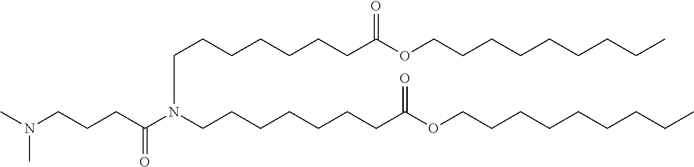 [SMILES: CCCCCCCCCOC(=O)CCCCCCCN(CCCCCCCC(=O)OCCCCCCCCC)C(=O)CCCN(C)C] 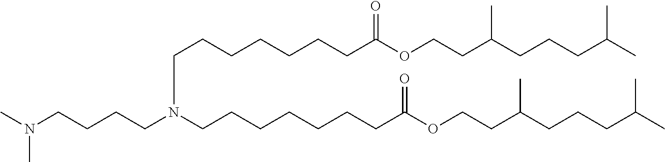 [SMILES: CC(C)CCCC(C)CCOC(=O)CCCCCCCN(CCCCCCCC(=O)OCCC(C)CCCC(C)C)CCCCN(C)C] 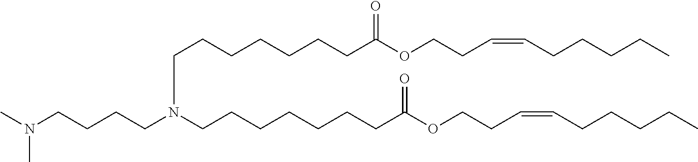 [SMILES: CCCCC/C=C\CCOC(=O)CCCCCCCN(CCCCCCCC(=O)OCC/C=C\CCCCC)CCCCN(C)C] 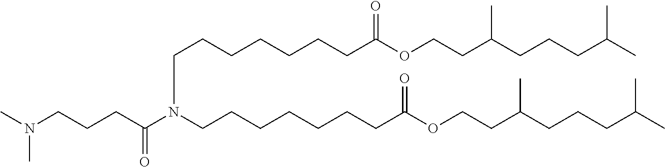 [SMILES: CC(C)CCCC(C)CCOC(=O)CCCCCCCN(CCCCCCCC(=O)OCCC(C)CCCC(C)C)C(=O)CCCN(C)C] 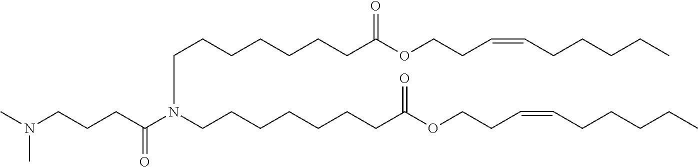 [SMILES: CCCCC/C=C\CCOC(=O)CCCCCCCN(CCCCCCCC(=O)OCC/C=C\CCCCC)C(=O)CCCN(C)C] 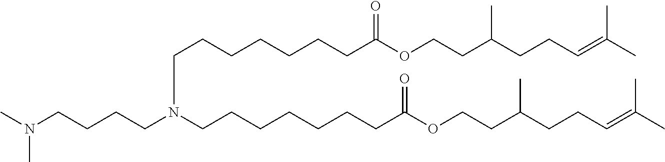 [SMILES: CC(C)=CCCC(C)CCOC(=O)CCCCCCCN(CCCCCCCC(=O)OCCC(C)CCC=C(C)C)CCCCN(C)C] 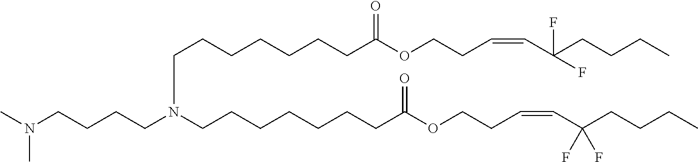 [SMILES: CCCCC(F)(F)/C=C\CCOC(=O)CCCCCCCN(CCCCCCCC(=O)OCC/C=C\C(F)(F)CCCC)CCCCN(C)C] 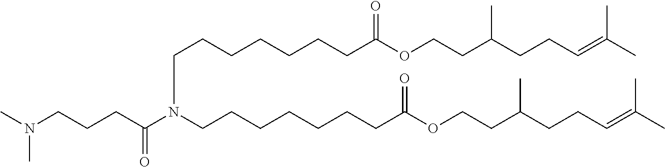 [SMILES: CC(C)=CCCC(C)CCOC(=O)CCCCCCCN(CCCCCCCC(=O)OCCC(C)CCC=C(C)C)C(=O)CCCN(C)C] 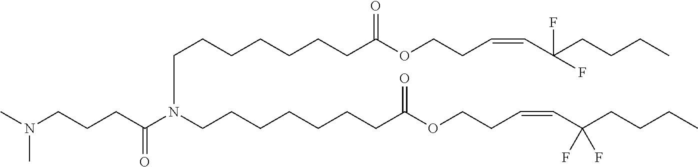 [SMILES: CCCCC(F)(F)/C=C\CCOC(=O)CCCCCCCN(CCCCCCCC(=O)OCC/C=C\C(F)(F)CCCC)C(=O)CCCN(C)C] 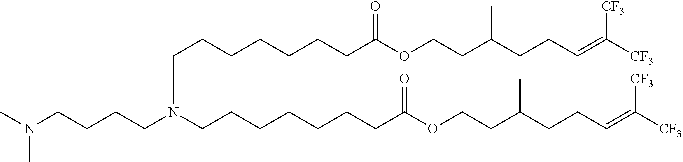 [SMILES: CC(CCC=C(C(F)(F)F)C(F)(F)F)CCOC(=O)CCCCCCCN(CCCCCCCC(=O)OCCC(C)CCC=C(C(F)(F)F)C(F)(F)F)CCCCN(C)C] 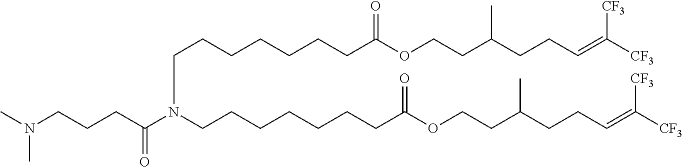 [SMILES: CC(CCC=C(C(F)(F)F)C(F)(F)F)CCOC(=O)CCCCCCCN(CCCCCCCC(=O)OCCC(C)CCC=C(C(F)(F)F)C(F)(F)F)C(=O)CCCN(C)C] 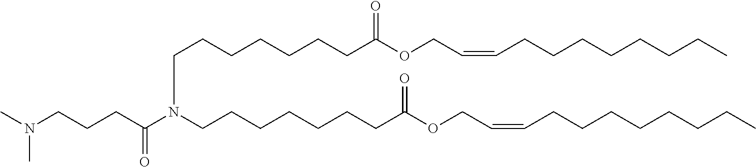 [SMILES: CCCCCCCC/C=C\COC(=O)CCCCCCCN(CCCCCCCC(=O)OC/C=C\CCCCCCCC)C(=O)CCCN(C)C] 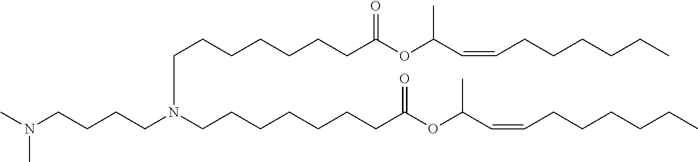 [SMILES: CCCCCC/C=C\C(C)OC(=O)CCCCCCCN(CCCCCCCC(=O)OC(C)/C=C\CCCCCC)CCCCN(C)C] 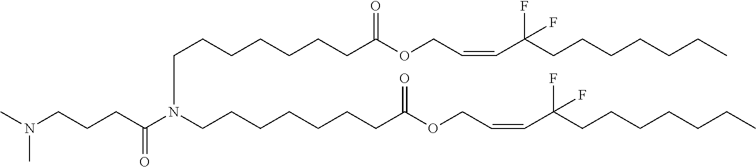 [SMILES: CCCCCCCC(F)(F)/C=C\COC(=O)CCCCCCCN(CCCCCCCC(=O)OC/C=C\C(F)(F)CCCCCCC)C(=O)CCCN(C)C] 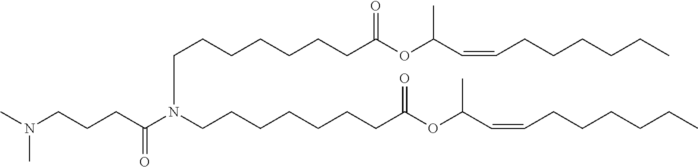 [SMILES: CCCCCC/C=C\C(C)OC(=O)CCCCCCCN(CCCCCCCC(=O)OC(C)/C=C\CCCCCC)C(=O)CCCN(C)C] 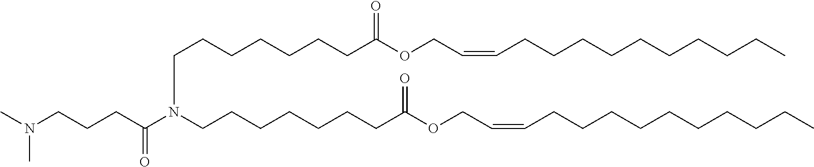 [SMILES: CCCCCCCCCC/C=C\COC(=O)CCCCCCCN(CCCCCCCC(=O)OC/C=C\CCCCCCCCCC)C(=O)CCCN(C)C] 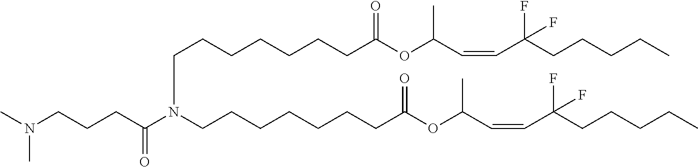 [SMILES: CCCCCC(F)(F)/C=C\C(C)OC(=O)CCCCCCCN(CCCCCCCC(=O)OC(C)/C=C\C(F)(F)CCCCC)C(=O)CCCN(C)C] 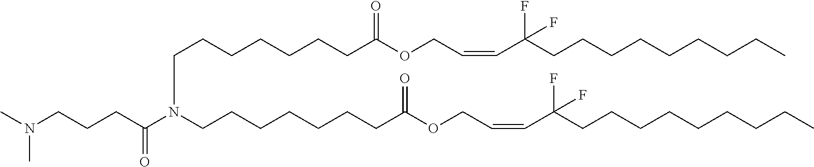 [SMILES: CCCCCCCCCC(F)(F)/C=C\COC(=O)CCCCCCCN(CCCCCCCC(=O)OC/C=C\C(F)(F)CCCCCCCCC)C(=O)CCCN(C)C] 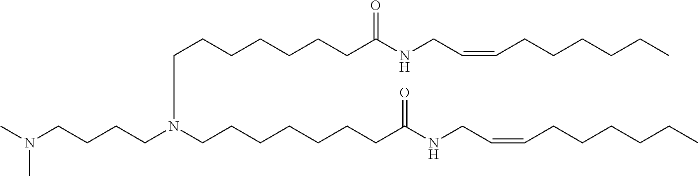 [SMILES: CCCCCC/C=C\CNC(=O)CCCCCCCN(CCCCCCCC(=O)NC/C=C\CCCCCC)CCCCN(C)C] 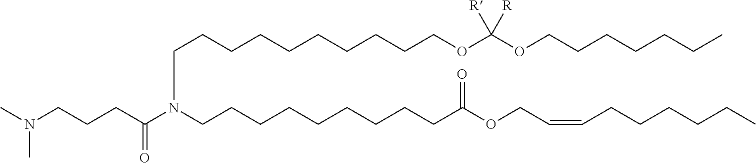 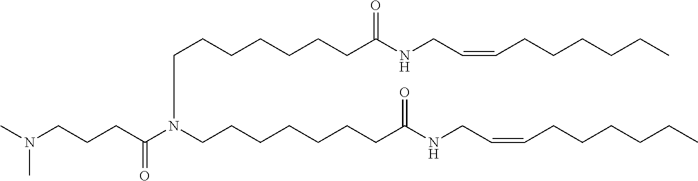 [SMILES: CCCCCC/C=C\CNC(=O)CCCCCCCN(CCCCCCCC(=O)NC/C=C\CCCCCC)C(=O)CCCN(C)C] 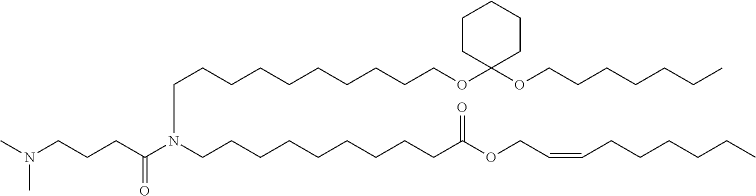 [SMILES: CCCCCC/C=C\COC(=O)CCCCCCCCCN(CCCCCCCCCCOC1(OCCCCCCC)CCCCC1)C(=O)CCCN(C)C]
and salts thereof.

128. A compound selected from:
Compound

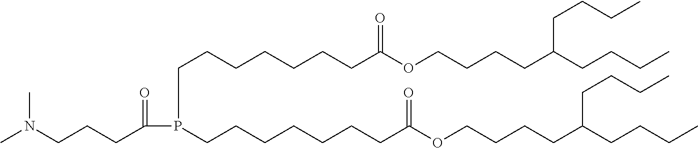 [SMILES: CCCCC(CCCC)CCCCOC(=O)CCCCCCCP(CCCCCCCC(=O)OCCCCC(CCCC)CCCC)C(=O)CCCN(C)C]

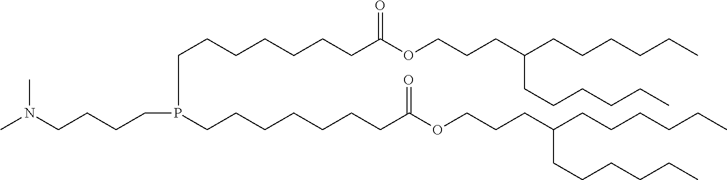 [SMILES: CCCCCCC(CCCCCC)CCCOC(=O)CCCCCCCP(CCCCCCCC(=O)OCCCC(CCCCCC)CCCCCC)CCCCN(C)C]

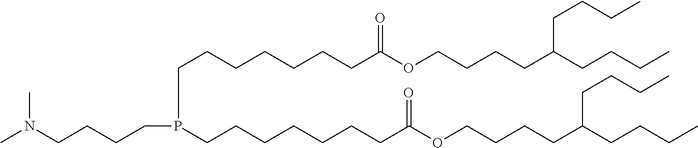 [SMILES: CCCCC(CCCC)CCCCOC(=O)CCCCCCCP(CCCCCCCC(=O)OCCCCC(CCCC)CCCC)CCCCN(C)C]

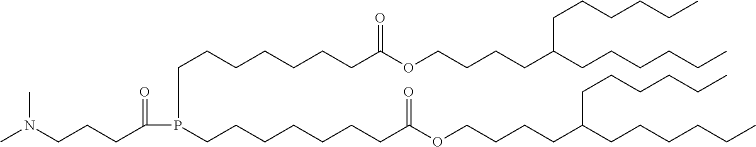 [SMILES: CCCCCCC(CCCCCC)CCCCOC(=O)CCCCCCCP(CCCCCCCC(=O)OCCCCC(CCCCCC)CCCCCC)C(=O)CCCN(C)C]

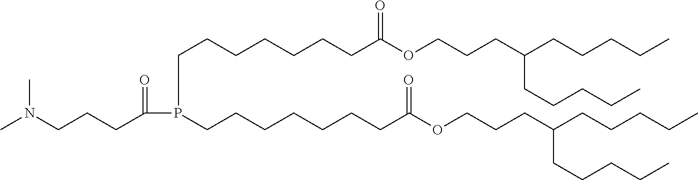 [SMILES: CCCCCC(CCCCC)CCCOC(=O)CCCCCCCP(CCCCCCCC(=O)OCCCC(CCCCC)CCCCC)C(=O)CCCN(C)C]

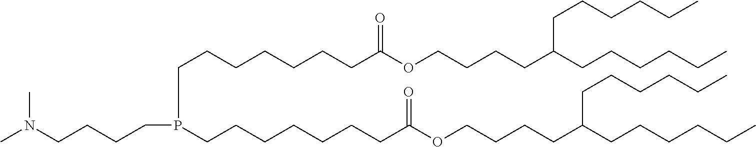 [SMILES: CCCCCCC(CCCCCC)CCCCOC(=O)CCCCCCCP(CCCCCCCC(=O)OCCCCC(CCCCCC)CCCCCC)CCCCN(C)C]

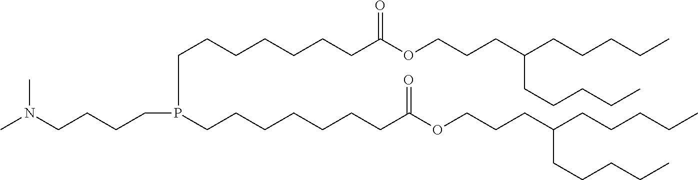 [SMILES: CCCCCC(CCCCC)CCCOC(=O)CCCCCCCP(CCCCCCCC(=O)OCCCC(CCCCC)CCCCC)CCCCN(C)C]

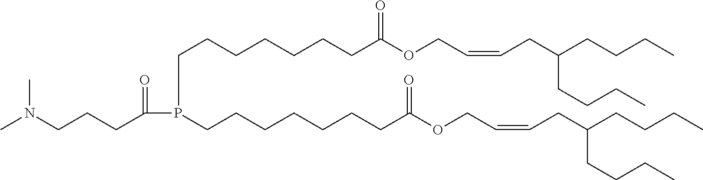 [SMILES: CCCCC(C/C=C\COC(=O)CCCCCCCP(CCCCCCCC(=O)OC/C=C\CC(CCCC)CCCC)C(=O)CCCN(C)C)CCCC]

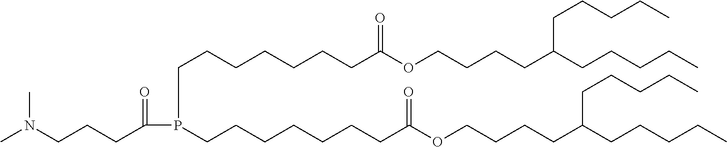 [SMILES: CCCCCC(CCCCC)CCCCOC(=O)CCCCCCCP(CCCCCCCC(=O)OCCCCC(CCCCC)CCCCC)C(=O)CCCN(C)C]

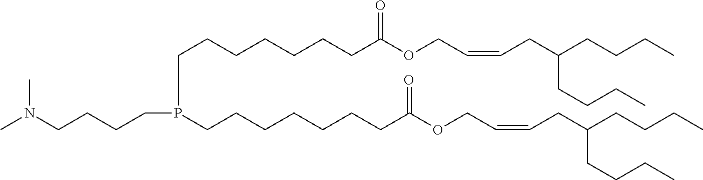 [SMILES: CCCCC(C/C=C\COC(=O)CCCCCCCP(CCCCCCCC(=O)OC/C=C\CC(CCCC)CCCC)CCCCN(C)C)CCCC]

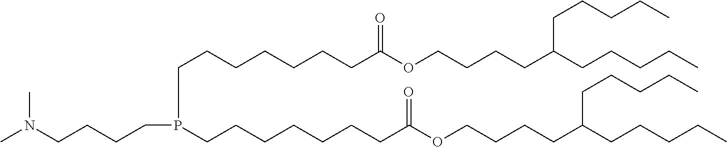 [SMILES: CCCCCC(CCCCC)CCCCOC(=O)CCCCCCCP(CCCCCCCC(=O)OCCCCC(CCCCC)CCCCC)CCCCN(C)C]

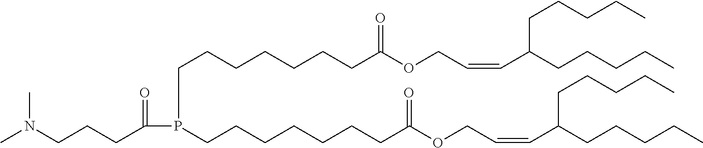 [SMILES: CCCCCC(/C=C\COC(=O)CCCCCCCP(CCCCCCCC(=O)OC/C=C\C(CCCCC)CCCCC)C(=O)CCCN(C)C)CCCCC]

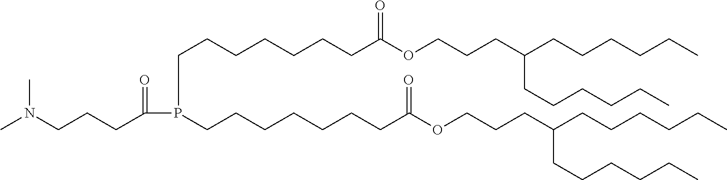 [SMILES: CCCCCCC(CCCCCC)CCCOC(=O)CCCCCCCP(CCCCCCCC(=O)OCCCC(CCCCCC)CCCCCC)C(=O)CCCN(C)C]

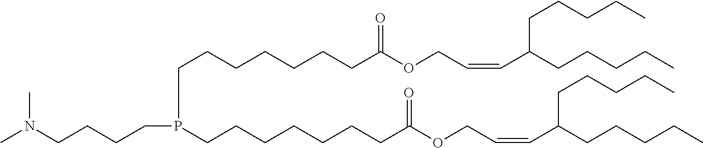 [SMILES: CCCCCC(/C=C\COC(=O)CCCCCCCP(CCCCCCCC(=O)OC/C=C\C(CCCCC)CCCCC)CCCCN(C)C)CCCCC]

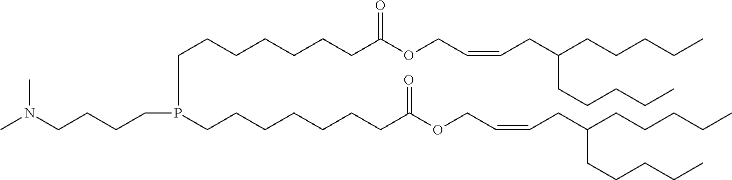 [SMILES: CCCCCC(C/C=C\COC(=O)CCCCCCCP(CCCCCCCC(=O)OC/C=C\CC(CCCCC)CCCCC)CCCCN(C)C)CCCCC]

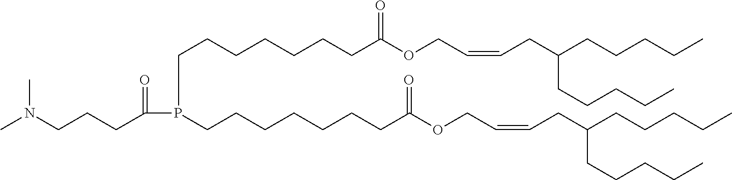 [SMILES: CCCCCC(C/C=C\COC(=O)CCCCCCCP(CCCCCCCC(=O)OC/C=C\CC(CCCCC)CCCCC)C(=O)CCCN(C)C)CCCCC]

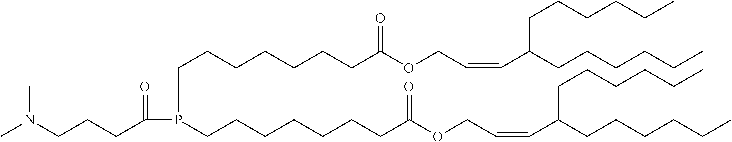 [SMILES: CCCCCCC(/C=C\COC(=O)CCCCCCCP(CCCCCCCC(=O)OC/C=C\C(CCCCCC)CCCCCC)C(=O)CCCN(C)C)CCCCCC]

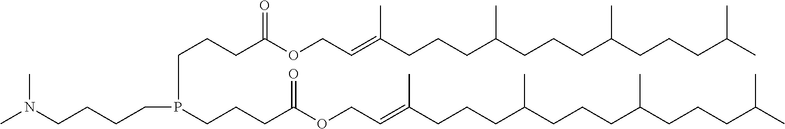 [SMILES: C/C(=C\COC(=O)CCCP(CCCCN(C)C)CCCC(=O)OC/C=C(\C)CCCC(C)CCCC(C)CCCC(C)C)CCCC(C)CCCC(C)CCCC(C)C]

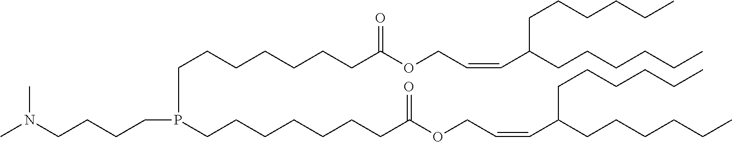 [SMILES: CCCCCCC(/C=C\COC(=O)CCCCCCCP(CCCCCCCC(=O)OC/C=C\C(CCCCCC)CCCCCC)CCCCN(C)C)CCCCCC]

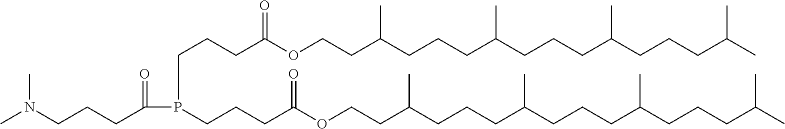 [SMILES: CC(C)CCCC(C)CCCC(C)CCCC(C)CCOC(=O)CCCP(CCCC(=O)OCCC(C)CCCC(C)CCCC(C)CCCC(C)C)C(=O)CCCN(C)C]

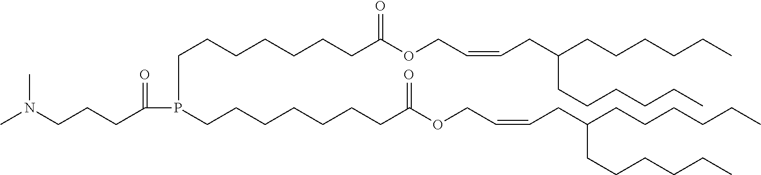 [SMILES: CCCCCCC(C/C=C\COC(=O)CCCCCCCP(CCCCCCCC(=O)OC/C=C\CC(CCCCCC)CCCCCC)C(=O)CCCN(C)C)CCCCCC]

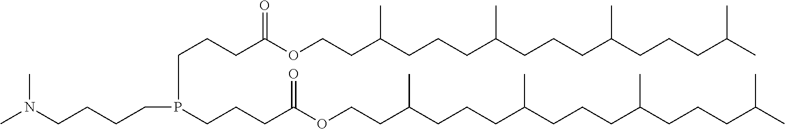 [SMILES: CC(C)CCCC(C)CCCC(C)CCCC(C)CCOC(=O)CCCP(CCCCN(C)C)CCCC(=O)OCCC(C)CCCC(C)CCCC(C)CCCC(C)C]

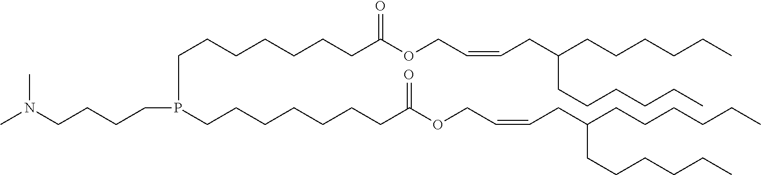 [SMILES: CCCCCCC(C/C=C\COC(=O)CCCCCCCP(CCCCCCCC(=O)OC/C=C\CC(CCCCCC)CCCCCC)CCCCN(C)C)CCCCCC]

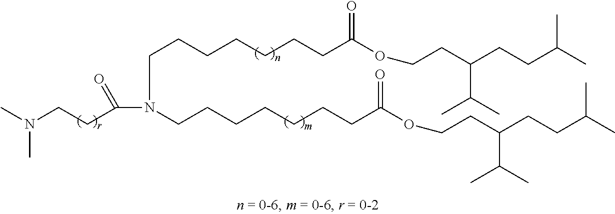

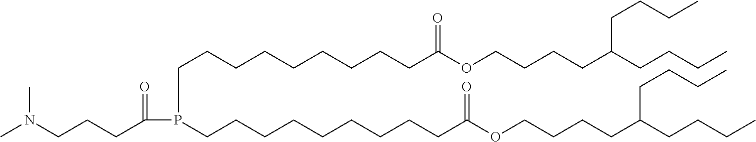 [SMILES: CCCCC(CCCC)CCCCOC(=O)CCCCCCCCCP(CCCCCCCCCC(=O)OCCCCC(CCCC)CCCC)C(=O)CCCN(C)C]

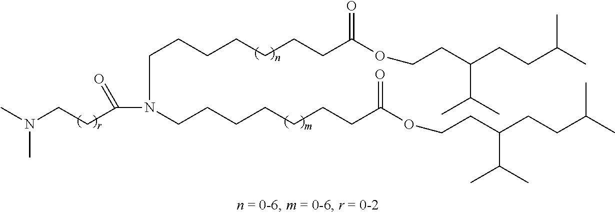

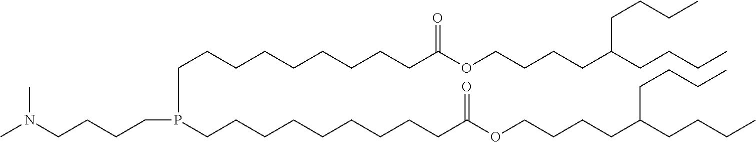 [SMILES: CCCCC(CCCC)CCCCOC(=O)CCCCCCCCCP(CCCCCCCCCC(=O)OCCCCC(CCCC)CCCC)CCCCN(C)C]

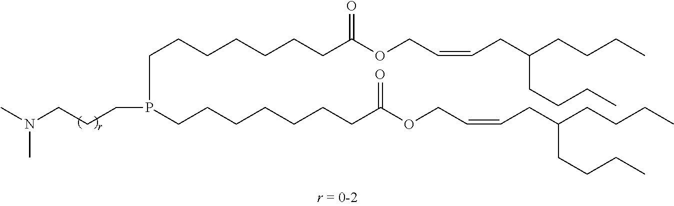

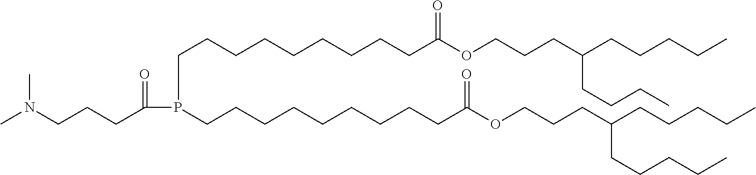 [SMILES: CCCCCC(CCCC)CCCOC(=O)CCCCCCCCCP(CCCCCCCCCC(=O)OCCCC(CCCCC)CCCCC)C(=O)CCCN(C)C]

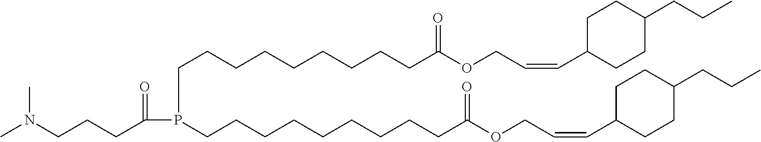 [SMILES: CCCC1CCC(/C=C\COC(=O)CCCCCCCCCP(CCCCCCCCCC(=O)OC/C=C\C2CCC(CCC)CC2)C(=O)CCCN(C)C)CC1]

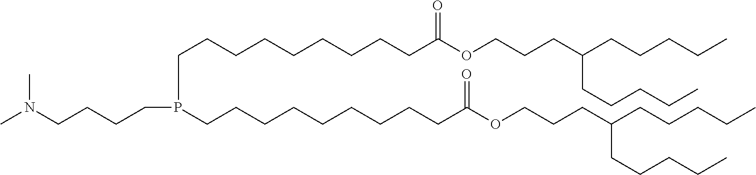 [SMILES: CCCCCC(CCCCC)CCCOC(=O)CCCCCCCCCP(CCCCCCCCCC(=O)OCCCC(CCCCC)CCCCC)CCCCN(C)C]

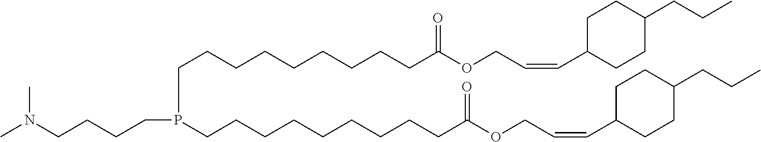 [SMILES: CCCC1CCC(/C=C\COC(=O)CCCCCCCCCP(CCCCCCCCCC(=O)OC/C=C\C2CCC(CCC)CC2)CCCCN(C)C)CC1]

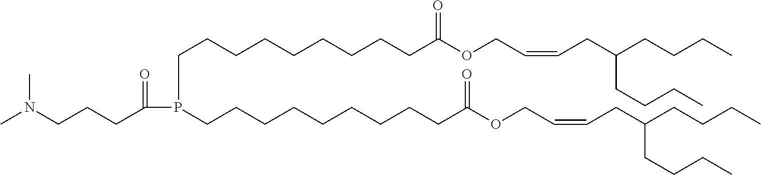 [SMILES: CCCCC(C/C=C\COC(=O)CCCCCCCCCP(CCCCCCCCCC(=O)OC/C=C\CC(CCCC)CCCC)C(=O)CCCN(C)C)CCCC]

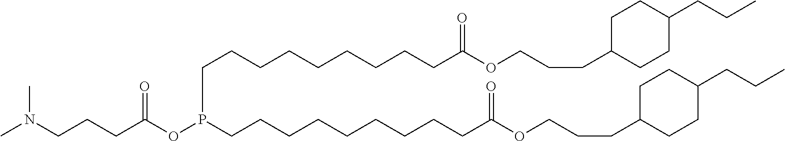 [SMILES: CCCC1CCC(CCCOC(=O)CCCCCCCCCP(CCCCCCCCCC(=O)OCCCC2CCC(CCC)CC2)OC(=O)CCCN(C)C)CC1]

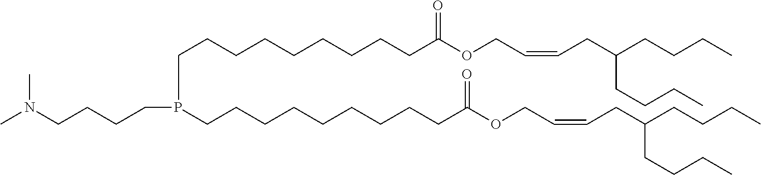 [SMILES: CCCCC(C/C=C\COC(=O)CCCCCCCCCP(CCCCCCCCCC(=O)OC/C=C\CC(CCCC)CCCC)CCCCN(C)C)CCCC]

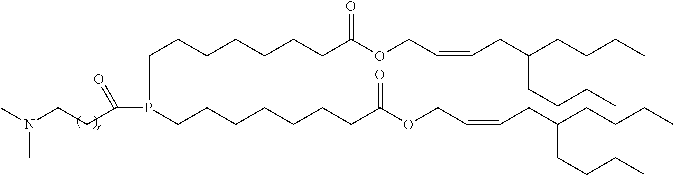

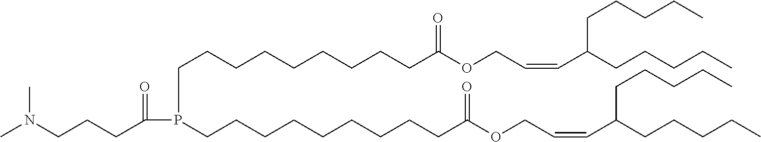 [SMILES: CCCCCC(/C=C\COC(=O)CCCCCCCCCP(CCCCCCCCCC(=O)OC/C=C\C(CCCCC)CCCCC)C(=O)CCCN(C)C)CCCCC]

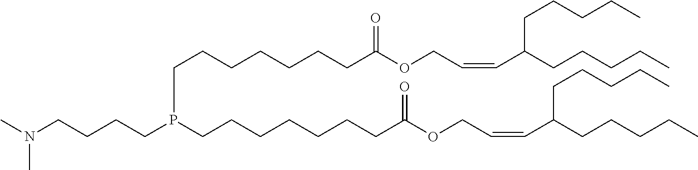 [SMILES: CCCCCC(/C=C\COC(=O)CCCCCCCP(CCCCCCCC(=O)OC/C=C\C(CCCCC)CCCCC)CCCCN(C)C)CCCCC]

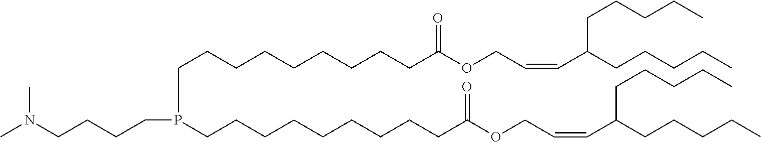 [SMILES: CCCCCC(/C=C\COC(=O)CCCCCCCCCP(CCCCCCCCCC(=O)OC/C=C\C(CCCCC)CCCCC)CCCCN(C)C)CCCCC]

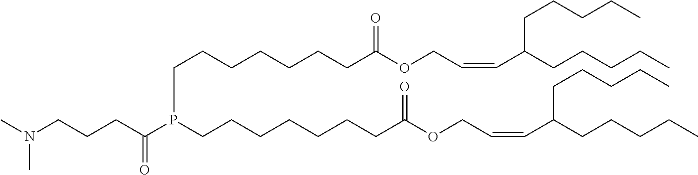 [SMILES: CCCCCC(/C=C\COC(=O)CCCCCCCP(CCCCCCCC(=O)OC/C=C\C(CCCCC)CCCCC)C(=O)CCCN(C)C)CCCCC]

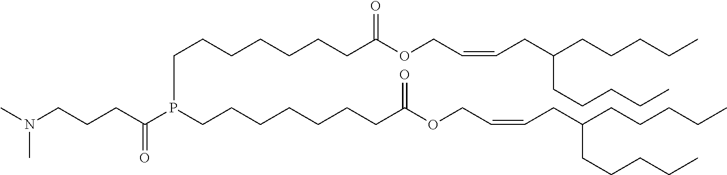 [SMILES: CCCCCC(C/C=C\COC(=O)CCCCCCCP(CCCCCCCC(=O)OC/C=C\CC(CCCCC)CCCCC)C(=O)CCCN(C)C)CCCCC]

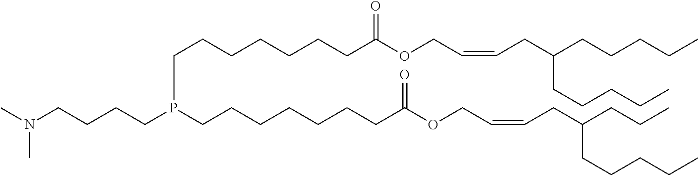 [SMILES: CCCCCC(C/C=C\COC(=O)CCCCCCCP(CCCCCCCC(=O)OC/C=C\CC(CCCCC)CCCCC)CCCCN(C)C)CCC]

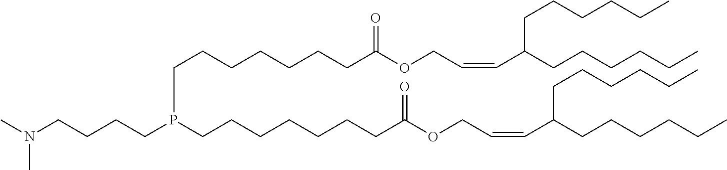 [SMILES: CCCCCCC(/C=C\COC(=O)CCCCCCCP(CCCCCCCC(=O)OC/C=C\C(CCCCCC)CCCCCC)CCCCN(C)C)CCCCCC]

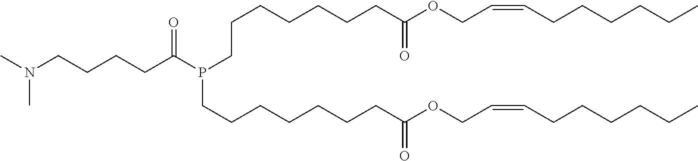 [SMILES: CCCCCC/C=C\COC(=O)CCCCCCCP(CCCCCCCC(=O)OC/C=C\CCCCCC)C(=O)CCCCN(C)C]

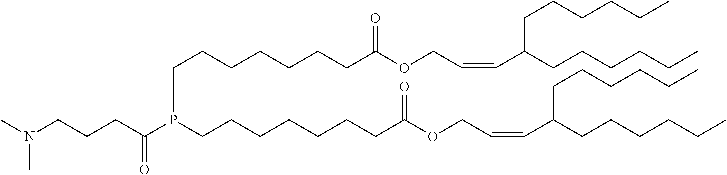 [SMILES: CCCCCCC(/C=C\COC(=O)CCCCCCCP(CCCCCCCC(=O)OC/C=C\C(CCCCCC)CCCCCC)C(=O)CCCN(C)C)CCCCCC]

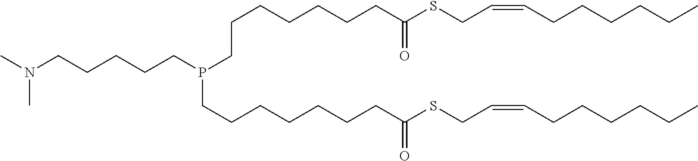 [SMILES: CCCCCC/C=C\CSC(=O)CCCCCCCP(CCCCCCCC(=O)SC/C=C\CCCCCC)CCCCCN(C)C]

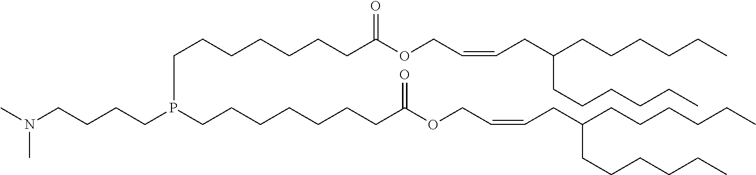 [SMILES: CCCCCCC(C/C=C\COC(=O)CCCCCCCP(CCCCCCCC(=O)OC/C=C\CC(CCCCCC)CCCCCC)CCCCN(C)C)CCCCCC]

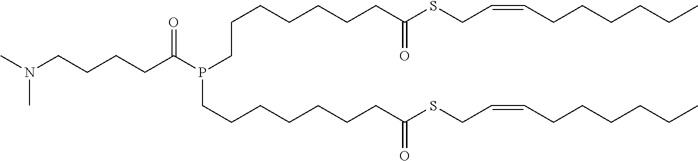 [SMILES: CCCCCC/C=C\CSC(=O)CCCCCCCP(CCCCCCCC(=O)SC/C=C\CCCCCC)C(=O)CCCCN(C)C]

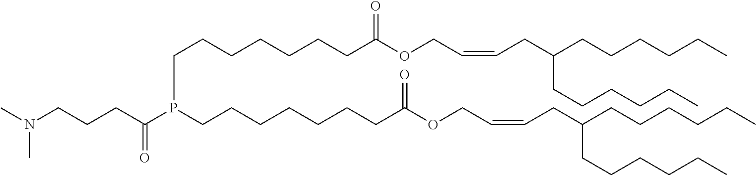 [SMILES: CCCCCCC(C/C=C\COC(=O)CCCCCCCP(CCCCCCCC(=O)OC/C=C\CC(CCCCCC)CCCCCC)C(=O)CCCN(C)C)CCCCCC]

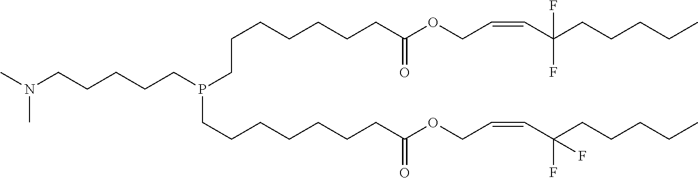 [SMILES: CCCCCC(F)(F)/C=C\COC(=O)CCCCCCCP(CCCCCCCC(=O)OC/C=C\C(F)(F)CCCCC)CCCCCN(C)C]

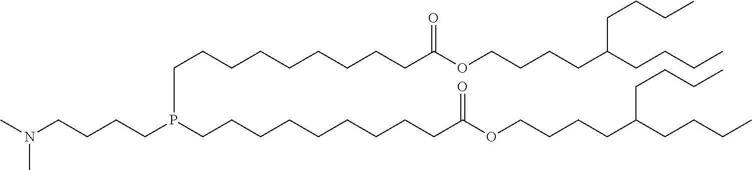 [SMILES: CCCCC(CCCC)CCCCOC(=O)CCCCCCCCCP(CCCCCCCCCC(=O)OCCCCC(CCCC)CCCC)CCCCN(C)C]

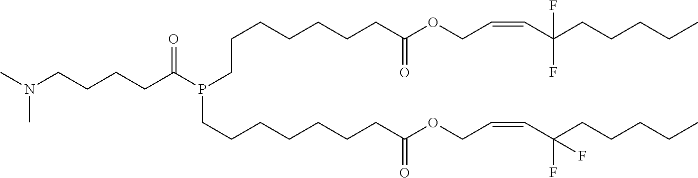 [SMILES: CCCCCC(F)(F)/C=C\COC(=O)CCCCCCCP(CCCCCCCC(=O)OC/C=C\C(F)(F)CCCCC)C(=O)CCCCN(C)C]

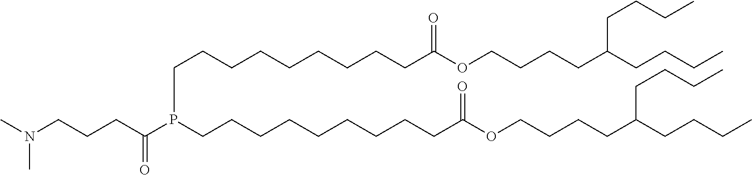 [SMILES: CCCCC(CCCC)CCCCOC(=O)CCCCCCCCCP(CCCCCCCCCC(=O)OCCCCC(CCCC)CCCC)C(=O)CCCN(C)C]

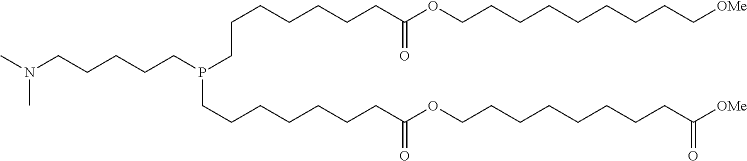 [SMILES: COCCCCCCCCCOC(=O)CCCCCCCP(CCCCCCCC(=O)OCCCCCCCCC(=O)OC)CCCCCN(C)C]

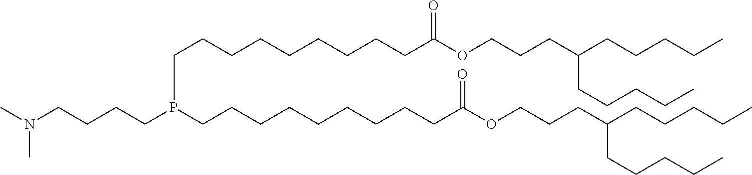 [SMILES: CCCCCC(CCCCC)CCCOC(=O)CCCCCCCCCP(CCCCCCCCCC(=O)OCCCC(CCCCC)CCCCC)CCCCN(C)C]

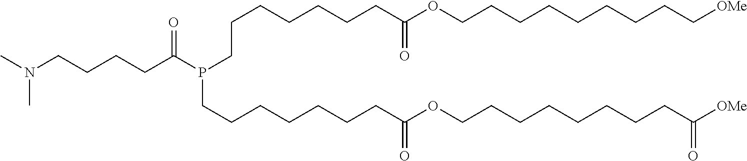 [SMILES: COCCCCCCCCCOC(=O)CCCCCCCP(CCCCCCCC(=O)OCCCCCCCCC(=O)OC)C(=O)CCCCN(C)C]

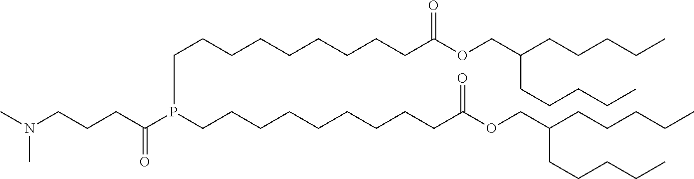 [SMILES: CCCCCC(CCCCC)COC(=O)CCCCCCCCCP(CCCCCCCCCC(=O)OCC(CCCCC)CCCCC)C(=O)CCCN(C)C]

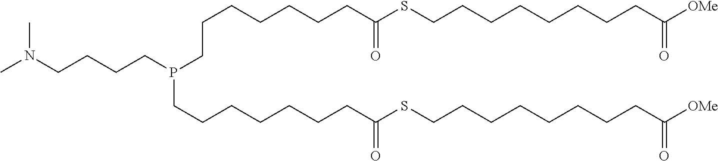 [SMILES: COC(=O)CCCCCCCCSC(=O)CCCCCCCP(CCCCCCCC(=O)SCCCCCCCCC(=O)OC)CCCCN(C)C]

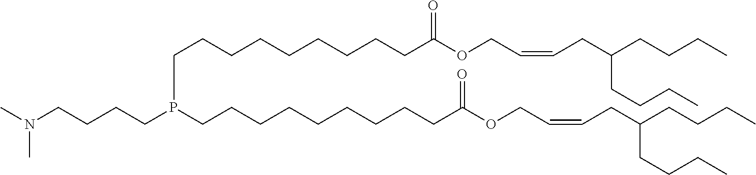 [SMILES: CCCCC(C/C=C\COC(=O)CCCCCCCCCP(CCCCCCCCCC(=O)OC/C=C\CC(CCCC)CCCC)CCCCN(C)C)CCCC]

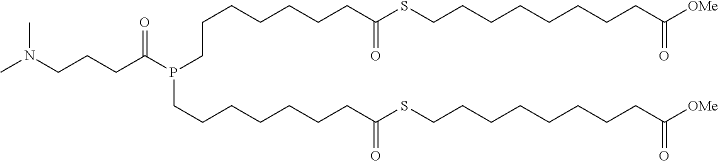 [SMILES: COC(=O)CCCCCCCCSC(=O)CCCCCCCP(CCCCCCCC(=O)SCCCCCCCCC(=O)OC)C(=O)CCCN(C)C]

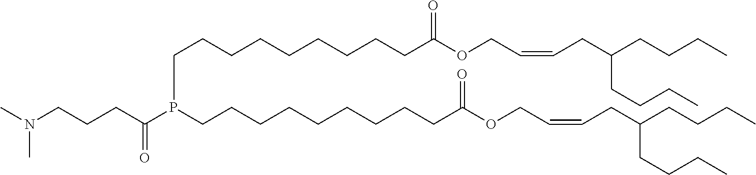 [SMILES: CCCCC(C/C=C\COC(=O)CCCCCCCCCP(CCCCCCCCCC(=O)OC/C=C\CC(CCCC)CCCC)C(=O)CCCN(C)C)CCCC]

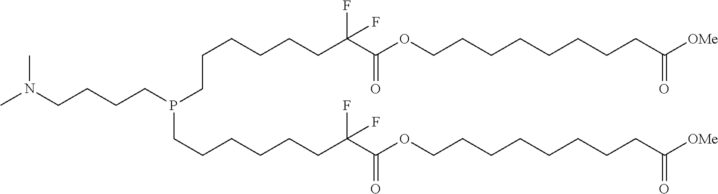 [SMILES: COC(=O)CCCCCCCCOC(=O)C(F)(F)CCCCCCP(CCCCCCC(F)(F)C(=O)OCCCCCCCCC(=O)OC)CCCCN(C)C]

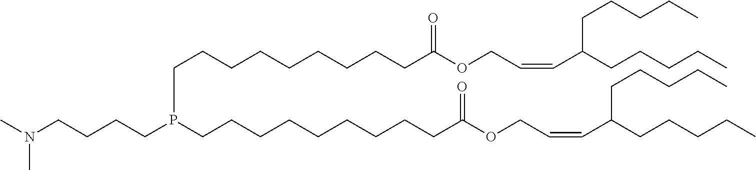 [SMILES: CCCCCC(/C=C\COC(=O)CCCCCCCCCP(CCCCCCCCCC(=O)OC/C=C\C(CCCCC)CCCCC)CCCCN(C)C)CCCCC]

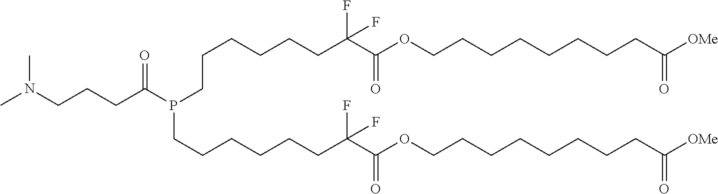 [SMILES: COC(=O)CCCCCCCCOC(=O)C(F)(F)CCCCCCP(CCCCCCC(F)(F)C(=O)OCCCCCCCCC(=O)OC)C(=O)CCCN(C)C]

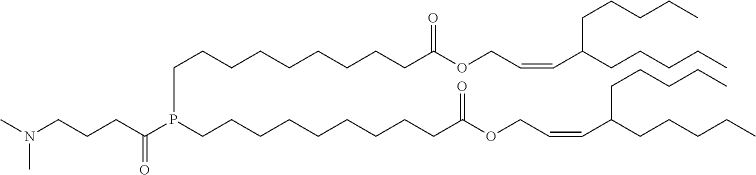 [SMILES: CCCCCC(/C=C\COC(=O)CCCCCCCCCP(CCCCCCCCCC(=O)OC/C=C\C(CCCCC)CCCCC)C(=O)CCCN(C)C)CCCCC]

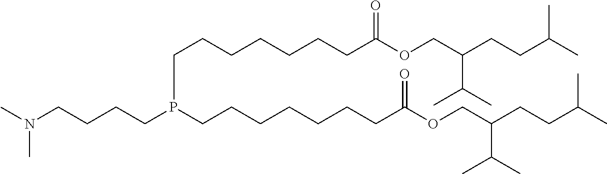 [SMILES: CC(C)CCC(COC(=O)CCCCCCCP(CCCCCCCC(=O)OCC(CCC(C)C)C(C)C)CCCCN(C)C)C(C)C]

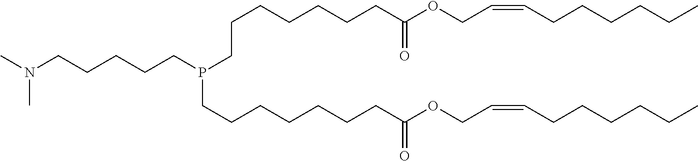 [SMILES: CCCCCC/C=C\COC(=O)CCCCCCCP(CCCCCCCC(=O)OC/C=C\CCCCCC)CCCCCN(C)C]

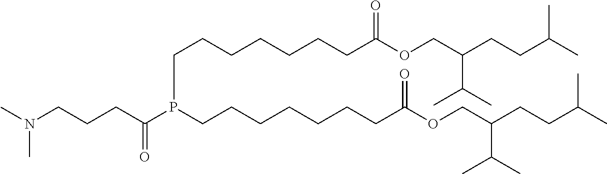 [SMILES: CC(C)CCC(COC(=O)CCCCCCCP(CCCCCCCC(=O)OCC(CCC(C)C)C(C)C)C(=O)CCCN(C)C)C(C)C]

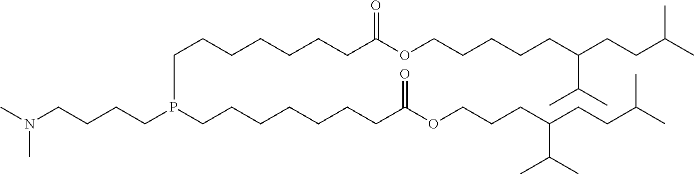 [SMILES: CC(C)CCC(CCCCCOC(=O)CCCCCCCP(CCCCCCCC(=O)OCCCC(CCC(C)C)C(C)C)CCCCN(C)C)C(C)C]

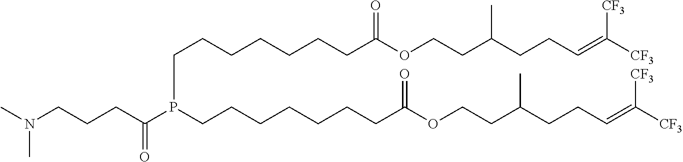 [SMILES: CC(CCC=C(C(F)(F)F)C(F)(F)F)CCOC(=O)CCCCCCCP(CCCCCCCC(=O)OCCC(C)CCC=C(C(F)(F)F)C(F)(F)F)C(=O)CCCN(C)C]

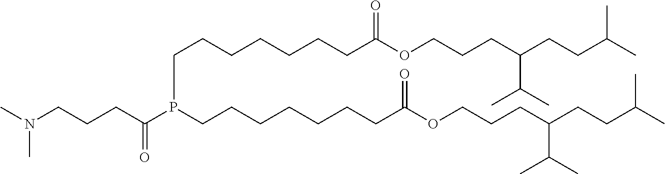 [SMILES: CC(C)CCC(CCCOC(=O)CCCCCCCP(CCCCCCCC(=O)OCCCC(CCC(C)C)C(C)C)C(=O)CCCN(C)C)C(C)C]

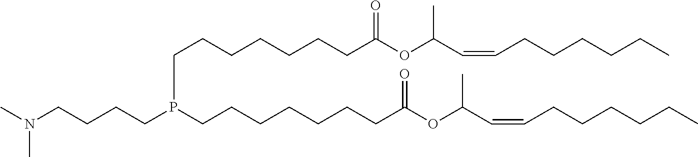 [SMILES: CCCCCC/C=C\C(C)OC(=O)CCCCCCCP(CCCCCCCC(=O)OC(C)/C=C\CCCCCC)CCCCN(C)C]

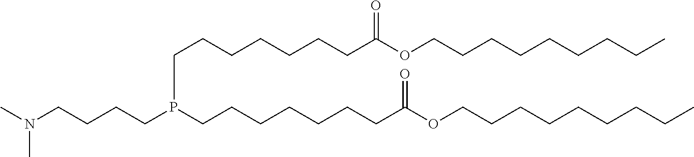 [SMILES: CCCCCCCCCOC(=O)CCCCCCCP(CCCCCCCC(=O)OCCCCCCCCC)CCCCN(C)C]

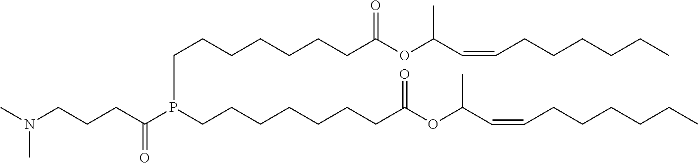 [SMILES: CCCCCC/C=C\C(C)OC(=O)CCCCCCCP(CCCCCCCC(=O)OC(C)/C=C\CCCCCC)C(=O)CCCN(C)C]

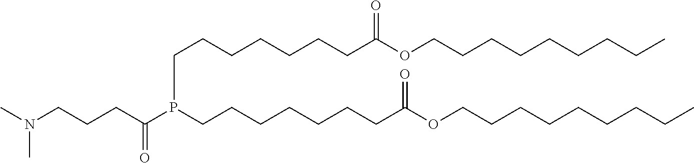 [SMILES: CCCCCCCCCOC(=O)CCCCCCCP(CCCCCCCC(=O)OCCCCCCCCC)C(=O)CCCN(C)C]

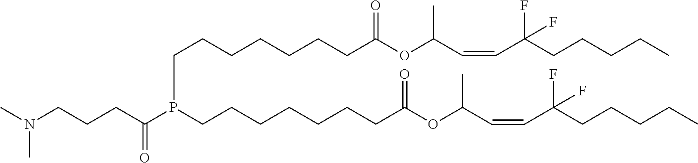 [SMILES: CCCCCC(F)(F)/C=C\C(C)OC(=O)CCCCCCCP(CCCCCCCC(=O)OC(C)/C=C\C(F)(F)CCCCC)C(=O)CCCN(C)C]

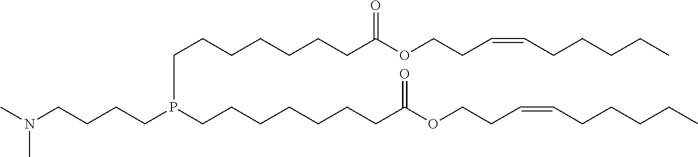 [SMILES: CCCCC/C=C\CCOC(=O)CCCCCCCP(CCCCCCCC(=O)OCC/C=C\CCCCC)CCCCN(C)C]

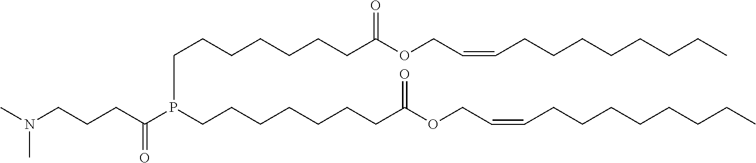 [SMILES: CCCCCCCC/C=C\COC(=O)CCCCCCCP(CCCCCCCC(=O)OC/C=C\CCCCCCCC)C(=O)CCCN(C)C]

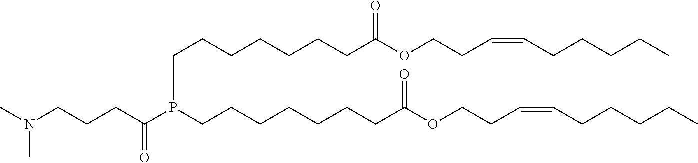 [SMILES: CCCCC/C=C\CCOC(=O)CCCCCCCP(CCCCCCCC(=O)OCC/C=C\CCCCC)C(=O)CCCN(C)C]

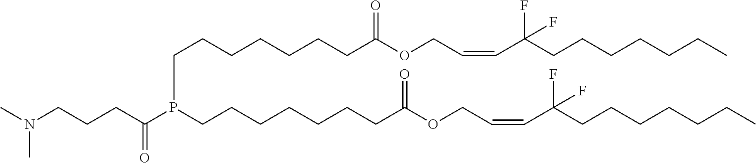 [SMILES: CCCCCCCC(F)(F)/C=C\COC(=O)CCCCCCCP(CCCCCCCC(=O)OC/C=C\C(F)(F)CCCCCCC)C(=O)CCCN(C)C]

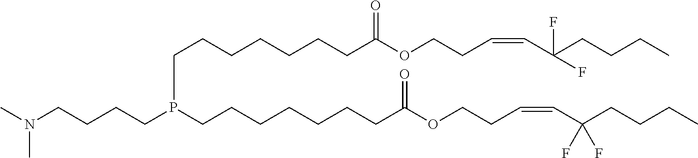 [SMILES: CCCCC(F)(F)/C=C\CCOC(=O)CCCCCCCP(CCCCCCCC(=O)OCC/C=C\C(F)(F)CCCC)CCCCN(C)C]

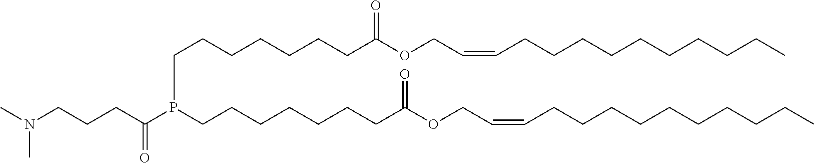 [SMILES: CCCCCCCCCC/C=C\COC(=O)CCCCCCCP(CCCCCCCC(=O)OC/C=C\CCCCCCCCCC)C(=O)CCCN(C)C]

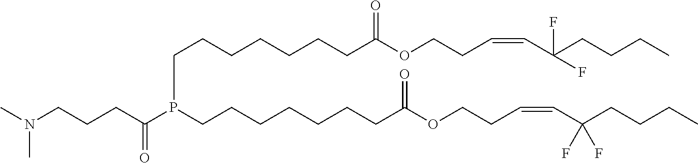 [SMILES: CCCCC(F)(F)/C=C\CCOC(=O)CCCCCCCP(CCCCCCCC(=O)OCC/C=C\C(F)(F)CCCC)C(=O)CCCN(C)C]

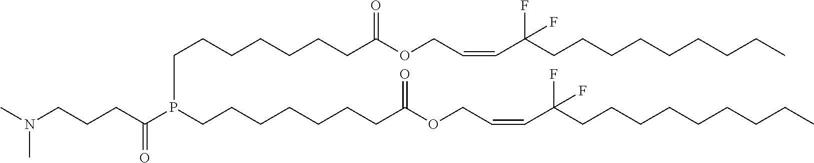 [SMILES: CCCCCCCCCC(F)(F)/C=C\COC(=O)CCCCCCCP(CCCCCCCC(=O)OC/C=C\C(F)(F)CCCCCCCCC)C(=O)CCCN(C)C]

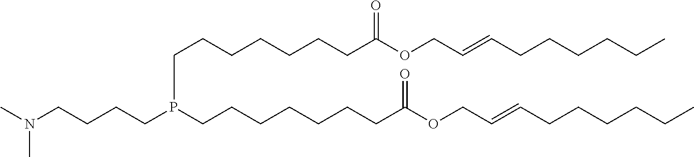 [SMILES: CCCCCC/C=C/COC(=O)CCCCCCCP(CCCCCCCC(=O)OC/C=C/CCCCCC)CCCCN(C)C]

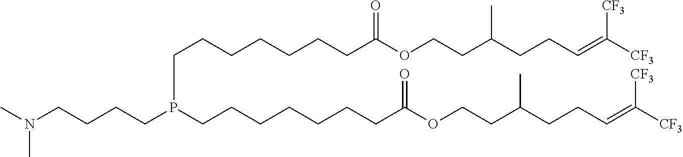 [SMILES: CC(CCC=C(C(F)(F)F)C(F)(F)F)CCOC(=O)CCCCCCCP(CCCCCCCC(=O)OCCC(C)CCC=C(C(F)(F)F)C(F)(F)F)CCCCN(C)C]

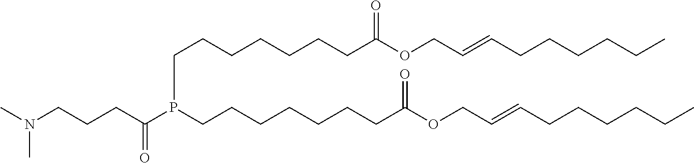 [SMILES: CCCCCC/C=C/COC(=O)CCCCCCCP(CCCCCCCC(=O)OC/C=C/CCCCCC)C(=O)CCCN(C)C]

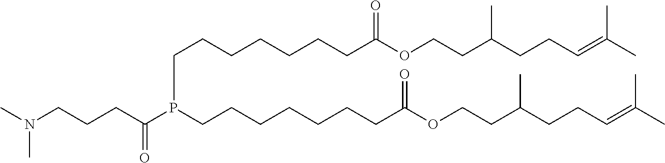 [SMILES: CC(C)=CCCC(C)CCOC(=O)CCCCCCCP(CCCCCCCC(=O)OCCC(C)CCC=C(C)C)C(=O)CCCN(C)C]

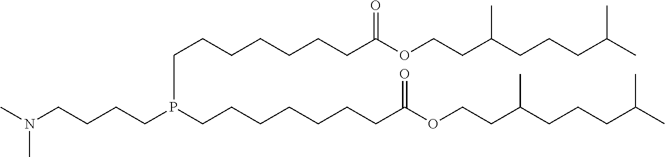 [SMILES: CC(C)CCCC(C)CCOC(=O)CCCCCCCP(CCCCCCCC(=O)OCCC(C)CCCC(C)C)CCCCN(C)C]

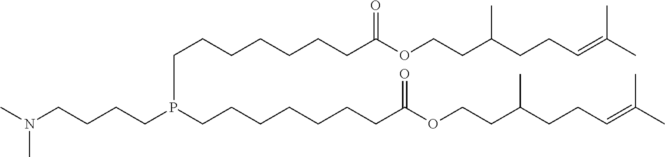 [SMILES: CC(C)=CCCC(C)CCOC(=O)CCCCCCCP(CCCCCCCC(=O)OCCC(C)CCC=C(C)C)CCCCN(C)C]

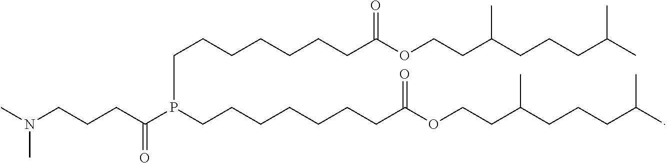

129. The compound of claim 114, wherein the compound is in the form of a pharmaceutically acceptable salt.

130. The compound of claim 114, wherein the compound is in the form of a cationic lipid.

131. A lipid particle comprising a neutral lipid, a lipid capable of reducing aggregation, and a cationic lipid of claim 130.

132. The lipid particle of claim 131, wherein the neutral lipid is selected from DSPC, DPPC, POPC, DOPE, or SM; the lipid capable of reducing aggregation is a PEG lipid; and the lipid particle further comprises a sterol.

133. The lipid particle of claim 131, wherein the cationic lipid is present in a mole percentage of about 20% and about 60%; the neutral lipid is present in a mole percentage of about 5% to about 25%; the sterol is present in a mole percentage of about 25% to about 55%; and the PEG lipid is PEG-DMA, PEG-DMG, or a combination thereof, and is present in a mole percentage of about 0.5% to about 15%.

134. The lipid particle of claim 131, further comprising an active agent.

135. The lipid particle of claim 134, wherein the active agent is a nucleic acid selected from a plasmid, an immunostimulatory oligonucleotide, an siRNA, an antisense oligonucleotide, a microRNA, an antagomir, an aptamer, and a ribozyme.

136. The lipid particle of claim 131, wherein the lipid particle has an in vivo half life (t_{1/2}) of less than about 3 hours.

137. The lipid particle of claim 131, wherein the lipid particle has an in vivo half life (t_{1/2}) of less than about 10% of that for a lipid particle containing the same cationic lipid without a biodegrable group.

138. A pharmaceutical composition comprising a lipid particle of claim 131 and a pharmaceutically acceptable carrier.

139. A method of modulating the expression of a target gene in a cell, comprising providing to the cell a lipid particle of claim 131.

140. The method of claim 139, wherein the active agent is a nucleic acid selected from a plasmid, an immunostimulatory oligonucleotide, an siRNA, an antisense oligonucleotide, a microRNA, an antagomir, an aptamer, and a ribozyme.

141. A method of treating a disease or disorder characterized by the overexpression of a polypeptide in a subject, comprising providing to the subject a pharmaceutical composition of claim 138, wherein the active agent is a nucleic acid selected from the group consisting of an siRNA, a microRNA, and an antisense oligonucleotide, and wherein the siRNA, microRNA, or antisense oligonucleotide includes a polynucleotide that specifically binds to a polynucleotide that encodes the polypeptide, or a complement thereof.

142. A method of treating a disease or disorder characterized by underexpression of a polypeptide in a subject, comprising providing to the subject a pharmaceutical composition of claim 138, wherein the active agent is a plasmid that encodes the polypeptide or a functional variant or fragment thereof.

143. A method of inducing an immune response in a subject, comprising providing to the subject a pharmaceutical composition of claim 138, wherein the active agent is an immunostimulatory oligonucleotide.

144. The method of claim 143, wherein the target gene is selected from the group consisting of Factor VII, EgS, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, SORT1 gene, XBP1 gene, topoisomerase I gene, topoisomerase II alpha gene, p73 gene, p21(WAF1/CIP1) gene, p27(KIP1) gene, PPM1D gene, RAS gene, caveolin I gene, MIB I gene, MTAI gene, M68 gene, tumor suppressor genes, and p53 tumor suppressor gene.

145. The method of claim 144, wherein the target gene contains one or more mutations.
